(19) United States
(12) Patent Application Publication
(10) Pub. No.: US 2021/0254006 A1
(43) Pub. Date: Aug. 19, 2021

(54) METHODS OF CULTURING AND/OR EXPANDING STEM CELLS AND/OR LINEAGE COMMITTED PROGENITOR CELLS USING LACTAM COMPOUNDS

(71) Applicant: IDEAYA BIOSCIENCES, INC., South San Francisco, CA (US)

(72) Inventors: Muzaffar ALAM, South San Francisco, CA (US); Fred ASWAD, South San Francisco, CA (US); Hilary Plake BECK, South San Francisco, CA (US); Michael Patrick DILLON, South San Francisco, CA (US); Marcos GONZALEZ-LOPEZ, South San Francisco, CA (US); Yujiro HATA, South San Francisco, CA (US); James Clifford SUTTON, Jr., South San Francisco, CA (US)

(21) Appl. No.: 16/972,181

(22) PCT Filed: Jun. 5, 2019
(86) PCT No.: PCT/US2019/035671

§ 371 Date: Dec. 4, 2020

Related U.S. Application Data
(60) Provisional application No. 62/681,561, filed on Jun. 6, 2018.

(51) Int. Cl. C12N 5/0789; A61K 35/28; A61L 29/16; A61L 31/16

(52) U.S. Cl. CPC C12N 5/0647; A61K 35/28; A61L 29/16; A61L 31/16; C12N 2501/999; C12N 2501/125; C12N 2501/71; C12N 2501/42; C12N 2501/2306; C12N 2501/26; C12N 2501/145; C12N 2501/15

(57) ABSTRACT

Provided are methods for expanding stem cells and/or lineage committed progenitor cells, at least in part, by using lactam compounds that antagonize AhR. The compounds are represented by formula (I):

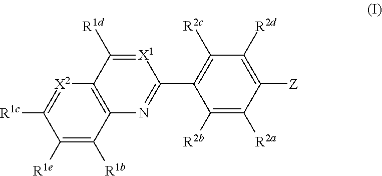

wherein the letters and symbols X¹, X², Z, R^{1b}, R^{1c}, R^{1d}, R^{1e}, R^{2a}, R^{2b}, R^{2c} and R^{2d} have the meanings provided below. Also provided are compositions comprising stem cells and/or lineage committed progenitor cells expanded by methods disclosed herein and methods for the treatment of diseases treatable by same.

### CROSS-REFERENCES TO RELATED APPLICATIONS

This application is the U.S. National Stage Entry under § 371 of International Application No. PCT/US2019/035671, filed Jun. 5, 2019, which claims the benefit priority to U.S. Provisional Application Ser. No. 62/681,561, filed Jun. 6, 2018, the disclosure of which is incorporated herein by reference in its entirety.

### BACKGROUND OF THE INVENTION

Embryonic stem cells (ESCs) are pluripotent stem cells (PSC) capable of regenerating cells of all tissues from an individual. Prior to generation of lineage-committed and terminally differentiated cells, ESCs undergo a series of division and differentiation events, gradually losing their pluripotent and proliferative capabilities. Other stem cells derived from ESCs such as hematopoietic stem cells (HSCs) and mesenchymal stem cells (MSCs), have themselves multipotent and replicative capabilities, albeit in a more lineage-committed fashion compared with ESCs. The self-renewal and multipotent properties of stem cells make these cells attractive candidates for the treatment of a wide range of diseases in the context of both cell and gene therapy strategies.

For example, HSCs can regenerate all cells of the blood compartment and are therefore of significant therapeutic value for the treatment of blood-based deficiencies resulting from congenital disorders and myeloablative treatments used to treat leukemias, lymphomas and other life-threatening conditions. HSCs can also be genetically modified to correct congenital defects that cause a range of hematological deficiencies such as anemia, sickle cell disease and hemophilia. Treatment with HSCs involves replacement of a patient's HSCs via autologous or allogeneic bone marrow transplantation (BMT) of HSCs or genetically modified HSCs. However, the success of a BMT is limited by the multipotency and number of HSCs available for transplantation. Therefore, there is a significant need for methods and reagents that can enhance the quality and quantity of HSCs.

The aryl hydrocarbon receptor (AhR) is a helix-loop-helix ligand-activated transcription factor that mediates biological responses to aromatic hydrocarbons. AhR is localized in the cytoplasm, where upon binding to a hydrocarbon-based ligand agonist, it migrates to the nucleus and forms a heterodimer with aryl hydrocarbon receptor nuclear translocator (ARNT). Formation of the AhR/ARNT complexes subsequently enables binding to and transcription of the xenobiotic response element (XRE) and associated genes. AhR can also activate non-XRE dependent protein-protein interaction pathways. Through its XRE-dependent and independent activity, AhR modulates numerous key pathways responsible for proliferation and differentiation of stem cells (SC).

SCs, including ESCs, MSCs, and HSCs amongst other cells analyzed, express high levels of AhR. Pharmacological inhibition of AhR significantly enhances maintenance of pluripotency during stem cell division and proliferation (see Boitano A. E. et al., Aryl hydrocarbon receptor antagonists promote the expansion of human hematopoietic stem cells. Science. 329, 1345-1348, 2010). For example, inhibition of AhR enhances the proliferative potential of stem cells such as MSC and HSCs (Boitano A. E infra). Taken together, these results demonstrate that pharmacological inhibition of AhR can be used to greatly expand stem cells while maintaining pluripotency. Additional factors such as inhibitors of TGF-beta, histone demethylase, histone deacetylation, p38 signaling, beta-catenin signaling and members of the ikaros family of transcription factors can also enhance HSC expansion (see de Almeida D. C., et al., Role of aryl hydrocarbon receptor in mesenchymal stromal cell activation: A minireview. World J. Stem Cells. 9, 152-158, 2017). Without being bound to theory, it is postulated that a combination of these factors with an AhR inhibitor would yield an additive or synergistic effect on HSC expansion. Other factors such as Notch agonists work in concert with AhR inhibitors such as SR-1 to enhance HSC expansion (see PCT Publication No. WO/2013/086436).

While AhR antagonists that can enhance HSC expansion are described in the art, there remains a need for AhR antagonists that exhibit a more potent and less cytotoxic profile for use in expansion of HSCs and stem cells broadly. The present disclosure fulfills this and related needs.

### BRIEF SUMMARY OF THE INVENTION

Provided are methods for expanding stem cells and/or lineage committed progenitor cells, at least in part, by using lactam compounds that antagonize AhR. The compounds are represented by formula (I):

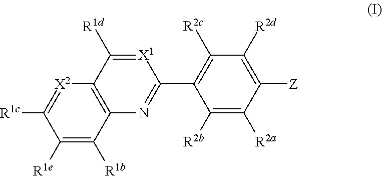

wherein the letters and symbols X¹, X², Z, R^{1b}, R^{1c}, R^{1d}, R^{1e}, R^{2a}, R^{2b}, R^{2c} and R^{2d} have the meanings provided below. Also provided are compositions comprising stem cells and/or lineage committed progenitor cells thereof expanded by methods disclosed herein and methods for the treatment of diseases treatable by same.

Also provided are methods for producing genetically modified stem cells comprising culturing naturally occurring stem cells in vitro or ex vivo with lactam compounds that antagonize AhR. The compounds are represented by formula (I) above.

### DETAILED DESCRIPTION OF THE INVENTION

Before the present invention is further described, it is to be understood that the invention is not limited to the particular embodiments set forth herein, and it is also to be understood that the terminology used herein is for the purpose of describing particular embodiments only and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention. Unless defined otherwise, all technical and scientific terms used herein have the same meaning as commonly understood by one of ordinary skill in the art to which this invention belongs.

It must be noted that as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology such as “solely,” “only” and the like in connection with the recitation of claim elements or use of a “negative” limitation.

The publications discussed herein are provided solely for their disclosure prior to the filing date of the present application. Further, the dates of publication provided may be different from the actual publication dates, which may need to be independently confirmed.

### Definitions

Unless otherwise indicated, the following terms are intended to have the meaning set forth below. Other terms are defined elsewhere throughout the specification.

“Ex vivo” as used herein, refers to a process in which cells are removed from a living organism and are propagated outside the organism. Methods of ex vivo culturing stem cells from different tissues are well known in the art of cell culturing. See for example, the text book “Culture of Animal Cells—A Manual of Basic Technique” by Freshney, Wiley-Liss, N.Y. (2016), Seventh Edition, the teachings of which are hereby incorporated by reference.

“In vitro” refers to a process by which cells known to propagate only in vitro, such as various cell lines, are cultured.

“Stem cell” as used herein, refers to a mammalian cell that can undergo self-renewal and can differentiate into multiple cell types. The term as used herein, encompasses naturally occurring and non-naturally occurring (for example induced pluripotent stems cells and genetically modified stem cells) pluripotent and multipotent stems cells, unless otherwise stated. Thus, the term stem cells covers pluripotent stem cells i.e., induced pluripotent stem cells (iPSCs) and embryonic stem cells (ESCs); multipotent stem cells i.e., hematopoietic stem cells (HSCs), mesenchymal stem cells (MSCs), neuronal stem cells (NSCs), and epidermal stem cell; and genetically modified variants of each of the aforementioned stem cell type. As used herein, “pluripotent,” “totipotent” or “omnipotent” stem cells refer to cells that can self-renew and differentiate to produce all of the cell types that make up the body. Multipotent stem cells also self-renew but unlike pluripotent stem cells, they form some but not all of the cell types in the body. In some embodiments, the stem cells are human cells.

“Multipotent” when used in reference to a “multipotent cell” refers to a cell that has the developmental potential to differentiate into multiple different cell types which may or may not be in the same lineage. For example, HSC can differentiate into multiple different types of blood cells (red, white, platelets, etc.).

“Induced pluripotent stem cell” or “iPSC” means PSC that is derived from a cell that is not a PSC (i.e., from a cell this is differentiated relative to a PSC). iPSCs can be derived from multiple different cell types, including terminally differentiated cells. iPSCs have an ES cell-like morphology and express one or more key pluripotency markers known by one of ordinary skill in the art, including but not limited to Alkaline Phosphatase, SSEA3, SSEA4, Sox2, Oct3/4, Nanog, TRA160, TRA181, TDGF 1, Dnmt3b, FoxD3, GDF3, Cyp26al, TERT, and zfp42. Examples of methods of generating and characterizing iPSCs may be found in, for example, U.S. Patent Publication Nos. US20090047263, US20090068742, US20090191159, US20090227032, US20090246875, and US20090304646, the disclosures of which are incorporated herein by reference.

“Hematopoietic stem cells” refers to immature blood cells having the capacity to self-renew and to differentiate into mature blood cells containing diverse lineages including but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Such cells may include CD34+ cells. In addition, HSCs also refer to long-term repopulating HSCs (LT-HSCs) and short-term repopulating HSCs (ST-HSCs). LT-HSCs and ST-HSCs are differentiated, based on functional potential and on cell surface marker expression. In addition, ST-HSCs are less quiescent and more proliferative than LT-HSCs under homeostatic conditions. However, LT-HSC have greater self-renewal potential (i.e., they survive throughout adulthood and can be serially transplanted through successive recipients) whereas ST-HSCs have limited self-renewal (i.e., they survive for only a limited period of time, and do not possess serial transplantation potential). Any of these HSCs can be used in the methods described herein.

“Mesenchymal stem cell” are cells that can renew and differentiate to give rise to cells of a mesenchymal cell lineage (e.g., bone, cartilage, muscle, and fat cells).

“Genetically modified stem cell(s)” refers to a naturally occurring stem cell that has exogenous nucleic acid introduced inside the cell and/or one or more endogenous genes, either completely or partially, deleted or replaced, and/or one or more endogenous gene mutated. All of the aforementioned modifications occur artificially i.e., are man-made.

“Naturally-occurring” as used herein and as applied to a nucleic acid, polypeptide, cell, or an organism, refers to a nucleic acid, polypeptide, cell, or organism that is found in nature. For example, a polypeptide or polynucleotide sequence that is commonly present in an organism (including viruses) that can be isolated from a source in nature and which has not been intentionally modified by a human in the laboratory is naturally occurring.

“Non-naturally occurring” as used herein in reference to a stem cell refers to a stem cell that has at least one genetic or physical alteration not normally found in a naturally occurring stem cell. Genetic alterations include, for example, modifications introducing expressible nucleic acids encoding polypeptides or signaling polypeptides (for example, fluorescent polypeptides), other nucleic acid additions, nucleic acid deletions and/or other functional disruption of genetic material. Such modifications include, for example, coding regions and functional fragments thereof, for heterologous, homologous or both heterologous and homologous polypeptides. Additional modifications include, for example, non-coding regulatory regions in which the modifications alter expression of a gene.

“Exogenous” refers to any material introduced from outside an organism, cell, or system.

“Endogenous” refers to any material that occurs naturally in an organism, cell, or system.

“Nucleic acid” or “polynucleotide” refers to any nucleic acid molecule, including, without limitation, DNA, RNA, and hybrids thereof. The nucleic acid bases that form nucleic acid molecules can be the bases A, T, G, C, and U, as well as derivatives thereof. Derivatives of these bases are well known in the art, and are exemplified in PCR Systems, Reagents and Consumables (Perkin Elmer Catalogue 1996-1997, Roche Molecular Systems, Inc., Branchburg, N.J., USA).

“Lineage committed progenitor cells” or “progenitor cells” means multipotent cells that can differentiate into one or more cells types in a particular lineage. For example, in the case of HSC stem cells, the lineage committed progenitor cells are lymphoid and myeloid progenitor cells. “Lymphoid and/or myeloid progenitor cells” may also be referred to herein as progen(ies) of HSC. Other lineage committed progenitor cells include, but are not limited to, hepatic progenitor cells, pancreatic progenitor cells, bronchiolar progenitor cells, alveolar progenitor cells, and endothelial progenitor cells. The phrase “stem cells and/or lineage committed progenitor cells” as used herein in reference to naturally occurring lineage committed progenitor cells includes lineage committed progenitor cells obtained directly from their source and/or derived from cultured stem cells, unless stated otherwise. For example, “hematopoetic stem cells and/or lineage committed hematopoetic progenitor cells” in reference to lineage committed hematopoetic progenitor cells include lineage committed hematopoetic progenitor cells that are obtained directly from their source (e.g., bone marrow, umbilical cord etc.) and/or derived from cultured hematopoetic stem cells. In a subembodiment, the progenitor cells are derived from cultured stem cells.

“Expansion” or “expanded” in the context of cells refers to increase in the number of a characteristic cell type or cell types relative to the number of corresponding cell type or cell types in the original population using any of the methods disclosed herein. Additionally, the phrase is used herein to describe a process of cell expansion that is substantially free of cell differentiation. “Substantially free” as used herein means less than about 95%, 90%, 85%, 80%, 75%, 70%, 65%, 60%, 55%, 50%, 45%, 40%, 35%, 30%, 25%, 20%, 15%, or 10% of expanded population of cells have undergone differentiation. “About” as used herein means + or −10% deviation from each of the above listed initial value.

“Differentiation” or “differentiate” or “differentiating” as used herein refers to the developmental process of lineage commitment. A “lineage” refers to a pathway of cellular development, in which precursor or progenitor cells undergo progressive physiological changes to become a specified cell type having a characteristic function (e.g., nerve cell, muscle cell, T cell, B cell, erythrocytes, or endothelial cell). Differentiation occurs in stages, whereby cells gradually become more specific until they reach full maturity, which is also referred to as “terminal differentiation.” A terminally differentiated cell is a cell that has committed to a specific lineage and has reached the end stage of differentiation (i.e., a cell that has fully matured).

“Maintaining functional potential of stem cell” or “maintain functional potential of stem cell” refers to functional properties of stem cells which include the ability to self-renew and to differentiate into their respective progeny cells. For example, a HSC will have the ability to 1) maintain its multipotency i.e., be able to differentiate into its progenitor cells (such as common erythroid progenitor, common lymphoid progenitor) and ultimately all types of blood cells including, but not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells), and 2) self-renew (which refers to the ability of hematopoietic stem cells to give rise to daughter cells that have equivalent potential as the mother cell, and further that this ability can repeatedly occur throughout the lifetime of an individual without exhaustion). In case of mesenchymal stem cells, the MSCs will maintain their ability to self-renew and to differentiate to give rise to their progeny cells e.g., connective tissue, bone, cartilage. As used herein, mesenchymal stem cells include, without limitation, CD34⁻ stem cells.

“Self-renewal” as used herein, refers to the process by which a stem cell divides to generate one (asymmetric division) or two (symmetric division) daughter cells having development potential indistinguishable from the mother cell. Self-renewal involves both proliferation and the maintenance of an undifferentiated state.

“Population of stem cells” as used herein means a collection of two or more stem cells. Preferably, the population consists of at least twenty cells, or at least one hundred cells, or at least one thousand cells, or more.

“Agent that reduces the activity of an ikaros family transcription factor” refers to any agent (e.g., a small molecule, proteins (including antibodies and peptides), miRNA, and interfering nucleic acids) that regulates the activity of any member of ikaros pathway which results in reduction in the activity of an ikaros family transcription factor in the cell, such as ikaros, aiolos, and helios. Exemplary agents capable of reducing the level of an ikaros transcription factor include compounds that activate ubiquitin ligases, such as cereblon-containing ubiquitin E3 ligases that mediate the polyubiquitination of an ikaros family transcription factor. Polyubiquitination of ikaros family transcription factors leads to degradation of the transcription factor.

“Agent that inhibits histone demethylation” refers to any agent (e.g., a small molecule, proteins (including antibodies and peptides), miRNA, and interfering nucleic acids) that inhibits the activity of a histone demethylase by directly binding to the histone demethylase or by modulating the activity of any member of histone demethylase pathway that results in histone demethylation. Histone demethylases include lysine-specific demethylases, such as LSD1 and LSD2, as well as other FAD-dependent histone demethylases. Histone demethylases also include dioxygenases that utilize divalent iron (Fe²⁺) to catalyze oxidative demethylation of histone residues, such as Alk B, and Jumonji C (JmjC) domain-containing histone demethylases, such as JHDM1, JHDM2, and members of the JMJD2 superfamily of histone demethylases. Other enzymes that convert methylated histone residues into reactive intermediates that subsequently undergo oxidative demethylation include monoamine oxidases. Exemplary assays that can be used to elucidate the biological activity of a histone demethylation inhibitor include, without limitation, cell-based growth inhibition assays and dissociation-enhanced lanthanide fluorescence assays as described in U.S. Pat. No. 8,735,622, time-resolved fluorescence resonance energy transfer assays as described in WO 2014/151945, as well as mass spectrometry-based assays and coupled-enzyme formaldehyde dehydrogenase assays as described in WO 2010/043866.

“Agent that inhibits TGFβ signaling” refers to any agent (e.g., a small molecule, proteins (including antibodies and peptides), miRNA and interfering nucleic acids) that reduces the activity of TGFβ receptor by directly binding to a TGFβ receptor or by modulating the activity of any member of TGFβ receptor pathway such as a SMAD, Activin, Nodal, bone morphogenetic protein (BMP), growth and differentiation factor (GDF), or Millerian inhibitory factor (MIF) or substances that promote the ubiquitination and degradation of above proteins. Exemplary assays that can be used to determine the inhibitory activity of a TGFβ signaling pathway inhibitor include, without limitation, electrophoretic mobility shift assays, antibody super shift assays, as well as TGFβ-inducible gene reporter assays, as described in WO 2006/012954.

“Agent that inhibits p38 signaling” refers to any agent (e.g., a small molecule, protein (including antibodies and peptides), miRNA, interfering nucleic acids) that reduces the activity of the p38 mitogen activated protein kinases (MAPKs, e.g., p38a, p38, p38y, or p388) by directly binding to a p38 kinase or by modulating the activity of any member of p38 kinase pathway. Exemplary assays that can be used to determine the inhibitory activity of an agent that inhibits the p38 signaling pathway include, without limitation, fluorescence anisotropy competitive binding assays, as well as time-resolved fluorescence resonance energy transfer assays, as described in WO 2006/012954.

“Agent that inhibits histone deacetylation” refers to any agent (e.g., a small molecule, proteins (including antibodies and peptides), miRNA, interfering nucleic acids) that reduces the activity of histone deacetylase by directly binding to a histone deacetylase or by modulating the activity of any member of histone deacetylase pathway that results in histone deacetylation. As used herein, the term “histone deacetylase” refer to any one of a family of enzymes that catalyze the removal of acetyl groups from the £-amino groups of lysine residues at the N-terminus of a histone. Unless otherwise indicated by context, the term “histone” is meant to refer to any histone protein, including HI, H2A, H2B, H3, H4, and H5, from any species. Human HDAC proteins or gene products, include, but are not limited to, HDAC-1, HDAC-2, HDAC-3, HDAC-4, HDAC-5, HDAC-6, HDAC-7, HDAC-8, HDAC-9, HDAC-10, and HDAC-11.

“Agent that inhibits a protein that promotes β-catenin degradation” refers to any agent (e.g., a small molecule, proteins (including antibodies and peptides), miRNA, interfering nucleic acids) that reduces the rate or extent of β-catenin degradation, e.g., by attenuating the catalysis of phosphorylation of serine and/or threonine residues that would otherwise render β-catenin a substrate for ubiquitination and proteasome-mediated degradation (for example, at residues Ser33, Ser37 and/or at Thr41). By extending the half-life of functional β-catenin, these agents promote a concomitant increase in the rate or extent of transcription of a gene that is transcribed due to the activity of the β-catenin transcription co-activator. Exemplary agents that inhibit β-catenin phosphorylation include agonists of the canonical β-catenin/Wnt signaling pathway, a signal transduction cascade that causes inhibition of glycogen synthase kinase 3 (GSK3) by providing a substrate that competes with β-catenin for phosphorylation.

“A Wnt signaling agonist” refers to an agonist of the canonical Wnt signaling pathway (e.g., a small molecule, protein (including antibodies and peptides), miRNA, interfering nucleic acids) and further includes Wnt proteins or other compounds that bind directly to the Frizzled and LRP5/6 co-receptor proteins to promote an increase in the concentration of β-catenin in the nucleus of a mammalian cell. Alternatively, a β-catenin/Wnt pathway agonist may function by inhibiting one or more secreted Frizzled-related proteins (SFRPs) or Wnt inhibitory protein (WIF), which bind and sequester Wnt proteins from the endogenous Wnt co-receptors. Exemplary methods that can be used to determine the activity of a β-catenin/Wnt pathway agonist include, without limitation, monitoring the expression of a reporter gene under the control of a TCF/LEF family transcription factor, as well as TOPflash luciferase reporter assays, as described in US 2014/0044763.

“Notch agonist” refers to an agent (e.g., a small molecule, proteins (including antibodies and peptides), miRNA, interfering nucleic acids) that promotes activation of Notch pathway function. The term “Notch pathway function” as used herein refers to a function mediated by the Notch signal transduction pathway including, but not limited to, nuclear translocation of the intra cellular domain of Notch, nuclear translocation of RBP-JK or its Drosophila homolog Suppressor of Hairless; activation of bHLH genes of the Enhancer of Split complex, e.g., Mastermind; activation of the HES-1 gene or the KBF2 (also referred to as CBF1) gene; inhibition of Drosophila neuroblast segregation; and binding of Notch to a Delta protein, a Jagged/Serrate protein, Fringe, Deltex or RBP-JK Suppressor of Hairless, or homologs or analogs thereof. The Notch signal transduction cascade and the phenotypes effected by Notch signaling are described, e.g., in Kopan et al. Cell 137:216 (2009) and Jarriault et al. Molecular Cell Biology 18:7423 (1998), the disclosures of each of which are incorporated herein by reference. Examples of Notch agonists are described, e.g., in US 2014/0369973 and in U.S. Pat. No. 7,399,633, the disclosures of each of which are incorporated herein by reference.

“Proteins”, “peptide”, or polypeptide” refers to a polymer of amino acids, including modified amino acids (e.g., phosphorylated, glycated, glycosylated, etc.) and amino acid analogs. Protein is often used in reference to relatively large polypeptides, whereas peptide is often used in reference to small polypeptides, but usage of these terms in the art overlaps.

“Interfering nucleic acids” refers to any double stranded or single stranded nucleic acid (e.g. RNA or DNA) sequence capable, either directly or indirectly (i.e., upon conversion), of inhibiting gene expression by mediating RNA interference and/or preventing gene transcription or translation. “Interfering RNA” includes, but is not limited to, small interfering RNA (“siRNA”) and small hairpin RNA (“shRNA”). RNA interference refers to the selective degradation of a sequence-compatible messenger RNA transcript. In other embodiments, an interfering nucleic acid is a DNA sequence (for example, an antisense oligonucleotide DNA sequence) capable, either directly or indirectly, of inhibiting gene expression or protein translation by binding to genomic DNA sequences or transcribed mRNA.

“shRNA” (small hairpin RNA) refers to an RNA molecule comprising an antisense region, a loop portion and a sense region, wherein the sense region has complementary nucleotides that base pair with the antisense region to form a duplex stem. Following post-transcriptional processing, the small hairpin RNA is converted into a small interfering RNA by a cleavage event mediated by the enzyme Dicer, which is a member of the RNase III family.

“RNAi” (RNA interference) refers to a post-transcriptional silencing mechanism initiated by small double-stranded RNA molecules that suppress expression of genes with sequence homology.

“miRNA” refers to small, non-coding, single stranded RNA, typically between 18-23 nucleotide in length. miRNA is capable of regulating gene expression by modulating the stability and translation of mRNA.

“Small molecule” refers to a synthetic organic molecule having a molecular weight less than 1,500 Da, more preferably less than 1,000 Da, most preferably less than 500 Da.

“Mammal” refers to a vertebrate such as a primate, rodent, domestic animal (such as a pet or livestock) or game animal, preferably a human.

“Administration”, “administer” and the like, as they apply to, for example, a subject, cell, tissue, organ, or biological fluid, refer to contact of, for example, expanded stem cells produced by the methods disclosed herein or a pharmaceutical composition comprising same to the subject, cell, tissue, organ, or biological fluid. In the context of a cell, administration includes contact (e.g., in vitro or ex vivo) of a reagent to the cell, as well as contact of a reagent to a fluid, where the fluid is in contact with the cell.

“Patient” or “subject” are used interchangeably to refer to a human or a non-human animal (e.g., a mammal). In one embodiment, the patient is human.

“Treat”, “treating”, treatment” and the like refer to a course of action (such as administering expanded stem cells produced by the methods disclosed herein or a pharmaceutical composition comprising same) initiated after a disease, disorder or condition, or a symptom thereof, has been diagnosed, observed, and the like so as to eliminate, reduce, suppress, mitigate, or ameliorate, either temporarily or permanently, at least one of the underlying causes of a disease, disorder, or condition afflicting a subject, or at least one of the symptoms associated with a disease, disorder, condition afflicting a subject. Thus, treatment includes inhibiting (e.g., arresting the development or further development of the disease, disorder or condition or clinical symptoms association therewith) an active disease.

“In need of treatment” as used herein refers to a judgment made by a physician or other caregiver that a subject requires or will benefit from treatment. This judgment is made based on a variety of factors that are in the realm of the physician's or caregiver's expertise.

“Prevent”, “preventing”, “prevention” and the like refer to a course of action (such as administering expanded stem cells produced by the methods disclosed herein or a pharmaceutical composition comprising same) initiated in a manner (e.g., prior to the onset of a disease, disorder, condition or symptom thereof) so as to prevent, suppress, inhibit or reduce, either temporarily or permanently, a subject's risk of developing a disease, disorder, condition or the like (as determined by, for example, the absence of clinical symptoms) or delaying the onset thereof, generally in the context of a subject predisposed to having a particular disease, disorder or condition. In certain instances, the terms also refer to slowing the progression of the disease, disorder or condition or inhibiting progression thereof to a harmful or otherwise undesired state.

“In need of prevention” as used herein refers to a judgment made by a physician or other caregiver that a subject requires or will benefit from preventative care. This judgment is made based on a variety of factors that are in the realm of a physician's or caregiver's expertise.

“Therapeutically effective amount” refers to the administration of expanded stem cells prepared by methods disclosed herein to a subject, either alone or as part of a pharmaceutical composition and either in a single dose or as part of a series of doses, in an amount capable of having any detectable, positive effect on any symptom, aspect, or characteristic of a disease, disorder or condition when administered to the subject. The therapeutically effective amount can be ascertained by measuring relevant physiological effects, and it can be adjusted in connection with the dosing regimen and diagnostic analysis of the subject's condition, and the like.

“In a sufficient amount to effect a change” means that there is a detectable difference between a level of an indicator measured before (e.g., a baseline level) and after administration of a particular therapy. Indicators include any objective parameter (e.g., serum concentration) or subjective parameter (e.g., a subject's feeling of well-being).

“Substantially pure” indicates that a component makes up greater than about 50% of the total content of the composition, and typically greater than about 60% of the total content. More typically, “substantially pure” refers to compositions in which at least 75%, at least 85%, at least 90% or more of the total composition is the component of interest. In some cases, the component of interest will make up greater than about 90%, or greater than about 95% of the total content of the composition.

“Inhibitors” and “antagonists”, or “activators” and “agonists” refer to inhibitory or activating molecules, respectively, for example, for the activation of, e.g., a ligand, receptor, cofactor, gene, cell, tissue, or organ. Inhibitors are molecules that decrease, inhibit, block, prevent, delay activation, inactivate, desensitize, or down-regulate, e.g., a gene, protein, ligand, receptor, or cell. Activators are molecules that increase, activate, facilitate, enhance activation, sensitize, or up-regulate, e.g., a gene, protein, ligand, receptor, or cell. An inhibitor may also be defined as a molecule that reduces, inhibits, blocks, or inactivates a constitutive activity. An “agonist” is a molecule that interacts with a target to cause or promote an increase in the activation of the target. An “antagonist” is a molecule that opposes the action(s) of an agonist. An antagonist prevents, reduces, inhibits, or neutralizes the activity of an agonist, and an antagonist can also prevent, inhibit, or reduce constitutive activity of a target, e.g., a target receptor, even where there is no identified agonist. An inhibitor can, unless stated otherwise, reduce the activity of the target protein either directly or indirectly by modulating the activity of a member of the signaling pathway in a manner that reduces the activity of the target protein. Direct inhibition can be obtained, for instance, by binding to a protein and preventing the protein from interacting with an endogenous molecule, such as an enzyme, a substrate, or other binding partner, thereby diminishing the activity of the protein. For instance, an inhibitor may bind an enzyme active site and sterically preclude binding of an endogenous substrate at this location, thus decreasing the enzymatic activity of the protein. Indirect inhibition can be obtained, for instance, by binding to a protein that promotes the activity of a target protein by inducing a conformational change or catalyzing a chemical modification of the target protein. For instance, indirect inhibition of a target protein may be achieved by binding and inactivating a kinase that catalyzes the phosphorylation of, and activates, the target protein.

“Reduce,” “reduction” or “decrease” or “inhibit” typically means the activity of the target protein observed in its active state is reduced by at least about 10%, 20%, 25%, 30%, 35%, 40%, 45%, 50%, 55%, 60%, 65%, 70%, 75%, 80%, 85%.

“Modulate” means to change or induce an alteration in a particular biological activity of a target protein either directly or indirectly. Modulation includes, but is not limited to, stimulating or inhibiting an activity (e.g., by activating a receptor so as to initiate a signal transduction cascade, by inhibiting a receptor so as to prevent or decrease a signal transduction cascade, by activating an endogenous inhibitor that attenuates a biological activity, or by inhibiting the activity of a protein that inhibits a particular biological function).

The “activity” of a molecule may describe or refer to the binding of the molecule to a ligand or to a receptor; to catalytic activity; to the ability to stimulate gene expression or cell signaling, differentiation, or maturation; to antigenic activity; to the modulation of activities of other molecules; and the like.

“Enriched” when used in the context of cell population refers to a cell population selected based on the presence of one or more cell surface markers, for example, CD34+.

“CD34+ cells” refers to cells that express on their surface CD34 marker. CD34+ cells can be detected and counted using for example flow cytometry and fluorescently labeled anti-CD34 antibodies.

“Enriched in CD34+ cells” means that a cell population has been selected based on the presence of CD34 marker. Accordingly, the percentage of CD34+ cells in the cell population after selection method is higher than the percentage of CD34+ cells in the initial cell population before selecting step based on CD34 markers. For example, CD34+ cells may represent at least 50%, 60%, 70%, 80% or at least 90% of the cells in a cell population enriched in CD34+ cells.

Medium refers to culture medium comprising nutritive substances.

“Alkyl”, by itself or as part of another substituent, means, unless otherwise stated, a saturated straight or branched chain hydrocarbon radical, having the number of carbon atoms designated (i.e. C₁₋₈ means one to eight carbons). Examples of alkyl groups include methyl, ethyl, n-propyl, isopropyl, n-butyl, t-butyl, isobutyl, sec-butyl, n-pentyl, n-hexyl, n-heptyl, n-octyl, and the like. The term “deuteroalkyl”, by itself or as part of another substituent, refers to an alkyl group wherein from one to five hydrogen atoms have been replaced by deuterium. An example of a “deuteroalkyl” group is —CD₃.

“Alkylene” refers to a divalent alkyl group as defined herein. Examples of alkylene include methylene, ethylene, and the like.

“Alkenyl”, by itself or as part of another substituent, means, unless otherwise stated, a straight or branched chain hydrocarbon radical containing one or two double bonds and having the number of carbon atoms designated (i.e. C₂₋₆ means two to six carbons). Examples of alkenyl groups include ethenyl, n-propenyl, isopropenyl, n-butenyl, and the like.

“Alkynyl”, by itself or as part of another substituent, means, unless otherwise stated, a straight or branched chain hydrocarbon radical containing a triple and having the number of carbon atoms designated (i.e. C₂₋₆ means two to six carbons). Examples of alkynyl groups include ethynyl, propynyl, and the like.

“Alkoxy,” “alkylamino” and “alkylthio” (or thioalkoxy) are used in their conventional sense and refer to those alkyl groups attached to the remainder of the molecule via an oxygen atom, an amino group, or a sulfur atom, respectively. The term “deuteroalkoxy” is used in its conventional sense, and refer to deuteroalkyl, as defined herein, that is attached to the remainder of the molecule via an oxygen atom.

“Aryl” means, unless otherwise stated, a polyunsaturated, typically aromatic, hydrocarbon group which can be a single ring or multiple rings (up to three rings) which are fused together or linked covalently. Non-limiting examples of aryl groups include phenyl, naphthyl and biphenyl.

“Cycloalkyl” refers to hydrocarbon rings having the indicated number of ring atoms (e.g., C₃₋₆ cycloalkyl) and being fully saturated or having no more than one double bond between ring vertices.

“Heterocycloalkyl” refers to a ring having the indicated number of vertices (C₃₋₇ refers to a 3- to 7-membered ring) and having from one to five heteroatoms selected from N, O, and S, which replace one to five of the carbon vertices, and wherein the nitrogen and sulfur atoms are optionally oxidized, and the nitrogen atom(s) are optionally quaternized. More specifically, the subscript refers to the total number of ring vertices including the carbon and heteroatom ring vertices. The heterocycloalkyl may be a monocyclic, a bicyclic or a polycyclic ring system. Non-limiting examples of heterocycloalkyl groups include pyrrolidine, imidazolidine, pyrazolidine, butyrolactam, valerolactam, imidazolidinone, hydantoin, dioxolane, phthalimide, piperidine, 1,4-dioxane, morpholine, thiomorpholine, thiomorpholine-S-oxide, thiomorpholine-S,S-oxide, piperazine, pyran, pyridone, 3-pyrroline, thiopyran, pyrone, tetrahydrofuran, tetrahydrothiophene, quinuclidine, and the like. A heterocycloalkyl group can be attached to the remainder of the molecule through a ring carbon or a heteroatom.

As used herein, a wavy line, “ [symbol] ”, that intersects a single, double or triple bond in any chemical structure depicted herein, represent the point attachment of the single, double, or triple bond to the remainder of the molecule. Additionally, a bond extending to the center of a ring (e.g., a phenyl ring) is meant to indicate attachment at any of the available ring vertices. One of skill in the art will understand that multiple substituents shown as being attached to a ring will occupy ring vertices that provide stable compounds and are otherwise sterically compatible. For a divalent component, a representation is meant to include either orientation (forward or reverse). For example, the group “—C(O)NH-” is meant to include a linkage in either orientation: —C(O)NH— or —NHC(O)—, and similarly, “—O—CH₂CH₂-” is meant to include both —O—CH₂CH₂— and —CH₂CH₂—O—.

“Halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “C₁₋₄ haloalkyl” is meant to include trifluoromethyl, 2,2,2-trifluoroethyl, 4-chlorobutyl, 3-bromopropyl, and the like.

“Heteroaryl” refers to a 5- to 10-membered aromatic ring (or fused ring system) that contains from one to five heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of heteroaryl groups include pyridyl, pyridazinyl, pyrazinyl, pyrimindinyl, triazinyl, quinolinyl, quinoxalinyl, quinazolinyl, cinnolinyl, phthalazinyl, benzotriazinyl, purinyl, benzimidazolyl, benzopyrazolyl, benzotriazolyl, benzisoxazolyl, isobenzofuryl, isoindolyl, indolizinyl, benzotriazinyl, thienopyridinyl, thienopyrimidinyl, pyrazolopyrimidinyl, imidazopyridines, benzothiaxolyl, benzofuranyl, benzothienyl, indolyl, quinolyl, isoquinolyl, isothiazolyl, pyrazolyl, indazolyl, pteridinyl, imidazolyl, triazolyl, tetrazolyl, oxazolyl, isoxazolyl, thiadiazolyl, pyrrolyl, thiazolyl, furyl, thienyl and the like.

As used herein, the term “heteroatom” is meant to include oxygen (O), nitrogen (N), sulfur (S) and silicon (Si).

“Pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al, “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers, regioisomers and individual isomers (e.g., separate enantiomers) are all intended to be encompassed within the scope of the present invention. When a stereochemical depiction is shown, it is meant to refer the compound in which one of the isomers is present and substantially free of the other isomer. ‘Substantially free of’ another isomer indicates at least an 80/20 ratio of the two isomers, more preferably 90/10, or 95/5 or more. In some embodiments, one of the isomers will be present in an amount of at least 99%.

The compounds of the present invention may also contain unnatural proportions of atomic isotopes at one or more of the atoms that constitute such compounds. Unnatural proportions of an isotope may be defined as ranging from the amount found in nature to an amount consisting of 100% of the atom in question. For example, the compounds may incorporate radioactive isotopes, such as for example tritium (³H), iodine-125 (¹²⁵I) or carbon-14 (¹⁴C), or non-radioactive isotopes, such as deuterium (²H) or carbon-13 (¹³C). Such isotopic variations can provide additional utilities to those described elsewhere within this application. For instance, isotopic variants of the compounds of the invention may find additional utility, including but not limited to, as diagnostic and/or imaging reagents, or as cytotoxic/radiotoxic therapeutic agents. Additionally, isotopic variants of the compounds of the invention can have altered pharmacokinetic and pharmacodynamic characteristics which can contribute to enhanced safety, tolerability or efficacy during treatment. All isotopic variations of the compounds of the present invention, whether radioactive or not, are intended to be encompassed within the scope of the present invention.

“Pharmaceutical composition” refers to the active agent in combination with a pharmaceutically acceptable carrier e.g. a carrier commonly used in the pharmaceutical industry.

“Pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Other than in the operating examples, or where otherwise indicated, all numbers expressing quantities of ingredients or reaction conditions used herein should be understood as modified in all instances by the term “about.” As used herein, the term “about” indicates a deviation of ±10%.

“Comprising” or “comprises” is used in reference to compositions, methods, and respective component(s) thereof, that are essential to the method or composition, yet open to the inclusion of unspecified elements, whether essential or not.

“Consisting of” refers to compositions, methods, and respective components thereof as described herein, which are exclusive of any element not recited in that description of the embodiment.

“Consisting essentially of” refers to those elements required for a given embodiment. The term permits the presence of elements that do not materially affect the basic and novel or functional characteristic(s) of that embodiment.

“About” as used herein means + or −10% deviation from each of the initial value listed unless indicated otherwise.

### Methods:

In one aspect, provided is a method of in vitro or ex-vivo culturing of stem cells and/or lineage committed progenitor cells comprising culturing a population of the stem cells and/or lineage committed progenitor cells in a medium comprising a compound of formula (I):

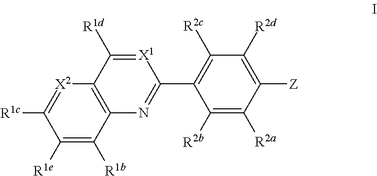

or a pharmaceutically acceptable salt, solvate or hydrate thereof, wherein:
each of ring vertices X¹ and X² is independently selected from the group consisting of C(R^{1a}) and N;
Z is selected from the group consisting of:

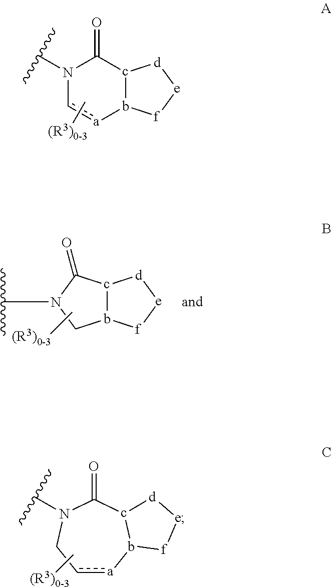

wherein the dashed bonds are single or double bonds, each of ring vertices a, b, c, d, e and f are independently selected from the group consisting of O, S, N, CH, C(R⁴) and N(R⁴), and the bonds joining the ring vertices are independently single or double bonds;
each R^{1a}, R^{1b}, R^{1c}, R^{1d} and R^{1e} is independently selected from the group consisting of hydrogen, deuterium, halogen, —CN, —NO₂, —R^{c}, —CO₂R^{a}, —CONR^{a}R^{b}, —C(O)R^{a}, —OC(O)NR^{a}R^{b}, —NR^{b}C(O)R^{a}, —NR^{b}C(O)₂R^{c}, —NR^{a}C(O)NR^{a}R^{b}, —NR^{a}R^{b}, —OR^{a}, and —S(O)₂NR^{a}R^{b}; wherein each R^{a} and R^{b} is independently selected from hydrogen, C₁₋₈ alkyl, C₃₋₆ cycloalkyl and C₁₋₈ haloalkyl, or when attached to the same nitrogen atom can be combined with the nitrogen atom to form a four-, five- or six-membered ring having from 0 to 2 additional heteroatoms as ring members selected from N, O, S, SO and SO₂; each R^{c} is independently selected from the group consisting of C₁₋₈ alkyl, C₁₋₈ haloalkyl, C₁₋₈ deuteroalkyl, C₂₋₆ alkenyl, C₂₋₆ alkynyl, and C₃₋₆ cycloalkyl, and wherein the aliphatic and cyclic portions of R^{a}, R^{b} and R^{c} can be further substituted with from one to three halogen, hydroxy, C₁₋₄ alkyl, C₁₋₄ alkoxy, amino, C₁₋₄ alkylamino, di C₁₋₄ alkylamino and carboxylic acid groups;
each R^{2a}, R^{2b}, R^{2c} and R^{2d} is independently selected from the group consisting of hydrogen, halogen, C₁₋₃ alkyl, C₁₋₃ deuteroalkyl, C₁₋₃ haloalkyl, C₁₋₃ alkoxy, C₁₋₃ deuteroalkoxy and C₁₋₃ haloalkoxy;
R³ is selected from the group consisting of hydrogen, deuterium, C₁₋₃ alkyl, C₁₋₃ deuteroalkyl, C₁₋₃ alkylene-OR^{d}, C₁₋₃ alkylene-CO₂R^{d}, C₁₋₃ alkylene-NR^{d}R^{e}, C₁₋₃ alkylene-CONR^{d}R^{e}, C₁₋₃ alkylene-OC(O)NR^{d}R^{e}, and C₁₋₃ alkylene-NR^{e}C(O)₂R^{f}; or two R³ groups are combined to form oxo (═O);
each R⁴ is independently selected from the group consisting of hydrogen, halogen, —CN, —R^{f}, —CO₂R^{d}, —CONR^{d}R^{e}, —C(O)R^{d}, —OC(O)NR^{d}R^{e}, —NR^{e}C(O)R^{d}, —NR^{e}C(O)₂R^{f}, —NR^{d}C(O)NR^{d}R^{e}, —NR^{d}R^{e}, —OR^{d}, —S(O)₂NR^{d}R^{e}, —X^{a}—CN, —X^{a}—CO₂R^{d}, —X^{a}—CONR^{d}R^{e}, —X^{a}—C(O)R^{d}, —X^{a}—OC(O)NR^{d}R^{e}, —X^{a}—NR^{e}C(O)R^{d}, —X^{a}—NR^{e}C(O)₂R^{f}, —X^{a}—NR^{d}C(O)NR^{d}R^{e}, —X^{a}—NR^{d}R^{e}, —X^{a}—OR^{d}, and —X^{a}—S(O)₂NR^{d}R^{e}; wherein each X^{a} is independently C₁₋₆alkylene; and
each R^{d} and R^{e} is independently selected from hydrogen, C₁₋₈ alkyl, and C₁₋₈ haloalkyl, or when attached to the same nitrogen atom can be combined with the nitrogen atom to form a four-, five- or six-membered ring having from 0 to 2 additional heteroatoms as ring members selected from N, O, S, SO and SO₂;
each R^{f} is independently selected from the group consisting of C₁₋₈ alkyl, C₁₋₈ haloalkyl, C₁₋₈ deuteroalkyl, C₃₋₆ cycloalkyl, C₃₋₆ heterocycloalkyl, phenyl and 5- or 6-membered heteroaryl;
and wherein the aliphatic and cyclic portions of R^{d}, R^{e} and R^{f} are can be further substituted with from one to three halogen, hydroxy, C₁₋₄ alkyl, C₁₋₄ alkoxy, amino, C₁₋₄ alkylamino, di C₁₋₄ alkylamino and carboxylic acid groups;
wherein the compound of formula (I) antagonizes the activity of aryl hydrocarbon receptor.

In a second aspect, provided is a method for producing an expanded population of stem cells and/or lineage committed progenitor cells comprising contacting a population of the stem cells and/or lineage committed progenitor cells with a compound of formula (I):

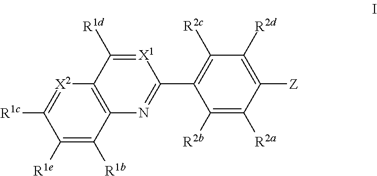

or a pharmaceutically acceptable salt, solvate or hydrate thereof, wherein:
each of ring vertices X¹ and X² is independently selected from the group consisting of C(R^{1a}) and N;
Z is selected from the group consisting of:

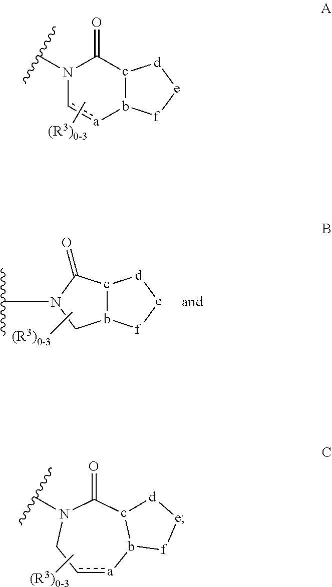

wherein the dashed bonds are single or double bonds, each of ring vertices a, b, c, d, e and f are independently selected from the group consisting of O, S, N, C, C(R⁴) and N(R⁴), and the bonds joining the ring vertices are independently single or double bonds;
each R^{1a}, R^{1b}, R^{1c}, R^{1d} and R^{1e} is independently selected from the group consisting of hydrogen, deuterium, halogen, —CN, —NO₂, —R^{c}, —CO₂R^{a}, —CONR^{a}R^{b}, —C(O)R^{a}, —OC(O)NR^{a}R^{b}, —NR^{b}C(O)R^{a}, —NR^{b}C(O)₂R^{c}, —NR^{a}C(O)NR^{a}R^{b}, —NR^{a}R^{b}, —OR^{a}, and —S(O)₂NR^{a}R^{b}; wherein each R^{a} and R^{b} is independently selected from hydrogen, C₁₋₈ alkyl, C₃₋₆ cycloalkyl and C₁₋₈ haloalkyl, or when attached to the same nitrogen atom can be combined with the nitrogen atom to form a four-, five- or six-membered ring having from 0 to 2 additional heteroatoms as ring members selected from N, O, S, SO and SO₂; each R^{c} is independently selected from the group consisting of C₁₋₈ alkyl, C₁₋₈ haloalkyl, C₁₋₈ deuteroalkyl, C₂₋₆ alkenyl, C₂₋₆ alkynyl, and C₃₋₆ cycloalkyl, and wherein the aliphatic and cyclic portions of R^{a}, R^{b} and R^{c} can be further substituted with from one to three halogen, hydroxy, C₁₋₄ alkyl, C₁₋₄ alkoxy, amino, C₁₋₄ alkylamino, di C₁₋₄ alkylamino and carboxylic acid groups;
each R^{2a}, R^{2b}, R^{2c} and R^{2d} is independently selected from the group consisting of hydrogen, halogen, C₁₋₃ alkyl, C₁₋₃ deuteroalkyl, C₁₋₃ haloalkyl, C₁₋₃ alkoxy, C₁₋₃ deuteroalkoxy and C₁₋₃ haloalkoxy;
R³ is selected from the group consisting of hydrogen, deuterium, C₁₋₃ alkyl, C₁₋₃ deuteroalkyl, C₁₋₃ alkylene-OR^{d}, C₁₋₃ alkylene-CO₂R^{d}, C₁₋₃ alkylene-NR^{d}R^{e}, C₁₋₃ alkylene-CONR^{d}R^{e}, C₁₋₃ alkylene-OC(O)NR^{d}R^{e}, and C₁₋₃ alkylene-NR^{e}C(O)₂R^{f}; or two R³ groups are combined to form oxo (═O);
each R⁴ is independently selected from the group consisting of hydrogen, halogen, —CN, —R^{f}, —CO₂R^{d}, —CONR^{d}R^{e}, —C(O)R^{d}, —OC(O)NR^{d}R^{e}, —NR^{e}C(O)R^{d}, —NR^{e}C(O)₂R^{f}, —NR^{d}C(O)NR^{d}R^{e}, —NR^{d}R^{e}, —OR^{d}, —S(O)₂NR^{d}R^{e}, —X^{a}—CN, —X^{a}—CO₂R^{d}, —X^{a}—CONR^{d}R^{e}, —X^{a}—C(O)R^{d}, —X^{a}—OC(O)NR^{d}R^{e}, —X^{a}—NR^{e}C(O)R^{d}, —X^{a}—NR^{e}C(O)₂R^{f}, —X^{a}—NR^{d}C(O)NR^{d}R^{e}, —X^{a}—NR^{d}R^{e}, —X^{a}—OR^{d}, and —X^{a}—S(O)₂NR^{d}R^{e}; wherein each X^{a} is independently C₁₋₆alkylene; and
each R^{d} and R^{e} is independently selected from hydrogen, C₁₋₈ alkyl, and C₁₋₈ haloalkyl, or when attached to the same nitrogen atom can be combined with the nitrogen atom to form a four-, five- or six-membered ring having from 0 to 2 additional heteroatoms as ring members selected from N, O, S, SO and SO₂;
each R^{f} is independently selected from the group consisting of C₁₋₈ alkyl, C₁₋₈ haloalkyl, C₁₋₈ deuteroalkyl, C₃₋₆ cycloalkyl, C₃₋₆ heterocycloalkyl, phenyl and 5- or 6-membered heteroaryl;
   and wherein the aliphatic and cyclic portions of R^{d}, R^{e} and R^{f} are can be further substituted with from one to three halogen, hydroxy, C₁₋₄ alkyl, C₁₋₄ alkoxy, amino, C₁₋₄ alkylamino, di C₁₋₄ alkylamino and carboxylic acid groups;

wherein the compound of formula (I) antagonizes the activity of aryl hydrocarbon receptor; and

the stem cells and/or progenitor cells are cultured under conditions allowing expansion of the stem cells and/or progenitor cells, respectively. In a first embodiment of the second aspect, the stem cells and/or lineage committed progenitor cells are expanded in vivo by administering to a patient in need thereof a therapeutically effective amount of a compound of formula (I).

In a second embodiment of the second aspect, the stem cells and/or lineage committed progenitor cells are expanded in vitro or ex vivo. For in vitro or ex vivo expansion, the method comprises culturing a population of the stem cells and/or lineage committed progenitor cells in a medium comprising a compound of formula (I).

In some embodiments, the stem cell population and/or lineage committed progenitor cells increases by about 10%, 15%, 20%, 25%, 30%, 35%, 40%, 45%, 59%, 55%, 60%, 65%, 70%, 75%, 80%, 85%, or 90% from the initial amount or increases by at least about a 2-fold, 3-fold, 4-fold, 5-fold or 10-fold, or greater from an initial amount.

In some embodiments of the first and second aspects and first and second embodiments contained with the second aspect, the stem cells and/or progenitor cells are embryonic stem cells and/or lineage committed embryonic progenitor cells.

In some embodiments of the first and second aspects and first and second embodiments contained with the second aspect, the stem cells and/or progenitor cells are induced pluripotent stem cells and/or lineage committed induced pluripotent progenitor cells.

In some embodiments of the first and second aspects and first and second embodiments contained with the second aspect, the stem cells and/or progenitor cells are hematopoietic stem cells and/or lineage committed hematopoietic progenitor cells.

In some embodiments of the first and second aspects and first and second embodiment contained with the second aspect, the stem cells are hematopoietic stem cells.

In some embodiments of the first and second aspects and first and second embodiments contained with the second aspect, the stem cells and/or progenitor cells are genetically modified hematopoietic stem cells and/or lineage committed genetically modified hematopoietic progenitor cells.

In some embodiments of the first and second aspects and first and second embodiment contained with the second aspect, the stem cells and/or progenitor cells are mesenchymal stem cells and/or lineage committed mesenchymal progenitor cells or genetically modified mesenchymal stem cells and/or lineage committed genetically modified mesenchymal progenitor cells.

Embodiments related to in vitro or ex vivo expansion of hematopoietic stem cells and/or lineage committed hematopoietic progenitor cells, genetically modified hematopoietic stems cells and/or lineage committed genetically modified hematopoietic progenitor cells, mesenchymal stem cells and/or lineage committed mesenchymal progenitor cells, and genetically modified mesenchymal stems cells and/or lineage committed genetically modified mesenchymal progenitor cells:

In some embodiments, wherein the stem cells and/or progenitor cells are (i) hematopoietic stem cells and/or lineage committed hematopoietic progenitor cells or (ii) genetically modified hematopoietic stems cells and/or lineage committed genetically modified hematopoietic progenitor cells, the method further includes culturing the hematopoietic stem cells and/or lineage committed hematopoietic progenitor cells or genetically modified hematopoietic stems cells and/or lineage committed genetically modified hematopoietic progenitor cells in the presence of one or more agent(s) selected from a Notch agonist, an agent that inhibits TGFβ signaling, an agent that reduces the activity of an ikaros family transcription factor, an agent that inhibits histone demethylation, an agent that inhibits p38 signaling, an agent that inhibits histone deacetylation, and an agent that inhibits a protein that promotes β-catenin degradation.

Representative examples of Notch agonists include those disclosed in, e.g., in US 2014/0369973 and in U.S. Pat. No. 7,399,633 and methods for expanding hematopoietic stems cells using AhR antagonists and Notch agonists are described in PCT application publication No. WO2013/086436, the disclosures of each of which are incorporated herein by reference. In some embodiments the Notch agonist is an extra cellular domain of a Delta protein or a Jagged/Serrate protein or a Notch-binding portion of any Delta protein or a Jagged protein optionally fused to a fusion partner such as Fc region of an IgG. In some embodiments, the Notch agonist is Delta-^{ext-IgG}.

Agents that can be used to inhibit TGF signaling include, for example, 2-[3-(6-methyl-2-pyridinyl)-1H-pyrazol-4-yl]-1,5-naphthyridine (ALK5 inhibitor I, also known as E-616452), 4-[3-(2-pyridinyl)-1H-pyrazol-4-yl]-quinoline (LY36494, also known as ALK5 Inhibitor I), 3-(6-methyl-2-pyridinyl)-N-phenyl-4-(4-quinolinyl)-1H-pyrazole-1-carbothioamide (also known as A83-01), (4-(5-benzol[1,3]dioxol-5-yl-4-pyridin-2-yl-1H-imidazol-2-yl)-benzamide hydrate (also known as SB431542), 4-[4-(1,3-benzodioxol-5-yl)-5-(2-pyridinyl)-1H-imidazol-2-yl]-benzamide hydrate, 4-[4-(3,4-methylene-dioxyphenyl)-5-(2-pyridyl)-1H-imidazol-2-yl]-benzamide hydrate, an Alk5 inhibitor), Galunisertib (LY2157299, an Alk5 inhibitor), 4-[2-[4-(2-pyridin-2-yl-5,6-dihydro-4H pyrrolo[1,2-b]pyrazol-3-yl)quinolin-7-yl]oxyethyl]morpholine (also known as LY2109761, an Alk5/TGF RII inhibitor), 6-[2-tert-butyl-5-(6-methylpyridin-2-yl)-1H-imidazol-4-yl]quinoxaline (also known as SB525334, an Alk5 inhibitor), N-(oxan-4-yl)-4-[4-(5-pyridin-2-yl-1H-pyrazol-4-yl)pyridin-2-yl]benzamide (also known as GW788388, an Alk5 inhibitor), 3-[6-amino-5-(3,4,5-trimethoxyphenyl)pyridin-3-yl]phenol (also known as K02288, an Alk4/Alk5 inhibitor), 2-(5-chloro-2-fluorophenyl)-N-pyridin-4-ylpteridin-4-amine (also known as SD-208, an Alk5 inhibitor), N-((4-([1,2,4]triazolo[1,5-a]pyridin-6-yl)-5-(6-methylpyridin-2-yl)-1H-imidazol-2-yl)methyl)-2-fluoroaniline (also known as EW-7197, an Alk4/Alk5 inhibitor), and 5-[6-[4-(1-piperazinyl)phenyl]-pyrazolo[1,5-a]pyrimidin-3-yl]-quinoline (also known as LDN-212854, an Alk4/Alk5 inhibitor).

Additional examples of agents that inhibit TGF signaling include, but are not limited to, SM16, a small molecule inhibitor of TGF receptor ALK5, (see Fu et al. Arteriosclerosis, Thrombosis and Vascular Biology 28:665 (2008)), SB-505124 (an Alk4/Alk5 inhibitor, disclosed in Dacosta Byfield et al. Molecular Pharmacology 65:744 (2004)), and 6-bromo-indirubin-3′-oxime (disclosed in U.S. Pat. No. 8,298,825), the disclosures of each of which are incorporated herein by reference. Additional examples of inhibitors of TGFβ signaling are described in, e.g., Callahan et al. Journal of Medicinal Chemistry 45:999 (2002); Sawyer et al. Journal of Medicinal Chemistry 46:3953 (2003); Gellibert et al. Journal of Medicinal Chemistry 47:4494 (2004); Tojo et al. Cancer Science 96:791 (2005); Petersen et al. Kidney International 73:705 (2008); Yingling et al. Nature Reviews Drug Discovery 3:1011 (2004); Byfield et al. Molecular Pharmacology 65:744 (2004); Dumont et al. Cancer Cell 3:531 (2003); WO 2002/094833; WO 2004/026865; WO 2004/067530; WO 2009/032667; WO 2004/013135; WO 2003/097639; WO 2007/048857; WO 2007/018818; WO 2006/018967; WO2005/039570; WO 2000/031135; WO 1999/058128; U.S. Pat. Nos. 6,509,318; 6,090,383; 6,419,928; 7,223,766; 6,476,031; 6,419,928; 7,030,125; 6,943,191; US 2005/0245520; US 2004/0147574; US 2007/0066632; US 2003/0028905; US 2005/0032835; US 2008/0108656; US 2004/015781; US 2004/0204431; US 2006/0003929; US 2007/0155722; US 2004/0138188; and US 2009/0036382, the disclosures of each which are incorporated herein by reference.

Additional agents that can be used to inhibit TGFβ signaling include modulators of bone morphogenetic protein (BMP) signaling. BMP is a member of the TGFβ superfamily of ligands, and modulators of BMP signaling, such as inhibitors of Alk2, Alk3, and Alk6, can be used e.g., to expand hematopoietic stem cells. Exemplary BMP inhibitors include 4-[6-(4-isopropoxyphenyl)pyrazolo[1,5-a]pyrimidin-3-yl]quinoline (also known as DMH1), 4-[6-[4-(1-Methylethoxy)phenyl]pyrazolo[1,5-a]pyrimidin-3-yl]-quinoline), 3-(6-amino-5-(3,4,5-trimethoxyphenyl)pyridin-3-yl)phenol (also known as K02288), 5-[6-[4-(1-piperazinyl)-phenyl]pyrazolo[1,5-a]pyrimidin-3-yl]-quinoline (also known as LDN-212854), 4-[6-[4-(1-piperazinyl)-phenyl]pyrazolo[1,5-a]pyrimidin-3-yl]-quinoline (also known as LDN-193189), 1-(4-(6-methyl-5-(3,4,5-trimethoxyphenyl)pyridin-3-yl)phenyl)piperazine (also known as LDN-214117), and (5-[6-(4-methoxyphenyl)-pyrazolo[1,5-a]pyrimidin-3-yl]quinoline (also known as ML347). Exemplary TGFβ antibodies include and Lerdelimumab and GC-1008. Additional agents that inhibit TGFβ signaling are disclosed in WO2016210292, the disclosure of which are incorporated herein by reference.

Agents that can be used to inhibit histone demethylation include 2-(1R,2S)-2-(4-(benzyloxy)-phenyl)cyclopropyl-amino)-1-(4-methylpiperazin-1-yl)ethanone, HCl (LSD1 inhibitor IV RN-1), 2-(2-(benzyloxy)-3,5-difluorophenyl)-cyclopropan-1-amine (LSD1 inhibitor II S2101), (1S,2S)—N-(1-(2,3-dihydrobenzo[b][1,4]dioxin-6-yl)ethyl)-2-phenylcyclopropan-1-amine (LSD1 inhibitor LSD1-C76), methyl-3-(4-(4-carbamimidoyl-benzoyl)-piperazine-1-carbonyl)-5-((4-carbamimidoylpiperazin-1-yl)methyl)benzoate (LSD1 inhibitor III CBB1007), (E,E)-1,1′-((propane-1,3-diylbis(azanediyl))bis(propane-3,1-diyl))bis(2,3-dimethylguanidine)(LSD1 inhibitor I), Tranylcypromine, phenelzine, propargylamine, and derivatives thereof described in US 2013/0095067, tranylcypromine derivatives (described in US 2014/0163041), BIX 01294 (2-(hexahydro-4-methyl-1H-1,4-diazepin-1-yl)-6,7-dimethoxy-N-[1-(phenylmethyl)-4-piperidinyl]-4-quinazolinamine trihydrochloride, described in WO 2014/057997); UNC 0638 (2-cyclohexyl-6-methoxy-N-[1-(1-methylethyl)-4-piperidinyl]-7-[3-(1-pyrrolidinyl)propoxy]-4-quinazolinamine, described in WO 2013/050422), and CARMI inhibitor (3,5-bis(3-bromo-4-hydroxy-benzylidene)-1-benzylpiperidin-4-one, a histone arginine methyltransferase inhibitor), the disclosures of each of which are incorporated herein by reference.

Agents that can be used to inhibit histone deacetylation include Trichostatin A ((N-hydroxy-N′-phenyl-octanediamide), valproic acid, butyrylhydroxamic acid, isotadax, Panobinostat, MS-275, M344, SAHA, N-(7-trifluoroacetylpropionyl)aniline, 2-[(4-acetylaminophenyl)-carbonylamino]aniline, LAQ824 (Dacinostat), AR-42, Belinostat (PXD101), CUDC-101, Scriptaid, Sodium Phenylbutyrate, Tasquinimod, Quisinostat (JNJ-26481585), Pracinostat (SB939), CUDC-907, Entinostat (MS-275), Mocetinostat (MGCD0103), Tubastatin A HCl, PCI-34051, Droxinostat, RGFP966, Rocilinostat (ACY-1215), CI994 (Tacedinaline), Tubacin, RG2833 (RGFP109), Resminostat, Tubastatin A, BRD73954, BG45, 4SC-202, CAY10603, LMK-235, Nexturastat A, TMP269, HPOB, Cambinol, and Anacardic Acid.

Examples of p38 inhibitor include 4-(4-fluorophenyl)-2-(4-methylsulfinylphenyl)-5-(4-pyridyl)-1H-imidazole (also known as SB203580), 4(4-fluorophenyl)-2-(4-hydroxyphenyl)-5-(4-pyridyl)1H-imidazole (also known as SB2021 90), SB203580, BIRB796 (doramapimod), VX702, SB202190, LY2228820, VX745, Vinorelbine (Navelbine), PH797804, pamapimod, CMPD-1, E01428, JX401, ML3403, RWJ67657, SB239063, SCI0469 hydrochloride, SKF86002 dihydrochloride, SX01, TAK715, e.g., as described in US 2014/0127231, the disclosure of which is incorporated herein by reference, and pexmetinib (ARRY-614), PH-797804 (3-(3-bromo-4-((2,4-difluorobenzyl)oxy)-6-methyl-2-oxopyridin-1(2H)-yl)-N,4-dimethylbenzamide), Losmapimod (GW856553X), and Skepinone-L.

Exemplary compounds that inhibit a protein that inhibits β-catenin degradation include GSK3 inhibitor CHIR99021, AR-A14418, and BIO (2′Z,3′E)-6-bromoindirubin-3′-oxime), lithium chloride, and FGF2.

Examples of agents that reduces the activity of an ikaros family member transcription factor include pomalidomide, lenalidomide, and thalidomide. Additional agents that inhibit ikaros family members are disclosed in WO2017161001, the disclosure of which is incorporated herein by reference. Inhibition of ikaros activity up-regulates expression of a Notch gene.

Other classes of molecules that can be used to reduce the activity of an ikaros family transcription factor, inhibit histone demethylation, p38 signaling, histone deacetylation, TGFβ signaling, or a protein that promotes β-catenin degradation include iRNAs, miRNAs, peptides, and antibodies. For example, endogenous proteins that modulate signal transduction events may be used to attenuate these events, in vitro and ex vivo and utilizing the protein sequence of the above targets, antibodies, mRNA and iRNA can be readily prepared by methods well known in the art. For instance, a variety of proteins that antagonize the TGF signaling cascade can be used, including Decorin, an extra cellular matrix proteoglycan that negatively regulates TGF activity, as well as Lefty1, Lefty2, Follistatin, Noggin, Chordin, Cerberus, Gremlin, Inhibin, Cystatin C, Recombinant Mouse Lefty-I (an ACVR2B inhibitor), as well as the Smad proteins Smad6 and Smad7, which serve to prevent the phosphorylation of the R-Smad proteins or recruit ubiquitin ligases to the TGF receptor type I so as to promote the degradation of the receptor. These proteins are described in detail in U.S. Pat. No. 8,298,825, the disclosure of which is incorporated herein by reference in its entirety.

In some embodiments, wherein the stem cells and/or progenitor cells are (i) hematopoietic stem cells and/or lineage committed hematopoietic progenitor cells or (ii) genetically modified hematopoietic stems cells and/or lineage committed genetically modified hematopoietic progenitor cells, the method further includes culturing the hematopoietic stem cells and/or lineage committed hematopoietic progenitor cells or genetically modified hematopoietic stems cells and/or lineage committed genetically modified hematopoietic progenitor cells in the presence of cytokines and growth factors, generally known in the art for hematopoietic stem cell and/or lineage committed progenitor cell expansion. Such cytokines and growth factors include without limitation IL-1, IL-3, IL-6, IL-11, G-CSF, GM-CSF, SCF, FlT3-L, thrombopoietin (TPO), erythropoietin, and analogs thereof. “Analogs” refers to any structural variants of the cytokines and growth factors having the biological activity as the naturally occurring forms, including without limitation, variants with enhanced or decreased biological activity when compared to the naturally occurring forms or cytokine receptor agonists such as an agonist antibody against the TPO receptor (for example, VB22B sc(Fv)2 as detailed in patent publication WO 2007/145227, and the like). Cytokine and growth factor combinations are chosen to expand HSC and/or lineage committed progenitor cells while limiting the production of terminally differentiated cells. In some embodiments, one or more cytokines and growth factors are selected from the group consisting of SCF, Flt3-L and TPO. In some embodiments, at least TPO is used in a serum-free medium under suitable conditions for expansion of HSC and/or lineage committed progenitor cells.

In some embodiments, wherein the stem cells and/or lineage committed progenitor cells are (i) mesenchymal stem cells and/or lineage committed mesenchymal progenitor cells or (ii) genetically modified mesenchymal stems cells and/or lineage committed genetically modified mesenchymal progenitor cells, the method further comprises culturing the cells the presence of nicotinamide.

In some embodiments, wherein the stem cells and/or lineage committed progenitor cells are (i) mesenchymal stem cells and/or lineage committed mesenchymal progenitor cells or (ii) genetically modified mesenchymal stems cells and/or lineage committed genetically modified mesenchymal progenitor cells, the method further comprises culturing the cells the presence of nicotinamide.

In some embodiments, wherein the stem cells and/or progenitor cells are (i) mesenchymal stem cells and/or lineage committed mesenchymal progenitor cells or (ii) genetically modified mesenchymal stems cells and/or lineage committed genetically modified mesenchymal progenitor cells, the method further comprises culturing the cells the presence of FGF-4. Additional methods of expanding MSCs are disclosed in US 20140023623, the disclosure of which is incorporated herein by reference in its entirety.

When more than one agent is used in any of the methods above, the stem cells and/or lineage committed progenitor cells can be contacted with the one or more agents simultaneously. Alternatively, the stem cells and/or lineage committed progenitor cells can be contacted with said one or more agents at different times (e.g., sequentially).

In some embodiments, in any of the in vitro or ex vivo methods above, the stem cells and/or lineage committed progenitor cells are cultured with from about 1 pM to about 100 μM or from about 100 pM to about 10 μM of a compound of formula (I).

In some embodiments, in any of the in vitro or ex vivo methods above, the stem cells and/or lineage committed progenitor cells are cultured in the presence of a compound of formula (I) from about 1 day to about 90 days or from 3 days to 90 days, or from about 2 to about 35 days or from about 7 to about 35 days or 5 days to 21 days.

In some embodiments, in any of the in vitro or ex vivo methods above, the stem cells and/or lineage committed progenitor cells are cultured in the presence of a compound of formula (I) from about 1 day to 3 days.

In some embodiments, in any of the in vitro or ex vivo methods above, the stem cells and/or lineage committed progenitor cells are cultured in the presence of a compound of formula (I) during a time sufficient for a 10 to 50,000-fold expansion or 100 to 10,000-fold expansion of the stem cells as compared to a population of stem cells and/or lineage committed progenitor cells thereof cultured under the same conditions in the absence of a compound of formula (I).

### Embodiments Related to Compounds of Formula (I) in Above Methods:

In some embodiments, in any of the methods above, the compounds of formula (I), are those wherein Z has subformula A (formula IA).

In some embodiments, in any of the methods above, the compounds of formula (I), are those wherein Z has subformula B (formula IB).

In some embodiments, in any of the methods above, the compounds of formula (I), are those wherein Z has subformula C (formula IC).

In those embodiments referred to herein as formula IA; in selected embodiments, Z is selected from the group consisting of:

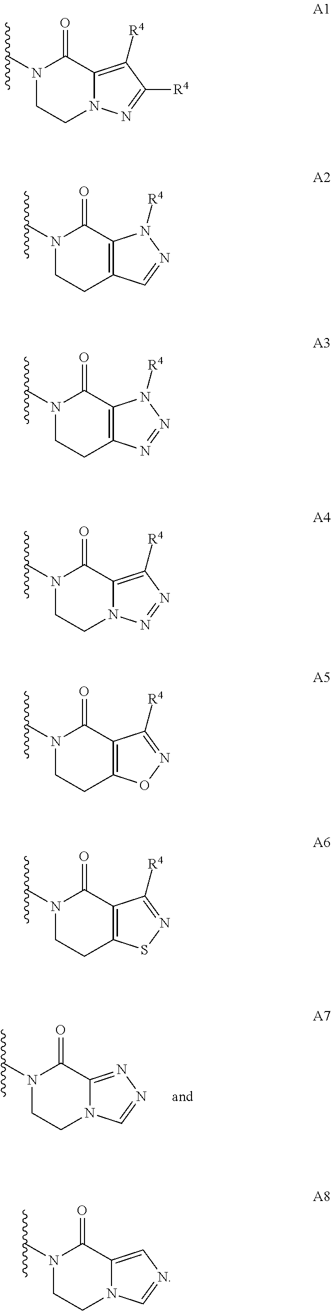

In further selected embodiments, Z is A1. In further selected embodiments, Z is A2. In further selected embodiments, Z is A5.

In those embodiments referred to herein as formula IB; in selected embodiments, Z is selected from the group consisting of:

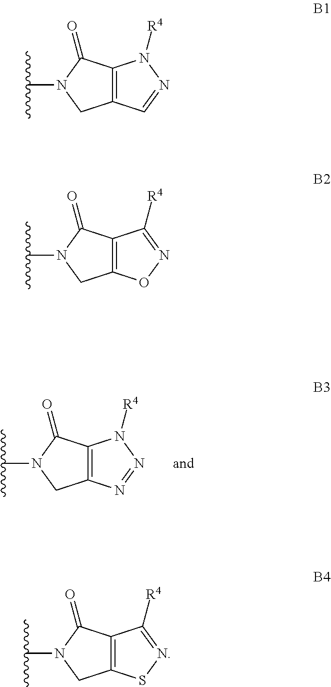

In further selected embodiments, Z is B1. In further selected embodiments, Z is B2.

In those embodiments referred to herein as formula IC; in selected embodiments, Z is selected from the group consisting of:

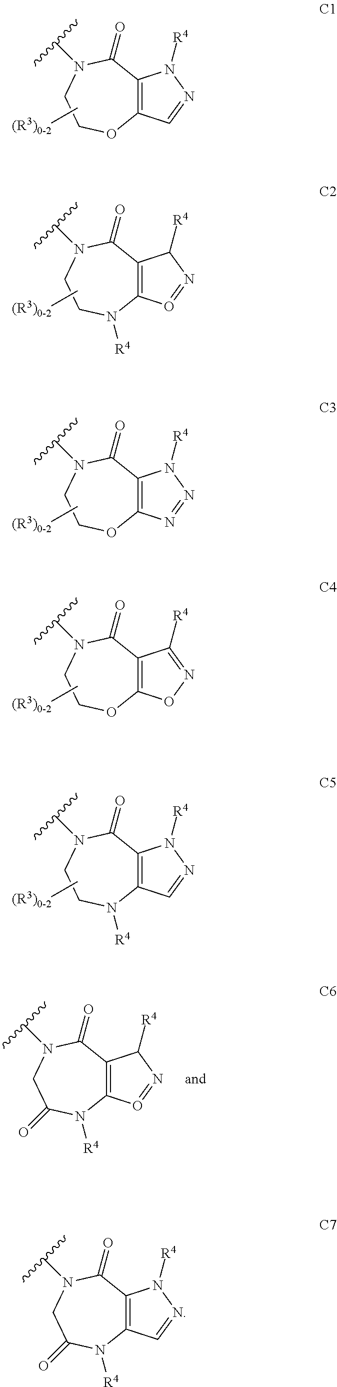

In further selected embodiments, Z is C1. In further selected embodiments, Z is C5.

In some selected embodiments, in any of the methods above, compounds of formula IA, B, IC, IA1, IA2, IA3, IA4, IA5, IA6, IA7, IA8, IB1, IB2, IB3, IB4, IC1, IC2, IC3, IC4, IC5, IC6, or IC7, are those wherein X¹ is N.

In some selected embodiments, in any of the methods above, compounds of formula IA, IB, IC, IA1, IA2, IA3, IA4, IA5, IA6, IA7, IA8, IB1, IB2, IB3, IB4, IC1, IC2, IC3, IC4, IC5, IC6, or IC7, are those wherein X² is N.

In some selected embodiments, in any of the methods above, compounds of formula IA, IB, IC, IA1, IA2, IA3, IA4, IA5, IA6, IA7, IA8, IB1, IB2, IB3, IB4, IC1, IC2, IC3, IC4, IC5, IC6, or IC7, are those wherein X¹ and X² are both N.

In some selected embodiments, in any of the methods above, compounds of formula IA, IB, IC, IA1, IA2, IA3, IA4, IA5, IA6, IA7, IA8, IB1, IB2, IB3, IB4, IC1, IC2, IC3, IC4, IC5, IC6, or IC7, are those wherein R^{1b} is selected from the group consisting of H, deuterium, C₁₋₄ alkyl and C₁₋₄ haloalkyl. In further selected embodiments, R^{1b} is selected from the group consisting of H, deuterium and CH₃.

In some selected embodiments, in any of the methods above, compounds of formula IA, IB, IC, IA1, IA2, IA3, IA4, IA5, IA6, IA7, IA8, IB1, IB2, IB3, IB4, IC1, IC2, IC3, IC4, IC5, IC6, or IC7, are those wherein R^{1c} is selected from the group consisting of H, deuterium, Ca alkyl and C₁₋₄ haloalkyl. In further selected embodiments, R^{1c} is selected from the group consisting of H, deuterium and CF₃.

In some selected embodiments, in any of the methods above, compounds of formula IA, IB, IC, IA1, IA2, IA3, IA4, IA5, IA6, IA7, IA8, IB1, IB2, IB3, IB4, IC1, IC2, IC3, IC4, IC5, IC6, or IC7, are those wherein R^{1d} and R^{1e} are each independently selected from the group consisting of H, deuterium, C₁₋₄ alkyl and C₁₋₄ haloalkyl.

In some selected embodiments, in any of the methods above, compounds of formula IA, IB, IC, IA1, IA2, IA3, IA4, IA5, IA6, IA7, IA8, IB1, IB2, IB3, IB4, IC1, IC2, IC3, IC4, IC5, IC6, or IC7, are those wherein R^{2a} is selected from the group consisting of H, deuterium, halogen, C₁₋₄ alkyl and C₁₋₄ haloalkyl.

In some selected embodiments, in any of the methods above, compounds formula IA, IB, IC, IA1, IA2, IA3, IA4, IA5, IA6, IA7, IA8, IB1, IB2, IB3, IB4, IC1, IC2, IC3, IC4, IC5, IC6, or IC7, are those wherein R^{2b} is selected from the group consisting of H, deuterium, halogen, C₁₋₄ alkyl and C₁₋₄ haloalkyl.

In some selected embodiments, in any of the methods above, compounds of formula IA, IB, IC, IA1, IA2, IA3, IA4, IA5, IA6, IA7, IA8, IB1, IB2, IB3, IB4, IC1, IC2, IC3, IC4, IC5, IC6, or IC7, are those wherein R^{2c} is selected from the group consisting of H, deuterium, halogen, C₁₋₄ alkyl and C₁₋₄ haloalkyl.

In some selected embodiments, in any of the methods above, compounds of formula IA, IB, IC, IA1, IA2, IA3, IA4, IA5, IA6, IA7, IA8, IB1, IB2, IB3, IB4, IC1, IC2, IC3, IC4, IC5, IC6, or IC7, are those wherein R^{2d} is selected from the group consisting of H, deuterium, halogen, C₁₋₄ alkyl and C₁₋₄ haloalkyl.

In some selected embodiments, in any of the methods above, compounds of formula IA, IB, IC, IA1, IA2, IA3, IA4, IA5, IA6, IA7, IA8, IB1, IB2, IB3, IB4, IC1, IC2, IC3, IC4, IC5, IC6, or IC7, are those wherein each of R^{2a}, R^{2b}, R^{2c} and R^{2d} are independently selected from the group consisting of H, deuterium, fluoro and CH₃.

In some selected embodiments, in any of the methods above, compounds of formula IC, are those wherein two R³ are combined to form oxo.

In some selected embodiments, in any of the methods above, compounds of formula IA, IB, IC, IA1, IA2, IA3, IA4, IA5, IA6, IA7, IA8, IB1, IB2, IB3, IB4, IC1, IC2, IC3, IC4, IC5, IC6, or IC7, are those wherein each R⁴ is independently selected from the group consisting of H, halogen, —R^{f}, —CO₂R^{d}, —CONR^{d}R^{e}, —C(O)R^{d}, —NR^{e}C(O)R^{d}, —NR^{e}C(O)₂R^{f}, —NR^{d}C(O)NR^{d}R^{e}, —NR^{d}R^{e}, —OR^{d}, —X^{a}—CN, —X^{a}—CO₂R^{d}, —X^{a}—CONR^{d}R^{e}, —X^{a}—C(O)R^{d}, —X^{a}—OC(O)NR^{d}R^{e}, —X^{a}—NR^{e}C(O)R^{d}, —X^{a}—NR^{e}C(O)₂R^{f}, —X^{a}—NR^{d}C(O)NR^{d}R^{e}, —X^{a}—NR^{d}R^{e}, and —X^{a}—OR^{d}; wherein each X^{a} is independently C₁₋₄alkylene. In further selected embodiments, each R⁴ is independently H, halogen, or R^{f}. In further selected embodiments, each R⁴ is independently H, halogen, C₁₋₆alkyl, C₁₋₆haloalkyl, C₁₋₆deuteroalkyl, or C₃₋₆cycloalkyl.

In some selected embodiments, in any of the methods above, compounds of formula IA, IB, IC, IA1, IA2, IA3, IA4, IA5, IA6, IA7, IA8, IB1, IB2, IB3, IB4, IC1, IC2, IC3, IC4, IC5, IC6, or IC7, are those wherein X¹ is N, X² is CH, R^{1b} is H or CH₃, R^{1c} is CF₃, R^{2a} is H, F or CH₃, R^{2b} is H, and R^{2c} is H or CH₃.

In some selected embodiments, in any of the methods above, compounds of formula IA, IB, IC, IA1, IA2, IA3, IA4, IA5, IA6, IA7, IA8, IB1, IB2, IB3, IB4, IC1, IC2, IC3, IC4, IC5, IC6, or IC7, are those wherein X¹ is N, X² is N, R^{1b} is H or CH₃, R^{1c} is CF₃, R^{2a} is H, F or CH₃, R^{2b} is H, and R^{2c} is H or CH₃.

In some selected embodiments, in any of the methods above, compounds of formula IA, IB, IC, IA1, IA2, IA3, IA4, IA5, IA6, IA7, IA8, IB1, IB2, IB3, IB4, IC1, IC2, IC3, IC4, IC5, IC6, or IC7, are those wherein X¹ is CH, X² is N, R^{1b} is H or CH₃, R^{1c} is CF₃, R^{2a} is H, F or CH₃, R^{2b} is H, and R^{2c} is H or CH₃.

In some selected embodiments, in any of the methods above, compounds of formula IA, IB, IC, IA1, IA2, IA3, IA4, IA5, IA6, IA7, IA8, IB1, IB2, IB3, IB4, IC1, IC2, IC3, IC4, IC5, IC6, or IC7, are those wherein X¹ is CH, X² is CH, R^{1b} is H or CH₃, R^{1c} is CF₃, R^{2a} is H, F or CH₃, R^{2b} is H, and R^{2c} is H or CH₃.

In still other selected embodiments, in any of the methods above, compounds of formula (I) has the structure formula (IIa), (IIb), (IIc), (IId), (Ie) or (If):

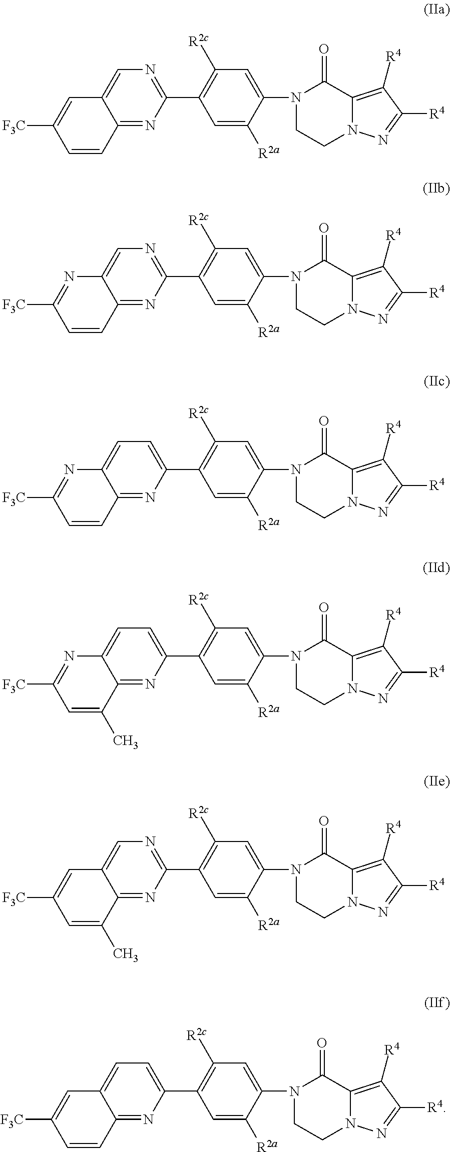

In further selected embodiments, each R⁴ is independently H, halogen, or R^{f}. In further selected embodiments, each R⁴ is independently H, halogen, C₁₋₆alkyl, C₁₋₆haloalkyl, C₁₋₆deuteroalkyl, or C₃₋₆cycloalkyl.

In still other selected embodiments, in any of the methods above, compounds of formula (I) has the structure formula (IIIa), (IIIb), (IIIc) or (IIId):

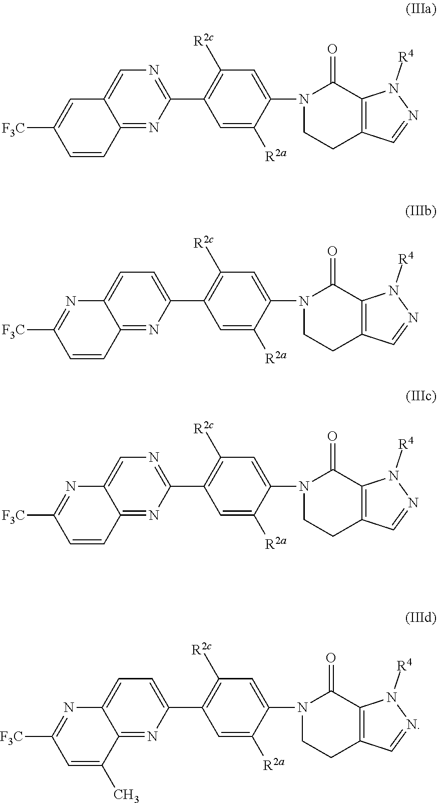

In further selected embodiments, each R⁴ is independently H, halogen, or R^{f}. In further selected embodiments, each R⁴ is independently H, halogen, C₁₋₆alkyl, C₁₋₆haloalkyl, C₁₋₆deuteroalkyl, or C₃₋₆cycloalkyl.

In still other selected embodiments, in any of the methods above, compounds of formula (I) has the structure formula (IVa), (IVb), (IVc) or (IVd):

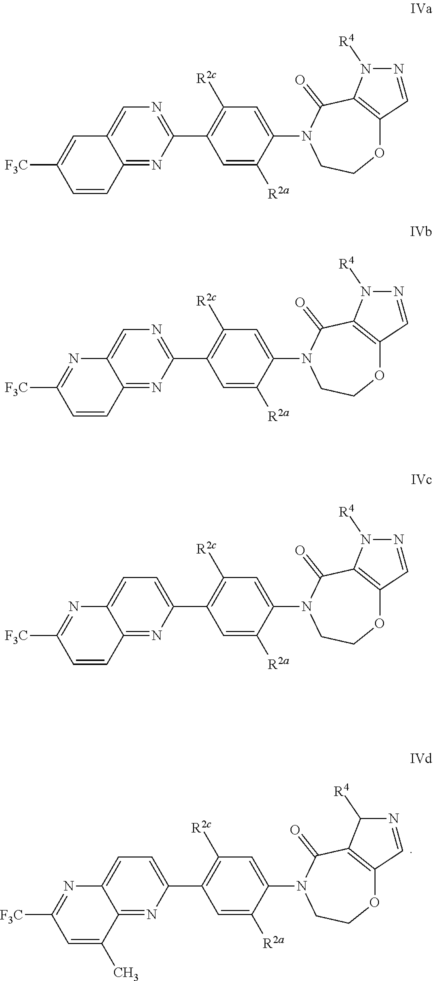

In further selected embodiments, each R⁴ is independently H, halogen, or R^{f}. In further selected embodiments, each R⁴ is independently H, halogen, C₁₋₆alkyl, C₁₋₆haloalkyl, C₁₋₆deuteroalkyl, or C₃₋₆cycloalkyl.

In still other selected embodiments, in any of the methods above, compounds of formula (I) are those disclosed in Table 1 having +++ or ++++ activity.

### Stem Cell Sources for In Vitro or Ex Vivo Expansion:

The starting stem cell and/or lineage committed progenitor cell population for use in the in vitro and ex vivo methods disclosed herein are either commercially available or can be isolated from various tissues and/or organs of a mammal or can be prepared by methods well known in the art.

For example, embryonic stem cells (ESCs) are derived from the inner cell mass of an embryo (see Thomson et. al, Science. 1998 Nov. 6; 282(5391):1145-7) whereas induced pluripotent stem cells (iPSCs) are derived from somatic cells (see Takahashi et. al, Cell. 2007 Nov. 30; 131(5):861-72; Takahashi et. al, Nat Protoc. 2007; 2(12):3081-9; Yu et. al, Science. 2007 Dec. 21; 318(5858):1917-20. Epub 2007 Nov. 20).

Hematopoietic stem cells, including lineage committed hematopoietic progenitor cells, can be isolated from bone marrow, umbilical cord, peripheral blood, liver, thymus, lymph, and spleen. Mesenchymal stem cells, including mesenchymal progenitor cells, can be isolated from bone marrow, umbilical cord, adipose tissue, molar cells, and amniotic fluid. In some embodiments, the stem cells and progenitor cells are isolated from a patient in need of stem cell therapy. Stems cells may be characterized by both the presence of specific markers (e.g., proteins, RNAs, etc.) and the absence of specific markers. Stem cells may also be identified by functional assays both in vitro and in vivo, particularly assays relating to the ability of stem cells to give rise to multiple differentiated progeny.

The aforementioned crude or un-fractionated sources containing the desired stem cells can be used in the methods disclosed herein directly after isolation or after purification and/or enrichment for a specific type of stem cell. The purification and/or enrichment can be based on presence or absence of specific cellular marker(s). Methods for enriching stem cells are known to those of skill in the art e.g., flow cytometry. In some embodiments, the starting population of hematopoietic stem cells are enriched in Endothelial Protein C Receptor (EPCR+) and/or CD34+, CD38+, CD90+, CD45RA+, CD133 and/or CD49f+ cells. In some embodiments, the hematopoietic stem cells are enriched in CD34+ cells and/or EPCR+ cells. In some embodiments, the hematopoietic stem cells are enriched in CD34+ cells. Enriched, as used above, means at least about 10%, 20% 30%, 50%, 60%, 70%, 80% or 90% of HSCs express one or more of above listed markers. In some embodiment, the starting population of cells for expansion for use in the methods disclosed herein consist essentially of CD34+ cells. With respect to MSCs, in some embodiments, the starting MSCs are positive for one or more of CD105, CD90, CD44, CD73, CD29, CD13, CD34, CD146, CD106, CD54 and CD166 markers. In some embodiments, the MSCs are at least over 30%, 50%, 60%, 70%, 80% or 90% are CD106+ and preferably CD45− cells. In some embodiments, an expanded stem cell and/or lineage committed progenitor cell population accordingly to the methods disclosed herein can also serve as a starting population for further expansion according to the methods disclosed herein.

“Genetically modified stem cell(s)” refers to a naturally occurring stem cell that has exogenous nucleic acid artificially introduced inside the cell and/or one or more endogenous gene(s) artificially, either completely or partially, deleted, replaced, and/or one or more endogenous gene(s) mutated. Genetically modified stem cells are prepared from naturally occurring stem cells by methods well known in the art. For example, exogenous nucleic acid can be introduced into the target stem cell by viral or non-viral gene transfer, including but not limited to, electroporation, lipofection, sonoporation, gene gun, plasmid transfer, phage integrase, transposons, AdV, AAV, and Lentivirus transfection, and ZNF, TALENs, and CRISPR/Cas9 methodologies. ZNF, TALENs, and CRISPR/Cas9 methodologies can also be used to partially or fully delete and/or replace a gene(s) from a target cell (see for example PCT Applications publication nos. WO 2015/057976 and WO2016/182959). In some embodiments, the genetically modified stem cells are made after culturing of naturally occurring stem cells with a compound of formula (I). In some embodiments, the genetically modified stem cells are made prior to culturing of naturally occurring stem cells with a compound of formula (I). In some embodiments, the genetically modified stem cells are made after expansion of naturally occurring stem cells according to the methods disclosed herein. In some embodiments, the genetically modified stem cells are made prior to expansion of naturally occurring stem cells according to the methods disclosed herein. In some embodiments, the exogenous nucleic acid is integrated (covalently linked) into the genome of the modified cells. In some embodiments, the exogenous nucleic acid is not integrated (covalently linked) into the genome of the modified cells. In some embodiments, the exogenous nucleic acid comprises one or more genes and is integrated into the genome of the modified cell. In some embodiments, the exogenous nucleic acid can be a complete gene(s) or a fragment thereof. In some embodiments, the exogenous nucleic acid encodes a molecule selected from the group consisting of a protein and RNA (e.g., mRNA, siRNA, miRNA, or shRNA).

In some embodiments, one or more endogenous genes that is a member of a pathway whose activity is inhibited (e.g., TGFβ pathway, histone demethylase pathway) in the methods disclosed herein is fully or partially deleted. In some embodiments, the exogenous nucleic acid comprises one or more genes that expresses a product (e.g., protein, RNAi, mRNA) having a therapeutic utility. For example, a nucleic acid that expresses RNAi or mRNA that can silence the expression of an endogenous gene involved in a disease pathway is inserted. In some embodiments, the exogenous nucleic acid, preferably a gene is introduced and or an endogenous gene is deleted or replaced using ZNF, TALENs, and/or CRISPR/Cas9 methodology.

In yet another aspect, provided is a method of making a genetically modified stem cell comprising culturing the naturally occurring stem cell with a compound of formula (I) (or any embodiment thereof disclosed herein). In some embodiment, the cell is modified after it is cultured with a compound of formula (I) (or any embodiment thereof disclosed herein). In some embodiment, the cell is modified before it is contacted with a compound of formula (I) (or any embodiment thereof disclosed herein). In any of the above embodiments, the stem cell that is HSC. In any of the above embodiments, the cell is modified using viral transfection, ZNF, TALENs, and/or CRISPR/Cas (preferably CRISPR/Cas9) methodology. In any of the above embodiments, the cell is modified for transplantation into a patient in need thereof.

### ADDITIONAL EMBODIMENTS

### Embodiment 1

Embodiment 1 is directed to the method of the first aspect described above. Within embodiment 1, in a first subembodiment, the method of Embodiment 1 is wherein the stem cells and/or lineage committed progenitor cells, are cultured under conditions that maintain functional potential of the stem cells and/or lineage committed progenitor cells.

### Embodiment 2

Embodiment 2 is directed a method for producing an expanded population of stem cells and/or lineage committed progenitor cells thereof in vitro or ex vivo comprising culturing a population of the stem cells and/or lineage committed progenitor cells in a medium comprising a compound of formula (I) wherein the compound of formula (I) antagonizes the activity of aryl hydrocarbon receptor; and the stem cells and/or progenitor cells are cultured under conditions allowing expansion of the stem cells and/or progenitor cells. Within embodiment 2, in a first subembodiment, the method of embodiment 2 further comprises differentiating the expanded stem cells to lineage committed progenitor cells thereof under conditions that cause differentiation of the expanded stem cells to lineage committed progenitor cells thereof.

### Embodiment 3

In embodiment 3, the method of any one of embodiments 1, 2, and subembodiments contained therein, is wherein the method is carried out ex vivo.

### Embodiment 4

In embodiment 4, the method of anyone of embodiments 1, 2, 3, and any subembodiments contained therein, is wherein the stem cells and lineage committed progenitor cells are human cells.

### Embodiment 5

In embodiment 5, the method of any one of embodiments 1 to 4, and any subembodiments contained therein, is wherein the stem cells and/or lineage committed progenitor cells are hematopoietic stem cells and/or lineage committed hematopoietic progenitor cells. In a subembodiment of embodiment 5, the stem cells and/or lineage committed progenitor cells are hematopoietic stem cells and lineage committed hematopoietic progenitor cells thereof.

### Embodiment 6

In embodiment 6, the method of any one of embodiments 1 to 4, and any subembodiments contained therein, is wherein the stem cells and/or lineage committed progenitor cells are genetically modified hematopoietic stem cells and/or lineage committed progenitor cells thereof. In one subembodiment of 6, the genetically modified hematopoietic stem cells and/or lineage committed progenitor cells thereof comprise an exogenous nucleic acid. In a second subembodiment of embodiment 6, the genetically modified hematopoietic stem cells and/or lineage committed progenitor cells thereof comprise an exogenous nucleic acid that is integrated in the genome of the modified cells.

### Embodiment 7

In embodiment 7, the method of any one of embodiments 5, 6, and any subembodiments contained therein, further comprises culturing the (i) hematopoietic stem cells and/or lineage committed hematopoietic progenitor cells or (ii) genetically modified hematopoietic stem cells and/or lineage committed genetically modified hematopoietic progenitor cells in the presence of a Notch agonist. Within embodiment 7, in a first subembodiment, the Notch agonist is an extra cellular domain of a Delta protein or a Jagged protein or a Notch-binding portion of extra cellular domain of a Delta protein or a Jagged protein optionally fused to Fc region of an IgG. Within embodiment 7, in a second subembodiment, the Notch agonist is Delta-^{ext-IgG}.

### Embodiment 8

In embodiment 8, the method of any one of embodiments 5 to 7, and any subembodiments contained therein, further comprises culturing the population of (i) hematopoietic stem cells and/or lineage committed hematopoietic progenitor cells or (ii) genetically modified hematopoietic stem cells and/or lineage committed genetically modified hematopoietic progenitor cells in the presence of an agent that inhibits TGFβ signaling. Within embodiment 8, in a first subembodiment, the agent is a TGFβ receptor inhibitor. Within embodiment 8, in a second subembodiment, the agent is a TGFβ receptor inhibitor selected from the group consisting of 2-[3-(6-methyl-2-pyridinyl)-1H-pyrazol-4-yl]-1,5-naphthyridine, 4-[3-(2-pyridinyl)-1H-pyrazol-4-yl]-quinoline, 4-[6-(4-isopropoxyphenyl)-pyrazolo[1,5-a]pyrimidin-3-yl]quinoline, and 3-(6-methyl-2-pyridinyl)-N-phenyl-4-(4-quinolinyl)-1H-pyrazole-1-carbothioamide. Within embodiment 8, in a third subembodiment, the TGFβ receptor inhibitor is 3-(6-methyl-2-pyridinyl)-N-phenyl-4-(4-quinolinyl)-1H-pyrazole-1-carbothioamide.

### Embodiment 9

In embodiment 9, the method of any one of embodiments 5 to 8 and any subembodiments contained therein, further comprises culturing the population of (i) hematopoietic stem cells and/or lineage committed hematopoietic progenitor cells or (ii) genetically modified hematopoietic stem cells and/or lineage committed genetically modified hematopoietic progenitor cells in the presence of an agent that inhibits histone demethylation. Within embodiment 9, in a subembodiment the agent is a histone demethylase inhibitor. Within embodiment 9, in a first subembodiment, the histone demethylase inhibitor is a LSD1 inhibitor. Within embodiment 9, in a second subembodiment, the histone demethylase inhibitor is selected from the group consisting of 2-(1R,2S)-2-(4-(benzyloxy)phenyl)cyclopropylamino)-1-(4-methylpiperazin-1-yl)ethanone, HCl (LSD1 inhibitor IV RN-1), 2-(2-(benzyloxy)-3,5-difluorophenyl)cyclopropan-1-amine (LSD1 inhibitor II S2101), (1S,2S)—N-(1-(2,3-dihydrobenzo[b][1,4]dioxin-6-yl)ethyl)-2-phenylcyclopropan-1-amine (LSD1 inhibitor LSD1-C76), methyl-3-(4-(4-carbamimidoylbenzoyl)piperazine-1-carbonyl)-5-((4-carbamimidoylpiperazin-1-yl)methyl)benzoate (LSD1 inhibitor III CBB1007), (E,E)-1,1′-((propane-1,3-diylbis(azanediyl))bis(propane-3,1-diyl))bis(2,3-dimethylguanidine) (LSD1 inhibitor I), and Tranylcypromine. Within embodiment 9, in a third subembodiment, the histone demethylase inhibitor is Tranylcypromine.

### Embodiment 10

In embodiment 10, the method of any one of embodiments 5 to 9 and any subembodiments contained therein, further comprises culturing the population of (i) hematopoietic stem cells and/or lineage committed hematopoietic progenitor cells or (ii) genetically modified hematopoietic stem cells and/or lineage committed genetically modified hematopoietic progenitor cells in the presence of an agent that inhibits histone acetylation. Within embodiment 10, in a subembodiment, the agent is a histone deacetylase inhibitor. Within embodiment 10, in a first subembodiment, the histone deacetylase inhibitor is selected from the group consisting of Trichostatin A, valproic acid, butyrylhydroxamic acid, and istodax. Within embodiment 10, in a second subembodiment, the histone deacetylase inhibitor is Trichostatin A.

### Embodiment 11

In embodiment 11, the method of anyone of embodiments 5 to 10 and any subembodiments contained therein, further comprises culturing the population of (i) hematopoietic stem cells and/or lineage committed hematopoietic progenitor cells or (ii) genetically modified hematopoietic stem cells and/or lineage committed genetically modified hematopoietic progenitor cells in the presence of an agent that inhibits p38 signaling; and/or an agent that inhibits a protein that promotes β-catenin degradation. Within embodiment 11, in a first subembodiment, the p38 inhibitor is 4-(4-fluorophenyl)-2-(4-methylsulfinylphenyl)-5-(4-pyridyl)-1H-imidazole (SB203580). Within embodiment 11, in a second embodiment, the agent that inhibits β-catenin degradation is 6-[[2-[[4-(2,4-dichlorophenyl)-5-(5-methyl-1H-imidazol-2-yl)-2-pyrimidinyl]amino]ethyl]amino]-3-pyridinecarbonitrile (CHIR99021), LiCl, BIO, or FGF2.

### Embodiment 12

In embodiment 12, the method of any one of embodiments 5 to 11, and any subembodiments contained therein, further comprises culturing the population of (i) hematopoietic stem cells and/or lineage committed hematopoietic progenitor cells or (ii) genetically modified hematopoietic stem cells and/or lineage committed genetically modified hematopoietic progenitor cells in the presence of an agent that reduces the activity of an ikaros family member transcription factor. With embodiment 12, in a first subembodiment, the agent that reduces the activity of an ikaros family member transcription factor is pomalidomide, lenalidomide, or thalidomide.

### Embodiment 13

In embodiment 13, the method of any one of embodiments 5 to 12 and any subembodiments contained therein, further comprises culturing the population of (i) hematopoietic stem cells and/or lineage committed hematopoietic progenitor cells or (ii) genetically modified hematopoietic stem cells and/or lineage committed genetically modified hematopoietic progenitor cells are cultured under conditions that maintain the functional potential of the hematopoietic stem cells and/lineage committed hematopoietic progenitor cells or genetically modified hematopoietic stems cells and/or lineage committed genetically modified hematopoietic progenitor cells, respectively.

### Embodiment 14

In embodiment 14, the method of any one of embodiments 5 to 13, and any subembodiments contained therein, is wherein the hematopoietic stem cells, including those for producing genetically modified hematopoietic stem cells, and/or lineage committed hematopoietic progenitor cells are from bone marrow, umbilical cord blood, or mobilized peripheral blood.

### Embodiment 15

In embodiment 15, the method of any one of embodiments 5 to 13, and any subembodiments contained therein, is wherein the hematopoietic stem cells, including those used for producing genetically modified hematopoietic stem cells, and/or lineage committed hematopoietic progenitor cells are from bone marrow, umbilical cord blood, or mobilized peripheral blood and are enriched in Endothelial Protein C Receptor (EPCR+) and/or CD34+, CD38+, CD90+, CD45RA+, CD133 and/or CD49f+. Within embodiment 15, in a first subembodiment, the hematopoietic stem cells are enriched in CD34+ cells and/or EPCR+. Within embodiment 15, in a second subembodiment, the hematopoietic stem cells are enriched in CD34+ cells.

### Embodiment 16

In embodiment 16, the method of any one of embodiments 5 to 15, and any subembodiments contained therein, is wherein the hematopoietic stem cells, including those used for producing genetically modified hematopoietic stem cells, consist essentially of CD34+ cells.

### Embodiment 17

In embodiment 17, the method of any one of embodiments 5 to 16, and any subembodiments contained therein, further comprising culturing the (i) hematopoietic stem cells and/or lineage committed hematopoietic progenitor cells or (ii) genetically modified hematopoietic stem cells and/or lineage committed genetically modified hematopoietic progenitor cells in the presence of a sufficient amount of one or more of IL6, Flt-3-L, TPO, and SCF. Within embodiment 17, in a first subembodiment, the cells are cultured in the presence of IL6, Flt-3-L, TPO, and SCF.

### Embodiment 18

In embodiment 18, the method of any one of embodiments 5 to 17, and any subembodiments contained therein, is wherein the amount of the compound of formula (I) in the cell culture medium is from about 1 pM to about 100 μM. Within embodiment 18, in a first subembodiment, the amount of the compound of formula (I) in the cell culture is from about 100 pM to about 10 μM.

### Embodiment 19

In embodiment 19, the method of any one of embodiments 5 to 18, and any subembodiments contained therein, is wherein the stem cells and/or lineage committed progenitor cells are cultured in the presence of a compound of formula (I) from about 1 day to about 90 days. Within embodiment 19, in a first subembodiment, the stem cells and/or lineage committed progenitor cells are cultured from about 2 to about 35 days. Within embodiment 19, in a second subembodiment, the stem cells and/or lineage committed progenitor cells are cultured from about 2 to about 35 days. Within embodiment 19, in a third subembodiment, the stem cells and/or lineage committed progenitor cells are cultured from about 3 days to about 90 days. Within embodiment 19, in a fourth subembodiment, the stem cells and/or lineage committed progenitor cells are cultured from about 7 to about 35 days. Within embodiment 19, in a fourth subembodiment, the stem cells and/or lineage committed progenitor cells are cultured from about 7 to about 21 days. Within embodiment 19, in a fourth subembodiment, the stem cells and/or lineage committed progenitor cells are cultured from about 1 to about 3 days.

### Embodiment 20

In embodiment 20, the method of any one of embodiments 5 to 19, and any subembodiments contained therein, is wherein the stem cells are cultured in the presence of a compound of formula (I) during a time sufficient for a about 2 to 50,000-fold, expansion of hematopoietic cells, preferably CD34+ cells, as compared to a population of hematopoietic in the absence of a compound of formula (I).

### Embodiment 21

In embodiment 21, the method of any one of embodiments 5 to 20, and any subembodiments contained therein, is wherein the hematopoietic stem cells and/or lineage committed hematopoietic progenitor cells or genetically modified hematopoietic progenitor cells and/or lineage committed genetically modified hematopoietic progenitor cells are contacted with said one or more agents in embodiments 7 to 12 and 17 simultaneously.

### Embodiment 22

In embodiment 22, the method of any one of embodiments 5 to 20, and any subembodiments contained therein, is wherein the hematopoietic stem cells and/or lineage committed hematopoietic progenitor cells or genetically modified hematopoietic progenitor cells and/or lineage committed genetically modified hematopoietic progenitor cells are contacted with said one or more agents in embodiments 7 to 12 and 17 at different times.

### Embodiment 23

In embodiment 23, the method of any one of embodiments 5 to 22 and any subembodiments contained therein, is wherein the hematopoietic stem cells, including those used to prepared genetically modified hematopoietic stem cells, and/or lineage committed hematopoietic progenitor cell are originally within a mononuclear cell fraction prior to treatment with said one or more agents.

### Embodiment 24

In embodiment 23, the method of any one of embodiments 5 to 22 and any subembodiments contained therein, is wherein the hematopoietic stem cells, including those used for producing genetically modified hematopoietic stem cells, and/or lineage committed hematopoietic progenitor cells, are originally within a CD34+, CD34+/CD38−, CD34+/CD38−/CD90+, CD34+/CD38−/CD90+/CD45RA−, or CD34+/CD38−/CD90+/CD45RA−/CD49F+ enriched cell fraction prior to contacting said one or more agents.

### Embodiment 25

In embodiment 25, the method of any one of embodiments 5 to 22 and any subembodiments contained therein, is wherein the hematopoietic stem cells, including those used for producing genetically modified hematopoietic stem cells, are originally within an un-enriched cell fraction prior to contacting said one or more agents.

### Embodiment 26

In embodiment 26, the method of any one of embodiments 1 to 25 and any subembodiments contained therein, is wherein the compound of formula (I) is wherein Z has subformula A.

### Embodiment 27

In embodiment 27, the method of any one of embodiments 1 to 25 and any subembodiments contained therein, is wherein the compound of formula (I) is wherein Z is selected from the group consisting of:

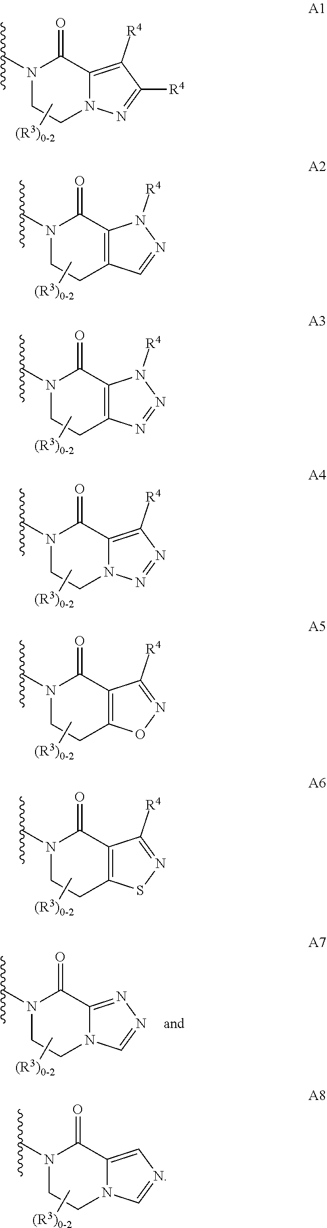

### Embodiment 28

In embodiment 28, the method of any one of embodiments 1 to 25 and any subembodiments contained therein, is wherein the compound of formula (I) is wherein Z has subformula B.

### Embodiment 29

In embodiment 29, the method of any one of embodiments 1 to 25 and any subembodiments contained therein, is wherein the compound of formula (I) is wherein Z is selected from the group consisting of:

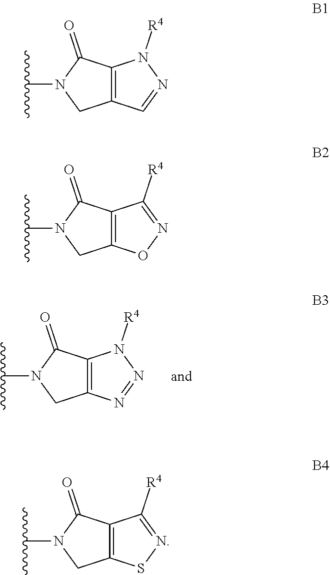

### Embodiment 30

In embodiment 30, the method of any one of embodiments 1 to 25 and any subembodiments contained therein, is wherein the compound of formula (I) is wherein Z has subformula C.

### Embodiment 31

In embodiment 31, the method of anyone of embodiments 1 to 25 and any subembodiments contained therein, is wherein the compound of formula (I) is wherein the compound of formula (I) is wherein Z is selected from the group consisting of:

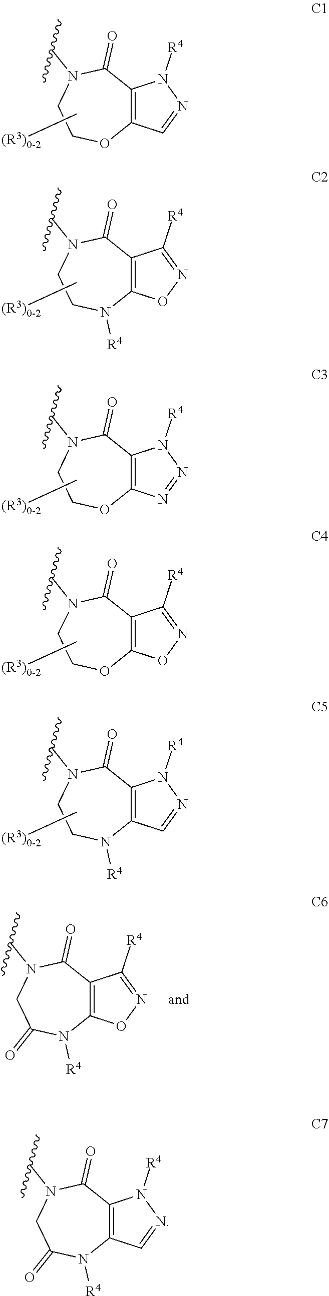

### Embodiment 32

In embodiment 32, the method of any one of embodiments 1 to 31 and any subembodiments contained therein, is wherein the compound of formula (I) is wherein X¹ is N.

### Embodiment 33

In embodiment 33, the method of any one of embodiments 1 to 31 and any subembodiments contained therein, is wherein the compound of formula (I) is wherein X² is N.

### Embodiment 34

In embodiment 34, the method of any one of embodiments 1 to 31 and any subembodiments contained therein, is wherein the compound of formula (I) is wherein X¹ and X² are both N.

### Embodiment 35

In embodiment 35, the method of any one of embodiments 1 to 34 and any subembodiments contained therein, is wherein the compound of formula (I) is wherein R^{1b} is selected from the group consisting of H, deuterium, C₁₋₄ alkyl and C₁₋₄ haloalkyl.

### Embodiment 36

In embodiment 36, the method of any one of embodiments 1 to 35 and any subembodiments contained therein, is wherein the compound of formula (I) is wherein R^{1d} and R^{1e} are each H.

### Embodiment 37

In embodiment 37, the method of any one of embodiments 1 to 36 and any subembodiments contained therein, is wherein the compound of formula (I) is wherein R^{1c} is selected from the group consisting of H, deuterium, C₁₋₄ alkyl and C₁₋₄ haloalkyl.

### Embodiment 38

In embodiment 38, the method of any one of embodiments 1 to 37 and any subembodiments contained therein, is wherein the compound of formula (I) is wherein R^{2a} is selected from the group consisting of H, deuterium, halogen, C₁₋₄ alkyl and C₁₋₄ haloalkyl.

### Embodiment 39

In embodiment 39, the method of any one of embodiments 1 to 38 and any subembodiments contained therein, is wherein the compound of formula (I) is wherein R^{2b} is selected from the group consisting of H, deuterium, halogen, C₁₋₄ alkyl and C₁₋₄ haloalkyl.

### Embodiment 40

In embodiment 40, the method of any one of embodiments 1 to 39 and any subembodiments contained therein, is wherein the compound of formula (I) is wherein R^{2c} is selected from the group consisting of H, deuterium, halogen, C₁₋₄ alkyl and C₁₋₄ haloalkyl.

### Embodiment 41

In embodiment 41, the method of any one of embodiments 1 to 40 and any subembodiments contained therein, is wherein the compound of formula (I) is wherein R^{1b} is selected from the group consisting of H, deuterium and CH₃.

### Embodiment 42

In embodiment 42, the method of any one of embodiments 2 to 41 and any subembodiments contained therein, is wherein the compound of formula (I) is wherein R^{1c} is selected from the group consisting of H, deuterium, and CF₃.

### Embodiment 43

In embodiment 43, the method of any one of embodiments 1 to 42 and any subembodiments contained therein, is wherein the compound of formula (I) is wherein each of R^{2a}, R^{2b}, R^{2c} and R^{2d} are independently selected from the group consisting of H, deuterium, fluoro and CH₃.

### Embodiment 44

In embodiment 44, the method of any one of embodiments 1 to 43 and any subembodiments contained therein, is wherein the compound of formula (I) is wherein each R⁴ is independently selected from the group consisting of H, halogen, —R^{f}, —CO₂R^{d}, —CONR^{d}R^{e}, —C(O)R^{d}, —NR^{e}C(O)R^{d}, —NR^{e}C(O)₂R^{f}, —NR^{d}C(O)NR^{d}R^{e}, —NR^{d}R^{e}, —OR^{d}, —X^{a}—CN, —X^{a}—CO₂R^{d}, —X^{a}—CONR^{d}R^{e}, —X^{a}—C(O)R^{d}, —X^{a}—OC(O)NR^{d}R^{e}, —X^{a}—NR^{e}C(O)R^{d}, —X^{a}—NR^{e}C(O)₂R^{f}, —X^{a}—NR^{d}C(O)NR^{d}R^{e}, —X^{a}—NR^{d}R^{e} and —X^{a}—OR^{d}; wherein each X^{a} is independently C₁₋₄alkylene.

### Embodiment 45

In embodiment 45, the method of any one of embodiments 1 to 44 and any subembodiments contained therein, is wherein the compound of formula (I) is wherein each R³ is methyl.

### Embodiment 46

In embodiment 46, the method of any one of embodiments 1 to 25 and any subembodiments contained therein, is wherein the compound of formula (I) is a compound of formula (IIa), (IIb), (IIc), (IId), (IIe) or (IIf):

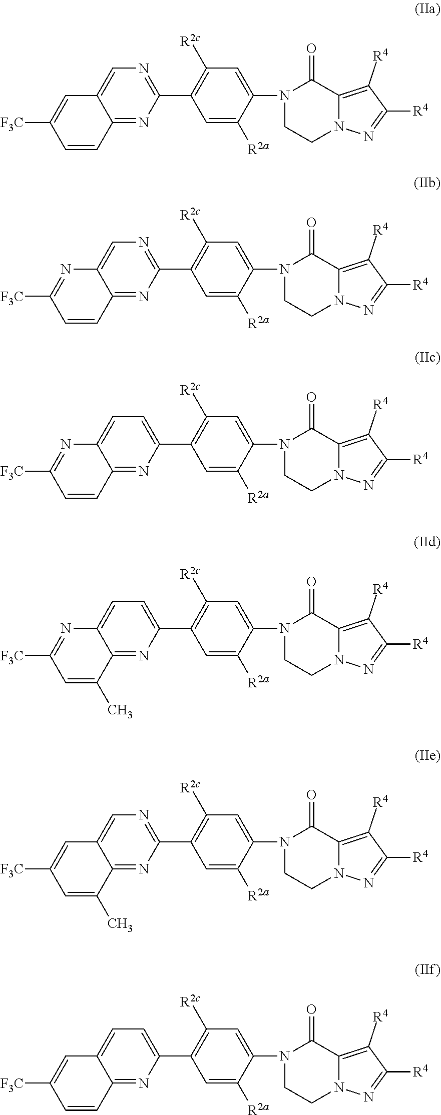

### Embodiment 47

In embodiment 47, the method of any one of embodiments 1 to 25 and any subembodiments contained therein, is wherein the compound of formula (I) is a compound of formula (IIIa), (IIIb), (IIIc), or (IIId):

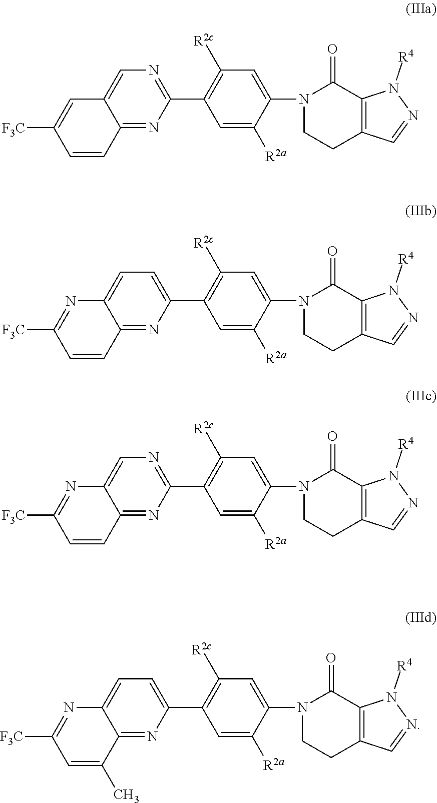

### Embodiment 48

In embodiment 48, the method of any one of embodiments 1 to 25 and any subembodiments contained therein, is wherein the compound of formula (I) is a compound of formula (IVa), (IVb), (IVc) or (IVd):

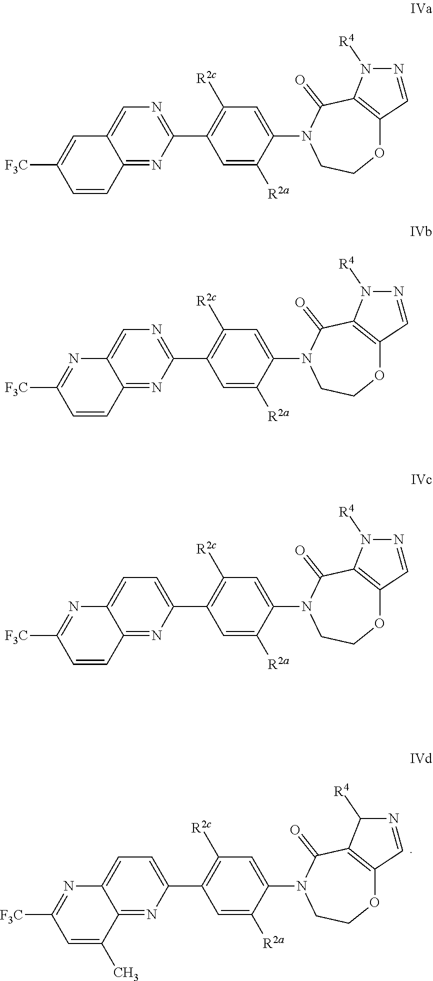

### Embodiment 49

In embodiment 49, the method of any one of embodiments 1 to 25 and any subembodiments contained therein, is wherein the compound of formula (I) is selected from Table 1.

### Embodiment 50

In embodiment 50, the method of any one of embodiments 1 to 25 and any subembodiments contained therein, is wherein the compound of formula (I) is selected from Table 1 and having +++ or ++++ activity.

### Embodiment 51

In embodiment 51, the method of anyone of embodiments 1 to 25 and any subembodiments contained therein, is wherein the compound of formula (I) is selected from Table 1 and having ++++ activity.

### Embodiment 52

An ex vivo or in vitro composition comprising a cell population of expanded stem cells and/or lineage committed progenitor cells thereof and a compound of formula (I) (and any embodiments thereof disclosed in embodiments 26 to 51). Within embodiment 52, in a first subembodiment the expanded stem cells are hematopoietic stem cells and/or lineage committed progenitor cells thereof. Within embodiment 52, in a first subembodiment the expanded stem cells are genetically modified hematopoietic stem cells and/or lineage committed progenitor cells thereof. Within embodiment 52, in a third subembodiment the expanded cells are hematopoietic stem cells and/or lineage committed hematopoietic progenitor cells.

### Embodiment 53

A composition comprising a cell population of expanded stem cells obtained or obtainable by culturing ex vivo a starting population of the stem cells and a compound of formula (I) (and any embodiments thereof disclosed in embodiments 26 to 51. Within embodiment 53, in a first subembodiment the expanded stem cells are hematopoietic stem cells and/or lineage committed progenitor cells thereof. Within embodiment 53, in a first subembodiment the expanded stem cells are genetically modified hematopoietic stem cells and/or lineage committed progenitor cells thereof. Within embodiment 53, in a third subembodiment the expanded cells are hematopoietic stem cells and/or lineage committed hematopoietic progenitor cells.

### Embodiment 54

A composition comprising a cell population of expanded hematopoietic stem cells and/or lineage committed progenitor cells thereof obtained or obtainable by culturing ex vivo a starting population of hematopoietic stem cells and/or lineage committed progenitor cells thereof according to the method of any one of the embodiments 2 to 51.

### Embodiment 55

In embodiment 55, the composition of any one of embodiments 52 to 54 is wherein the composition further comprises a pharmaceutically acceptable medium. In one subembodiment of embodiment 55, the composition comprising the stem cells is substantially free of a compound of formula (I) and/or any other component of the cell culture. “Substantially free” as used herein means the composition comprising the stem cells contains less than about 10%, 20%, 30%, 40%, or 50%, preferably less than about 10% or 20% of formula (I) and/or any other component of the culture medium, more preferably less than about 10%, preferably less than about 5% of a compound of formula (I) and/or any other component of the culture medium.

### Embodiment 56

In embodiment 56, the composition of any one of embodiments 52 to 54 is wherein the composition is suspended in a pharmaceutically acceptable medium suitable for transplantation into a patient, preferably the patient is a human. In a subembodiment of embodiment 56, the patient is administered a therapeutically effective amount of stem cells. In a first subembodiment of embodiment 53, the patient is administered a therapeutically effective amount of stem cells. In a second subembodiment of embodiment 53, the composition comprising the stem cells is substantially free of a compound of formula (I), (II), (II), or (IV) and/or any other component of the cell culture. “Substantially free” as used herein means the composition comprising the stem cells contains less than about 10%, 20%, 30%, 40%, or 50%, preferably less than about 10% or 20% of formula a compound of formula (I), (II), (II), or (IV) and/or any other component of the cell culture, more preferably less than about 10% of a compound of formula (I) (I), (II), (II), or (IV) and/or any other component of the cell culture.

### Embodiment 57

Embodiment 57 is directed to a method of treating a disease treatable by hematopoietic stem cell and/or lineage committed progenitor cells comprising administering to a patient in need thereof a composition of any one of embodiments 54 to 56.

### Embodiment 58

In embodiment 58 the method of embodiment 57 is wherein the disease is an immunodeficiency disease, an autoimmune disorder, or a hematopoietic disorder.

### Embodiment 59

In embodiment 59 the method of embodiment 57 is wherein the disease is selected from the group consisting of Acute Lymphoblastic Leukemia (ALL), Acute Myelogenous Leukemia (AML), Chronic Myelogenous Leukemia (CML), Chronic Lymphocytic Leukemia (CLL), Hodgkin Lymphoma (HL), Non-Hodgkin Lymphoma (NHL), Myelodysplastic Syndrome (MDS), Multiple myeloma, Aplastic anemia, Bone marrow failure, Myeloproliferative disorders such as Myelofibrosis, Essential thrombocytopenia or Polycythemia vera, Fanconi anemia, Dyskeratosis congenita, Common variable immune deficiency (CVID, such as CVID 1, CVID 2, CVID 3, CVID 4, CVID 5, and CVID 6), Human immunodeficiency virus (HIV), Hemophagocytic lymphohistiocystosis, Amyloidosis, Solid tumors such as Neuroblastoma, Germ cell tumors, Breast cancer, Wilms' tumor, Medulloblastoma, and Neuroectodermal tumors, Autoimmune diseases such as Scleroderma, Multiple sclerosis, Ulcerative colitis, Systemic lupus erythematosus and Type I diabetes, or protein deficiencies such as Adrenoleukodystrophy (ALD), Metachromatic leukodystrophy (MLD), Hemophilia A & B, Hurler syndrome, Hunter syndrome, Fabry disease, Gaucher disease, Epidermolysis bullosa, Globoid Cell Leukodystrophy, Sanfillipo syndrome, and Morquio syndrome.

### Embodiment 60

In embodiment 60 the method of embodiment 57 is wherein the disease is Sickle cell anemia, Alpha thalassemia, Beta thalassemia, Delta thalassemia, Hemoglobin E/thalassemia, Hemoglobin S/thalassemia, Hemoglobin C/thalassemia, Hemoglobin D/thalassemia, Chronic granulomatous disease (X-linked Chronic granulomatous disease, autosomal recessive (AR) chronic granulomatous disease, chronic granulomatous disease ARI NCF1, Chronic granulomatous disease AR CYBA, Chronic granulomatous disease AR II NCF2, Chronic granulomatous disease AR III NCF4, X-linked Severe Combined Immune Deficiency (SCID), ADA SCID, 1L7-RA SCID, CD3 SCID, Rag1/Rag2 SCID, Artemis SCID, CD45 SCID, Jak3 SCID, Congenital agranulocytosis, Congenital agranulocytosis-congenital neutropenia-SCN1, Congenital agranulocytosis-congenital neutropenia-SCN2, Familial hemophagocytic lymphohistiocystosis (FHL), Familial hemophagocytic lymphohistiocytosis type 2 (FHL2, perforin mutation), Agammaglobulinemia (X-linked Agammaglobulinemia), Wiskott-Aldrich syndrome, Chediak-Higashi syndrome, Hemolytic anemia due to red cell pyruvate kinase deficiency, Paroxysmal nocturnal hemoglobinuria, X-linked Adrenoleukodystrophy (X-ALD), X-linked lymphoproliferative disease, Unicentric Castleman's Disease, Multicentric Castleman's Disease, Congenital amegakaryocytic, thrombocytopenia (CAMT) type I, Reticular dysgenesis, Fanconi anemia, Acquired idiopathic sideroblastic anemia, Systemic mastocytosis, Von Willebrand disease (VWD), Congenital dyserythropoietic anemia type 2, Cartilage-hair hypoplasia syndrome, Hereditary spherocytosis, Blackfan-Diamond syndrome, Shwachman-Diamond syndrome, Thrombocytopenia-absent radius syndrome, Osteopetrosis, Infantile osteopetrosis, Mucopolysaccharidoses, Lesch-Nyhan syndrome, Glycogen storage disease, Congenital mastocytosis, Omenn syndrome, X-linked Immunodysregulation, polyendocrinopathy, and enteropathy (IPEX), IPEX characterized by mutations in FOXP3, X-linked syndrome of polyendocrinopathy, immune dysfunction, and diarrhea (XPID), X-Linked Autoimmunity-Allergic Dysregulation Syndrome (XLAAD), IPEX-like syndrome, Hyper IgM type 1, Hyper IgM type 2, Hyper IgM type 3, Hyper IgM type 4, Hyper IgM type 5, X linked hyperimmunoglobulin M, Bare lymphocyte Syndrome type I, and Bare lymphocyte Syndrome type II (Bare lymphocyte Syndrome type IL, MHC class I deficiency; Bare lymphocyte Syndrome type II, complementation group A; Bare lymphocyte Syndrome type II, complementation group C; Bare lymphocyte Syndrome type II complementation group D; Bare lymphocyte Syndrome type IL, complementation group E).

### Embodiment 61

An ex vivo or in vitro composition comprising a cell population of stem cells and/or lineage committed progenitor cells and a compound of formula (I) (and any embodiments thereof disclosed in embodiments 26 to 51. Within embodiment 52, in a first subembodiment the stem cells are hematopoietic stem cells and/or lineage committed progenitor cells thereof. Within embodiment 61, in a second subembodiment the expanded cells are hematopoietic stem cells and/or lineage committed hematopoietic progenitor cells.

### Embodiment 62

A catheter comprising a cell population of expanded hematopoietic stem cells and/or lineage committed hematopoietic progenitor cells thereof obtained or obtainable by culturing ex vivo a starting population of cells comprising hematopoietic stem cells and/or lineage committed progenitor cells thereof and a compound of formula (I) according to the method of any one of embodiments 2 to 51.

### Embodiment 63

A syringe comprising a cell population of expanded hematopoietic stem cells and/or lineage committed hematopoietic progenitor cells obtained or obtainable by culturing ex vivo a starting population of cells comprising hematopoietic stem cells and/or lineage committed progenitor cells thereof and a compound of formula (I) according to the method of any one of embodiments 2 to 51.

### Embodiment 64

Embodiment 62 is directed to a method of preparing genetically modified stem cells and/or lineage committed progenitor hematopoietic cells comprising culturing naturally occurring stem cells and/or lineage committed progenitor cells thereof and a compound of formula (I) (and any embodiments thereof disclosed in embodiments 26 to 51). Within embodiment 64, in a first subembodiment the stem cells are hematopoietic stem cells and/or lineage committed progenitor cells thereof.

### Embodiment 65

Embodiment 65, the method comprises culturing the hematopoietic stem cells and/or lineage committed progenitor cells thereof with a compound of formula (I) (and any embodiments thereof disclosed in embodiments 26 to 51) from 1 to 3 days prior to genetically modification the stem cells. In one subembodiment of embodiment 65, the hematopoietic cells are modified using CRISPR/Cas 9 method.

### Compositions and Therapeutic Uses:

The expanded stem cells produced by any of the methods disclosed herein can be used for treatment of various diseases treatable by stem cell-based therapy. The expanded stem cells produced by in vitro or ex vivo methods disclosed herein may be used without further purification (i.e., directly after expansion) or after further purification and/or selection step(s). For example, the expanded stem cells may be washed to remove the compound of formula (I) and/or one or more agents used in the expansion methods disclosed herein and then resuspended in an appropriate cell suspension medium for short term use or in a long-term storage medium or in a pharmaceutically acceptable medium suitable for administration to a patient. Accordingly, in one aspect provided is an ex vivo or in vitro composition comprising a cell population of expanded stem cells and/or lineage committed progenitor cells and a compound of formula (I) (or any embodiment thereof disclosed herein). In another aspect, provided is an ex vivo or in vitro composition comprising a cell population of expanded stem cells and/or lineage committed progenitor cells substantively free of one or more agents used for the expansion of the stem cells in the methods disclosed herein (e.g., compound of formula (I) Notch agonist, an agent that inhibits TGFβ signaling, TPO, etc.). In yet another aspect, provided is a composition comprising a cell population of expanded stem cells and/or lineage committed progenitor cells thereof obtained or obtainable by any of the in vitro or ex vivo expansion methods disclosed herein. In yet another aspect, provided is a composition comprising a cell population of expanded stem cells and/or lineage committed progenitor cells obtained or obtainable by any of the in vitro or ex vivo expansion methods disclosed herein that is substantially free of one or more agents used for the expansion of the stem cells in the methods disclosed herein. Substantially free as used herein means the composition comprises less than about 5%, 10%, 20%, 30%, 40%, or 50%, preferably less than about 5% of one or more agents used in the expansion methods disclosed herein. “About” applies to each of the stated value.

In some embodiments, the expanded stem cells of any of aforementioned compositions are HSCs. In some embodiments, the expanded stem cells of any of aforementioned compositions are MSCs. In some embodiments the expanded stem cells of any of aforementioned compositions are genetically modified HSCs.

In some embodiments, any of the aforementioned compositions comprising expanded stem cells is resuspended in a pharmaceutically acceptable medium suitable for administration to a subject in need of stem cell therapy.

Accordingly, also provided herein are methods of treating diseases treatable by stem cell therapy comprising administering to a patient in need thereof a therapeutically effective amount of expanded stem cells prepared according to the stem cell expansion methods disclosed herein, optionally in a pharmaceutically acceptable medium. In a non-limiting example, the stem cells are transplanted to a subject in need of transplantation of stem cells. As used herein, the term “subject in need of transplantation of stem cells” include, e.g., subjects suffering from a disorder that can be treated by, i.e., can benefit from, transplantation of stem cells. Transplantation of the expanded stem cell population of the present disclosure may be by any suitable means of administration, for example, by subcutaneous, intravenous, intramuscular, or intracisternal injection or infusion techniques (e.g., cell suspensions) in a pharmaceutically acceptable medium. A pharmaceutically acceptable carrier for infusion of a composition comprising cells into a patient may comprise, for example, buffered saline with 5% HSA or supplemented basal medium or medium as known in the art. In other embodiments, transplantation can be facilitated via the use of catheter-based systems, pumps, or syringes.

It will be understood, however, that the therapeutic amount of expanded stem cells and frequency of administration for any particular subject may be varied and will depend upon a variety of factors including the activity and viability of the specific cells employed, and the age, body weight, general health, sex, diet, mode and time of administration, rate of engraftment, drug combination, the severity of the particular condition, and the host undergoing therapy. In some embodiments, a therapeutically effective amount of expanded cells transplanted into a subject is an amount capable of engrafting such transplanted cells in the subject, whereby such cells ameliorate of the symptoms of the disease by eliciting the desired biological response. In certain embodiments, stem cells administered to a subject can be in an amount between 1×10⁶ to 5×10⁸ cells/kg body weight, which can be delivered in single or multiple doses. In other embodiments, stem cells provided to a subject can be in an amount of about 1×10⁶, 2×10⁶, 5×10⁶, 1×10⁷, 5×10⁷, 1×10⁸ cells/kg body weight, or an amount between any of the above listed values. The cells may be administered on a regimen of 1 to 5 times per day, 1-3 times per day, once or twice per day, or every other day.

For methods where the stem cells are expanded in vivo, the compounds of formula (I) (and any embodiments thereof) provided herein may be in the form of compositions suitable for administration to a subject. In general, such compositions are “pharmaceutical compositions” comprising a compound of formula (I) (and any embodiments thereof) and one or more pharmaceutically acceptable or physiologically acceptable diluents, carriers or excipients. In certain embodiments, the compound of formula (I) (and any embodiments thereof) is present in a therapeutically acceptable amount.

The pharmaceutical compositions can be formulated to be compatible with the intended method or route of administration; exemplary routes of administration are set forth herein. Furthermore, the pharmaceutical compositions may be used in combination with other therapeutically active agents or compounds as described herein in order to treat or prevent the diseases, disorders and conditions as contemplated by the present invention.

The pharmaceutical compositions containing the active ingredient (e.g., of formula (I) (and any embodiments thereof)) may be in a form suitable for oral use, for example, as tablets, capsules, troches, lozenges, aqueous or oily suspensions, dispersible powders or granules, emulsions, hard or soft capsules, or syrups, solutions, microbeads or elixirs. Pharmaceutical compositions intended for oral use may be prepared according to any method known to the art for the manufacture of pharmaceutical compositions, and such compositions may contain one or more agents such as, for example, sweetening agents, flavoring agents, coloring agents and preserving agents in order to provide pharmaceutically elegant and palatable preparations. Tablets, capsules and the like contain the active ingredient in admixture with non-toxic pharmaceutically acceptable excipients which are suitable for the manufacture of tablets. These excipients may be, for example, diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc.

The tablets, capsules and the like suitable for oral administration may be uncoated or coated by known techniques to delay disintegration and absorption in the gastrointestinal tract and thereby provide a sustained action. For example, a time-delay material such as glyceryl monostearate or glyceryl distearate may be employed. They may also be coated by techniques known in the art to form osmotic therapeutic tablets for controlled release. Additional agents include biodegradable or biocompatible particles or a polymeric substance such as polyesters, polyamine acids, hydrogel, polyvinyl pyrrolidone, polyanhydrides, polyglycolic acid, ethylenevinylacetate, methylcellulose, carboxymethylcellulose, protamine sulfate, or lactide/glycolide copolymers, polylactide/glycolide copolymers, or ethylenevinylacetate copolymers in order to control delivery of an administered composition. For example, the oral agent can be entrapped in microcapsules prepared by coacervation techniques or by interfacial polymerization, by the use of hydroxymethylcellulose or gelatin-microcapsules or poly (methylmethacrylate) microcapsules, respectively, or in a colloid drug delivery system. Colloidal dispersion systems include macromolecule complexes, nano-capsules, microspheres, microbeads, and lipid-based systems, including oil-in-water emulsions, micelles, mixed micelles, and liposomes. Methods for the preparation of the above-mentioned formulations will be apparent to those skilled in the art.

Formulations for oral use may also be presented as hard gelatin capsules wherein the active ingredient is mixed with an inert solid diluent, for example, calcium carbonate, calcium phosphate, kaolin or microcrystalline cellulose, or as soft gelatin capsules wherein the active ingredient is mixed with water or an oil medium, for example peanut oil, liquid paraffin, or olive oil.

Aqueous suspensions contain the active materials in admixture with excipients suitable for the manufacture thereof. Such excipients can be suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents, for example a naturally-occurring phosphatide (e.g., lecithin), or condensation products of an alkylene oxide with fatty acids (e.g., polyoxy-ethylene stearate), or condensation products of ethylene oxide with long chain aliphatic alcohols (e.g., for heptadecaethyleneoxycetanol), or condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyethylene sorbitan monooleate). The aqueous suspensions may also contain one or more preservatives.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified herein.

The pharmaceutical compositions of the present invention may also be in the form of oil-in-water emulsions. The oily phase may be a vegetable oil, for example olive oil or arachis oil, or a mineral oil, for example, liquid paraffin, or mixtures of these. Suitable emulsifying agents may be naturally occurring gums, for example, gum acacia or gum tragacanth; naturally occurring phosphatides, for example, soy bean, lecithin, and esters or partial esters derived from fatty acids; hexitol anhydrides, for example, sorbitan monooleate; and condensation products of partial esters with ethylene oxide, for example, polyoxyethylene sorbitan monooleate.

The pharmaceutical compositions typically comprise a therapeutically effective amount of formula (I) (and any embodiments thereof) and one or more pharmaceutically and physiologically acceptable formulation agents. Suitable pharmaceutically acceptable or physiologically acceptable diluents, carriers or excipients include, but are not limited to, antioxidants (e.g., ascorbic acid and sodium bisulfate), preservatives (e.g., benzyl alcohol, methyl parabens, ethyl or n-propyl, p-hydroxybenzoate), emulsifying agents, suspending agents, dispersing agents, solvents, fillers, bulking agents, detergents, buffers, vehicles, diluents, and/or adjuvants. For example, a suitable vehicle may be physiological saline solution or citrate buffered saline, possibly supplemented with other materials common in pharmaceutical compositions for parenteral administration. Neutral buffered saline or saline mixed with serum albumin are further exemplary vehicles. Those skilled in the art will readily recognize a variety of buffers that can be used in the pharmaceutical compositions and dosage forms contemplated herein. Typical buffers include, but are not limited to, pharmaceutically acceptable weak acids, weak bases, or mixtures thereof. As an example, the buffer components can be water soluble materials such as phosphoric acid, tartaric acids, lactic acid, succinic acid, citric acid, acetic acid, ascorbic acid, aspartic acid, glutamic acid, and salts thereof. Acceptable buffering agents include, for example, a Tris buffer, N-(2-hydroxyethyl)piperazine-N′-(2-ethanesulfonic acid) (HEPES), 2-(N-morpholino)ethane-sulfonic acid (MES), 2-(N-Morpholino)ethanesulfonic acid sodium salt (MES), 3-(N-Morpholino)propanesulfonic acid (MOPS), and N-tris[Hydroxymethyl]methyl-3-aminopropanesulfonic acid (TAPS).

After a pharmaceutical composition has been formulated, it may be stored in sterile vials as a solution, suspension, gel, emulsion, solid, or dehydrated or lyophilized powder. Such formulations may be stored either in a ready-to-use form, a lyophilized form requiring reconstitution prior to use, a liquid form requiring dilution prior to use, or other acceptable form. In some embodiments, the pharmaceutical composition is provided in a single-use container (e.g., a single-use vial, ampoule, syringe, or autoinjector (similar to, e.g., an EpiPen®)), whereas a multi-use container (e.g., a multi-use vial) is provided in other embodiments.

Formulations can also include carriers to protect the composition against rapid degradation or elimination from the body, such as a controlled release formulation, including liposomes, hydrogels, prodrugs and microencapsulated delivery systems. For example, a time delay material such as glyceryl monostearate or glyceryl stearate alone, or in combination with a wax, may be employed. Any drug delivery apparatus may be used to deliver an AhR modulator, including syringes, implants (e.g., implantable pumps) and catheter systems, slow injection pumps and devices, all of which are well known to the skilled artisan.

Depot injections, which are generally administered subcutaneously or intramuscularly, may also be utilized to release the AhR modulators disclosed herein over a defined period of time. Depot injections are usually either solid- or oil-based and generally comprise at least one of the formulation components set forth herein. One of ordinary skill in the art is familiar with possible formulations and uses of depot injections.

The pharmaceutical compositions may be in the form of a sterile injectable aqueous or oleagenous suspension. This suspension may be formulated according to the known art using those suitable dispersing or wetting agents and suspending agents mentioned herein. The sterile injectable preparation may also be a sterile injectable solution or suspension in a non-toxic parenterally-acceptable diluent or solvent, for example, as a solution in 1,3-butane diol. Acceptable diluents, solvents and dispersion media that may be employed include water, Ringer's solution, isotonic sodium chloride solution, Cremophor EL™ (BASF, Parsippany, N.J.) or phosphate buffered saline (PBS), ethanol, polyol (e.g., glycerol, propylene glycol, and liquid polyethylene glycol), and suitable mixtures thereof. In addition, sterile, fixed oils are conventionally employed as a solvent or suspending medium. For this purpose, any bland fixed oil may be employed, including synthetic mono- or diglycerides. Moreover, fatty acids such as oleic acid, find use in the preparation of injectables. Prolonged absorption of particular injectable formulations can be achieved by including an agent that delays absorption (e.g., aluminum monostearate or gelatin).

The present invention contemplates the administration of the compound of formula (I) (and any embodiments thereof) in the form of suppositories for rectal administration. The suppositories can be prepared by mixing the drug with a suitable non-irritating excipient which is solid at ordinary temperatures but liquid at the rectal temperature and will therefore melt in the rectum to release the drug. Such materials include, but are not limited to, cocoa butter and polyethylene glycols.

The compound of formula (I) (and any embodiments thereof) contemplated by the present invention may also be in the form of any other suitable pharmaceutical composition (e.g., sprays for nasal or inhalation use) currently known or developed in the future.

For in vivo expansion, compounds of formula (I) (and any embodiments thereof) and compositions thereof can be administered in any appropriate manner. Suitable routes of administration include oral, parenteral (e.g., intramuscular, intravenous, subcutaneous (e.g., injection or implant), intraperitoneal, intracisternal, intraarticular, intraperitoneal, intracerebral (intraparenchymal) and intracerebroventricular), nasal, vaginal, sublingual, intraocular, rectal, topical (e.g., transdermal), buccal and inhalation. Depot injections, which are generally administered subcutaneously or intramuscularly, may also be utilized to release the AhR modulators disclosed herein over a defined period of time. Particular embodiments of the present invention contemplate oral administration.

Compounds of formula (I) (and any embodiments thereof) may be administered to a subject in an amount that is dependent upon, for example, the goal of administration (e.g., the degree of resolution desired); the age, weight, sex, and health and physical condition of the subject to which the formulation is being administered; the route of administration; and the nature of the disease, disorder, condition or symptom thereof. The dosing regimen may also take into consideration the existence, nature, and extent of any adverse effects associated with the agent(s) being administered. Effective dosage amounts and dosage regimens can readily be determined from, for example, safety and dose-escalation trials, in vivo studies (e.g., animal models), and other methods known to the skilled artisan.

In general, dosing parameters dictate that the dosage amount be less than an amount that could be irreversibly toxic to the subject (the maximum tolerated dose (MTD)) and not less than an amount required to produce a measurable effect on the subject. Such amounts are determined by, for example, the pharmacokinetic and pharmacodynamic parameters associated with ADME, taking into consideration the route of administration and other factors.

An effective dose (ED) is the dose or amount of an agent that produces a therapeutic response or desired effect in some fraction of the subjects taking it. The “median effective dose” or ED₅₀ of an agent is the dose or amount of an agent that produces a therapeutic response or desired effect in 50% of the population to which it is administered. Although the ED₅₀ is commonly used as a measure of reasonable expectance of an agent's effect, it is not necessarily the dose that a clinician might deem appropriate taking into consideration all relevant factors.

Thus, in some situations the effective amount is more than the calculated ED₅₀, in other situations the effective amount is less than the calculated ED₅₀, and in still other situations the effective amount is the same as the calculated ED₅₀.

In addition, an effective dose of a compound of formula (I) (and any embodiments thereof) may be an amount that, when administered in one or more doses to a subject, produces a desired result relative to a healthy subject. For example, for a subject experiencing a particular disorder, an effective dose may be one that improves a diagnostic parameter, measure, marker and the like of that disorder by at least about 5%, at least about 10%, at least about 20%, at least about 25%, at least about 30%, at least about 40%, at least about 50%, at least about 60%, at least about 70%, at least about 80%, at least about 90%, or more than 90%, where 100% is defined as the diagnostic parameter, measure, marker and the like exhibited by a normal subject.

In certain embodiments, compounds of formula (I) (and any embodiments thereof) contemplated by the present invention may be administered (e.g., orally) at dosage levels of about 0.01 mg/kg to about 50 mg/kg, or about 1 mg/kg to about 25 mg/kg, of subject body weight per day, one or more times a day, to obtain the desired therapeutic effect.

For administration of an oral agent, the compositions can be provided in the form of tablets, capsules and the like containing from 1.0 to 1000 milligrams of the active ingredient, particularly 1.0, 3.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient.

In certain embodiments, the dosage of the desired compound of formula (I) (and any embodiments thereof) is contained in a “unit dosage form”. The phrase “unit dosage form” refers to physically discrete units, each unit containing a predetermined amount of the AhR modulator, either alone or in combination with one or more additional agents, sufficient to produce the desired effect. It will be appreciated that the parameters of a unit dosage form will depend on the particular agent and the effect to be achieved.

The disease to be treated by the expanded stem cells according to the methods disclosed herein, including in vivo expansion, will depend on the specific type of stem cell. For example, expanded HSCs can be used for subjects whose bone marrow was destroyed by chemotherapy or radiation therapy used to treat some cancers. Expanded HSCs can be also used for subjects suffering from impaired hematopoiesis. In some embodiments the patient is suffering from a disease selected from the group consisting of Acute Lymphoblastic Leukemia (ALL), Acute Myelogenous Leukemia (AML), Chronic Myelogenous Leukemia (CML), Chronic Lymphocytic Leukemia (CLL), Hodgkin Lymphoma (HL), Non-Hodgkin Lymphoma (NHL), Myelodysplastic Syndrome (MDS), Multiple myeloma, Aplastic anemia, Bone marrow failure, Myeloproliferative disorders such as Myelofibrosis, Essential thrombocytopenia or Polycythemia vera, Fanconi anemia, Dyskeratosis congenita, Common variable immune deficiency (CVID, such as CVID 1, CVID 2, CVID 3, CVID 4, CVID 5, and CVID 6), Human immunodeficiency virus (HIV), Hemophagocytic lymphohistiocystosis, Amyloidosis, Solid tumors such as Neuroblastoma, Germ cell tumors, Breast cancer, Wilms' tumor, Medulloblastoma, and Neuroectodermal tumors, Autoimmune diseases such as Scleroderma, Multiple sclerosis, Ulcerative colitis, Systemic lupus erythematosus and Type I diabetes, or protein deficiencies such as Adrenoleukodystrophy (ALD), Metachromatic leukodystrophy (MLD), Hemophilia A & B, Hurler syndrome, Hunter syndrome, Fabry disease, Gaucher disease, Epidermolysis bullosa, Globoid Cell Leukodystrophy, Sanfillipo syndrome, and Morquio syndrome. In some embodiments, the patient is suffering from Sickle cell anemia, Alpha thalassemia, Beta thalassemia, Delta thalassemia, Hemoglobin E/thalassemia, Hemoglobin S/thalassemia, Hemoglobin C/thalassemia, Hemoglobin D/thalassemia, Chronic granulomatous disease (X-linked Chronic granulomatous disease, autosomal recessive (AR) chronic granulomatous disease, chronic granulomatous disease ARI NCF1, Chronic granulomatous disease AR CYBA, Chronic granulomatous disease AR II NCF2, Chronic granulomatous disease AR III NCF4, X-linked Severe Combined Immune Deficiency (SCID), ADA SCID, IL7-RA SCID, CD3 SCID, Rag1/Rag2 SCID, Artemis SCID, CD45 SCID, Jak3 SCID, Congenital agranulocytosis, Congenital agranulocytosis-congenital neutropenia-SCN1, Congenital agranulocytosis-congenital neutropenia-SCN2, Familial hemophagocytic lymphohistiocystosis (FHL), Familial hemophagocytic lymphohistiocytosis type 2 (FHL2, perforin mutation), Agammaglobulinemia (X-linked Agammaglobulinemia), Wiskott-Aldrich syndrome, Chediak-Higashi syndrome, Hemolytic anemia due to red cell pyruvate kinase deficiency, Paroxysmal nocturnal hemoglobinuria, X-linked Adrenoleukodystrophy (X-ALD), X-linked lymphoproliferative disease, Unicentric Castleman's Disease, Multicentric Castleman's Disease, Congenital amegakaryocytic, thrombocytopenia (CAMT) type I, Reticular dysgenesis, Fanconi anemia, Acquired idiopathic sideroblastic anemia, Systemic mastocytosis, Von willebrand disease (VWD), Congenital dyserythropoietic anemia type 2, Cartilage-hair hypoplasia syndrome, Hereditary spherocytosis, Blackfan-Diamond syndrome, Shwachman-Diamond syndrome, Thrombocytopenia-absent radius syndrome, Osteopetrosis, Infantile osteopetrosis, Mucopolysaccharidoses, Lesch-Nyhan syndrome, Glycogen storage disease, Congenital mastocytosis, Omenn syndrome, X-linked Immunodysregulation, polyendocrinopathy, and enteropathy (IPEX), IPEX characterized by mutations in FOXP3, X-linked syndrome of polyendocrinopathy, immune dysfunction, and diarrhea (XPID), X-Linked Autoimmunity-Allergic Dysregulation Syndrome (XLAAD), IPEX-like syndrome, Hyper IgM type 1, Hyper IgM type 2, Hyper IgM type 3, Hyper IgM type 4, Hyper IgM type 5, X linked hyperimmunoglobulin M, Bare lymphocyte Syndrome type I, and Bare lymphocyte Syndrome type II (Bare lymphocyte Syndrome type II, MHC class I deficiency; Bare lymphocyte Syndrome type II, complementation group A; Bare lymphocyte Syndrome type II, complementation group C; Bare lymphocyte Syndrome type II complementation group D; Bare lymphocyte Syndrome type II, complementation group E). Stem cells for use in the present disclosure for treatment may be the subject's own cells (autologous transplantation) or those of a donor (allogeneic transplantation). Accordingly, the disclosure further provides expanded HSCs or its composition for use in allogeneic or autologous stem cell transplantation in a subject.

Diseases treatable by MSCs include bone or cartilage disease, a neurodegenerative disease, a cardiac disease, a hepatic disease, cancer, nerve damage, wound healing, autoimmune disease, graft versus host disease, spinal cord injury and tissue regeneration. Bone defects suitable for treatment include, but are not limited to osteogenesis imperfecta, fracture, congenital bone defects, and the like. Further, the expanded mesenchymal stem cells of the present disclosure can be implanted in a subject to provide osseous and connective tissue Support of orthopedic and other (e.g. dental) prosthetic devices. Such as joint replacements and/or tooth implants. The mesenchymal stem cells of the present disclosure can be used to treat CNS diseases.

Representative examples of CNS diseases or disorders include, but are not limited to, a pain disorder, a motion disorder, a dissociative disorder, a mood disorder, an affective disorder, a neurodegenerative disease or disorder and a convulsive disorder. More specific examples of such conditions include, but are not limited to, Parkinson's, ALS, Multiple Sclerosis, Huntingdon's disease, autoimmune encephalomyelitis, diabetic neuropathy, glaucomatous neuropathy, macular degeneration, action tremors and tardive dyskinesia, panic, anxiety, depression, alcoholism, insomnia, manic behavior, Alzheimer's and epilepsy. As mentioned, since MSCs can differentiate into cartilage, the mesenchymal stem cells of the present disclosure may be suitable for the treatment of joint conditions including, but not limited to osteoarthritis, rheumatoid arthritis, inflammatory arthritis, chondromalacia, avascular necrosis, traumatic arthritis and the like.

Bone marrow-derived mesenchymal stem cells (MSCs) are known to interact with hematopoietic stem cells (HSCs) and immune cells and represent potential cellular therapy to enhance allogeneic hematopoietic engraftment and prevent graft-versus-host disease (GVHD).

In any of the methods above, when the stem cells are hematopoietic stems cells or progenitors thereof, they may be administered in combination with one or more mobilizing agents selected from the group consisting of a CXCR4 antagonist (e.g., AMD3100), GCSF, and GRO. A “mobilizing agent” is an agent capable of inducing the migration of expanded stem cells from the bone marrow of a subject to the peripheral blood.

### Assays

### Inhibition of AhR and Stem Cell Expansion:

The ability of compounds of formula (I) to antagonize AhR and facilitate expansion of stem cells can be assessed by using, for example, an art-accepted assay or model, examples of which are will be apparent to the skilled artisan. The ability of the compounds described herein to expand a population of hematopoietic stem cells is set forth in the Experimental section below.

### Exemplary Embodiments

Embodiment 1. A method of in vitro or ex-vivo culturing of stem cells and/or lineage committed progenitor cells comprising culturing a population of the stem cells and/or lineage committed progenitor cells in a medium comprising a compound of formula (I):

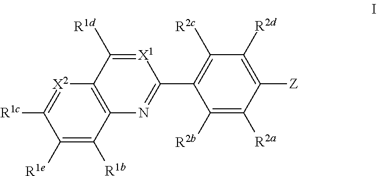

or a pharmaceutically acceptable salt, solvate or hydrate thereof, wherein: each of ring vertices X¹ and X² is independently selected from the group consisting of C(Ria) and N; Z is selected from the group consisting of:

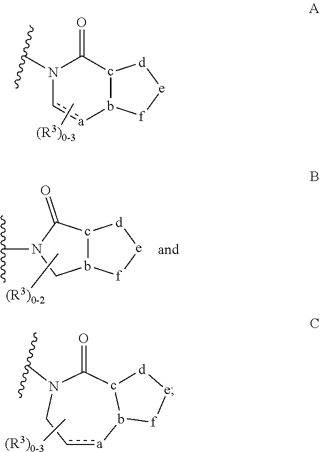

wherein the dashed bonds are single or double bonds, each of ring vertices a, b, c, d, e and f are independently selected from the group consisting of O, S, N, C, C(R⁴) and N(R⁴), and the bonds joining the ring vertices are independently single or double bonds; each R^{1a}, R^{1b}, R^{1c}, R^{1d} and R^{1e} is independently selected from the group consisting of hydrogen, deuterium, halogen, —CN, —NO₂, —R^{c}, —CO₂R^{a}, —CONR^{a}R^{b}, —C(O)R^{a}, —OC(O)NR^{a}R^{b}, —NR^{b}C(O)R^{a}, —NR^{b}C(O)₂R^{c}, —NR^{a}C(O)NR^{a}R^{b}, —NR^{a}R^{b}, —OR^{a}, and —S(O)₂NR^{a}R^{b}; wherein each R^{a} and R^{b} is independently selected from hydrogen, C₁₋₈ alkyl, C₃₋₆ cycloalkyl and C₁₋₈ haloalkyl, or when attached to the same nitrogen atom can be combined with the nitrogen atom to form a four-, five- or six-membered ring having from 0 to 2 additional heteroatoms as ring members selected from N, O, S, SO and SO₂; each R^{c} is independently selected from the group consisting of C₁₋₈ alkyl, C₁₋₈ haloalkyl, C₁₋₈ deuteroalkyl, C₂₋₆ alkenyl, C₂₋₆ alkynyl, and C₃₋₆ cycloalkyl, and wherein the aliphatic and cyclic portions of R^{a}, R^{b} and R^{c} can be further substituted with from one to three halogen, hydroxy, C₁₋₄ alkyl, C₁₋₄ alkoxy, amino, C₁₋₄ alkylamino, di C₁₋₄ alkylamino and carboxylic acid groups; each R^{2a}, R^{2b}, R^{2c} and R^{2d} is independently selected from the group consisting of hydrogen, halogen, C₁₋₃ alkyl, C₁₋₃ deuteroalkyl, C₁₋₃ haloalkyl, C₁₋₃ alkoxy, C₁₋₃ deuteroalkoxy and C₁₋₃ haloalkoxy; R³ is selected from the group consisting of hydrogen, deuterium, C₁₋₃ alkyl, C₁₋₃ deuteroalkyl, C₁₋₃ alkylene-OR^{d}, C₁₋₃ alkylene-CO₂R^{d}, C₁₋₃ alkylene-NR^{d}R^{e}, C₁₋₃ alkylene-CONR^{d}R^{e}, C₁₋₃ alkylene-OC(O)NR^{d}R^{e}, and C₁₋₃ alkylene-NR^{e}C(O)₂R^{f}; or two R³ groups are combined to form oxo (═O); each R⁴ is independently selected from the group consisting of hydrogen, halogen, —CN, —R^{f}, —CO₂R^{d}, —CONR^{d}R^{e}, —C(O)R^{d}, —OC(O)NR^{d}R^{e}, —NR^{e}C(O)R^{d}, —NR^{e}C(O)₂R^{f}, —NR^{d}C(O)NR^{d}R^{e}, —NR^{d}R^{e}, —OR^{d}, —S(O)₂NR^{d}R^{e}, —X^{a}—CN, —X^{a}—CO₂R^{d}, —X^{a}—CONR^{d}R^{e}, —X^{a}—C(O)R^{d}, —X^{a}—OC(O)NR^{d}R^{e}, —X^{a}—NR^{e}C(O)R^{d}, —X^{a}—NR^{e}C(O)₂R^{f}, —X^{a}—NR^{d}C(O) NR^{d}R^{e}, —X^{a}—NR^{d}R^{e}, —X^{a}—OR^{d}, and —X^{a}—S(O)₂NR^{d}R^{e}; wherein each X^{a} is independently C₁₋₆alkylene; and each R^{d} and R^{e} is independently selected from hydrogen, C₁₋₈ alkyl, and C₁₋₈ haloalkyl, or when attached to the same nitrogen atom can be combined with the nitrogen atom to form a four-, five- or six-membered ring having from 0 to 2 additional heteroatoms as ring members selected from N, O, S, SO and SO₂; each R^{f} is independently selected from the group consisting of C₁₋₈ alkyl, C₁₋₈ haloalkyl, C₁₋₈ deuteroalkyl, C₃₋₆ cycloalkyl, C₃₋₆ heterocycloalkyl, phenyl and 5- or 6-membered heteroaryl; and wherein the aliphatic and cyclic portions of R^{d}, R^{e} and R^{f} are can be further substituted with from one to three halogen, hydroxy, C₁₋₄ alkyl, C₁₋₄ alkoxy, amino, C₁₋₄ alkylamino, di C₁₋₄ alkylamino and carboxylic acid groups; wherein the compound of formula (I) antagonizes the activity of aryl hydrocarbon receptor.

Embodiment 2. The method of embodiment 1 wherein the stem cell and/or lineage committed progenitor cells are cultured under conditions that maintain functional potential of the stem cells and/or lineage committed progenitor cells. Preferably, the stem cells and progenitor cells are human cells. Preferably, the stem cells and progenitor cells are human cells and the method is carried out in ex vivo.

Embodiment 3. A method for producing an expanded population of stem cells and/or lineage committed progenitor cells in vitro or ex vivo comprising culturing a population of the stem cells and/or lineage committed progenitor cells in a medium comprising a compound of formula (I):

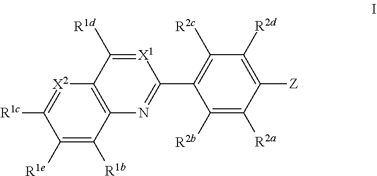

or a pharmaceutically acceptable salt, solvate or hydrate thereof, wherein: each of ring vertices X¹ and X² is independently selected from the group consisting of C(R^{1a}) and N; Z is selected from the group consisting of:

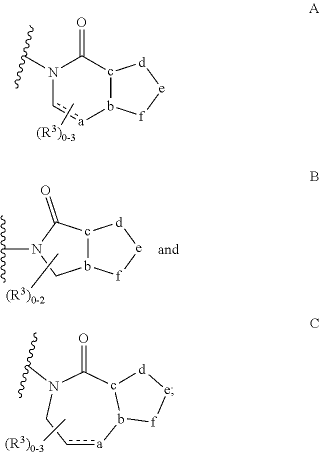

wherein the dashed bonds are single or double bonds, each of ring vertices a, b, c, d, e and f are independently selected from the group consisting of O, S, N, C, C(R⁴) and N(R⁴), and the bonds joining the ring vertices are independently single or double bonds; each R^{1a}, R^{1b}, R^{1c}, R^{1d} and R^{1e} is independently selected from the group consisting of hydrogen, deuterium, halogen, —CN, —NO₂, —R^{c}, —CO₂R^{a}, —CONR^{a}R^{b}, —C(O)R^{a}, —OC(O)NR^{a}R^{b}, —NR^{b}C(O)R^{a}, —NR^{b}C(O)₂R^{c}, —NR^{a}C(O)NR^{a}R^{b}, —NR^{a}R^{b}, —OR^{a}, and —S(O)₂NR^{a}R^{b}; wherein each R^{a} and R^{b} is independently selected from hydrogen, C₁₋₈ alkyl, C₃₋₆ cycloalkyl and C₁₋₈ haloalkyl, or when attached to the same nitrogen atom can be combined with the nitrogen atom to form a four-, five- or six-membered ring having from 0 to 2 additional heteroatoms as ring members selected from N, O, S, SO and SO₂; each R^{c} is independently selected from the group consisting of C₁₋₈ alkyl, C₁₋₈ haloalkyl, C₁₋₈ deuteroalkyl, C₂₋₆ alkenyl, C₂₋₆ alkynyl, and C₃₋₆ cycloalkyl, and wherein the aliphatic and cyclic portions of R^{a}, R^{b} and R^{c} can be further substituted with from one to three halogen, hydroxy, C₁₋₄ alkyl, C₁₋₄ alkoxy, amino, C₁₋₄ alkylamino, di C₁₋₄ alkylamino and carboxylic acid groups; each R^{2a}, R^{2b}, R^{2c} and R^{2d} is independently selected from the group consisting of hydrogen, halogen, C₁₋₃ alkyl, C₁₋₃ deuteroalkyl, C₁₋₃ haloalkyl, C₁₋₃ alkoxy, C₁₋₃ deuteroalkoxy and C₁₋₃ haloalkoxy; R³ is selected from the group consisting of hydrogen, deuterium, C₁₋₃ alkyl, C₁₋₃ deuteroalkyl, C₁₋₃ alkylene-OR^{d}, C₁₋₃ alkylene-CO₂R^{d}, C₁₋₃ alkylene-NR^{d}R^{e}, C₁₋₃ alkylene-CONR^{d}R^{e}, C₁₋₃ alkylene-OC(O)NR^{d}R^{e}, and C₁₋₃ alkylene-NR^{e}C(O)₂R^{f}; or two R³ groups are combined to form oxo (═O); each R⁴ is independently selected from the group consisting of hydrogen, halogen, —CN, —R^{f}, —CO₂R^{d}, —CONR^{d}R^{e}, —C(O)R^{d}, —OC(O)NR^{d}R^{e}, —NR^{e}C(O)R^{d}, —NR^{e}C(O)₂R^{f}, —NR^{d}C(O)NR^{d}R^{e}, —NR^{d}R^{e}, —OR^{d}, —S(O)₂NR^{d}R^{e}, —X^{a}—CN, —X^{a}—CO₂R^{d}, —X^{a}—CONR^{d}R^{e}, —X^{a}—C(O)R^{d}, —X^{a}—OC(O)NR^{d}R^{e}, —X^{a}—NR^{e}C(O)R^{d}, —X^{a}—NR^{e}C(O)₂R^{f}, —X^{a}—NR^{d}C(O) NR^{d}R^{e}, —X^{a}—NR^{d}R^{e}, —X^{a}—OR^{d}, and —X^{a}—S(O)₂NR^{d}R^{e}; wherein each X^{a} is independently C₁₋₆alkylene; and each R^{d} and R^{e} is independently selected from hydrogen, C₁₋₈ alkyl, and C₁₋₈ haloalkyl, or when attached to the same nitrogen atom can be combined with the nitrogen atom to form a four-, five- or six-membered ring having from 0 to 2 additional heteroatoms as ring members selected from N, O, S, SO and SO₂; each R^{f} is independently selected from the group consisting of C₁₋₈ alkyl, C₁₋₈ haloalkyl, C₁₋₈ deuteroalkyl, C₃₋₆ cycloalkyl, C₃₋₆ heterocycloalkyl, phenyl and 5- or 6-membered heteroaryl; wherein the aliphatic and cyclic portions of R^{d}, R^{e} and R^{f} are can be further substituted with from one to three halogen, hydroxy, C₁₋₄ alkyl, C₁₋₄ alkoxy, amino, C₁₋₄ alkylamino, di C₁₋₄ alkylamino and carboxylic acid groups; and wherein the compound of formula (I) antagonizes the activity of aryl hydrocarbon receptor; and the stem cells and/or progenitor cells are cultured under conditions allowing expansion of the stem cells and/or lineage committed progenitor cells.

Embodiment 4. The method of embodiment 3, further comprising differentiating the expanded stem cells to lineage committed progenitor cells thereof under conditions that cause differentiation of the expanded stem cells to lineage committed progenitor cells thereof.

Embodiment 5. The method of embodiments 3 or 4, wherein the method is carried out ex vivo.

Embodiment 6. The method of any one of embodiments 3 to 5, wherein the stem cells and/or lineage committed progenitor cells are human cells.

Embodiment 7. The method of embodiment 1 or 2 wherein the stem cells and/or lineage committed progenitor cells are hematopoietic stem cells and/or lineage committed hematopoietic progenitor cells.

Embodiment 8. The method of embodiment 1 or 2 wherein the stem cells and/or lineage committed progenitor cells are genetically modified hematopoietic stem cells and/or lineage committed hematopoietic progenitor cells thereof.

Embodiment 9. The method of embodiment 8 wherein the genetically modified hematopoietic stem cells and/or lineage committed hematopoietic progenitor cells thereof comprise an exogenous nucleic acid.

Embodiment 10. The method of any one of embodiments 3 to 6 wherein the stem cells and/or lineage committed hematopoietic progenitor cells are hematopoietic stem cells and/or lineage committed hematopoietic progenitor cells.

Embodiment 11. The method of any one of embodiments 3 to 6 wherein the stem cells and/or lineage committed hematopoietic progenitor cells are genetically modified hematopoietic stem cells and/or lineage committed hematopoietic progenitor cells thereof.

Embodiment 12. The method of embodiment 11 wherein the genetically modified hematopoietic stem cells and/or lineage committed hematopoietic progenitor cells thereof comprise an exogenous nucleic acid.

Embodiment 13. The method of anyone of embodiments 10-12 further comprising culturing the population of: (i) hematopoietic stem cells and/or lineage committed hematopoietic progenitor cells; or (ii) genetically modified hematopoietic stem cells and/or lineage committed genetically modified hematopoietic progenitor cells; in the presence of an agent that inhibits TGFβ signaling, preferably an agent that inhibits TGFβ receptor, more preferably the TGFβ receptor inhibitor is selected from the group consisting of 2-[3-(6-methyl-2-pyridinyl)-1H-pyrazol-4-yl]-1,5-naphthyridine, 4-[3-(2-pyridinyl)-1H-pyrazol-4-yl]-quinoline, 4-[6-(4-isopropoxyphenyl)-pyrazolo[1,5-a]pyrimidin-3-yl]quinoline, and 3-(6-methyl-2-pyridinyl)-N-phenyl-4-(4-quinolinyl)-1H-pyrazole-1-carbothioamide, even more preferably 3-(6-methyl-2-pyridinyl)-N-phenyl-4-(4-quinolinyl)-1H-pyrazole-1-carbothioamide.

Embodiment 14. The method of anyone of embodiments 10-13 further comprising culturing the population of: (i) hematopoietic stem cells and/or lineage committed hematopoietic progenitor cells; or (ii) genetically modified hematopoietic stem cells and/or lineage committed genetically modified hematopoietic progenitor cells; in the presence of a histone demethylase inhibitor, preferably, the histone demethylase inhibitor is a LSD1 inhibitor, preferably the LSD1 inhibitor is selected from the group consisting of 2-(1R,2S)-2-(4-(benzyloxy)phenyl)-cyclopropyl-amino)-1-(4-methylpiperazin-1-yl)ethanone, HCl, 2-(2-(benzyloxy)-3,5-difluorophenyl)-cyclopropan-1-amine, (1S,2S)—N-(1-(2,3-dihydrobenzo[b][1,4]dioxin-6-yl)ethyl)-2-phenylcyclopropan-1-amine, methyl-3-(4-(4-carbamimidoylbenzoyl)-piperazine-1-carbonyl)-5-((4-carbamimidoylpiperazin-1-yl)methyl)benzoate, (E,E)-1,1′-((propane-1,3-diylbis(azanediyl))bis(propane-3,1-diyl))bis(2,3-dimethylguanidine), and Tranylcypromine, more preferably the LSD1 inhibitor is Tranylcypromine.

Embodiment 15. The method of anyone of embodiments 10-14, further comprising culturing the population of: (i) hematopoietic stem cells and/or lineage committed hematopoietic progenitor cells; or (ii) genetically modified hematopoietic stem cells and/or lineage committed genetically modified hematopoietic progenitor cells; in the presence of a histone deacetylation inhibitor, preferably the histone deacetylase inhibitor is selected from the group consisting of Trichostatin A, valproic acid, butyrylhydroxamic acid, and istodax, more preferably the histone deacetylase inhibitor is Trichostatin A.

Embodiment 16. The method of anyone of embodiments 10-15, further comprising culturing the population of: (i) hematopoietic stem cells and/or lineage committed hematopoietic progenitor cells; or (ii) genetically modified hematopoietic stem cells and/or lineage committed genetically modified hematopoietic progenitor cells; in the presence of an agent that inhibits p38 signaling, preferably the p38 inhibitor is 4-(4-fluorophenyl)-2-(4-methylsulfinylphenyl)-5-(4-pyridyl)-1H-imidazole); and/or an agent that inhibits a protein that promotes β-catenin degradation, preferably the agent that inhibits β-catenin degradation is CHIR99021, LiCl, BIO, or FGF2.

Embodiment 17. The method of anyone of embodiments 10-16, further comprising culturing the population of: (i) hematopoietic stem cells and/or lineage committed hematopoietic progenitor cells; or (ii) genetically modified hematopoietic stem cells and/or lineage committed genetically modified hematopoietic progenitor cells; in the presence of an agent that reduces the activity of an ikaros family member transcription factor.

Embodiment 18. The method of embodiment 17, wherein the agent that reduces the activity of an ikaros family member transcription factor is pomalidomide, lenalidomide, or thalidomide.

Embodiment 19. The method of anyone of embodiments 10-18 further comprising culturing the population of hematopoietic stem cells and/or lineage committed hematopoietic progenitor cells or genetically modified hematopoietic stem cells and/or lineage committed genetically modified hematopoietic progenitor cells in the presence of a Notch agonist, preferably wherein the Notch Agonist is Delta-^{ext-IgG}.

Embodiment 20. The method of any one of embodiments 10 to 19 wherein the population of: (i) hematopoietic stem cells and/or lineage committed hematopoietic progenitor cells; or (ii) genetically modified stems cells and/or lineage committed genetically modified hematopoietic progenitor cells thereof, are cultured under conditions that maintain the functional potential of the hematopoietic stem cells and/or lineage committed hematopoietic progenitor cells or genetically modified stems cells and/or lineage committed genetically modified hematopoietic progenitor cells thereof.

Embodiment 21. The method of any one of embodiments 8-20 wherein the hematopoietic stem cells are from bone marrow, umbilical cord blood, or mobilized peripheral blood.

Embodiment 22. The method of any one of embodiments 10-21 wherein the hematopoietic stem cells and genetically modified hematopoietic stem cells are enriched in Endothelial Protein C Receptor (EPCR+) and/or CD34+, CD38+, CD90+, CD45RA+, CD133 and/or CD49f+, preferably CD34+ cells and/or EPCR+, more preferably CD34+ cells.

Embodiment 23. The method of embodiment 22 wherein the hematopoietic stem cells and genetically modified hematopoietic stem cells consist essentially of CD34+ cells.

Embodiment 24. The method of anyone of embodiments 10 to 23 further comprising culturing the population of: (i) hematopoietic stem cells and/or lineage committed hematopoietic progenitor cells; or (ii) genetically modified hematopoietic stem cells and/or lineage committed genetically modified hematopoietic progenitor cells; in the presence of a sufficient amount of one or more of IL6, Flt-3-L, TPO, and SCF, preferably in the presence of IL6, Flt-3-L, TPO, and SCF.

Embodiment 25. The method of any one of embodiments 1-24 wherein the amount of compound of formula (I) in the cell culture is from about 1 pM to about 100 μM, preferably from about 100 pM to about 10 μM.

Embodiment 26. The method of any one of embodiments 1 to 25 wherein the stem cells and/or lineage committed progenitor cells are cultured in the presence of a compound of formula (I), from about 1 days to about 90 days, preferably from about 2 to about 35 days, more preferably 5 days to 35 days, more preferably 5 days to 21 days.

Embodiment 27. The method of any one of embodiments 3 to 25 wherein the starting cell population is cultured in the presence of a compound of formula (I) during a time sufficient for about 2 to 50,000 fold expansion of hematopoietic stem cells and/or lineage committed hematopoietic progenitor cells or genetically modified hematopoietic stem cells and/or lineage committed genetically modified hematopoietic progenitor cells, preferably CD34+ cells, as compared to a population of hematopoietic stem cells and/or lineage committed hematopoietic progenitor cells or genetically modified hematopoietic stem cells and/or lineage committed genetically modified hematopoietic progenitor cells thereof cultured under the same conditions in the absence of a compound of formula (I).

Embodiment 28. The method of any one of embodiments 10-27, wherein the hematopoietic stem cells and/or lineage committed hematopoietic progenitor cells or genetically modified hematopoietic stem cells and/or lineage committed genetically modified hematopoietic progenitor cells thereof are contacted with said one or more agents simultaneously.

Embodiment 29. The method of any one of embodiments 10-27, wherein said the hematopoietic stem cells and/or lineage committed hematopoietic progenitor cells or genetically modified hematopoietic stem cells and/or lineage committed genetically modified hematopoietic progenitor cells thereof are contacted with said one or more agents at different times.

Embodiment 30. The method of any one of embodiments 10-21 and 24-29, wherein said hematopoietic stem cells and/or lineage committed hematopoietic progenitor cells are originally within a mononuclear cell fraction prior to treatment with said one or more agents.

Embodiment 31. The method of any one of embodiments 10-21 and 24 to 29, wherein said hematopoietic stem cells are originally within a CD34+, CD34+/CD38−, CD34+/CD38−/CD90+, CD34+/CD38−/CD90+/CD45RA−, or CD34+/CD38−/CD90+/CD45RA−/CD49F+ enriched cell fraction prior to contacting said one or more agents.

Embodiment 32. The method of any one of embodiments 10-21 and 24-29, wherein said hematopoietic stem cells and/or lineage committed hematopoietic progenitor cells are originally within an un-enriched cell fraction prior to contacting said one or more agents.

Embodiment 33. The method of any one of embodiments 1 to 32 wherein the compound of formula (I) is wherein Z has subformula A.

Embodiment 34. The method of embodiment 33 wherein the compound of formula (I) is wherein Z is selected from the group consisting of:

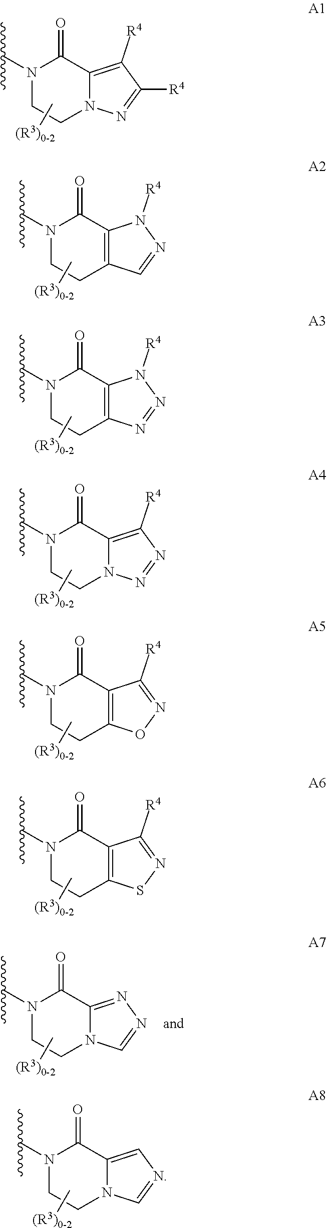

Embodiment 35. The method of any one of embodiments 1 to 32 wherein the compound of formula (I) is wherein Z has subformula B.

Embodiment 36. The method of embodiment 35 wherein Z is selected from the group consisting of:

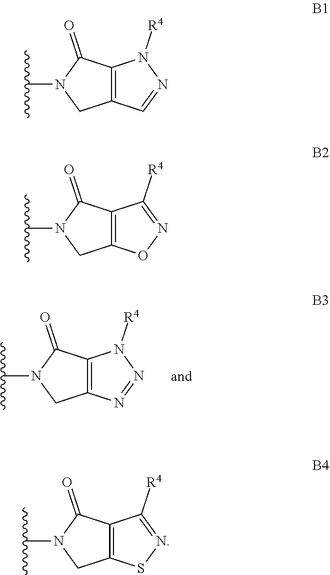

Embodiment 37. The method of any one of embodiments 1 to 32 wherein the compound of formula (I) is wherein Z has subformula C.

Embodiment 38. The method of embodiment 37 wherein Z is selected from the group consisting of:

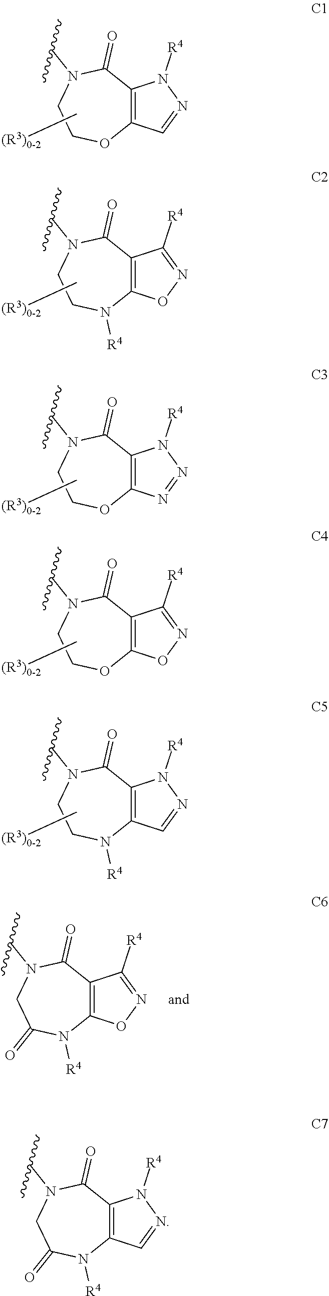

Embodiment 39. The method of any one of embodiments 1 to 38 wherein the compound of formula (I) is wherein X¹ is N.

Embodiment 40. The method of any one of embodiments 1 to 38 wherein the compound of formula (I) is wherein X² is N.

Embodiment 41. The method of anyone of embodiments 1 to 38 wherein the compound of formula (I) is wherein X¹ and X² are both N.

Embodiment 42. The method of any one of embodiments 1 to 41 wherein the compound of formula (I) is wherein R^{1b} is selected from the group consisting of H, deuterium, C₁₋₄ alkyl and C₁₋₄ haloalkyl.

Embodiment 43. The method of any one of embodiments 1 to 42 wherein the compound of formula (I) is wherein R^{1d} and R^{1e} are each H.

Embodiment 44. The method of any one of embodiments 1 to 43 wherein the compound of formula (I) is wherein R^{1c} is selected from the group consisting of H, deuterium, C₁₋₄ alkyl and C₁₋₄ haloalkyl.

Embodiment 45. The method of any one of embodiments 1 to 44 wherein the compound of formula (I) is wherein R² is selected from the group consisting of H, deuterium, halogen, C₁₋₄ alkyl and C₁₋₄ haloalkyl.

Embodiment 46. The method of any one of embodiments 1 to 45 wherein the compound of formula (I) is wherein R^{2b} is selected from the group consisting of H, deuterium, halogen, C₁₋₄ alkyl and C₁₋₄ haloalkyl.

Embodiment 47. The method of any one of embodiments 1 to 46 wherein the compound of formula (I) is wherein R^{2c} is selected from the group consisting of H, deuterium, halogen, C₁₋₄ alkyl and C₁₋₄ haloalkyl.

Embodiment 48. The method of any one of embodiments 1 to 47 wherein the compound of formula (I) is wherein R^{1b} is selected from the group consisting of H, deuterium and CH₃.

Embodiment 49. The method of any one of embodiments 1 to 48 wherein the compound of formula (I) is wherein R^{1c} is selected from the group consisting of H, deuterium, and CF₃.

Embodiment 50. The method of any one of embodiments 1 to 49 wherein the compound of formula (I) is wherein each of R^{2a}, R^{2b}, R^{2c} and R^{2d} are independently selected from the group consisting of H, deuterium, fluoro and CH₃.

Embodiment 51. The method of anyone of embodiments 1 to 50 wherein the compound of formula (I) is wherein each R⁴ is independently selected from the group consisting of H, halogen, —R^{f}, —CO₂R^{d}, —CONR^{d}R^{e}, —C(O)R^{d}, —NR^{e}C(O)R^{d}, —NR^{e}C(O)₂R^{f}, —NR^{d}C(O)NR^{d}R^{e}, —NR^{d}R^{e}, —OR^{d}, —X^{a}—CN, —X^{a}—CO₂R^{d}, —X^{a}—CONR^{d}R^{e}, —X^{a}—C(O)R^{d}, —X^{a}—OC(O)NR^{d}R^{e}, —X^{a}—NR^{e}C(O)R^{d}, —X^{a}—NR^{e}C(O)₂R^{f}, —X^{a}—NR^{d}C(O)NR^{d}R^{e}, —X^{a}—NR^{d}R^{e} and —X^{a}—OR^{d}; wherein each X^{a} is independently C₁₋₄alkylene.

Embodiment 52. The method of anyone of embodiments 1 to 51 wherein the compound of formula (I) is wherein each R³ is methyl.

Embodiment 53. The method of any one of embodiments 1 to 32 wherein the compound of formula (I) is a compound of formula (IIa), (IIb), (IIc), (IId), (IIe) or (IIf):

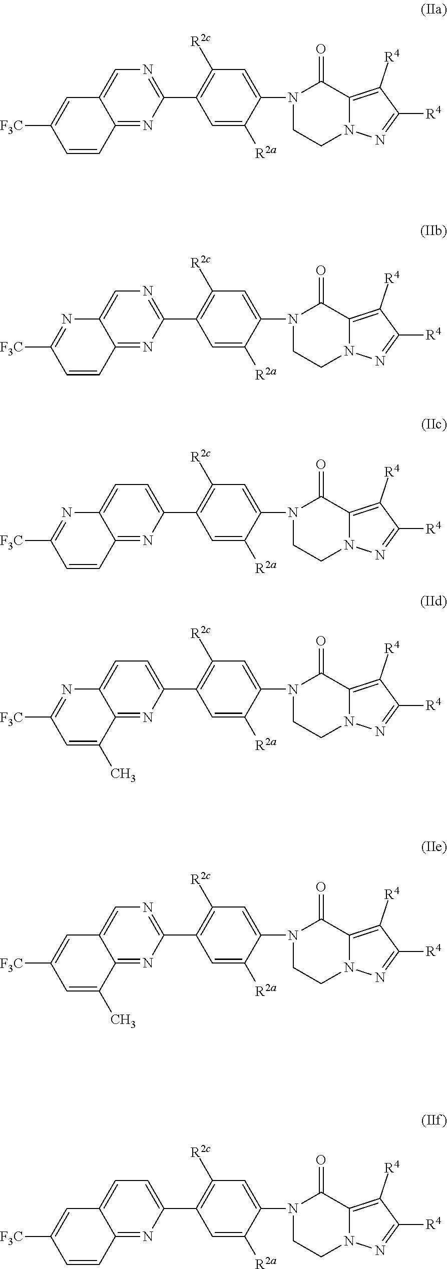

Embodiment 54. The method of any one of embodiments 1 to 32 wherein the compound of formula (I) is a compound of formula (IIIa), (IIIb), (IIIc), or (IIId):

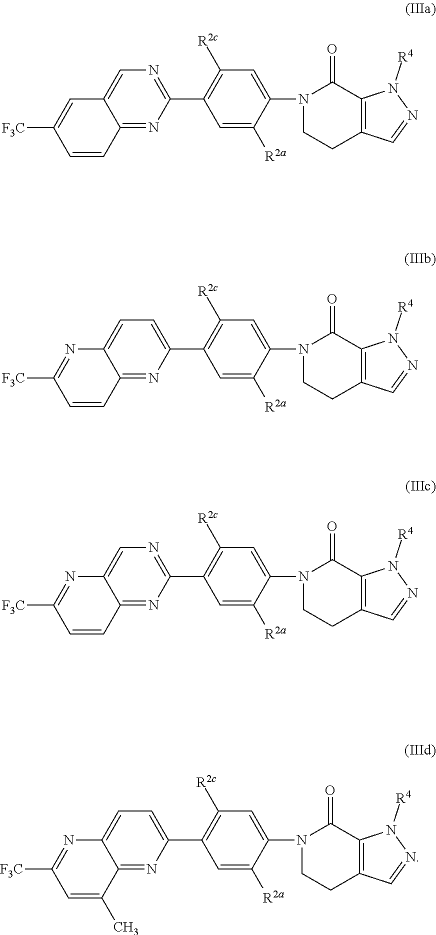

Embodiment 55. The method of any one of embodiments 1 to 32 wherein the compound of formula (I) is a compound of formula (IVa), (IVb), (IVc) or (IVd):

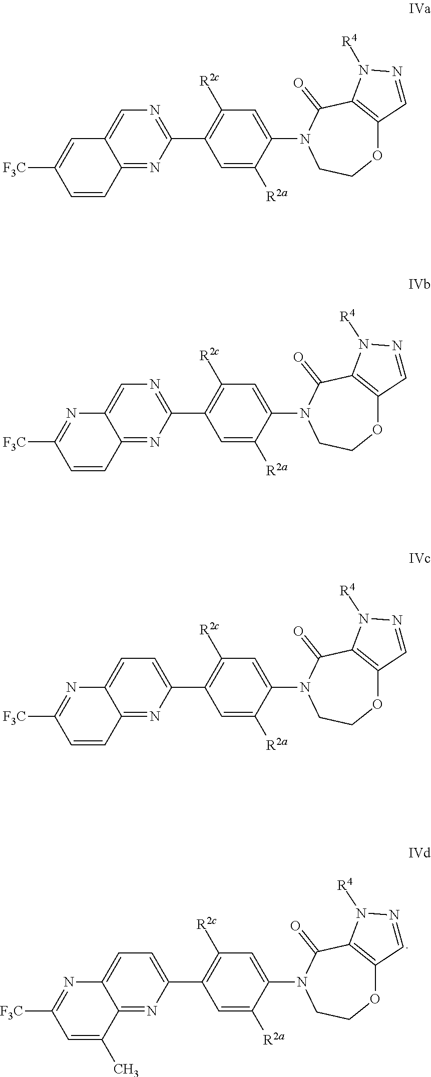

Embodiment 56. The method of any one of embodiments 1 to 32 wherein the compound of formula (I) is selected from Table 1.

Embodiment 57. The method of any one of embodiments 1 to 32 wherein the compound of formula (I) is selected from Table 1 and having +++ or ++++ activity.

Embodiment 58. The method of any one of embodiments 1 to 32 wherein the compound of formula (I) is selected from Table 1 and having ++++ activity.

Embodiment 59. An ex vivo or in vitro composition comprising a cell population of expanded hematopoietic stem cells and/or lineage committed progenitor cells thereof and a compound of formula (I), (IIa), (IIb), (IIc), (IId), (IIe), (IIf) (IIIa), (IIIb), (IIIc), (IIId), (IVa), (IVb), (IVc), (IVd) or a compound disclosed in Table 1.

Embodiment 60. A composition comprising a cell population of expanded hematopoietic stem cells and/or lineage committed hematopoietic progenitor cells obtained or obtainable by culturing ex vivo a starting population of cells comprising hematopoietic stem cells and/or lineage committed hematopoietic progenitor cells and a compound of formula (I) according to the method of any one of embodiments 10 and 13 to 58.

Embodiment 61. A composition comprising a cell population of expanded genetically modified hematopoietic stem cells and/or lineage committed genetically modified hematopoietic progenitor cells thereof obtained or obtainable by culturing ex vivo from a starting population of cells comprising genetically modified hematopoietic stem cells and/or lineage committed genetically modified hematopoietic progenitor cells thereof according to the method of any one of embodiments 11 to 58.

Embodiment 62. The composition of embodiment 60 or 61 substantially free of a compound of formula (I) and/or any other component of the cell culture medium.

Embodiment 63. The composition of any one of embodiments 59 to 62 further comprising a pharmaceutically acceptable medium.

Embodiment 64. The composition of any one of embodiments 59 to 62 suspended in a pharmaceutically acceptable medium suitable for transplantation into a patient in need thereof, preferably the patient is a human.

Embodiment 65. A method of treating a disease treatable by hematopoietic stem cell and/or lineage committed hematopoietic progenitor cell therapy comprising administering to a patient in need thereof a composition of any one of embodiments 59 to 64.

Embodiment 66. The method of embodiment 65 wherein the disease is selected from the group consisting of Acute Lymphoblastic Leukemia (ALL), Acute Myelogenous Leukemia (AML), Chronic Myelogenous Leukemia (CML), Chronic Lymphocytic Leukemia (CLL), Hodgkin Lymphoma (HL), Non-Hodgkin Lymphoma (NHL), Myelodysplastic Syndrome (MDS), Multiple myeloma, Aplastic anemia, Bone marrow failure, Myeloproliferative disorders such as Myelofibrosis, Essential thrombocytopenia or Polycythemia vera, Fanconi anemia, Dyskeratosis congenita, Common variable immune deficiency (CVID, such as CVID 1, CVID 2, CVID 3, CVID 4, CVID 5, and CVID 6), Human immunodeficiency virus (HIV), Hemophagocytic lymphohistiocystosis, Amyloidosis, Solid tumors such as Neuroblastoma, Germ cell tumors, Breast cancer, Wilms' tumor, Medulloblastoma, and Neuroectodermal tumors, Autoimmune diseases such as Scleroderma, Multiple sclerosis, Ulcerative colitis, Systemic lupus erythematosus and Type I diabetes, or protein deficiencies such as Adrenoleukodystrophy (ALD), Metachromatic leukodystrophy (MLD), Hemophilia A & B, Hurler syndrome, Hunter syndrome, Fabry disease, Gaucher disease, Epidermolysis bullosa, Globoid Cell Leukodystrophy, Sanfillipo syndrome, and Morquio syndrome.

Embodiment 67. The method of embodiment 65 wherein the disease is selected from Sickle cell anemia, Alpha thalassemia, Beta thalassemia, Delta thalassemia, Hemoglobin E/thalassemia, Hemoglobin S/thalassemia, Hemoglobin C/thalassemia, Hemoglobin D/thalassemia, Chronic granulomatous disease (X-linked Chronic granulomatous disease, autosomal recessive (AR) chronic granulomatous disease, chronic granulomatous disease ARI NCF1, Chronic granulomatous disease AR CYBA, Chronic granulomatous disease AR II NCF2, Chronic granulomatous disease AR III NCF4, X-linked Severe Combined Immune Deficiency (SCID), ADA SCID, IL7-RA SCID, CD3 SCID, Rag1/Rag2 SCID, Artemis SCID, CD45 SCID, Jak3 SCID, Congenital agranulocytosis, Congenital agranulocytosis-congenital neutropenia-SCN1, Congenital agranulocytosis-congenital neutropenia-SCN2, Familial hemophagocytic lymphohistiocystosis (FHL), Familial hemophagocytic lymphohistiocytosis type 2 (FHL2, perforin mutation), Agammaglobulinemia (X-linked Agammaglobulinemia), Wiskott-Aldrich syndrome, Chediak-Higashi syndrome, Hemolytic anemia due to red cell pyruvate kinase deficiency, Paroxysmal nocturnal hemoglobinuria, X-linked Adrenoleukodystrophy (X-ALD), X-linked lymphoproliferative disease, Unicentric Castleman's Disease, Multicentric Castleman's Disease, Congenital amegakaryocytic, thrombocytopenia (CAMT) type I, Reticular dysgenesis, Fanconi anemia, Acquired idiopathic sideroblastic anemia, Systemic mastocytosis, Von willebrand disease (VWD), Congenital dyserythropoietic anemia type 2, Cartilage-hair hypoplasia syndrome, Hereditary spherocytosis, Blackfan-Diamond syndrome, Shwachman-Diamond syndrome, Thrombocytopenia-absent radius syndrome, Osteopetrosis, Infantile osteopetrosis, Mucopolysaccharidoses, Lesch-Nyhan syndrome, Glycogen storage disease, Congenital mastocytosis, Omenn syndrome, X-linked Immunodysregulation, polyendocrinopathy, and enteropathy (IPEX), IPEX characterized by mutations in FOXP3, X-linked syndrome of polyendocrinopathy, immune dysfunction, and diarrhea (XPID), X-Linked Autoimmunity-Allergic Dysregulation Syndrome (XLAAD), IPEX-like syndrome, Hyper IgM type 1, Hyper IgM type 2, Hyper IgM type 3, Hyper IgM type 4, Hyper IgM type 5, X linked hyperimmunoglobulin M, Bare lymphocyte Syndrome type I, and Bare lymphocyte Syndrome type II (Bare lymphocyte Syndrome type IL, MHC class I deficiency; Bare lymphocyte Syndrome type II, complementation group A; Bare lymphocyte Syndrome type II, complementation group C; Bare lymphocyte Syndrome type II complementation group D; Bare lymphocyte Syndrome type IL, complementation group E).

Embodiment 68. A catheter comprising a cell population of expanded hematopoietic stem cells and/or lineage committed hematopoietic progenitor cells obtained or obtainable by culturing ex vivo a starting population of cells comprising hematopoietic stem cells and/or lineage committed hematopoietic progenitor cells and a compound of formula (I) according to the method of any one of embodiments 10 and 13 to 58.

Embodiment 69. A syringe comprising a cell population of expanded hematopoietic stem cells and/or lineage committed hematopoietic progenitor cells obtained or obtainable by culturing ex vivo a starting population of cells comprising hematopoietic stem cells and/or lineage committed hematopoietic progenitor cells and a compound of formula (I) according to the method of any one of embodiments 10 and 13 to 58.

### EXAMPLES

The following examples are put forth so as to provide those of ordinary skill in the art with a complete disclosure and description of how to make and use the present invention, and are not intended to limit the scope of what the inventors regard as their invention, nor are they intended to represent that the experiments below were performed or that they are all of the experiments that may be performed. It is to be understood that exemplary descriptions written in the present tense were not necessarily performed, but rather that the descriptions can be performed to generate data and the like of a nature described therein. Efforts have been made to ensure accuracy with respect to numbers used (e.g., amounts, temperature, etc.), but some experimental errors and deviations should be accounted for.

Unless indicated otherwise, parts are parts by weight, molecular weight is weight average molecular weight, temperature is in degrees Celsius (° C.), and pressure is at or near atmospheric. Standard abbreviations are used, including the following: μg=microgram; μl or μL=microliter; mM=millimolar; μM=micromolar; aa=amino acid(s); Ac₂O=acetic anhydride; AcCl=acetylchloride; ACN=acetonitrile; AIBN=2,2′-Azobis(2-methylpropionitrile); BID=twice daily; BINAP=2,2′-bis(diphenylphosphino)-1,1′-binaphthyl; Boc₂O or (Boc)₂O=di-tert-butyl dicarbonate; bp=base pair(s); BSA=bovine serum albumin; BW=body weight; d=doublet; dd=doublet of doublets; DEAD=diethyl azodicarboxylate; DIBAL=diisobutylaluminium hydride DIEA=N,N-diisopropylethylamine; DIPEA=N,N-diisopropylethylamine; dl or dL=deciliter; DMA=dimethylacetamide; DMAP=dimethylaminopyridine; DME=1,2-dimethoxyethane; DMEM=Dulbeco's Modification of Eagle's Medium; DMF=N,N-dimethylformamide; DMSO=dimethylsulfoxide; dppf=1,1′-Bis(diphenylphosphino)ferrocene; DTT=dithiothreitol; EDTA=ethylenediaminetetraacetic acid; ES=electrospray; EtOAc=ethyl acetate; EtOH=ethanol; g=gram; h or hr=hour(s); HATU=2-(1H-7-azabenzotriazol-1-yl)-1,1,3,3-tetramethyluronium hexafluorophosphate; HEPES=4-(2-hydroxyethyl)-1-piperazineethylanesulfonic acid; HOAc=acetic acid; HPLC=high performance liquid chromatography; HPLC=high pressure liquid chromatography; i.m.=intramuscular(ly); i.p.=intraperitoneal(ly); IHC=immunohistochemistry; IPA=isopropyl alcohol; kb=kilobase(s); kDa=kilodalton; kg=kilogram; 1 or L=liter; LC=liquid chromatography; LCMS=liquid chromatography and mass spectrometry; m/z=mass to charge ratio; M=molar; m=multiplet; MCN=acetonitrile; MeOH=methanol; MeSO₂Cl=methanesulfonylchloride; mg=milligram; min=minute(s); min=minutes; ml or mL=milliliter; mM=millimolar; MS=mass spectrometry; MsCl=methanesulfonylchloride; N=normal; NADPH=nicotinamide adenine dinucleotide phosphate; NBS=N-bromosuccinamide; ng=nanogram; nm=nanometer; nM=nanomolar; NMP=N-methylpyrrolidone; NMR=nuclear magnetic resonance; ns=not statistically significant; nt=nucleotides(s); PBS=phosphate-buffered saline; Pd/C=palladium on carbon; Pd₂(dba)₃=Tris(debenzylideneactone) dipalladium; Pd(dppf)Cl₂=1,1′-bis(diphenylphosphino)ferrocene-palladium(II) dichloride; PE=petroleum ether; QD=daily; QM=monthly; QW=weekly; rac=racemic; Rt=retention time; s=singlet; s or sec=second(s); sat.=saturated; SC or SQ=subcutaneous(ly); t=triplet; TBAB=tetra-n-butylammonium bromide; TEA=triethylamine; TFA=trifluoroacetic acid; THF=tetrahydrofuran; TLC=thin layer chromatography; TMSCl=trimethylsilylchloride; TsOH=p-toluenesulfonic acid; U=unit; wt=wildtype.

### Example 1

### Synthesis of 1-methyl-7-(2-methyl-4-(6-(trifluoromethyl)pyrido[3,2-d]pyrimidin-2-yl)phenyl)-6,7-dihydro-1H-pyrazolo[3,4-f][1,4]oxazepin-8(5H)-one

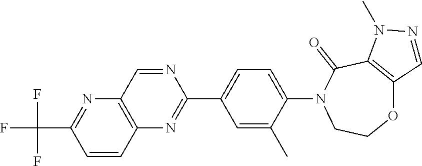 [SMILES: Cc1cc(-c2ncc3nc(C(F)(F)F)ccc3n2)ccc1N1CCOc2cnn(C)c2C1=O]

### Step 1: Synthesis of 2-bromo-6-(trifluoromethyl)pyridin-3-amine

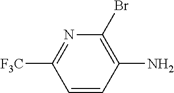 [SMILES: Nc1ccc(C(F)(F)F)nc1Br]

Into a 15-L 3-necked round-bottom flask, was placed 6-(trifluoromethyl)pyridin-3-amine (200 g, 1.23 mol, 1 equiv) and ACN (8 L) NBS (219.6 g, 1.23 mol, 1 equiv) was added in portions. The resulting solution was stirred for 3 h at 0° C. The reaction mixture was then quenched with water and the resulting solution was extracted with ethyl acetate. The organic layer was concentrated, and the residue was applied onto a silica gel column and eluted with ethyl acetate/petroleum ether (1/10) to give (146 g, 48.99%) of the title compound as a yellow solid. LC-MS: (ES, m/z): [M+H]⁺ 241.

### Step 2: Synthesis of 3-amino-6-(trifluoromethyl)picolinonitrile

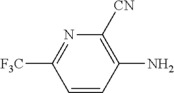 [SMILES: N#Cc1nc(C(F)(F)F)ccc1N]

Into a 3-L 3-necked round-bottom flask under N atmosphere, was placed 2-bromo-6-(trifluoromethyl)pyridin-3-amine (146 g, 605.7 mmol, 1 equiv), CuCN (271 g, 3029.5 mmol, 5 equiv), and DMSO (1400 mL). The resulting solution was stirred for 2 h at 120° C. The reaction was then quenched with water and the solids were filtered. The resulting solution was extracted with ethyl acetate and the organic layer concentrated. The residue was applied onto a silica gel column and eluted with ethyl acetate/petroleum ether (1/1) to give 85 mg of the title compound as a yellow solid. LC-MS: (ES, m/z): [M+H]⁺ 187.

### Step 3: Synthesis of 6-(trifluoromethyl)pyrido[3,2-d]pyrimidine-2,4(1H,3H)-dione

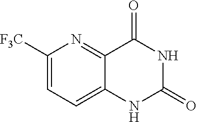 [SMILES: O=c1[nH]c(=O)c2nc(C(F)(F)F)ccc2[nH]1]

Into a 2-L 3-necked round-bottom flask, was placed 3-amino-6-(trifluoromethyl)picolinonitrile (30 g, 160.32 mmol, 1 equiv), DMF (300 mL), and DBU (73.2 g, 480.96 mmol, 3 equiv) under CO₂ atmosphere. The resulting solution was stirred for 14 h at 100° C. The reaction mixture was quenched with water. The pH value of the solution was adjusted to 5 with HCl (1 mol/L) and the solids were filtered to give (15 g, 40.54%) of the title compound as a yellow solid. LC-MS: (ES, m/z): [M+H]⁺ 231.

### Step 4: Synthesis of 2,4-dichloro-6-(trifluoromethyl)pyrido[3,2-d]pyrimidine

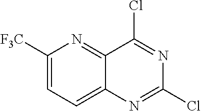 [SMILES: FC(F)(F)c1ccc2nc(Cl)nc(Cl)c2n1]

Into a 100 mL 3-necked round-bottom flask, was placed 6-(trifluoromethyl)pyrido[3,2-d]pyrimidine-2,4(1H,3H)-dione (15 g, 64.91 mmol, 1 equiv), POCl₃ (75 mL), and PCl₅ (67.5 g, 325.05 mmol, 5 equiv). The resulting solution was stirred for 6 h at 105° C. The resulting mixture was concentrated under vacuum, quenched with water/ice, and extracted with MTBE. The combined organic layers were concentrated, and the residue was applied onto a silica gel column and eluted with ethyl acetate/petroleum ether (1/50) to give (9.5 g, 54.59%) of the title compound as a white solid.

### Step 5: Synthesis of 2-chloro-6-(trifluoromethyl)pyrido[3,2-d]pyrimidine

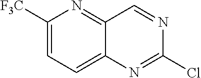 [SMILES: FC(F)(F)c1ccc2nc(Cl)ncc2n1]

Into a 250-mL 3-necked round-bottom flask under N₂ atmosphere, was placed 2,4-dichloro-6-(trifluoromethyl)pyrido[3,2-d]pyrimidine (9.5 g, 35.58 mmol, 1 equiv), THF (100 mL), PPh₃ (13.93 g, 53.37 mmol, 1.5 equiv), SnBu₃H (10.29 g 35.48 mmol, 1.1 equiv), and Pd(PPh₃)₄ (4.09 g, 3.54 mmol, 0.1 equiv). The resulting solution was stirred for 2 h at 0° C. The resulting mixture was concentrated under vacuum and then applied onto a silica gel column and eluted with ethyl acetate/petroleum ether (1/50) to give (4.5 g, 54.28%) of the title compound as a red solid.

### Step 6: Synthesis of 4-(2-(benzyloxy)ethoxy)-1-methyl-1H-pyrazole-5-carboxylic Acid

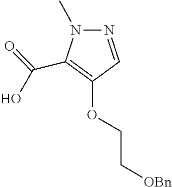 [SMILES: Cn1ncc(OCCOCc2ccccc2)c1C(=O)O]

To a stirred mixture of 4-bromo-1-methyl-1H-pyrazole-5-carboxylic acid (60 g, 292.67 mmol, 1 equiv) in 2-(benzyloxy)ethan-1-ol (300 mL) were added Cs₂CO₃ (286.1 g, 878.00 mmol, 3.00 equiv) and CuCl₂ (3.9 g, 29.27 mmol, 0.10 equiv). The resulting mixture was stirred for overnight at 130° C. under nitrogen atmosphere. The reaction was quenched with water and extracted with EtOAc. The aqueous phase was acidified to pH 2 with conc. HCl and the resulting mixture was extracted with EtOAc. The combined organic layers were concentrated under reduced pressure. The residue was purified by reverse flash chromatography under the following conditions: column, C18 silica gel; mobile phase, MeCN in water, 0% to 35% gradient in 25 min; detector, UV 254 nm to give (19.2 g, 23.74%) of the title compound as a white solid. LC-MS: (ES, m/z): [M+H]⁺ 277.

### Step 7: Synthesis of 4-(2-(benzyloxy)ethoxy)-1-methyl-1H-pyrazole-5-carbonyl Chloride

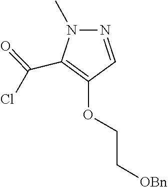 [SMILES: Cn1ncc(OCCOCc2ccccc2)c1C(=O)Cl]

To a stirred solution of 4-(2-(benzyloxy)ethoxy)-1-methyl-H-pyrazole-5-carboxylic acid (10 g, 36.19 mmol, 1 equiv) and DMF (0.3 g, 3.62 mmol, 0.10 equiv) in DCM (200 mL) was added (COCl)₂ (6.9 g, 54.29 mmol, 1.5 equiv) dropwise at 0° C. under nitrogen atmosphere. The resulting mixture was stirred for 3 h at room temperature under nitrogen atmosphere and then concentrated under vacuum to give (11.5 g, crude) of the title compound as a white solid.

### Step 8: Synthesis of 4-(2-(benzyloxy)ethoxy)-N-(4-bromo-2-methylphenyl)-1-methyl-1H-pyrazole-5-carboxamide

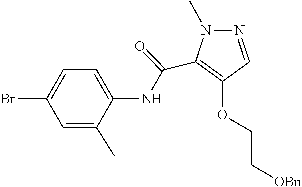 [SMILES: Cc1cc(Br)ccc1NC(=O)c1c(OCCOCc2ccccc2)cnn1C]

To a stirred solution of 4-bromo-2-methylaniline (6.2 g, 33.59 mmol, 1.1 equiv) and Et₃N (4.6 g, 45.80 mmol, 1.5 equiv) in DCM (200 mL) was added 4-(2-(benzyloxy)ethoxy)-1-methyl-1H-pyrazole-5-carbonyl chloride (9 g, 30.54 mmol, 1 equiv) in portions at 0° C. The resulting mixture was stirred for 1 h at room temperature and then quenched with water. The resulting mixture was extracted with CH₂Cl₂ and the combined organic layers were concentrated under reduced pressure. The crude product was re-crystallized from MeOH (50 mL) to afford (13 g, 95.82%) of the title compound as a white solid. LC-MS: (ES, m/z): [M+H]⁺ 444, 446.

### Step 9: Synthesis of N-(4-bromo-2-methylphenyl)-4-(2-hydroxyethoxy)-1-methyl-1H-pyrazole-5-carboxamide

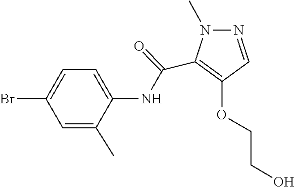 [SMILES: Cc1cc(Br)ccc1NC(=O)c1c(OCCO)cnn1C]

To a stirred solution of 4-(2-(benzyloxy)ethoxy)-N-(4-bromo-2-methylphenyl)-1-methyl-1H-pyrazole-5-carboxamide (13 g, 29.26 mmol, 1 equiv) in DCM (130 mL) was added BCl₃ (44 mL, 561.38 mmol, 19.19 equiv) dropwise at 0° C. The resulting mixture was stirred for 5 h at room temperature. The reaction was quenched with NaHCO₃ (aq.) (300 mL), extracted with CH₂Cl₂, dried over anhydrous sodium sulfate, and filtered. The combined organic layers were concentrated to give 10.5 g (crude) of the title compound as a yellow solid. LC-MS: (ES, m/z): [M+H]⁺ 354, 356.

### Step 10: Synthesis of 2-((5-((4-bromo-2-methylphenyl)carbamoyl)-1-methyl-1H-pyrazol-4-yl)oxy)ethyl Methanesulfonate

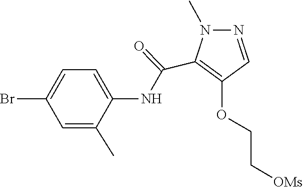 [SMILES: Cc1cc(Br)ccc1NC(=O)c1c(OCCOS(C)(=O)=O)cnn1C]

To a stirred solution of N-(4-bromo-2-methylphenyl)-4-(2-hydroxyethoxy)-1-methyl-1H-pyrazole-5-carboxamide (10.5 g, 29.64 mmol, 1 equiv) and Et₃N (9.0 g, 88.93 mmol, 3 equiv) in DCM (100 mL) was added MsCl (6.8 g, 59.29 mmol, 2 equiv) dropwise at 0° C. The resulting mixture was stirred for 5 h at room temperature. The reaction mixture was quenched with water and the resulting mixture was extracted with CH₂Cl₂. The combined organic layers were washed with water, dried over anhydrous Na₂SO₄. After filtration, the filtrate was concentrated under reduced pressure to give (13.4 g, 104.57%) of the title compound as a yellow solid. LC-MS: (ES, m/z): [M+H]⁺ 432, 434.

### Step 11: Synthesis of 7-(4-bromo-2-methylphenyl)-1-methyl-6,7-dihydro-1H-pyrazolo[3,4-f][1,4]-oxazepin-8(5H)-one

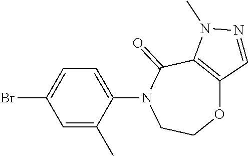 [SMILES: Cc1cc(Br)ccc1N1CCOc2cnn(C)c2C1=O]

To a stirred solution of 2-((5-((4-bromo-2-methylphenyl)carbamoyl)-1-methyl-1H-pyrazol-4-yl)-oxy)ethyl methanesulfonate (13.4 g, 31.00 mmol, 1 equiv) in DMF (200 mL, 2584.35 mmol, 83.37 equiv) were added NaH (1.86 g, 77.51 mmol, 2.50 equiv) in portions at 0° C. The resulting mixture was stirred for 3 h at room temperature. The reaction was quenched with sat. NH₄Cl (aq.) and the resulting mixture was extracted with EtOAc. The combined organic layers were concentrated under reduced pressure. The residue was purified by silica gel column chromatography with PE/EtOAc (5/1) as eluent to afford (8 g, 76.77%) of the title compound as a yellow solid. LC-MS: (ES, m/z): [M+H]⁺ 336, 338.

### Step 12: Synthesis of 1-methyl-7-(2-methyl-4-(4,4,5,5-tetramethyl-1,3,2-dioxaborolan-2-yl)phenyl)-6,7-dihydro-1H-pyrazolo[3,4-f][1,4]oxazepin-8(5H)-one

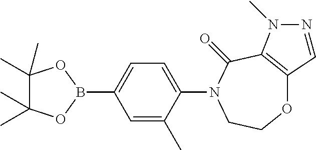 [SMILES: Cc1cc(B2OC(C)(C)C(C)(C)O2)ccc1N1CCOc2cnn(C)c2C1=O]

To a stirred mixture of 7-(4-bromo-2-methylphenyl)-1-methyl-6,7-dihydro-1H-pyrazolo[3,4-f]-[1,4]oxazepin-8(5H)-one (8 g, 23.80 mmol, 1 equiv) and B₂Pin₂(7.3 g, 28.56 mmol, 1.2 equiv) in dioxane (160 mL) were added KOAc (4.7 g, 47.59 mmol, 2 equiv) and Pd(dppf)Cl₂ (1.7 g, 2.38 mmol, 0.1 equiv). The resulting mixture was stirred for overnight at 80° C. under nitrogen atmosphere and the mixture was concentrated under reduced pressure. The residue was purified by silica gel column chromatography with PE/EtOAc (3/1) as eluent to afford (7.1 g, 77.85%) of the title compound as a white solid. LC-MS: (ES, m/z): [M+H]⁺ 384.

### Step 13: Synthesis of 1-methyl-7-(2-methyl-4-(6-(trifluoromethyl)pyrido[3,2-d]pyrimidin-2-yl)phenyl)-6,7-dihydro-1H-pyrazolo[3,4-f][1,4]oxazepin-8(5H)-one

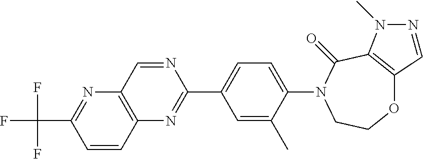 [SMILES: Cc1cc(-c2ncc3nc(C(F)(F)F)ccc3n2)ccc1N1CCOc2cnn(C)c2C1=O]

To a stirred solution of 1-methyl-7-(2-methyl-4-(4,4,5,5-tetramethyl-1,3,2-dioxaborolan-2-yl)phenyl)-6,7-dihydro-1H-pyrazolo[3,4-f][1,4]oxazepin-8(5H)-one (140 mg, 0.37 mmol, 1 equiv) and 2-chloro-6-(trifluoromethyl)pyrido[3,2-d]pyrimidine (140 mg, 0.60 mmol, 1.64 equiv) in t-BuOH (3 mL) were added H₂O (0.33 mL), K₂CO₃ (151.5 mg, 1.10 mmol, 3 equiv) and AMPhosPdCl₂ (77.4 mg, 0.11 mmol, 0.3 equiv). The resulting mixture was stirred for overnight at 80° C. under nitrogen atmosphere and then diluted with water. The resulting mixture was extracted with EtOAc and the combined organic layers were concentrated under reduced pressure. The residue was purified by Prep-TLC (CH₂Cl₂/MeOH=50/1) to afford (33.5 mg, 20.18%) of the title compound as a yellow solid. LC-MS: (ES, m/z): [M+H]⁺ 455; ¹H-NMR: (300 MHz, CD₃Cl, ppm): δ2.44 (s, 3H), δ4.00-4.02 (m, 2H), δ4.18 (s, 3H), δ4.53-4.55 (m, 2H), δ7.26-7.36 (m, 2H), δ8.14-8.17 (d, 1H), δ8.59-8.66 (m, 2H), 9.83 (s, 1H).

### Example 2

### Synthesis of 3-methyl-5-(2-methyl-4-(6-(trifluoromethyl)quinazolin-2-yl)phenyl)-6,7-dihydropyrazolo[1,5-a]pyrazin-4(5H)-one

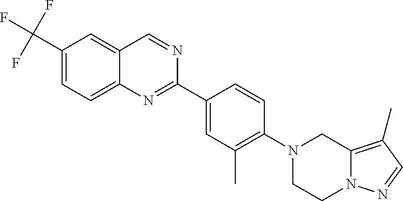 [SMILES: Cc1cc(-c2ncc3cc(C(F)(F)F)ccc3n2)ccc1N1CCn2ncc(C)c2C1]

### Step 1: Synthesis of 6-(trifluoromethyl)quinazoline-2,4(1H,3H)-dione

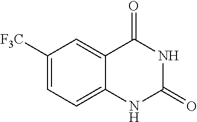 [SMILES: O=c1[nH]c(=O)c2cc(C(F)(F)F)ccc2[nH]1]

To a stirred solution of 2-amino-5-(trifluoromethyl)benzonitrile (30 g, 161.17 mmol, 1 equiv) in DMF (900 mL) was added DBU (73.6 g, 483.46 mmol, 3.00 equiv). The resulting mixture was stirred overnight at 100° C. under carbon dioxide atmosphere and then diluted with water. The reaction mixture was acidified to pH 5 with HCl (aq.), filtered, and the filter cake was washed with water. The resulting solid was dried in an oven under reduced pressure to give (32 g, 86.27%) of the title compound as a light yellow solid. LC-MS: (ES, m/z): [M+H]⁺ 230.

### Step 2: Synthesis of 2,4-dichloro-6-(trifluoromethyl)quinazoline

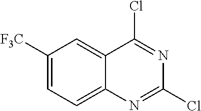 [SMILES: FC(F)(F)c1ccc2nc(Cl)nc(Cl)c2c1]

Into a 1 L 3-necked round-bottom flask was added 6-(trifluoromethyl)quinazoline-2,4(1H,3H)-dione (1:1) (57.8 g, 256.36 mmol, 1 equiv), PC15 (266.9 g, 1281.80 mmol, 5.00 equiv) and POCl₃ (295 mL). The resulting mixture was stirred for overnight at 120° C. and then concentrated under reduced pressure. The residue was quenched with water/ice at 0° C. and the resulting mixture was extracted with MTBE. The combined organic layers were dried over anhydrous Na₂SO₄. After filtration, the filtrate was concentrated under reduced pressure. The residue was purified by silica gel column chromatography with PE as eluent to afford (50.0 g, 74.79%) of the title compound as a light yellow solid. GC-MS: (ES, m/z): [M] 266.

### Step 3: Synthesis of 2-chloro-6-(trifluoromethyl)quinazoline

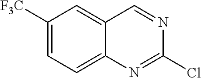 [SMILES: FC(F)(F)c1ccc2nc(Cl)ncc2c1]

To a stirred solution of 2,4-dichloro-6-(trifluoromethyl)quinazoline (58 g, 218.05 mmol, 1 equiv) and tributylstannane (74.7 g, 256.64 mmol, 1.10 equiv) in THF (623 mL) were added Pd(pph₃)₄ (27.0 g, 23.33 mmol, 0.1 equiv) under nitrogen atmosphere. The resulting mixture was stirred overnight at room temperature under nitrogen atmosphere. The resulting mixture was concentrated under reduced pressure and the residue was purified by silica gel column chromatography with PE/EtOAc (10/1) as eluent to afford the title compound (28 g, 55.36%) as a yellow solid. LC-MS: (ES, m/z): [M+H]⁺ 233.

### Step 4: Synthesis of ethyl 1-(2-(benzyloxy)ethyl)-4-methyl-1H-pyrazole-5-carboxylate

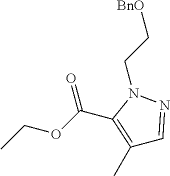 [SMILES: CCOC(=O)c1c(C)cnn1CCOCc1ccccc1]

Into a 50-mL 3-necked round-bottom flask purged under nitrogen atmosphere, was placed ethyl 4-methyl-1H-pyrazole-5-carboxylate (2 g, 12.97 mmol, 1 equiv), 2-(benzyloxy)ethan-1-ol (2.0 g 13.14 mmol, 1.01 equiv), DIAD (3.9 g, 19.46 mmol, 1.5 equiv), and PPh₃ (5.1 g, 19.46 mmol, 1.5 equiv) in THF (20 mL). The resulting solution was stirred for overnight at room temperature. After concentrating the reaction mixture under vacuum, the residue was applied onto a silica gel column and eluted with ethyl acetate/petroleum ether (1/50) to give (3 g, 80.20%) of the title compound as colorless oil. LC-MS: (ES, m/z): [M+H]⁺ 288.

### Step 5: Synthesis of 1-(2-(benzyloxy)ethyl)-4-methyl-1H-pyrazole-5-carboxylic Acid

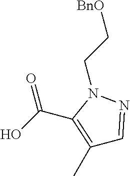 [SMILES: Cc1cnn(CCOCc2ccccc2)c1C(=O)O]

Into a 50-mL 3-necked round-bottom flask, was placed ethyl 1-(2-(benzyloxy)ethyl)-4-methyl-1H-pyrazole-5-carboxylate (2.9 g, 10.06 mmol, 1 equiv), NaOH (4M, 2 equiv), and EtOH (15 mL). The resulting solution was stirred for 2 h at 50° C. and then diluted with H₂O. The resulting mixture was concentrated to remove EtOH. The pH value of the solution was adjusted to 4 with HCl (1 mol/L). The resulting solution was extracted with ethyl acetate, dried over anhydrous sodium sulfate, and concentrated to give (2.1 g, 80.22%) of the title compound as a white solid. LC-MS: (ES, m/z): [M+H]⁺ 260.

### Step 6: Synthesis of 1-(2-(benzyloxy)ethyl)-N-(4-bromo-2-methylphenyl)-4-methyl-1H-pyrazole-5-carboxamide

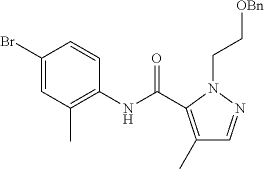 [SMILES: Cc1cc(Br)ccc1NC(=O)c1c(C)cnn1CCOCc1ccccc1]

Into a 50-mL 3-necked round-bottom flask, was placed 1-(2-(benzyloxy)ethyl)-4-methyl-1H-pyrazole-5-carboxylic acid (1.2 g, 4.61 mmol, 1 equiv), 4-bromo-2-methylaniline (1.3 g, 6.99 mmol, 1.52 equiv), HATU (2.6 g, 6.92 mmol, 1.5 equiv), DIEA (1.2 g, 9.22 mmol, 2 equiv), and DMF (15 mL). The resulting solution was stirred for overnight at room temperature. The resulting solution was diluted with EtOAc and washed with H₂O. The resulting mixture was concentrated under vacuum and the residue was applied onto a silica gel column and eluted with ethyl acetate/petroleum ether (1/10) to give (1.6 g, 81.03%) of the title compound as a white solid. LC-MS: (ES, m/z): [M+H]⁺ 428, 430.

### Step 7: Synthesis of N-(4-bromo-2-methylphenyl)-1-(2-hydroxyethyl)-4-methyl-1H-pyrazole-5-carboxamide

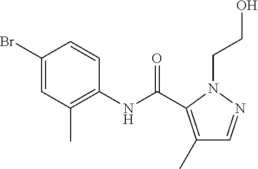 [SMILES: Cc1cc(Br)ccc1NC(=O)c1c(C)cnn1CCO]

Into a 50-mL 3-necked round-bottom flask under nitrogen atmosphere, was placed 1-(2-(benzyloxy)ethyl)-N-(4-bromo-2-methylphenyl)-4-methyl-1H-pyrazole-5-carboxamide (1 g, 1 equiv), and BCl₃ (1 mol/mL, 5 mL) was added dropwise at 0° C. The resulting solution was stirred for 1 h at 0° C. and then quenched with NaHCO₃. The resulting solution was extracted with ethyl acetate and the organic layers were combined. The organic layer was washed with H₂O, dried over anhydrous sodium sulfate and concentrated to give (512 mg, 67.65%) of the title compound as a white solid. LC-MS: (ES, m/z): [M+H]⁺ 338, 340.

### Step 8: Synthesis of 2-(5-((4-bromo-2-methylphenyl)carbamoyl)-4-methyl-1H-pyrazol-1-yl)ethyl methanesulfonate

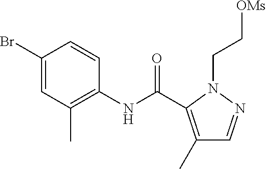 [SMILES: Cc1cc(Br)ccc1NC(=O)c1c(C)cnn1CCOS(C)(=O)=O]

Into a 25-mL 3-necked round-bottom flask, was placed N-(4-bromo-2-methylphenyl)-1-(2-hydroxyethyl)-4-methyl-1H-pyrazole-5-carboxamide (312 mg, 0.92 mmol, 1 equiv), DCM (5 mL), and MsCl (158.5 mg, 1.38 mmol, 1.5 equiv) was added dropwise at 0° C. The resulting solution was stirred for 1 h at RT and then quenched with water. The resulting solution was extracted with dichloromethane and the organic layers combined. The resulting mixture was washed with H₂O, dried, filtered and concentrated to give (416 mg) of the title compound as a crude product. LC-MS: (ES, m/z): [M+H]⁺ 416, 418.

### Step 9: Synthesis of 5-(4-bromo-2-methylphenyl)-3-methyl-6,7-dihydropyrazolo[1,5-a]pyrazin-4(5H)-one

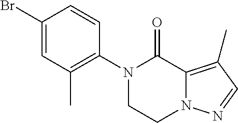 [SMILES: Cc1cc(Br)ccc1N1CCn2ncc(C)c2C1=O]

Into a 25-mL 3-necked round-bottom flask under N₂ atmosphere, was placed 2-(5-((4-bromo-2-methylphenyl)carbamoyl)-4-methyl-1H-pyrazol-1-yl)ethyl methanesulfonate (416 mg, 1.00 mmol, 1 equiv), DMF (5 mL, 64.61 mmol, 64.65 equiv), and NaH (36.0 mg, 1.50 mmol, 1.5 equiv) was added in portions at 0° C. The resulting solution was stirred for 1 h at 0° C. The reaction mixture was then quenched by added to 20 mL of NH₄Cl and extracted with ethyl acetate. The organic layer was washed with H₂O and then concentrated. The residue was applied onto a prep-TLC with ethyl acetate/petroleum ether (1/5) to give (300 mg, 93.76%) of the title compound as a white solid. LC-MS: (ES, m/z): [M+H]⁺ 320, 322.

### Step 10: Synthesis of 3-methyl-5-(2-methyl-4-(4,4,5,5-tetramethyl-1,3,2-dioxaborolan-2-yl)phenyl)-6,7-dihydropyrazolo[1,5-a]pyrazin-4(5H)-one

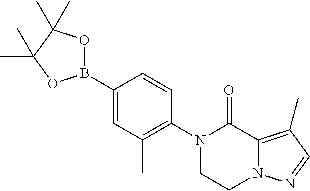 [SMILES: Cc1cc(B2OC(C)(C)C(C)(C)O2)ccc1N1CCn2ncc(C)c2C1=O]

Into a 8-mL sealed tube purged under nitrogen atmosphere, was placed 5-(4-bromo-2-methylphenyl)-3-methyl-6,7-dihydropyrazolo[1,5-a]pyrazin-4(5H)-one (280 mg, 0.87 mmol, 1 equiv), 4,4,5,5-tetramethyl-2-(4,4,5,5-tetramethyl-1,3,2-dioxaborolan-2-yl)-1,3,2-dioxaborolane (333.1 mg, 1.31 mmol, 1.5 equiv), Pd(dppf)Cl₂ (96.0 mg, 0.13 mmol, 0.15 equiv), and KOAc (171.6 mg, 1.75 mmol, 2 equiv), in dioxane (3 mL). The resulting solution was stirred for overnight at 80° C. The resulting mixture was concentrated under vacuum and the residue was applied onto a silica gel column and eluted with ethyl acetate/petroleum ether (1/5) to give (204 mg, 63.52%) of the title compound as a white solid. LC-MS: (ES, m/z): [M+H]⁺ 367.

### Step 11: Synthesis of 2-methyl-5-(2-methyl-4-(6-(trifluoromethyl)quinazolin-2-yl)phenyl)-6,7-dihydropyrazolo[1,5-a]pyrazin-4(5H)-one

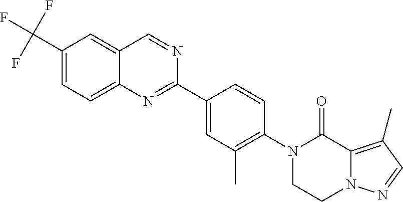 [SMILES: Cc1cc(-c2ncc3cc(C(F)(F)F)ccc3n2)ccc1N1CCn2ncc(C)c2C1=O]

Into a 50-mL 3-necked round-bottom flask under nitrogen atmosphere, was placed 3-methyl-5-(2-methyl-4-(4,4,5,5-tetramethyl-1,3,2-dioxaborolan-2-yl)phenyl)-6,7-dihydro-pyrazolo[1,5-a]pyrazin-4(5H)-one (180 mg, 0.49 mmol, 1 equiv), 2-chloro-6-(trifluoromethyl)-quinazoline (114.0 mg, 0.49 mmol, 1.00 equiv), Pd(PPh₃)₄ (85.0 mg, 0.07 mmol, 0.15 equiv), and K₂CO₃ (203.2 mg, 1.47 mmol, 3.00 equiv) in toluene (5.4 mL) and EtOH (2.7 mL). The resulting solution was stirred for overnight at 80° C. The resulting solution was diluted with H₂O, extracted with ethyl acetate, and the organic layers were combined. The residue was applied onto a silica gel column and eluted with ethyl acetate/petroleum ether (1/2) to give (31.8 mg, 14.83%) of the title compound as a light yellow solid. LC-MS: (ES, m/z): [M+H]⁺ 438; H-NMR: (300 MHz, DMSO-d6, ppm): δ 9.89 (s, 1H), 8.71 (s, 1H), 8.54 (d, 1H), 8.47 (d, 1H), 8.27 (d, 2H), 7.54 (d, 1H), 7.46 (d, 1H), 4.51 (t, 2H), 4.29 (m, 7.1 Hz, 1H), 3.91 (m, 1H), 2.33 (s, 3H), 2.24 (s, 3H).

### Example 3

### Synthesis of 5-(2,5-dimethyl-4-(6-(trifluoromethyl)quinazolin-2-yl)phenyl)-3-methyl-6,7-dihydropyrazolo[1,5-a]pyrazin-4(5H)-one

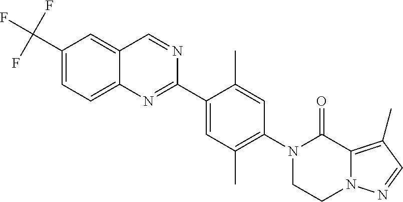 [SMILES: Cc1cc(N2CCn3ncc(C)c3C2=O)c(C)cc1-c1ncc2cc(C(F)(F)F)ccc2n1]

### Step 1: Synthesis of 1-(2-(benzyloxy)ethyl)-4-methyl-1H-pyrazole-5-carbonyl Chloride

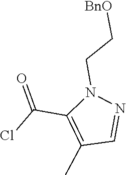 [SMILES: Cc1cnn(CCOCc2ccccc2)c1C(=O)Cl]

Into a 250-mL 3-necked round-bottom flask under N₂ atmosphere, was placed 1-(2-(benzyloxy)ethyl)-4-methyl-1H-pyrazole-5-carboxylic acid (6.4 g, 24.59 mmol, 1 equiv), prepared as described in Example 2 above, DCM (60 mL, 943.80 mmol, 38.39 equiv), DMF (0.2 g, 2.74 mmol, 0.11 equiv.), and (COCl)₂ (3.4 g, 27.05 mmol, 1.1 equiv) was added dropwise to the above solution at 0° C. The resulting solution was stirred for 1 h at RT and then concentrated under vacuum to give 6.9 g of the title compound as a crude product.

### Step 2: Synthesis of 1-(2-(benzyloxy)ethyl)-N-(4-bromo-2,5-dimethylphenyl)-4-methyl-1H-pyrazole-5-carboxamide

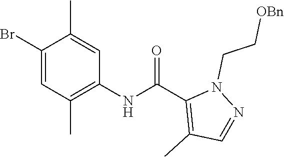 [SMILES: Cc1cc(NC(=O)c2c(C)cnn2CCOCc2ccccc2)c(C)cc1Br]

Into a 100 mL 3-necked round-bottom flask was added 4-bromo-2,5-dimethylaniline (3.3 g, 16.50 mmol, 1 equiv), DCM (50 mL), and Et₃N (3.3 g, 32.61 mmol, 1.98 equiv). To the above mixture 1-(2-(benzyloxy)ethyl)-4-methyl-1H-pyrazole-5-carbonyl chloride (4.6 g, 16.50 mmol, 1 equiv) in 20 mL of DCM was added dropwise at 0° C. The resulting mixture was stirred for additional 1 h at room temperature. The reaction mixture was quenched with water and the aqueous layer was extracted with CH₂Cl₂. The combined organic layer was dried over anhydrous sodium sulfate and concentrated under vacuum. The crude product was re-crystallized from MeOH (50 mL) to afford (5.4 g, 73.97%) of the title compound as a white solid. LC-MS: (ES, m/z): [M+H]⁺ 442.

### Step 3: Synthesis of N-(4-bromo-2,5-dimethylphenyl)-1-(2-hydroxyethyl)-4-methyl-1H-pyrazole-5-carboxamide

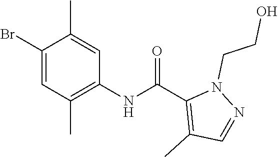 [SMILES: Cc1cc(NC(=O)c2c(C)cnn2CCO)c(C)cc1Br]

Into a 250-mL 3-necked round-bottom flask under nitrogen atmosphere was placed 1-(2-(benzyloxy)ethyl)-N-(4-bromo-2,5-dimethylphenyl)-4-methyl-1H-pyrazole-5-carboxamide (5.4 g, 1 equiv), DCM (50 mL), and BCl₃ (18.3 mL, 1M in DCM, 1.5 equiv) was added dropwise at 0° C. The resulting solution was stirred for 1 h at RT. After quenching with NaHCO₃ (aq.), the reaction mixture was extracted with dichloromethane. The combined organic layer was washed with H₂O, dried over anhydrous sodium sulfate and concentrated under vacuum to give (4.1 g, 95.35%) of the title compound as a white solid. LC-MS: (ES, m/z): [M+H]⁺ 352.

### Step 4: Synthesis of 2-(5-((4-bromo-2,5-dimethylphenyl)carbamoyl)-4-methyl-1H-pyrazol-1-yl)ethyl Methanesulfonate

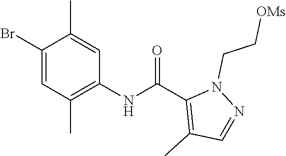 [SMILES: Cc1cc(NC(=O)c2c(C)cnn2CCOS(C)(=O)=O)c(C)cc1Br]

Into a 100-mL 3-necked round-bottom flask under nitrogen atmosphere, was placed N-(4-bromo-2,5-dimethylphenyl)-1-(2-hydroxyethyl)-4-methyl-1H-pyrazole-5-carboxamide (4.1 g, 11.64 mmol, 1 equiv), DCM (40 mL), Et₃N (2.4 g, 23.72 mmol, 2.04 equiv), and MsCl (2.0 g, 17.46 mmol, 1.50 equiv) was added dropwise to the above mixture at 0° C. The resulting solution was stirred for 1 h at RT. The reaction mixture was then quenched with water. The resulting solution was extracted with dichloromethane and the organic layers combined. The organic layer was washed with H₂O, dried over anhydrous sodium sulfate and concentrated under vacuum to give 4.75 g of the title compound as a crude product. LC-MS: (ES, m/z): [M+H]⁺ 430.

### Step 5: Synthesis of 5-(4-bromo-2,5-dimethylphenyl)-3-methyl-6,7-dihydropyrazolo[1,5-a]pyrazin-4(5H)-one

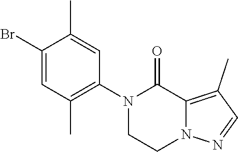 [SMILES: Cc1cc(N2CCn3ncc(C)c3C2=O)c(C)cc1Br]

Into a 100-mL 3-necked round-bottom flask under nitrogen atmosphere, was added 2-(5-((4-bromo-2,5-dimethylphenyl)carbamoyl)-4-methyl-1H-pyrazol-1-yl)ethyl methanesulfonate (4.75 g, 11.04 mmol, 1 equiv), and DMF (50 mL), and NaH (0.4 g, 16.67 mmol, 1.51 equiv) was added at 0° C. The resulting solution was stirred for 1 h at RT and then quenched with NH₄Cl (aq.). The resulting solution was extracted with ethyl acetate and the combined organic layer was washed with H₂O and concentrated. The residue was applied onto a silica gel column and eluted with ethyl acetate/petroleum ether (1:5) to give (3.1 g, 84.03%) of the title compound as a white solid. LC-MS: (ES, m/z): [M+H]⁺ 334.

### Step 6: Synthesis of 5-(2,5-dimethyl-4-(4,4,5,5-tetramethyl-1,3,2-dioxaborolan-2-yl)phenyl)-3-methyl-6,7-dihydropyrazolo[1,5-a]pyrazin-4(5H)-one

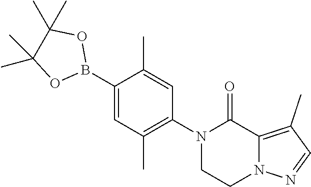 [SMILES: Cc1cc(N2CCn3ncc(C)c3C2=O)c(C)cc1B1OC(C)(C)C(C)(C)O1]

Into a 100-mL 3-necked round-bottom flask under nitrogen atmosphere, was placed 5-(4-bromo-2,5-dimethylphenyl)-3-methyl-6,7-dihydropyrazolo[1,5-a]pyrazin-4(5H)-one (3.1 g, 9.28 mmol, 1 equiv), B₂Pin₂ (3.54 g 13.92 mmol, 1.5 equiv), dioxane (30 mL), KOAc (1.8 g, 18.34 mmol, 1.98 equiv), and Pd(dppf)Cl₂ (2.0 g 2.78 mmol, 0.3 equiv). The resulting solution was stirred overnight at 80° C. The resulting mixture was concentrated under vacuum. The residue was applied onto a silica gel column and eluted with ethyl acetate/petroleum ether (1:5) to give (3 g, 84.83%) of the title compound as a white solid. LC-MS: (ES, m/z): [M+H]⁺ 382.

### Step 7: Synthesis of 5-(2,5-dimethyl-4-(6-(trifluoromethyl)quinazolin-2-yl)phenyl)-3-methyl-6,7-dihydropyrazolo[1,5-a]pyrazin-4(5H)-one

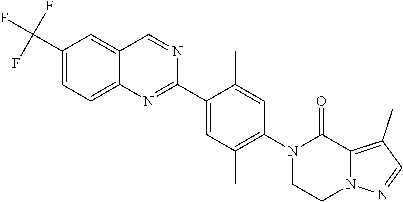 [SMILES: Cc1cc(N2CCn3ncc(C)c3C2=O)c(C)cc1-c1ncc2cc(C(F)(F)F)ccc2n1]

Into a 50-mL 3-necked round-bottom flask under nitrogen atmosphere, was placed 5-(2,5-dimethyl-4-(4,4,5,5-tetramethyl-1,3,2-dioxaborolan-2-yl)phenyl)-3-methyl-6,7-dihydropyrazolo[1,5-a]pyrazin-4(5H)-one (1 g, 2.62 mmol, 1 equiv), 2-chloro-6-(trifluoromethyl)quinazoline (0.7 g, 2.89 mmol, 1.1 equiv), K₂CO₃ (1.1 g 7.87 mmol, 3 equiv), toluene (14 mL), EtOH (7 mL), and Pd(PPh₃)₄ (0.5 g 0.39 mmol, 0.15 equiv). The resulting solution was stirred for 12 h at 80° C. The resulting mixture was diluted with and extracted with ethyl acetate. The combined organic layer was concentrated under reduced pressure. The residue was applied onto a silica gel column and eluted with ethyl acetate/petroleum ether (1:2) to give (307.1 mg, 25.94%) of the title compound as a white solid. LC-MS: (ES, m/z): [M+H]⁺ 452; ¹H-NMR: (300 MHz, DMSO-d6, ppm): δ 9.91 (s, 1H), 8.75 (s, 1H), 8.24-8.32 (m, 2H), 7.95 (s, 1H), 7.39-7.48 (m, 2H), 4.51-4.54 (m, 2H), 4.26-4.32 (m, 1H), 3.90-3.95 (m, 1H), 2.50-2.60 (m, 3H), 2.29 (s, 6H).

### Example 4

### Synthesis of 1-methyl-6-(2-methyl-4-(6-(trifluoromethyl)pyrido[3,2-d]pyrimidin-2-yl)phenyl)-1,4,5,6-tetrahydro-7H-pyrazolo[3,4-c]pyridin-7-one

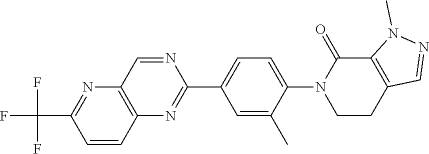 [SMILES: Cc1cc(-c2ncc3nc(C(F)(F)F)ccc3n2)ccc1N1CCc2cnn(C)c2C1=O]

### Step 1: Synthesis of methyl 4-allyl-1-methyl-H-pyrazole-5-carboxylate

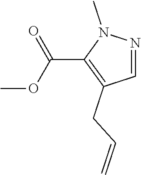

To a stirred solution of methyl 4-bromo-1-methyl-H-pyrazole-5-carboxylate (100 g, 456.54 mmol, 1 equiv) and tributyl(prop-2-en-1-yl)stannane (166.3 g, 502.20 mmol, 1.1 equiv) in DMF (1000 mL) was added Pd(PPh₃)₄ (26.4 g, 22.83 mmol, 0.05 equiv). The resulting mixture was stirred overnight at 100° C. under nitrogen atmosphere and then was quenched with water. The resulting mixture was extracted with EtOAc. The combined organic layers were concentrated under reduced pressure. The residue was purified by silica gel column chromatography with PE/EtOAc (50/1) as eluent to afford (80 g, 97.24%) of the title compound as a yellow oil. LC-MS: (ES, m/z): [M+H]⁺ 181.

### Step 2. Synthesis of 4-allyl-1-methyl-1H-pyrazole-5-carboxylic Acid

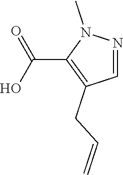

Into a 2-L 3-necked round-bottom flask, was placed methyl 4-allyl-1-methyl-1H-pyrazole-5-carboxylate (84 g, 466.13 mmol, 1 equiv), MeOH (840 mL, 20747.08 mmol, 44.51 equiv), NaOH (37.3 g, 932.57 mmol, 2.00 equiv). The resulting solution was stirred for 2 h at 50° C. and then diluted with H₂O. The resulting mixture was concentrated under vacuum to remove CH₃OH. The pH of the solution was adjusted to 5 with HCl (1 mol/L). The resulting solution was extracted with ethyl acetate and the organic layers combined and concentrated to give (61 g, 78.75%) of the title compound as a white solid. LC-MS: (ES, m/z): [M+H]⁺ 167.

### Step 3: Synthesis of 4-allyl-1-methyl-H-pyrazole-5-carbonyl Chloride

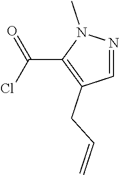

Into a 500-mL round-bottom flask was placed 4-allyl-1-methyl-H-pyrazole-5-carboxylic acid (20 g, 120.35 mmol, 1 equiv) in DCM (200 mL, 3146.01 mmol, 26.14 equiv), and DMF (0.9 g, 12.04 mmol, 0.1 equiv). Oxalyl chloride (22.9 g, 180.42 mmol, 1.50 equiv) was added dropwise over 10 min at 0° C. The resulting reaction mixture was concentrated to give (21 g, 94.51%) of the title compound as a crude product.

### Step 4: Synthesis of 4-allyl-N-(4-bromo-2-methylphenyl)-1-methyl-1H-pyrazole-5-carboxamide

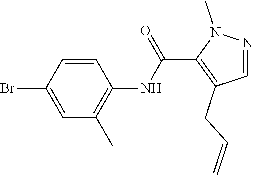 [SMILES: C=CCc1cnn(C)c1C(=O)Nc1ccc(Br)cc1C]

Into a 500-mL 3-necked round-bottom flask, was placed 4-bromo-2-methylaniline (25.4 g, 136.52 mmol, 1.20 equiv), Et₃N (17.3 g, 170.62 mmol, 1.5 equiv), and DCM. 4-Allyl-1-methyl-1H-pyrazole-5-carbonyl chloride (21 g, 113.75 mmol, 1 equiv) was added dropwise with stirring at 0° C. The resulting solution was stirred for 30 min at rt and then quenched with water. The resulting solution was extracted with DCM and the organic layers combined and concentrated. The crude product was purified by re-crystallization from CH₃OH to give (33 g, 86.81%) of the title compound as a white solid. LC-MS: (ES, m/z): [M+H]⁺ 334.

### Step 5: Synthesis of N-(4-bromo-2-methylphenyl)-4-(2,3-dihydroxypropyl)-1-methyl-1H-pyrazole-5-carboxamide

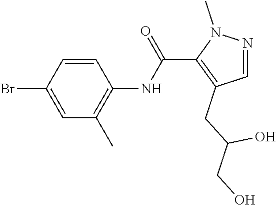 [SMILES: Cc1cc(Br)ccc1NC(=O)c1c(CC(O)CO)cnn1C]

Into a 500-mL 3-necked round-bottom flask was placed 4-allyl-N-(4-bromo-2-methylphenyl)-1-methyl-1H-pyrazole-5-carboxamide (21 g, 62.83 mmol, 1 equiv), NMO (22.1 g, 188.50 mmol, 3 equiv), THF (210 mL, 2.91 mmol, 0.05 equiv), H₂O (21 mL, 1165.68 mmol, 18.55 equiv), and OsO₄ (0.8 g, 3.15 mmol, 0.05 equiv). The resulting solution was stirred for 30 min at room temperature. The reaction mixture was quenched with 300 mL of NaS₂O₄. The resulting solution was extracted with ethyl acetate, water and then concentrated to give (37 g, 159.92%) of the title compound as a crude product. LC-MS: (ES, m/z): [M+H]⁺ 368.

### Step 6: Synthesis of N-(4-bromo-2-methylphenyl)-1-methyl-4-(2-oxoethyl)-1H-pyrazole-5-carboxamide

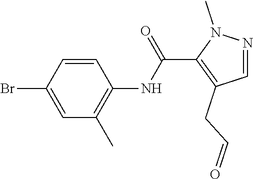 [SMILES: Cc1cc(Br)ccc1NC(=O)c1c(CC=O)cnn1C]

Into a 500-mL 3-necked round-bottom flask, was placed N-(4-bromo-2-methylphenyl)-4-(2,3-dihydroxypropyl)-1-methyl-1H-pyrazole-5-carboxamide (37 g, 100.48 mmol, 1 equiv), MeOH (370 mL), H₂O (37 mL), and NaIO₄ (43.0 g, 201.04 mmol, 2.00 equiv). The resulting solution was stirred for 30 min at room temperature, diluted with 370 mL of H₂O, and the resulting mixture was concentrated. The solids were filtrated, washed with H₂O and dried to give 35 g (crude) of the title compound as a white solid. LC-MS: (ES, m/z): [M+H]⁺ 336.

### Step 7: Synthesis of N-(4-bromo-2-methylphenyl)-4-(2-hydroxyethyl)-1-methyl-1H-pyrazole-5-carboxamide

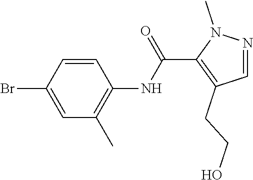 [SMILES: Cc1cc(Br)ccc1NC(=O)c1c(CCO)cnn1C]

Into a 1 L 3-necked round-bottom flask was placed N-(4-bromo-2-methylphenyl)-1-methyl-4-(2-oxoethyl)-1H-pyrazole-5-carboxamide (35 g, 104.11 mmol, 1 equiv) in MeOH (350 mL). NaBH₄ (4.7 g, 124.23 mmol, 1.19 equiv) was added in portions at 0° C. The resulting solution was stirred for 30 min at rt and then quenched with NH₄Cl solution (350 mL). The resulting mixture was concentrated and the solids were collected by filtration, washed with H₂O, dried to give (30 g, 85.20%) of the title compound as a white solid. LC-MS: (ES, m/z): [M+H]⁺ 338.

### Step 8: Synthesis of 2-(5-((4-bromo-2-methylphenyl)carbamoyl)-1-methyl-1H-pyrazol-4-yl)ethyl Methanesulfonate

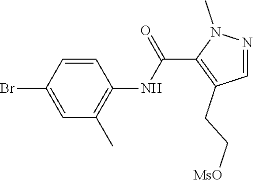 [SMILES: Cc1cc(Br)ccc1NC(=O)c1c(CCOS(C)(=O)=O)cnn1C]

Into a 1-L round-bottom flask, was placed N-(4-bromo-2-methylphenyl)-4-(2-hydroxyethyl)-1-methyl-1H-pyrazole-5-carboxamide (30 g, 88.70 mmol, 1 equiv), DCM (300 mL), and Et₃N (13.5 g, 133.41 mmol, 1.50 equiv). Methanesulfonyl chloride (15.2 g, 132.69 mmol, 1.50 equiv) was added dropwise with stirring at 0° C. The resulting solution was stirred for 30 min at rt and then quenched with water. The resulting solution was extracted with dichloromethane and concentrated to give (42 g, 113.74%) of the title compound as a white solid. LC-MS: (ES, m/z): [M+H]⁺ 416.

### Step 9: Synthesis of 6-(4-bromo-2-methylphenyl)-1-methyl-1,4,5,6-tetrahydro-7H-pyrazolo[3,4-c]pyridin-7-one

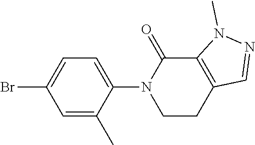 [SMILES: Cc1cc(Br)ccc1N1CCc2cnn(C)c2C1=O]

Into a 1 L 3-necked round-bottom flask was placed 2-(5-((4-bromo-2-methylphenyl)carbamoyl)-1-methyl-1H-pyrazol-4-yl)ethyl methanesulfonate (42 g, 100.89 mmol, 1 equiv), and DMF (420 mL, 5427.14 mmol, 53.79 equiv). NaH (3.6 g, 150.01 mmol, 1.49 equiv) was added in portions at 0° C. The resulting solution was stirred for 30 min at room temperature and then quenched with NH₄Cl. The solids were collected by filtration. The crude product was purified by re-crystallization from PE to give (23 g, 71.20%) of the title compound as a white solid. LC-MS: (ES, m/z): [M+H]⁺.

### Step 10: Synthesis of 1-methyl-6-(2-methyl-4-(4,4,5,5-tetramethyl-1,3,2-dioxaborolan-2-yl)phenyl)-1,4,5,6-tetrahydro-7H-pyrazolo[3,4-c]pyridin-7-one

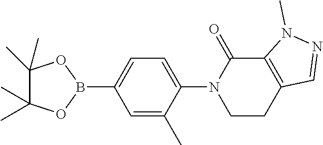 [SMILES: Cc1cc(B2OC(C)(C)C(C)(C)O2)ccc1N1CCc2cnn(C)c2C1=O]

Into a 500-mL 4-necked round-bottom flask under N atmosphere, was placed B₂Pin₂ (23.8 g, 93.72 mmol, 1.50 equiv), 6-(4-bromo-2-methylphenyl)-1-methyl-1,4,5,6-tetrahydro-7H-pyrazolo[3,4-c]pyridin-7-one (20 g, 62.46 mmol, 1 equiv), Pd(dppf)Cl₂ (2.3 g, 3.12 mmol, 0.05 equiv), and KOAc (12.3 g, 124.93 mmol, 2 equiv) in dioxane (400 mL). The resulting solution was stirred overnight at 80° C. The resulting mixture was concentrated and the residue was applied onto a silica gel column and eluted with ethyl acetate/petroleum ether (1/5) to give (13 g, 56.67%) of the title compound as a white solid. LC-MS: (ES, m/z): [M+H]⁺ 368.

### Step 11: Synthesis of 1-methyl-6-(2-methyl-4-(6-(trifluoromethyl)pyrido[3,2-d]pyrimidin-2-yl)phenyl)-1,4,5,6-tetrahydro-7H-pyrazolo[3,4-c]pyridin-7-one

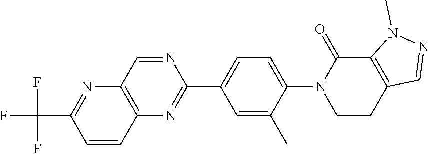 [SMILES: Cc1cc(-c2ncc3nc(C(F)(F)F)ccc3n2)ccc1N1CCc2cnn(C)c2C1=O]

To a stirred mixture of 1-methyl-6-(2-methyl-4-(4,4,5,5-tetramethyl-1,3,2-dioxaborolan-2-yl)phenyl)-1,4,5,6-tetrahydro-7H-pyrazolo[3,4-c]pyridin-7-one (2 g, 5.45 mmol, 1 equiv) and 2-chloro-6-(trifluoromethyl)pyrido[3,2-d]pyrimidine (1.5 g, 6.53 mmol, 1.2 equiv), prepared as described in Example 1 above, in dioxane (200 mL) were added H₂O (40 mL), K₃PO₄ (2.3 g, 10.89 mmol, 2 equiv) and AMPhosPdCl₂ (1.2 g, 1.63 mmol, 0.3 equiv). The resulting mixture was stirred overnight at 50° C. under nitrogen atmosphere and then diluted with water. The organics were extracted with EtOAc and the combined organic layers were concentrated under reduced pressure. The residue was purified by silica gel column chromatography, eluted with PE/EtOAc (3/1) to give crude product which was re-crystallized from MeOH (50 mL) to afford (706.7 mg, 29.60%) of the title compound as a yellow solid. LC-MS: (ES, m/z): [M+H]⁺ 439; ¹H-NMR: (400 MHz, DMSO, ppm): δ2.35 (s, 3H), δ2.90-3.05 (m, 2H), δ3.73-3.79 (m, 1H), δ4.02-4.14 (m, 4H), δ7.49 (s, 1H), δ7.53-7.55 (d, 1H), δ8.46-8.49 (m, 2H), δ8.55 (s, 1H), δ8.80-8.82 (d, 1H), δ9.95 (s, 1H).

### Example 5

### Synthesis of 7-(2,5-dimethyl-4-(8-methyl-6-(trifluoromethyl)-1,5-naphthyridin-2-yl)phenyl)-1-methyl-6,7-dihydro-1H-pyrazolo[3,4-f][1,4]oxazepin-8(5H)-one

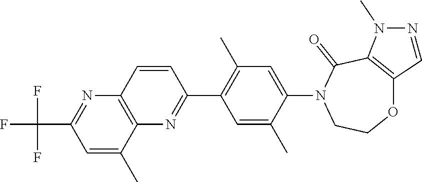 [SMILES: Cc1cc(N2CCOc3cnn(C)c3C2=O)c(C)cc1-c1ccc2nc(C(F)(F)F)cc(C)c2n1]

### Step 1: Synthesis of 4-methyl-5-nitro-2-(trifluoromethyl)pyridine

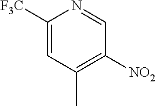

Into a 2 L 3-necked round-bottom flask under N atmosphere, was placed 2-bromo-4-methyl-5-nitropyridine (100 g, 460.79 mmol, 1 equiv), methyl 2,2-difluoro-2-(fluorosulfonyl)acetate (177.0 g, 921.57 mmol, 2 equiv), and CuI (70.2 g, 368.60 mmol, 0.800 equiv) in DMF (1 L). The resulting solution was stirred for 14 h at 120° C. and then diluted with NH₄Cl (3 L), NH₄OH (0.5 L). The resulting solution was extracted with ethyl acetate and the combined organic layer was concentrated under reduced pressure. The residue was applied onto a silica gel column and eluted with petroleum ether to give (40 g, 41.94%) of the title compound as a red oil. GC-MS: (ES, m/z): [M]⁺ 206.

### Step 2: Synthesis of 4-methyl-6-(trifluoromethyl)pyridin-3-amine

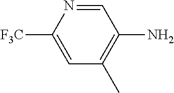

Into a 1 L 3-necked round-bottom flask was placed 4-methyl-5-nitro-2-(trifluoromethyl)pyridine (60 g, 291.26 mmol, 1 equiv), Fe (48.9 g, 873.79 mmol, 3 equiv), NH₄Cl (77.2 g, 1456.31 mmol, 5 equiv), and H₂O (500 mL). The resulting mixture was stirred for 2 h at 80° C. The resulting solution was extracted with ethyl acetate. The residue was applied onto a silica gel column and eluted with ethyl acetate/petroleum ether (1/10) to give (27 g, 60.76%) of the title compound as a white solid. LC-MS: (ES, m/z): [M+H]⁺ 177.

### Step 3: Synthesis of 2-bromo-4-methyl-6-(trifluoromethyl)pyridin-3-amine

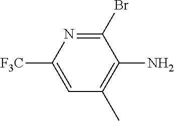

To a stirred solution 4-methyl-6-(trifluoromethyl)pyridin-3-amine (27 g, 153.41 mmol, 1 equiv) in DCM (270 mL) was added NBS (27.1 g, 153.41 mmol, 1 equiv) in portions at 0° C. under nitrogen atmosphere. The resulting mixture was stirred for 2 h at 0° C. under nitrogen atmosphere. The reaction mixture was quenched with water at room temperature and the resulting mixture was extracted with CH₂Cl₂. The combined organic layers were dried over anhydrous Na₂SO₄. After filtration, the filtrate was concentrated under reduced pressure. The residue was purified by silica gel column chromatography with PE/EtOAc (30/1) as eluent to afford (36.4 g, 96.45%) of the title compound as a red solid. LC-MS: (ES, m/z): [M+H]⁺ 255.

### Step 4: Synthesis of ethyl 3-(3-amino-4-methyl-6-(trifluoromethyl)pyridin-2-yl)acrylate

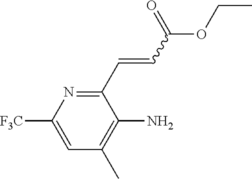 [SMILES: CCOC(=O)C=Cc1nc(C(F)(F)F)cc(C)c1N]

To a stirred mixture of 2-bromo-4-methyl-6-(trifluoromethyl)pyridin-3-amine (36 g, 141.16 mmol, 1 equiv) and ethyl prop-2-enoate (28.3 g, 282.67 mmol, 2.00 equiv) in DMF (720 mL) were added P(o-tol)3 (8.6 g, 28.23 mmol, 0.2 equiv), Et₃N (42.9 g, 423.47 mmol, 3 equiv) and Pd(OAc)₂ (3.2 g, 14.12 mmol, 0.1 equiv). The resulting mixture was stirred overnight at 100° C. under nitrogen atmosphere. The reaction was quenched with water at room temperature. The resulting mixture was extracted with EtOAc and the combined organic layers were washed with NaCl (aq.). The resulting organic layer was concentrated under reduced pressure and the residue was purified by silica gel column chromatography, eluted with PE/EtOAc (20/1) to afford (23.9 g, 61.74%) of the title compound as a yellow solid. LC-MS: (ES, m/z): [M+H]⁺ 275.

### Step 5: Synthesis of 8-methyl-6-(trifluoromethyl)-1,5-naphthyridin-2(1H)-one

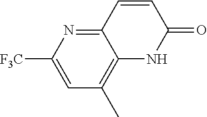 [SMILES: Cc1cc(C(F)(F)F)nc2ccc(=O)[nH]c12]

Into a 500 mL 3-necked round-bottom flask, was placed ethyl 3-(3-amino-4-methyl-6-(trifluoromethyl)pyridin-2-yl)acrylate (23.9 g, 87.23 mmol, 1 equiv), 1,4-dioxane (120 mL), and HCl (6M) (120 mL). The resulting solution was stirred overnight at 100° C. The resulting mixture was diluted with water and the solids were collected by filtration to give (18.8 g, 94.52%) of the title compound as a grey solid. LC-MS: (ES, m/z): [M+H]⁺ 229.

### Step 6: Synthesis of 6-chloro-4-methyl-2-(trifluoromethyl)-1,5-naphthyridine

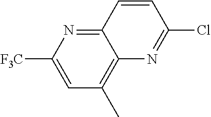 [SMILES: Cc1cc(C(F)(F)F)nc2ccc(Cl)nc12]

Into a 250-mL 3-necked round-bottom flask, was placed 8-methyl-6-(trifluoromethyl)-1,5-naphthyridin-2(1H)-one (18.75 g, 82.24 mmol, 1 equiv), benzene phosphorus oxydichloride (95 mL). The resulting solution was heated to reflux for 3 h and then diluted with water. The solids were collected by filtration to give (15.7123 g, 77.67%) of the title compound as a grey solid. LC-MS: (ES, m/z): [M+H]⁺ 247.

### Step 7. Synthesis of 4-(2-(benzyloxy)ethoxy)-N-(4-bromo-2,5-dimethylphenyl)-1-methyl-1H-pyrazole-5-carboxamide

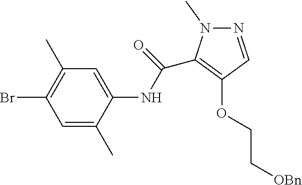 [SMILES: Cc1cc(NC(=O)c2c(OCCOCc3ccccc3)cnn2C)c(C)cc1Br]

Into a 100-mL 3-necked round-bottom flask under N₂ atmosphere, was placed 4-bromo-2,5-dimethylaniline (2.5 g, 12.72 mmol, 1.5 equiv), Et₃N (1.3 g, 12.72 mmol, 1.5 equiv), DCM (25 mL). 4-(2-(Benzyloxy)ethoxy)-1-methyl-1H-pyrazole-5-carbonyl chloride_(2.5 g, 8.48 mmol, 1 equiv), prepared as described in Example 1, in 25 mL of DCM was added dropwise at 0° C. The resulting solution was stirred for 2 h at 0° C. The resulting mixture was concentrated under vacuum and the crude product was purified by re-crystallization from MeOH to give (3.78 g, 97.23%) of the title compound as a yellow solid. LC-MS: (ES, m/z): [M+H]⁺ 458.

### Step 8: Synthesis of N-(4-bromo-2,5-dimethylphenyl)-4-(2-hydroxyethoxy)-1-methyl-1H-pyrazole-5-carboxamide

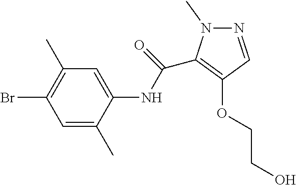 [SMILES: Cc1cc(NC(=O)c2c(OCCO)cnn2C)c(C)cc1Br]

Into a 100-mL 3-necked round-bottom flask under N₂ atmosphere, was placed 4-(2-(benzyloxy)-ethoxy)-N-(4-bromo-2,5-dimethylphenyl)-1-methyl-1H-pyrazole-5-carboxamide (3.78 g, 8.25 mmol, 1 equiv), DCM (40 mL), and BCl₃ (1.4 g, 12.07 mmol, 1.46 equiv) was added at 0° C. The resulting solution was stirred for 30 min at 0° C. and then quenched with 50 mL of NaHCO₃ (aq.). The resulting solution was extracted with dichloromethane. The combined organic layer was dried over anhydrous sodium sulfate and concentrated under vacuum to give (3 g, 98.79%) of the title compound as a yellow solid. (ES, m/z): [M+H]⁺ 368.

### Step 9: Synthesis of 2-((5-((4-bromo-2,5-dimethylphenyl)carbamoyl)-1-methyl-1H-pyrazol-4-yl)oxy)ethyl methanesulfonate

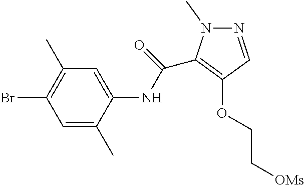 [SMILES: Cc1cc(NC(=O)c2c(OCCOS(C)(=O)=O)cnn2C)c(C)cc1Br]

Into a 100-mL 3-necked round-bottom flask under N₂ atmosphere, was placed N-(4-bromo-2,5-dimethylphenyl)-4-(2-hydroxyethoxy)-1-methyl-1H-pyrazole-5-carboxamide (3 g, 8.15 mmol, 1 equiv), Et₃N (1.2 g, 12.22 mmol, 1.5 equiv), DCM (30 mL), and MsCl (1.4 g 12.22 mmol, 1.5 equiv) was added dropwise at 0° C. The resulting solution was stirred for 1 h at 0° C. The reaction was then quenched with water and the resulting solution was extracted with dichloromethane. The combined organic layer was dried over anhydrous sodium sulfate and concentrated to give (3.5 g, 96.25%) of the title compound as a yellow solid. LC-MS: (ES, m/z): [M+H]⁺ 446.

### Step 10: Synthesis of 7-(4-bromo-2,5-dimethylphenyl)-1-methyl-6,7-dihydro-1H-pyrazolo[3,4-f][1,4]oxazepin-8(5H)-one

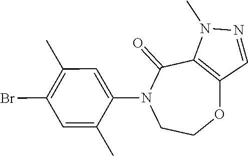 [SMILES: Cc1cc(N2CCOc3cnn(C)c3C2=O)c(C)cc1Br]

Into a 100-mL 3-necked round-bottom flask under N₂ atmosphere, was placed 2-((5-((4-bromo-2,5-dimethylphenyl)carbamoyl)-1-methyl-1H-pyrazol-4-yl)oxy)ethyl methanesulfonate (3.7 g, 8.29 mmol, 1 equiv), DMF (40 mL), and NaH (0.3 g, 12.50 mmol, 1.51 equiv) was added at 0° C. The resulting solution was stirred for 1 h at 0° C. and then quenched with NH₄Cl (aq.). The resulting solution was extracted with ethyl acetate. The combined organic layer was concentrated under reduced pressure. The residue was applied onto a silica gel column and eluted with ethyl acetate/petroleum ether (1:20) to give (2 g, 68.89%) of the title compound as a yellow solid. LC-MS: (ES, m/z): [M+H]⁺ 350.

### Step 11: Synthesis of 7-(2,5-dimethyl-4-(4,4,5,5-tetramethyl-1,3,2-dioxaborolan-2-yl)phenyl)-1-methyl-6,7-dihydro-1H-pyrazolo[3,4-f][1,4]oxazepin-8(5H)-one

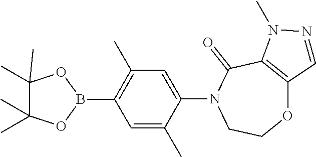 [SMILES: Cc1cc(N2CCOc3cnn(C)c3C2=O)c(C)cc1B1OC(C)(C)C(C)(C)O1]

Into a 50-mL 3-necked round-bottom flask under N₂ atmosphere, was placed 7-(4-bromo-2,5-dimethylphenyl)-1-methyl-6,7-dihydro-1H-pyrazolo[3,4-f][1,4]-oxazepin-8(5H)-one (2 g, 5.71 mmol, 1 equiv), B₂Pin₂ (2.18 g, 8.60 mmol, 1.5 equiv), KOAc (1.7 g, 17.13 mmol, 3 equiv), dioxane (20 mL), and Pd(dppf)Cl₂ (0.6 g, 0.86 mmol, 0.15 equiv). The resulting solution was stirred for 12 h at 80° C. The resulting mixture was concentrated under vacuum. The residue was applied onto a silica gel column and eluted with ethyl acetate/petroleum ether (1:20) to give (2.2 g, 96.97%) of the title compound as a white solid. LC-MS: (ES, m/z): [M+H]⁺ 398.

### Step 12: Synthesis of 7-(2,5-dimethyl-4-(8-methyl-6-(trifluoromethyl)-1,5-naphthyridin-2-yl)phenyl)-1-methyl-6,7-dihydro-1H-pyrazolo[3,4-f][1,4]oxazepin-8(5H)-one

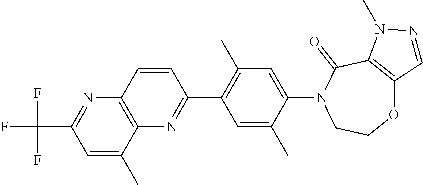 [SMILES: Cc1cc(N2CCOc3cnn(C)c3C2=O)c(C)cc1-c1ccc2nc(C(F)(F)F)cc(C)c2n1]

Into a 50-mL 3-necked round-bottom flask under N₂ atmosphere, was placed 7-(2,5-dimethyl-4-(4,4,5,5-tetramethyl-1,3,2-dioxaborolan-2-yl)phenyl)-1-methyl-6,7-dihydro-1H-pyrazolo[3,4-f][1,4]oxazepin-8(5H)-one (800 mg, 2.01 mmol, 1 equiv), 6-chloro-4-methyl-2-(trifluoromethyl)-1,5-naphthyridine (595.9 mg, 2.42 mmol, 1.2 equiv), Na₂CO₃ (640.3 mg, 6.04 mmol, 3 equiv), DME (14 mL), H₂O (3.5 mL), and Pd(PPh₃)₄ (349.0 mg, 0.30 mmol, 0.15 equiv). The resulting solution was stirred for 12 h at 80° C. The reaction was then quenched with water. The resulting solution was extracted with ethyl acetate and the combined organic layer was concentrated under reduced pressure. The residue was applied onto a silica gel column and eluted with dichloromethane/methanol (50:1) to give (529.9 mg, 54.65%) of the title compound as a yellow solid. LC-MS: (ES, m/z): [M+H]⁺ 482.

### Example 6

### Synthesis of 3-methyl-5-(2-methyl-4-(6-(trifluoromethyl)-1,5-naphthyridin-2-yl)phenyl)-6,7-dihydropyrazolo[1,5-a]pyrazin-4(5H)-one

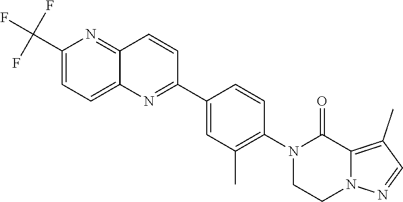 [SMILES: Cc1cc(-c2ccc3nc(C(F)(F)F)ccc3n2)ccc1N1CCn2ncc(C)c2C1=O]

### Step 1: Synthesis of 2-bromo-6-(trifluoromethyl)pyridin-3-amine

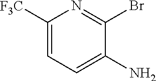 [SMILES: Nc1ccc(C(F)(F)F)nc1Br]

To a stirred solution of 6-(trifluoromethyl)pyridin-3-amine (5 g, 30.84 mmol, 1 equiv) in acetonitrile (200 mL) were added NBS (5.49 g, 30.84 mmol, 1 equiv) in portions at 0° C. under nitrogen atmosphere. The resulting mixture was stirred at 0° C. for 30 mins and then quenched with water. The resulting mixture was extracted with EtOAc. The combined organic layers were concentrated under reduced pressure. The residue was purified by silica gel column chromatography, eluted with EtOAc/PE (1/10) to afford (3.14 g, 42.24%) of the title compound as a yellow solid. LC-MS: (ES, m/z): [M+H]⁺ 241.

### Step 2: Synthesis of ethyl 3-(3-amino-6-(trifluoromethyl)pyridin-2-yl)acrylate

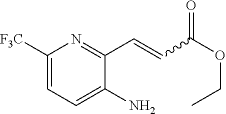 [SMILES: CCOC(=O)C=Cc1nc(C(F)(F)F)ccc1N]

To a stirred solution of 2-bromo-6-(trifluoromethyl)pyridin-3-amine (3.14 g, 13.03 mmol, 1 equiv) and ethyl prop-2-enoate (2.61 g, 26.06 mmol, 2 equiv) in DMF (60 mL) were added Et₃N (3.95 g, 39.09 mmol, 3 equiv), P(o-tol)₃ (0.40 g, 1.30 mmol, 0.1 equiv) and Pd(OAc)₂ (0.15 g, 0.65 mmol, 0.05 equiv). The resulting mixture was stirred under nitrogen at 120° C. overnight. The reaction mixture was quenched with water and extracted with EtOAc. The combined organic layers were concentrated under reduced pressure. The residue was purified by silica gel column chromatography and eluted with EtOAc/PE (1/5) to afford (3.3 g, 97.34%) of the title compound as a yellow solid. LC-MS: (ES, m/z): [M+H]⁺ 261.

### Step 3: Synthesis of 6-(trifluoromethyl)-1,5-naphthyridin-2(1H)-one

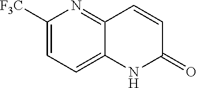 [SMILES: O=c1ccc2nc(C(F)(F)F)ccc2[nH]1]

To a stirred solution of ethyl 3-(3-amino-6-(trifluoromethyl)pyridin-2-yl)acrylate (1.5 g, 5.76 mmol, 1 equiv) in 1,4-dioxane (15 mL) was added HCl (30 mL). The resulting mixture was stirred at 100° C. overnight. The reaction mixture was basified to pH 8 with saturated NaHCO₃ and extracted with EtOAc. The combined organic layers were concentrated under reduced pressure to give the title compound (1.1 g, 89.11%) as a yellow solid. LC-MS: (ES, m/z): [M+H]⁺ 215.

### Step 4: Synthesis of 2-chloro-6-(trifluoromethyl)-1,5-naphthyridine

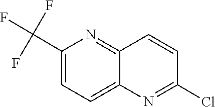 [SMILES: FC(F)(F)c1ccc2nc(Cl)ccc2n1]

A solution of 6-(trifluoromethyl)-1,5-naphthyridin-2(1H)-one (1.18 g, 5.51 mmol, 1 equiv) in POCl₃ (12 mL, 128.74 mmol, 23.364 equiv) was stirred at 120° C. for 30 min. The reaction mixture was quenched with water/ice and then basified to pH 8 with saturated NaHCO₃. The resulting mixture was extracted with EtOAc. The combined organic layers were concentrated under reduced pressure to give (900 mg, 70.22%) of the title compound as a grey solid.

### Step 5: Synthesis of 3-methyl-5-(2-methyl-4-(6-(trifluoromethyl)-1,5-naphthyridin-2-yl)phenyl)-6,7-dihydropyrazolo[1,5-a]pyrazin-4(5H)-one

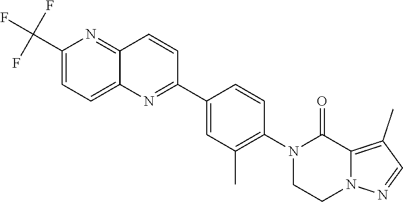 [SMILES: Cc1cc(-c2ccc3nc(C(F)(F)F)ccc3n2)ccc1N1CCn2ncc(C)c2C1=O]

Into a 25-mL 3-necked round-bottom flask under N₂ atmosphere, was placed 2-chloro-6-(trifluoromethyl)-1,5-naphthyridine (500 mg, 2.15 mmol, 1 equiv), 3-methyl-5-(2-methyl-4-(4,4,5,5-tetramethyl-1,3,2-dioxaborolan-2-yl)phenyl)-6,7-dihydropyrazolo[1,5-a]pyrazin-4(5H)-one (789.5 mg, 2.15 mmol, 1.00 equiv), prepared as described in Example 2 above, Na₂CO₃ (683.5 mg, 6.45 mmol, 3 equiv), DME (10 mL, 103.32 mmol, 48.06 equiv), H₂O (2.5 mL, 138.77 mmol, 64.55 equiv), and Pd(PPh₃)₄ (372.6 mg, 0.32 mmol, 0.15 equiv). The resulting solution was stirred for 12 h at 80° C. The reaction was then quenched with water and the resulting solution was extracted with ethyl acetate. After concentration of the organic layer, the residue was applied onto a silica gel column and eluted with dichloromethane/methanol (20/1) to give (601.5 mg, 63.97%) of the title compound as a yellow solid. LC-MS: (ES, m/z): [M+H]⁺ 438; ¹H-NMR: (300 MHz, DMSO, ppm): δ2.26-2.35 (s, 3H), δ2.50-2.51 (s, 3H), δ3.90-3.94 (m, 1H), δ4.26-4.35 (m, 1H), δ4.51-4.55 (m, 2H), δ7.48 (s, 1H), δ7.55-7.57 (d, 1H), δ8.24-8.27 (d, 2H), δ8.33 (s, 1H), δ8.59-8.62 (d, 1H), δ8.68-8.77 (d, 1H), δ8.80 (d, 1H).

### Example 7

### Synthesis of 5-(2-fluoro-5-methyl-4-(6-(trifluoromethyl)-1,5-naphthyridin-2-yl)phenyl)-3-methyl-6,7-dihydropyrazolo[1,5-a]pyrazin-4(5H)-one

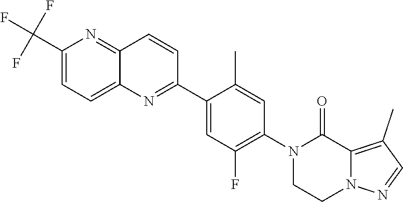 [SMILES: Cc1cc(N2CCn3ncc(C)c3C2=O)c(F)cc1-c1ccc2nc(C(F)(F)F)ccc2n1]

### Step 1: Synthesis of ethyl 1-(2-(benzyloxy)ethyl)-4-methyl-1H-pyrazole-5-carboxylate

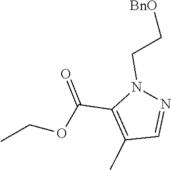 [SMILES: CCOC(=O)c1c(C)cnn1CCOCc1ccccc1]

To a stirred solution of ethyl 4-methyl-H-pyrazole-5-carboxylate (50 g, 324.32 mmol, 1 equiv), DIAD (137.7 g 681.07 mmol, 2.1 equiv) and 2-(benzyloxy)ethan-1-ol (49.4 g, 324.59 mmol, 1 equiv) in THF (500 mL) was added PPh₃ (221.2 g, 843.23 mmol, 2.6 equiv) in portions at 0° C. under nitrogen atmosphere. The resulting mixture was stirred overnight at room temperature. The residue was purified by silica gel column chromatography with PE/EtOAc (50/1) as eluent to afford the title compound (73 g, 78.06%) as a yellow oil. LC-MS: (ES, m/z): [M+H]⁺ 289. Ethyl 1-(2-(benzyloxy)ethyl)-4-methyl-1H-pyrazole-5-carboxylate was converted to 1-(2-(benzyloxy)ethyl)-4-methyl-1H-pyrazole-5-carboxylic acid as described in Example 2, Step 5 above.

### Step 2: Synthesis of 1-(2-(benzyloxy)ethyl)-4-methyl-1H-pyrazole-5-carbonyl Chloride

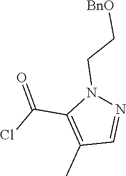 [SMILES: Cc1cnn(CCOCc2ccccc2)c1C(=O)Cl]

To a stirred solution of 1-(2-(benzyloxy)ethyl)-4-methyl-H-pyrazole-5-carboxylic acid (15 g, 57.63 mmol, 1 equiv) and DMF (0.4 g, 5.76 mmol, 0.1 equiv) in DCM (300 mL) was added (COCl)₂(11.0 g, 86.44 mmol, 1.5 equiv) dropwise at 0° C. The resulting mixture was stirred for 3 h at room temperature and then concentrated under reduced pressure to give the title compound (17 g, crude) as a white solid.

### Step 3: Synthesis of 1-(2-(benzyloxy)ethyl)-N-(4-bromo-2-fluoro-5-methylphenyl)-4-methyl-1H-pyrazole-5-carboxamide

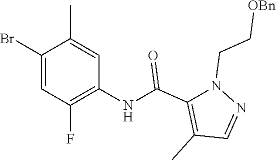 [SMILES: Cc1cc(NC(=O)c2c(C)cnn2CCOCc2ccccc2)c(F)cc1Br]

To a stirred solution of 4-bromo-2-fluoro-5-methylaniline (18.4 g, 90.41 mmol, 1.2 equiv) and Et₃N (15.2 g, 150.68 mmol, 2 equiv) in DCM (200 mL) was added 1-(2-(benzyloxy)ethyl)-4-methyl-1H-pyrazole-5-carbonyl chloride (21 g, 75.34 mmol, 1 equiv) dropwise at 0° C. under nitrogen atmosphere. The resulting mixture was stirred for 1 h at RT under nitrogen atmosphere and then diluted with water. The aqueous layer was extracted with CH₂Cl₂ and the organics were concentrated under reduced pressure. The crude product was re-crystallized from MeOH to afford (26 g, 77.32%) of the title compound as a white solid. LC-MS: (ES, m/z): [M+H]⁺ 446.

### Step 4: Synthesis of N-(4-bromo-2-fluoro-5-methylphenyl)-1-(2-hydroxyethyl)-4-methyl-1H-pyrazole-5-carboxamide

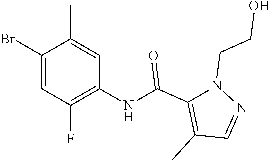 [SMILES: Cc1cc(NC(=O)c2c(C)cnn2CCO)c(F)cc1Br]

By proceeding analogously as described in Example 3, Step 3 above, but substituting 1-(2-(benzyloxy)ethyl)-N-(4-bromo-2-fluoro-5-methylphenyl)-4-methyl-1H-pyrazole-5-carboxamide (26 g, 58.25 mmol, 1 equiv) for 1-(2-(benzyloxy)ethyl)-N-(4-bromo-2,5-dimethylphenyl)-4-methyl-1H-pyrazole-5-carboxamide, (19.1 g, 95.82%) of the title compound was obtained as a white solid. LC-MS: (ES, m/z): [M+H]⁺ 356.

### Step 5: Synthesis of 2-(5-((4-bromo-2-fluoro-5-methylphenyl)carbamoyl)-4-methyl-1H-pyrazol-1-yl)ethyl Methanesulfonate

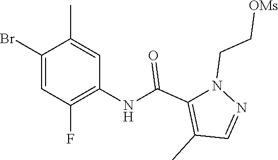 [SMILES: Cc1cc(NC(=O)c2c(C)cnn2CCOS(C)(=O)=O)c(F)cc1Br]

To a stirred solution of N-(4-bromo-2-fluoro-5-methylphenyl)-1-(2-hydroxyethyl)-4-methyl-1H-pyrazole-5-carboxamide (19.1 g, 53.62 mmol, 1 equiv) and Et₃N (10.9 g, 107.24 mmol, 2 equiv) in DCM (190 mL) was added MsCl (9.2 g, 80.43 mmol, 1.5 equiv) dropwise at 0° C. The resulting mixture was stirred for 30 min at room temperature and then diluted with water. The resulting mixture was extracted with CH₂Cl₂ and the combined organic layers were dried over anhydrous Na₂SO₄. After filtration, the filtrate was concentrated under reduced pressure. The crude product was re-crystallized from EA to afford (20.5 g, 88.03%) of the title compound as a white solid. LC-MS: (ES, m/z): [M+H]⁺ 434.

### Step 6: Synthesis of 5-(4-bromo-2-fluoro-5-methylphenyl)-3-methyl-6,7-dihydropyrazolo[1,5-a]pyrazin-4(5H)-one

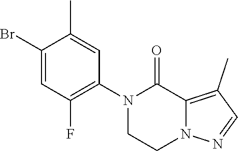 [SMILES: Cc1cc(N2CCn3ncc(C)c3C2=O)c(F)cc1Br]

To a stirred solution of 2-(5-((4-bromo-2-fluoro-5-methylphenyl)carbamoyl)-4-methyl-1H-pyrazol-1-yl)ethyl methanesulfonate (20.9 g, 48.13 mmol, 1 equiv) in DMF (209 mL) was added NaH (60%) (2.9 g, 72.19 mmol, 1.5 equiv) at 0° C. under nitrogen atmosphere. The resulting mixture was stirred for 0.5 h at 0° C. under nitrogen atmosphere. The reaction mixture was quenched by the addition of sat. NH₄Cl (aq.) (200 mL) at room temperature. The resulting mixture was extracted with EtOAc. The combined organic layers were dried over anhydrous Na₂SO₄. After filtration, the filtrate was concentrated under reduced pressure to give the title compound (16.2 g, 99.54%) as a white solid. LC-MS: (ES, m/z): [M+H]⁺ 338.

### Step 7: Synthesis of 5-(2-fluoro-5-methyl-4-(4,4,5,5-tetramethyl-1,3,2-dioxaborolan-2-yl)phenyl)-3-methyl-6,7-dihydropyrazolo[1,5-a]pyrazin-4(5H)-one

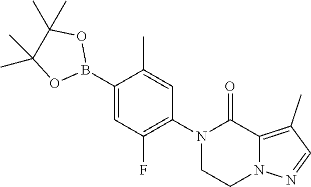 [SMILES: Cc1cc(N2CCn3ncc(C)c3C2=O)c(F)cc1B1OC(C)(C)C(C)(C)O1]

To a stirred solution of 5-(4-bromo-2-fluoro-5-methylphenyl)-3-methyl-6,7-dihydropyrazolo[1,5-a]pyrazin-4(5H)-one (16.1 g, 47.61 mmol, 1 equiv) and B₂Pin₂ (18.1 g, 71.41 mmol, 1.5 equiv) in dioxane (161 mL) were added KOAc (9.3 g, 95.22 mmol, 2 equiv) and Pd(dppf)Cl₂(3.5 g, 4.76 mmol, 0.1 equiv). The resulting mixture was stirred overnight at 80° C. under nitrogen atmosphere. The resulting mixture was concentrated under reduced pressure. The residue was purified by silica gel column chromatography with PE/EtOAc (5/1) as eluent to afford the title compound (15.8 g, 86.15%) as a white solid. LC-MS: (ES, m/z): [M+H]⁺ 386.

### Step 8: Synthesis of 5-(2-fluoro-5-methyl-4-(6-(trifluoromethyl)-1,5-naphthyridin-2-yl)phenyl)-3-methyl-6,7-dihydropyrazolo[1,5-a]pyrazin-4(5H)-one

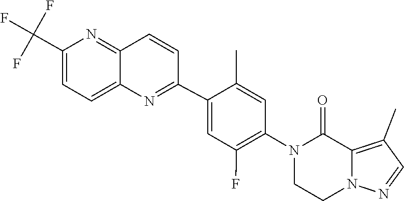 [SMILES: Cc1cc(N2CCn3ncc(C)c3C2=O)c(F)cc1-c1ccc2nc(C(F)(F)F)ccc2n1]

To a stirred solution of 2-chloro-6-(trifluoromethyl)-1,5-naphthyridine (500 mg, 2.15 mmol, 1 equiv) and 5-(2-fluoro-5-methyl-4-(4,4,5,5-tetramethyl-1,3,2-dioxaborolan-2-yl)phenyl)-3-methyl-6,7-dihydropyrazolo[1,5-a]pyrazin-4(5H)-one (828.2 mg, 2.15 mmol, 1 equiv) in DME (10 mL) and H₂O (2.5 mL) was added Na₂CO₃ (683.5 mg, 6.45 mmol, 3 equiv) and Pd(PPh₃)₄ (248.4 mg, 0.21 mmol, 0.1 equiv). The resulting mixture was stirred overnight at 80° C. under nitrogen atmosphere. The reaction mixture was diluted with water at room temperature and the resulting mixture was extracted with EtOAc. The combined organic layers were concentrated under reduced pressure and the residue was purified by silica gel column chromatography, eluted with PE/EtOAc (1/2) to afford the title compound (701.3 mg, 71.63%) as a grey solid. LC-MS: (ES, m/z): [M+H]⁺ 456. ¹H-NMR: (400 MHz, DMSO, ppm): (400 MHz, DMSO, ppm): δ2.27 (s, 3H), δ2.43 (s, 3H), δ4.17-4.20 (t, 2H), δ4.50-4.53 (t, 2H), δ7.50 (s, 1H), δ7.55-7.63 (m, 2H), δ8.20-8.30 (m, 2H), δ8.71-8.80 (m, 2H).

### Example 8

### Synthesis of 3-methyl-5-(2-methyl-4-(6-(trifluoromethyl)quinolin-2-yl)phenyl)-6,7-dihydropyrazolo-[1,5-a]pyrazin-4(5H)-one

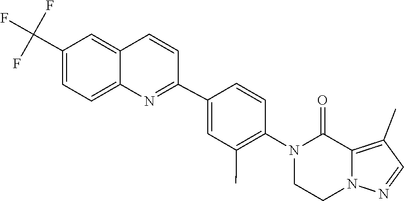 [SMILES: Cc1cc(-c2ccc3cc(C(F)(F)F)ccc3n2)ccc1N1CCn2ncc(C)c2C1=O]

### Step 1: Synthesis of ethyl 3-(2-amino-5-(trifluoromethyl)phenyl)acrylate

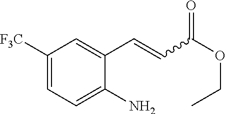 [SMILES: CCOC(=O)C=Cc1cc(C(F)(F)F)ccc1N]

Proceeding analogously as described in Example 6, Step 2 above but substituting 2-bromo-6-(trifluoromethyl)pyridin-3-amine with 2-bromo-4-trifluoromethylaniline provided (34 g, 92.27%) of the title compound as a yellow solid LC-MS: (ES, m/z): [M+H]⁺ 260.

### Step 2: Synthesis of 6-(trifluoromethyl)quinolin-2(1H)-one

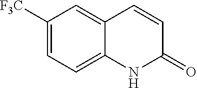 [SMILES: O=c1ccc2cc(C(F)(F)F)ccc2[nH]1]

Into a 2 L round-bottom flask, was placed ethyl 3-(2-amino-5-(trifluoromethyl)phenyl)-acrylate (34 g, 131.27 mmol, 1.00 equiv), dioxane (340 mL), and HCl (4M) (340 mL). The resulting solution was stirred for 12 h at 100° C. and then quenched with H₂O. The resulting solution was extracted with ethyl acetate. The combined organic layers were dried over anhydrous sodium sulfate and concentrated. The crude product was purified by re-crystallization from PE/EA (10/1) to give (27 g, 96.57%) of the title compound as a white solid. LC-MS: (ES, m/z): [M+H]⁺ 214.

### Step 3: Synthesis of 2-chloro-6-(trifluoromethyl)quinoline

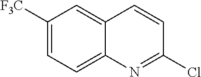 [SMILES: FC(F)(F)c1ccc2nc(Cl)ccc2c1]

Into a 500-mL 3-necked round-bottom flask was placed 6-(trifluoromethyl)quinolin-2(1H)-one (31 g, 145.54 mmol, 1.00 equiv), and POCl₃ (155 mL). The resulting solution was stirred for 3 h at 120° C. The resulting mixture was concentrated under vacuum. The residue was then quenched with water/ice and extracted with ethyl acetate. The mixture was dried over anhydrous sodium sulfate and concentrated to give (28 g, 83.28%) of the title compound as a tan solid. LC-MS: (ES, m/z): [M+H]⁺ 232.

### Step 4: Synthesis of 3-methyl-5-(2-methyl-4-(6-(trifluoromethyl)quinolin-2-yl)phenyl)-6,7-dihydropyrazolo[1,5-a]pyrazin-4(5H)-one

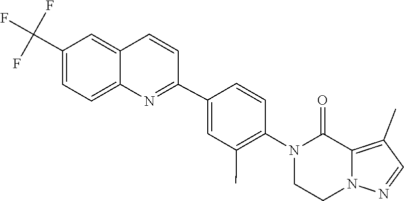 [SMILES: Cc1cc(-c2ccc3cc(C(F)(F)F)ccc3n2)ccc1N1CCn2ncc(C)c2C1=O]

To a stirred mixture of 3-methyl-5-(2-methyl-4-(4,4,5,5-tetramethyl-1,3,2-dioxaborolan-2-yl)phenyl)-6,7-dihydropyrazolo[1,5-a]pyrazin-4(5H)-one (1 g, 2.72 mmol, 1 equiv) and 2-chloro-6-(trifluoromethyl)quinoline (632 mg, 2.72 mmol, 1.00 equiv) in DME (12.64 mL) were added Na₂CO₃ (866.5 mg, 8.18 mmol, 3.00 equiv), Pd(PPh₃)₄ (314.4 mg, 0.27 mmol, 0.10 equiv) and H₂O (3.16 mL, 175.41 mmol, 64.42 equiv). The resulting solution was stirred for 3 h at 80° C. under nitrogen atmosphere. The reaction mixture was diluted with 50 mL of water and extracted with EtOAc. The combined organic layer was concentrated under reduced pressure. The residue was purified by silica gel column chromatography with PE/EtOAc (5/1) as eluent to afford the title compound (0.7792 g, 65.57%) as a white solid. LC-MS: (ES, m/z): [M+H]⁺ 437. ¹H-NMR: (400 MHz, DMSO, ppm): δ2.26 (s, 3H), δ2.35 (s, 3H), δ3.90-3.94 (m, 1H), δ4.26-4.33 (m, 1H), δ4.51-4.54 (t, 2H), δ7.48 (s, 1H), δ7.53-7.55 (d, 1H), δ8.01-8.04 (m, 1H), δ8.20-8.23 (m, 1H), δ8.27-8.29 (m, 2H), δ8.34-8.37 (d, 1H), δ8.54 (s, 1H), δ8.68-8.70 (d, 1H).

### Example 9

### Synthesis of 3-methyl-5-(2-methyl-4-(8-methyl-6-(trifluoromethyl)quinazolin-2-yl)phenyl)-6,7-dihydropyrazolo[1,5-a]pyrazin-4(5H)-one

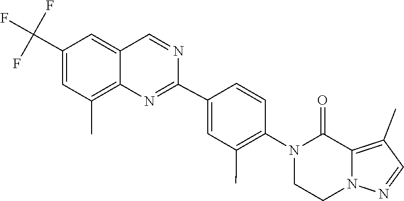 [SMILES: Cc1cc(-c2ncc3cc(C(F)(F)F)cc(C)c3n2)ccc1N1CCn2ncc(C)c2C1=O]

### Step 1: Synthesis of 2-methyl-4-(trifluoromethyl)aniline

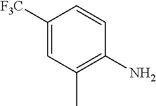

To a stirred mixture of 2-bromo-4-(trifluoromethyl)aniline (100 g, 416.63 mmol, 1 equiv) and trimethyl-1,3,5,2,4,6-trioxatriborinane (210.2 g, 1674.50 mmol, 4.02 equiv) in DMF were added Pd(PPh₃)₄ (24.2 g, 20.94 mmol, 0.05 equiv) and K₂CO₃ (144.4 g, 1044.82 mmol, 2.51 equiv). The resulting mixture was stirred overnight at 100° C. under nitrogen atmosphere. The reaction was diluted with water and extracted with EtOAc. The combined organic layer was concentrated under reduced pressure. The residue was purified by silica gel column chromatography with PE/EtOAc (10:1) as eluent to afford the title compound (60 g, 81.94%) as a white solid. LC-MS: (ES, m/z): [M+H]⁺ 176.

### Step 2: Synthesis of 2-bromo-6-methyl-4-(trifluoromethyl)aniline

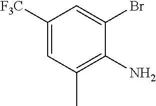

To a stirred solution of 2-methyl-4-(trifluoromethyl)aniline (60 g, 342.86 mol, 1 equiv) in DCM (1200 mL) was added NBS (60.7 g, 342.86 mmol, 1 equiv) in portions at 0° C. under nitrogen atmosphere. The resulting mixture was stirred for 2 h at 0° C. under nitrogen atmosphere. The reaction mixture was quenched with water at room temperature. The aqueous layer was extracted with DCM and the combined organic layers were concentrated under reduced pressure. The residue was purified by silica gel column chromatography with PE/EtOAc (30/1) as eluent to afford the title compound (49 g, 67.57%) as yellow oil. LC-MS: (ES, m/z): [M+H]⁺ 254.

### Step 3: Synthesis of 2-amino-3-methyl-5-(trifluoromethyl)benzonitrile

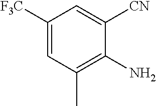

To a stirred solution of 2-bromo-6-methyl-4-(trifluoromethyl)aniline (49 g, 193.68 mol, 1 equiv) and Zn(CN)₂ (44.9 g, 141.34 mmol, 2.00 equiv) in DMF (1000 mL) was added Pd(PPh₃)₄ (11.2 g, 9.68 mmol, 0.05 equiv) at room temperature under nitrogen atmosphere. The resulting mixture was stirred overnight at 120° C. under nitrogen atmosphere. The reaction was quenched with water at room temperature and the aqueous layer was extracted with EtOAc. The combined organic layers were concentrated under reduced pressure. The residue was purified by silica gel column chromatography with PE/EtOAc (10/1) as eluent to afford the title compound (37.6 g, 97.16%) as a yellow solid. LC-MS: (ES, m/z): [M+H]⁺ 201.

### Step 4: Synthesis of 8-methyl-6-(trifluoromethyl)quinazoline-2,4(1H,3H)-dione

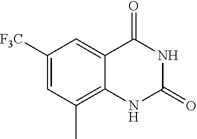 [SMILES: Cc1cc(C(F)(F)F)cc2c(=O)[nH]c(=O)[nH]c12]

To a stirred mixture of 2-amino-3-methyl-5-(trifluoromethyl)benzonitrile (37.6 g, 188.00 mmol, 1 equiv) in DMF (1200 mL) were added DBU (139.3 g, 564.00 mmol, 3.00 equiv). The resulting mixture was stirred overnight at 100° C. under CO₂ atmosphere. The reaction was quenched with water at room temperature. The mixture was acidified to pH 5 with HCl (5M). The precipitated solids were collected by filtration and washed with water. The resulting solid was dried under infrared light to give the title compound (40 g, 87.20%) as a white solid. LC-MS: (ES, m/z): [M+H]⁺ 245.

### Step 5: Synthesis of 2,4-dichloro-8-methyl-6-(trifluoromethyl)quinazoline

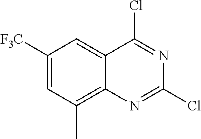 [SMILES: Cc1cc(C(F)(F)F)cc2c(Cl)nc(Cl)nc12]

To a stirred mixture of 8-methyl-6-(trifluoromethyl)quinazoline-2,4(1H,3H)-dione (29 g, 118.85 mmol, 1 equiv) in POCl₃ (150 mL) was added PCl₅ (123.6 g, 594.26 mmol, 5 equiv). The resulting mixture was stirred for 8 hours at 120° C. under nitrogen atmosphere. The resulting mixture was concentrated under vacuum and the residue was quenched with water/ice at room temperature. The aqueous layer was extracted with MTBE and the combined organic layers were washed with H₂O. The resulting organic phase was dried with anhydrous sodium sulfate and concentrated under reduced pressure. The residue was purified by silica gel column chromatography with PE/EtOAc (100/1) as eluent to afford the title compound (15.5 g, 46.57%) as a white solid. GC-MS: (ES, m/z): [M]⁺ 280.

### Step 6: Synthesis of 2-chloro-8-methyl-6-(trifluoromethyl)quinazoline

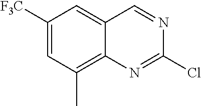 [SMILES: Cc1cc(C(F)(F)F)cc2cnc(Cl)nc12]

To a stirred mixture of 2,4-dichloro-8-methyl-6-(trifluoromethyl)quinazoline (34.8 g, 124.28 mmol, 1 equiv) in THF (350 mL) was added Bn₃SnH (39.9 g, 136.93 mmol, 1.10 equiv). Pd(PPh₃)₄ (14.4 g, 12.43 mmol, 0.10 equiv) was added at 0° C. under nitrogen atmosphere. The resulting mixture was stirred overnight at room temperature under nitrogen atmosphere. The reaction mixture was concentrated under reduced pressure and the residue was purified by silica gel column chromatography, eluted with PE/EtOAc (100/1) to afford the title compound (15.1308 g 49.49%) as a light yellow solid. GC-MS: (ES, m/z): [M]⁺ 246.

### Step 7: Synthesis of 3-methyl-5-(2-methyl-4-(8-methyl-6-(trifluoromethyl)quinazolin-2-yl)phenyl)-6,7-dihydropyrazolo[1,5-a]pyrazin-4(5H)-one

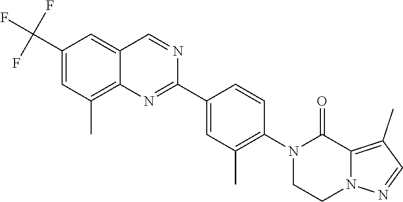 [SMILES: Cc1cc(-c2ncc3cc(C(F)(F)F)cc(C)c3n2)ccc1N1CCn2ncc(C)c2C1=O]

To a stirred solution of 3-methyl-5-(2-methyl-4-(4,4,5,5-tetramethyl-1,3,2-dioxaborolan-2-yl)phenyl)-6,7-dihydropyrazolo[1,5-a]pyrazin-4(5H)-one (1.2 g, 3.27 mmol, 1 equiv) and 2-chloro-8-methyl-6-(trifluoromethyl)quinazoline (0.8 g, 3.27 mol, 1.00 equiv) in toluene (24 mL) was added EtOH (12 mL), K₂CO₃ (1.4 g, 9.80 mmol, 3 equiv) and Pd(PPh₃)₄ (0.4 g, 0.33 mmol, 0.1 equiv). The resulting mixture was stirred overnight at 80° C. under nitrogen atmosphere. The resulting mixture was diluted with and extracted with EtOAc. The combined organic layer was concentrated under reduced pressure. The residue was purified by silica gel column chromatography with PE/EtOAc (1/1) as eluent to the title compound (661 mg, 44.81%) as a white solid. LC-MS: (ES, m/z): [M+H]⁺ 452. H-NMR: (300 MHz, DMSO, ppm): δ2.21 (s, 3H), δ2.36 (s, 3H), δ2.88 (s, 3H), δ3.90-3.93 (m, 1H), δ4.27-4.33 (m, 1H), δ4.51-4.55 (m, 2H), δ7.48-7.57 (m, 2H), δ8.17 (s, 1H), δ8.50-8.60 (m, 3H), δ9.86 (s, 1H).

### Example 10

### Synthesis of 6-(2,5-dimethyl-4-(6-(trifluoromethyl)-1,5-naphthyridin-2-yl)phenyl)-1-methyl-1,4,5,6-tetrahydro-7H-pyrazolo[3,4-c]pyridin-7-one

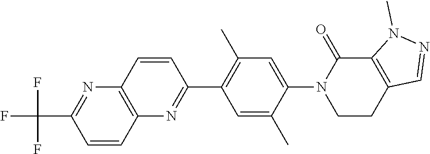 [SMILES: Cc1cc(N2CCc3cnn(C)c3C2=O)c(C)cc1-c1ccc2nc(C(F)(F)F)ccc2n1]

### Step 1: Synthesis of methyl 4-allyl-1-methyl-H-pyrazole-5-carboxylate

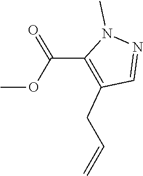

Into a 250-mL 3-necked round-bottom flask under N₂ atmosphere, was placed methyl 4-bromo-1-methyl-1H-pyrazole-5-carboxylate (5 g, 22.83 mmol, 1 equiv), tributyl(prop-2-en-1-yl)stannane (8.3 g, 25.07 mmol, 1.10 equiv), DMF (50 mL), Pd(PPh₃)₄ (2.6 g, 2.25 mmol, 0.10 equiv). The resulting solution was stirred overnight at 100° C. The reaction mixture was then quenched with water and extracted with ethyl acetate and the organic layers combined. The resulting mixture was concentrated and the residue was applied onto a silica gel column and eluted with ethyl acetate/petroleum ether (1:50) to give (3.2 g, 77.79%) of the title compound as yellow oil. LC-MS: (ES, m/z): [M+H]⁺ 181.

### Step 2: Synthesis of 4-allyl-1-methyl-H-pyrazole-5-carboxylic Acid

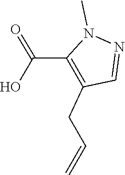

Into a 50-mL 3-necked round-bottom flask, was placed methyl 4-allyl-1-methyl-1H-pyrazole-5-carboxylate (3.2 g, 17.76 mmol, 1 equiv), THF (15 mL, 185.14 mmol, 10.43 equiv), and LiOH (0.9 g, 37.58 mmol, 2.12 equiv) in H₂O (7 mL). The resulting solution was stirred overnight at room temperature and then diluted with H₂O. The resulting mixture was concentrated and the resulting solution was extracted with ethyl acetate. The pH value of the aqueous layer was adjusted to 4 with HCl (1 mol/L) and the organics were extracted with ethyl acetate. The combined organic layer was dried over anhydrous sodium sulfate and concentrated under reduced pressure to give (2.1 g, 71.16%) of the title compound as a white solid. GC-MS: (ES, m/z): [M]⁺ 166.

### Step 3: Synthesis of 4-allyl-N-(4-bromo-2,5-dimethylphenyl)-1-methyl-H-pyrazole-5-carboxamide

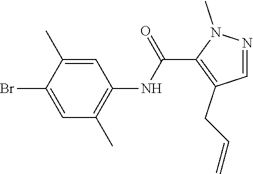 [SMILES: C=CCc1cnn(C)c1C(=O)Nc1cc(C)c(Br)cc1C]

Into a 25-mL 3-necked round-bottom flask under N₂ atmosphere, was placed 4-allyl-1-methyl-1H-pyrazole-5-carboxylic acid (0.843 g, 5.07 mmol, 1 equiv), 4-bromo-2,5-dimethylaniline (1.0 g, 5.00 mmol, 0.99 equiv), HATU (2.9 g, 7.63 mmol, 1.50 equiv), DIEA (1.3 g, 10.15 mmol, 2 equiv) and DMF (10 mL). The resulting solution was stirred overnight at room temperature. The reaction was then quenched with water and the resulting solution was extracted with ethyl acetate. The combined organic layer was concentrated under reduced pressure. The residue was applied onto a silica gel column and eluted with ethyl acetate/petroleum ether (1:10) to give 1.2 g (67.93%) of title compound as a white solid. LC-MS: (ES, m/z): [M+H]⁺ 348.

### Step 4: Synthesis of N-(4-bromo-2,5-dimethylphenyl)-4-(2,3-dihydroxypropyl)-1-methyl-1H-pyrazole-5-carboxamide

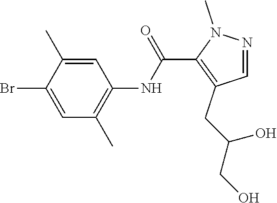 [SMILES: Cc1cc(NC(=O)c2c(CC(O)CO)cnn2C)c(C)cc1Br]

Into a 50-mL 3-necked round-bottom flask under N atmosphere, was placed 4-allyl-N-(4-bromo-2,5-dimethylphenyl)-1-methyl-1H-pyrazole-5-carboxamide (1.2 g, 3.45 mmol, 1 equiv), THF (12 mL, 148.12 mmol, 42.98 equiv), NMO (1.2 g 10.24 mmol, 2.97 equiv), H₂O (6 mL), and OsO₄ (43.8 mg, 0.17 mmol, 0.05 equiv). The resulting solution was stirred for 4 h at room temperature and then quenched with 50 mL NaS₂O₈ (aq.). The resulting solution was extracted with ethyl acetate. The combined organic layer was washed with H₂O, dried over anhydrous sodium sulfate and concentrated under vacuum to give 1.41 g of the title compound as a crude product. LC-MS: (ES, m/z): [M+H]⁺ 382.

### Step 5: Synthesis of N-(4-bromo-2,5-dimethylphenyl)-1-methyl-4-(2-oxoethyl)-1H-pyrazole-5-carboxamide

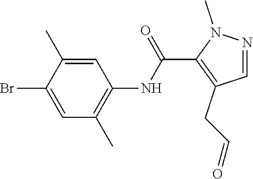 [SMILES: Cc1cc(NC(=O)c2c(CC=O)cnn2C)c(C)cc1Br]

Into a 50-mL 3-necked round-bottom flask purged was placed N-(4-bromo-2,5-dimethylphenyl)-4-(2,3-dihydroxypropyl)-1-methyl-1H-pyrazole-5-carboxamide (1.41 g, 3.69 mmol, 1 equiv), MeOH (15 mL), H₂O (1.5 mL), and NaIO₄ (1.6 g, 7.48 mmol, 2.03 equiv). The resulting solution was stirred for 2 h at room temperature and then quenched with H₂O. The resulting mixture was concentrated to remove MeOH and the resulting solution was extracted with ethyl acetate. The combined organic layer was washed with H₂O, dried over anhydrous sodium sulfate and concentrated under reduced pressure to give (1.2 g, 92.89%) the title compound as a solid. LC-MS: (ES, m/z): [M+H]⁺ 350.

### Step 6: Synthesis of N-(4-bromo-2,5-dimethylphenyl)-4-(2-hydroxyethyl)-1-methyl-1H-pyrazole-5-carboxamide

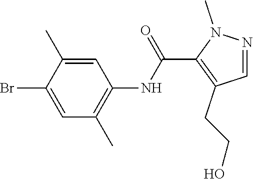 [SMILES: Cc1cc(NC(=O)c2c(CCO)cnn2C)c(C)cc1Br]

Into a 50-mL 3-necked round-bottom flask under N₂ atmosphere, was placed N-(4-bromo-2,5-dimethylphenyl)-1-methyl-4-(2-oxoethyl)-1H-pyrazole-5-carboxamide (1.2 g, 3.43 mmol, 1 equiv), MeOH (12 mL, 296.39 mmol, 86.50 equiv). NaBH₄ (0.2 g, 5.29 mmol, 1.54 equiv) was added at 0° C. The resulting solution was stirred for 2 h at RT and then quenched with NH₄Cl (aq.). The resulting solution was extracted with ethyl acetate. The combined organic layer was washed with H₂O, dried over anhydrous sodium sulfate and concentrated under reduced pressure to give (1.22 g) the title compound as crude product. LC-MS: (ES, m/z): [M+H]⁺ 352.

### Step 7: Synthesis of 2-(5-((4-bromo-2,5-dimethylphenyl)carbamoyl)-1-methyl-1H-pyrazol-4-yl)ethyl Methanesulfonate

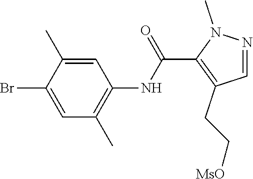 [SMILES: Cc1cc(NC(=O)c2c(CCOS(C)(=O)=O)cnn2C)c(C)cc1Br]

Into a 50-mL 3-necked round-bottom flask under N₂ atmosphere, was placed N-(4-bromo-2,5-dimethylphenyl)-4-(2-hydroxyethyl)-1-methyl-1H-pyrazole-5-carboxamide (1.3 g, 3.69 mmol, 1 equiv), DCM (15 mL), Et₃N (0.7 g, 6.92 mmol, 1.87 equiv). MsCl (0.6 g, 5.24 mmol, 1.42 equiv) was added dropwise at 0° C. The resulting solution was stirred for 2 h at room temperature. The reaction was then quenched with 50 mL of NaHCO₃ (aq.). The resulting solution was extracted with ethyl acetate and the organic layers combined. The resulting mixture was washed with H₂O, dried over anhydrous sodium sulfate and concentrated under vacuum to give 1.41 g of the title compound as a crude product. LC-MS: (ES, m/z): [M+H]⁺ 430.

### Step 8: Synthesis of 6-(4-bromo-2,5-dimethylphenyl)-1-methyl-1,4,5,6-tetrahydro-7H-pyrazolo[3,4-c]pyridin-7-one

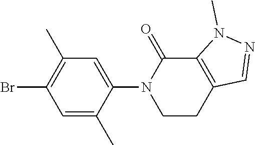 [SMILES: Cc1cc(N2CCc3cnn(C)c3C2=O)c(C)cc1Br]

Into a 50-mL 3-necked round-bottom flask under N₂ atmosphere, was placed 2-(5-((4-bromo-2,5-dimethylphenyl)carbamoyl)-1-methyl-1H-pyrazol-4-yl)ethyl methanesulfonate (1.41 g, 3.28 mmol, 1 equiv), DMF (15 mL). NaH (0.1 g, 4.17 mmol, 1.27 equiv) was added at 0° C. The resulting solution was stirred for 2 h at room temperature. The reaction was then quenched by the addition of 50 mL of NH₄Cl (aq.). The resulting solution was extracted with ethyl acetate and the organic layers combined. The resulting mixture was washed with H₂O. The residue was applied onto a silica gel column and eluted with ethyl acetate/petroleum ether (1:5) to give (843 mg, 76.98%) of the title compound as a white solid. LC-MS (ES, m/z): [M+H]⁺ 334.

### Step 9: Synthesis of 1-methyl-6-(2,5-dimethyl-4-(4,4,5,5-tetramethyl-1,3,2-dioxaborolan-2-yl)phenyl)-1,4,5,6-tetrahydro-7H-pyrazolo[3,4-c]pyridin-7-one

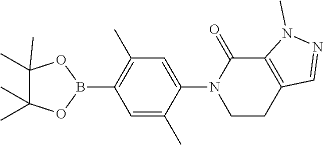 [SMILES: Cc1cc(N2CCc3cnn(C)c3C2=O)c(C)cc1B1OC(C)(C)C(C)(C)O1]

Into a 50-mL 3-necked round-bottom flask under N₂ atmosphere, was placed 6-(4-bromo-2,5-dimethylphenyl)-1-methyl-1,4,5,6-tetrahydro-7H-pyrazolo[3,4-c]pyridin-7-one (843 mg, 2.52 mmol, 1 equiv), dioxane (10 mL), KOAc (742.6 mg, 7.57 mmol, 3 equiv), and Pd(dppf)Cl₂ (553.7 mg, 0.76 mmol, 0.30 equiv). The resulting solution was stirred for overnight at 80° C. The resulting mixture was concentrated under vacuum. The residue was applied onto a silica gel column and eluted with ethyl acetate/petroleum ether (1:5) to give (520 mg, 54.07%) of the title compound as a white solid. LC-MS: (ES, m/z): [M+H]⁺ 382.

### Step 10: Synthesis of 6-(2,5-dimethyl-4-(6-(trifluoromethyl)-1,5-naphthyridin-2-yl)phenyl)-1-methyl-1,4,5,6-tetrahydro-7H-pyrazolo[3,4-c]pyridin-7-one

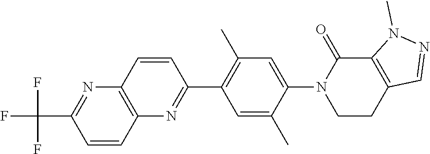 [SMILES: Cc1cc(N2CCc3cnn(C)c3C2=O)c(C)cc1-c1ccc2nc(C(F)(F)F)ccc2n1]

Proceeding analogously as described in Example 7, Step 8 above, but substituting 5-(2-fluoro-5-methyl-4-(4,4,5,5-tetramethyl-1,3,2-dioxaborolan-2-yl)phenyl)-3-methyl-6,7-dihydropyrazolo[1,5-a]pyrazin-4(5H)-one with 1-methyl-6-(2,5-dimethyl-4-(4,4,5,5-tetramethyl-1,3,2-dioxaborolan-2-yl)phenyl)-1,4,5,6-tetrahydro-7H-pyrazolo[3,4-c]pyridin-7-one gave (300 mg, 63.34%) of the title compound as a white solid. LC-MS: (ES, m/z): [M+H]⁺ 452; ¹H-NMR: (300 MHz, DMSO-d6, ppm): δ 8.75-8.78 (d, 1H), 8.67-8.70 (d, 1H), 8.26-8.28 (d, 1H), 8.16-8.19 (d, 1H), 7.55 (s, 1H), 7.48 (s, 1H), 7.34 (s, 1H), 4.01-4.09 (m, 4H), 3.71-3.79 (m, 1H), 2.90-3.04 (m, 2H), 2.40 (s, 3H), 2.25 (s, 3H).

### Example 11

### Synthesis of 6-(2-fluoro-5-methyl-4-(6-(trifluoromethyl)-1,5-naphthyridin-2-yl)phenyl)-1-methyl-1,4,5,6-tetrahydro-7H-pyrazolo[3,4-c]pyridin-7-one

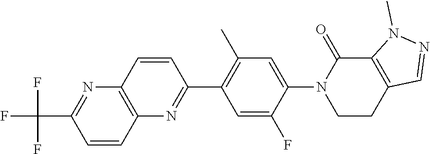 [SMILES: Cc1cc(N2CCc3cnn(C)c3C2=O)c(F)cc1-c1ccc2nc(C(F)(F)F)ccc2n1]

### Step 1: Synthesis of 4-ally-1-methyl-1H-pyrazole-5-carboxylic Acid

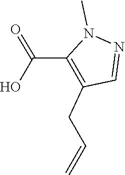

To a stirred solution of methyl 4-allyl-1-methyl-1H-pyrazole-5-carboxylate (5.5 g, 30.52 mmol, 1 equiv) in CH₃OH (55 mL) was added NaOH (2.45 g, 61.25 mmol, 2.01 equiv) at room temperature under nitrogen atmosphere. The resulting mixture was concentrated under reduced pressure. The residue was acidified to pH 6 with HCl (aq.). The resulting mixture was filtered and the filter cake was washed with water. The aqueous layer was extracted with EtOAc and the combined organic layers were dried over anhydrous Na₂SO₄. After filtration, the filtrate was concentrated under reduced pressure to give the title compound (4.5 g, 88.72%) as a white solid. LC-MS: (ES, m/z): [M+H]⁺ 167.

Methyl 4-allyl-1-methyl-1H-pyrazole-5-carboxylate was converted to 4-allyl-1-methyl-1H-pyrazole-5-carbonyl chloride as described in Example 4, Step 3 above.

### Step 2: Synthesis of 4-allyl-N-(4-bromo-2-fluoro-5-methylphenyl)-1-methyl-1H-pyrazole-5-carboxamide

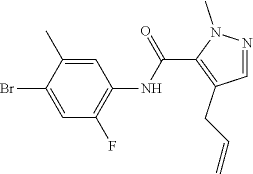 [SMILES: C=CCc1cnn(C)c1C(=O)Nc1cc(C)c(Br)cc1F]

Proceeding analogously as described in Example 4, Step 4, but substituting 4-bromo-2-methylaniline with 4-bromo-2-fluoro-5-methylaniline provided the title compound (68.49% yield) as a pink solid. LC-MS: (ES, m/z): [M+H]⁺ 352, 354.

### Step 3: Synthesis of N-(4-bromo-2-fluoro-5-methylphenyl)-4-(2,3-dihydroxypropyl)-1-methyl-1H-pyrazole-5-carboxamide

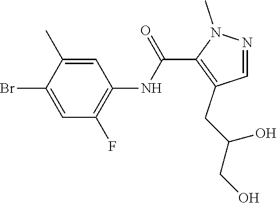 [SMILES: Cc1cc(NC(=O)c2c(CC(O)CO)cnn2C)c(F)cc1Br]

Into a 100 mL 3-necked round-bottom flask was added 4-allyl-N-(4-bromo-2-fluoro-5-methyl-phenyl)-1-methyl-1H-pyrazole-5-carboxamide (3.92 g, 11.13 mmol, 1 equiv), THF (39.2 mL), H₂O (3.92 mL), NMO (3.9 g, 33.39 mmol, 3.00 equiv) and OsO₄ (0.1 g, 0.39 mmol, 0.04 equiv). The resulting mixture was stirred for 4 h at room temperature and then quenched with Na₂S₂O₄ (aq.). The aqueous layer was extracted with EtOAc, and the resulting mixture was concentrated under reduced pressure to afford the title compound (4.5 g, 104.69%) as a white solid. The crude product was used in the next step directly without further purification. LC-MS: (ES, m/z): [M+H]⁺ 386, 388.

### Step 4: Synthesis of N-(4-bromo-2-fluoro-5-methylphenyl)-4-(2-oxoethyl)-1-methyl-1H-pyrazole-5-carboxamide

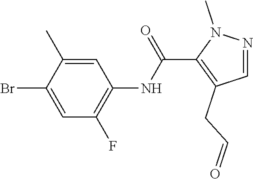 [SMILES: Cc1cc(NC(=O)c2c(CC=O)cnn2C)c(F)cc1Br]

Into a 100 mL 3-necked round-bottom flask was added N-(4-bromo-2-fluoro-5-methylphenyl)-4-(2,3-dihydroxypropyl)-1-methyl-1H-pyrazole-5-carboxamide (4.5 g, 11.65 mmol, 1 equiv), MeOH (41 mL), H₂O (41 mL) and NaIO₄ (5.0 g, 23.38 mmol, 2.01 equiv). The resulting mixture was stirred for 3 h at room temperature and then diluted with water. The resulting mixture was concentrated under reduced pressure. The aqueous layer was extracted with EtOAc and the organic layer was concentrated to afford the title compound (2.83 g, 68.58%) as a yellow solid. The crude product was used in the next step directly without further purification. LC-MS: (ES, m/z): [M+H]⁺ 354, 356.

### Step 5: Synthesis of N-(4-bromo-2-fluoro-5-methylphenyl)-4-(2-hydroxyethyl)-1-methyl-1H-pyrazole-5-carboxamide

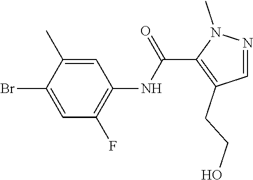 [SMILES: Cc1cc(NC(=O)c2c(CCO)cnn2C)c(F)cc1Br]

Into a 50 mL 3-necked round-bottom flask was added N-(4-bromo-2-fluoro-5-methylphenyl)-4-(2-oxoethyl)-1-methyl-H-pyrazole-5-carboxamide (2.83 g, 7.99 mmol, 1 equiv), MeOH (28.3 mL) and NaBH₄ (0.4 g, 10.57 mmol, 1.32 equiv) was added at 0° C. The resulting mixture was stirred for 3 h at room temperature under nitrogen atmosphere. The reaction was quenched by the addition of sat. NH₄Cl (aq.) (40 mL) and the resulting mixture was concentrated under reduced pressure. The aqueous layer was extracted with EtOAc and the organic layer was concentrated under reduced pressure. The residue was purified by silica gel column chromatography with PE/EtOAc (1:1) as eluent to afford the title compound (1.3 g, 45.68%) as a yellow solid. LC-MS: (ES, m/z): [M+H]⁺ 356, 358.

### Step 6. Synthesis of 2-(5-((4-bromo-2-fluoro-5-methylphenyl)carbamoyl)-1-methyl-1H-pyrazol-4-yl)ethyl methanesulfonate

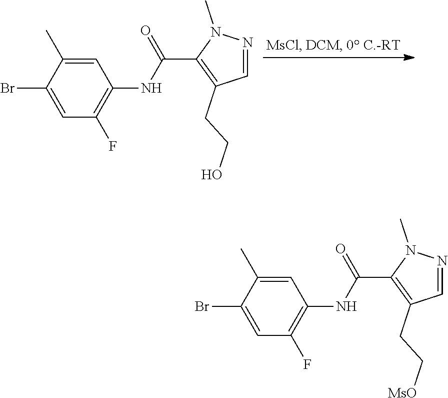 [SMILES: Cc1cc(NC(=O)c2c(CCO)cnn2C)c(F)cc1Br ; Cc1cc(NC(=O)c2c(CCOS(C)(=O)=O)cnn2C)c(F)cc1Br]

Into a 50 mL 3-necked round-bottom flask containing N-(4-bromo-2-fluoro-5-methylphenyl)-4-(2-hydroxyethyl)-1-methyl-H-pyrazole-5-carboxamide (1.3 g, 3.65 mmol, 1 equiv), and DCM (26 mL), Et₃N (0.6 g, 5.47 mmol, 1.50 equiv) MsCl (0.6 g, 5.24 mmol, 1.44 equiv) was added at 0° C. The resulting mixture was stirred for 2 h at room temperature and then diluted with water. The aqueous layer was extracted with CH₂Cl₂ and the organic layer was concentrated under vacuum to afford the title compound (1.75 g, 110.41%) as a yellow solid. The crude product was used in the next step directly without further purification. LC-MS: (ES, m/z): [M+H]⁺ 434, 436.

### Step 7: Synthesis of 6-(4-bromo-2-fluoro-5-methylphenyl)-1-methyl-1,4,5,6-tetrahydro-7H-pyrazolo[3,4-c]pyridin-7-one

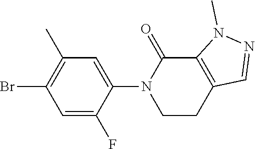 [SMILES: Cc1cc(N2CCc3cnn(C)c3C2=O)c(F)cc1Br]

Proceeding as described in Example 4, Step 9, but substituting 2-(5-((4-bromo-2-fluoro-5-methylphenyl)carbamoyl)-1-methyl-1H-pyrazol-4-yl)ethyl methanesulfonate for 2-(5-((4-bromo-2-methylphenyl)carbamoyl)-1-methyl-1H-pyrazol-4-yl)ethyl methanesulfonate the title compound was obtained (1.0 g, 73.38%) as a light yellow solid. LC-MS: (ES, m/z): [M+H]⁺ 338, 340.

### Step 8: Synthesis of 6-(2-fluoro-5-methyl-4-(4,4,5,5-tetramethyl-1,3,2-dioxaborolan-2-yl)phenyl)-1-methyl-1,4,5,6-tetrahydro-7H-pyrazolo[3,4-c]pyridin-7-one

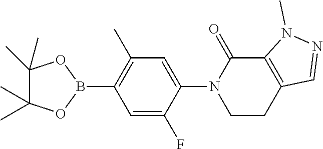 [SMILES: Cc1cc(N2CCc3cnn(C)c3C2=O)c(F)cc1B1OC(C)(C)C(C)(C)O1]

Proceeding as described in Example 4, Step 10 above, but substituting 6-(4-bromo-2-fluoro-5-methylphenyl)-1-methyl-1,4,5,6-tetrahydro-7H-pyrazolo[3,4-c]pyridin-7-one for 6-(4-bromo-2-methylphenyl)-1-methyl-1,4,5,6-tetrahydro-7H-pyrazolo[3,4-c]pyridin-7-one (1.01 g, 98.51%) of the title compound was obtained as a light yellow solid. LC-MS: (ES, m/z): [M+H]⁺ 386.

### Step 9: Synthesis of 6-(2-fluoro-5-methyl-4-(6-(trifluoromethyl)-1,5-naphthyridin-2-yl)phenyl)-1-methyl-1,4,5,6-tetrahydro-7H-pyrazolo[3,4-c]pyridin-7-one

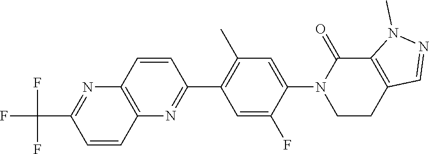 [SMILES: Cc1cc(N2CCc3cnn(C)c3C2=O)c(F)cc1-c1ccc2nc(C(F)(F)F)ccc2n1]

Into a 50 mL 3-necked round-bottom flask were added 6-(2-fluoro-5-methyl-4-(4,4,5,5-tetramethyl-1,3,2-dioxaborolan-2-yl)phenyl)-1-methyl-1,4,5,6-tetrahydro-7H-pyrazolo[3,4-c]pyridin-7-one (1 g, 2.60 mmol, 1 equiv), 2-chloro-6-(trifluoromethyl)-1,5-naphthyridine (0.6 g, 2.58 mmol, 0.99 equiv), Na₂CO₃ (0.8 g, 7.79 mmol, 3 equiv), DME (20 mL), H₂O (5 mL) and Pd(PPh₃)₄ (0.3 g, 0.26 mmol, 0.1 equiv). The resulting mixture was stirred for overnight at 80° C. under nitrogen atmosphere. The resulting mixture was concentrated under vacuum and the residue was purified by silica gel column chromatography, eluted with PE/EtOAc (1:1) to afford the title compound (596.1 mg, 50.43%) as a white solid. LC-MS: (ES, m/z): [M+H]⁺ 456; ¹H-NMR: (300 MHz, DMSO, ppm): δ2.5 (s, 3H), δ2.9 (t, 2H), δ3.9 (t, 2H), δ4.09 (s, 3H), δ7.5 (m, 2H), δ7.6 (m, 1H), δ8.2 (d, 1H), δ8.3 (d, 1H), δ8.7 (d, 1H), δ8.8 (d, 1H).

### Example 12

### Synthesis of 7-(2-fluoro-5-methyl-4-(6-(trifluoromethyl)-1,5-naphthyridin-2-yl)phenyl)-1-methyl-6,7-dihydro-1H-pyrazolo[3,4-f][1,4]oxazepin-8(5H)-one

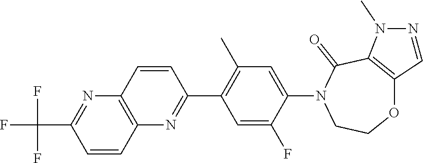 [SMILES: Cc1cc(N2CCOc3cnn(C)c3C2=O)c(F)cc1-c1ccc2nc(C(F)(F)F)ccc2n1]

### Step 1: Synthesis of 4-(2-(benzyloxy)ethoxy)-N-(4-bromo-2-fluoro-5-methylphenyl)-1-methyl-1H-pyrazole-5-carboxamide

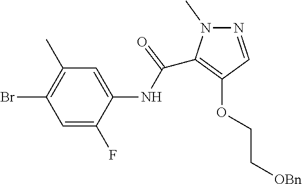 [SMILES: Cc1cc(NC(=O)c2c(OCCOCc3ccccc3)cnn2C)c(F)cc1Br]

To a stirred solution of 4-(2-(benzyloxy)ethoxy)-1-methyl-H-pyrazole-5-carbonyl chloride (0.9 g, 3.05 mmol, 1 equiv) and 4-bromo-2-fluoro-5-methylaniline (0.6 g, 3.05 mmol, 1 equiv) in DCM was added Et₃N (0.5 g, 4.58 mmol, 1.5 equiv) at 0° C. under nitrogen atmosphere. The resulting solution was stirred for 30 min. The reaction was quenched with water at room temperature. The aqueous layer was extracted with CH₂Cl₂ and the organic layer was concentrated under reduced pressure to give the title compound (1 g, 70.84%) as a white solid. LC-MS: (ES, m/z): [M+H]⁺ 462.

### Step 2. Synthesis of N-(4-bromo-2-fluoro-5-methylphenyl)-4-(2-hydroxyethoxy)-1-methyl-1H-pyrazole-5-carboxamide

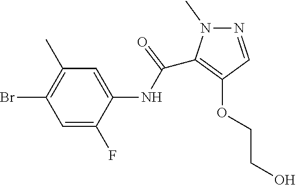 [SMILES: Cc1cc(NC(=O)c2c(OCCO)cnn2C)c(F)cc1Br]

To a stirred solution of 4-(2-(benzyloxy)ethoxy)-N-(4-bromo-2-fluoro-5-methylphenyl)-1-methyl-1H-pyrazole-5-carboxamide (1 g, 2.16 mmol, 1 equiv) in DCM was added BCl₃ (0.5 g, 4.27 mmol, 1.98 equiv) at 0° C. under nitrogen atmosphere. The resulting solution was stirred for 3 h. The reaction was quenched with water at room temperature. The aqueous layer was extracted with DCM and the resulting mixture was concentrated under reduced pressure to give the title compound (0.8 g, 99.37%) as a light yellow solid. LC-MS: (ES, m/z): [M+H]⁺ 372.

### Step 3: Synthesis of 2-((5-((4-bromo-2-fluoro-5-methylphenyl)carbamoyl)-1-methyl-1H-pyrazol-4-yl)oxy)ethyl methanesulfonate

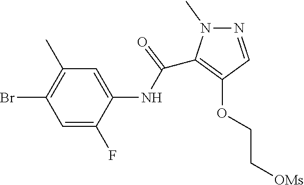 [SMILES: Cc1cc(NC(=O)c2c(OCCOS(C)(=O)=O)cnn2C)c(F)cc1Br]

To a stirred solution of N-(4-bromo-2-fluoro-5-methylphenyl)-4-(2-hydroxyethoxy)-1-methyl-1H-pyrazole-5-carboxamide (800 mg, 2.15 mmol, 1 equiv) in DCM was added Et₃N (327 mg, 3.23 mmol, 1.50 equiv) and MsCl (369 mg, 3.22 mmol, 1.50 equiv) was added at 0° C. under nitrogen atmosphere. The reaction mixture was quenched with water at room temperature. The aqueous layer was extracted with DCM and the combined organic layers were dried over anhydrous Na₂SO₄. After filtration, the filtrate was concentrated under reduced pressure to give the title compound (950 mg, 98.16%) as a white solid. LC-MS: (ES, m/z): [M+H]⁺ 450.

### Step 4: Synthesis of 7-(4-bromo-2-fluoro-5-methylphenyl)-1-methyl-6,7-dihydro-1H-pyrazolo[3,4-f][1,4]oxazepin-8(5H)-one

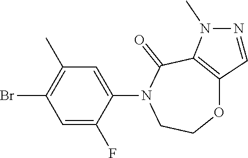 [SMILES: Cc1cc(N2CCOc3cnn(C)c3C2=O)c(F)cc1Br]

To a stirred solution of 2-((5-((4-bromo-2-fluoro-5-methylphenyl)carbamoyl)-1-methyl-1H-pyrazol-4-yl)oxy)ethyl methanesulfonate_(950 mg, 2.11 mmol, 1 equiv) in DMF (19 mL) was added NaH (127 mg, 5.29 mmol, 1.5 equiv) at 0° C. The reaction was quenched with NH₄Cl (aq.) at room temperature and the resulting mixture was extracted with EtOAc. The combined organic layers were concentrated under reduced pressure to give the title compound (618 mg, 82.70%) as a light yellow solid. LC-MS: (ES, m/z): [M+H]⁺ 354.

### Step 5: Synthesis of 1-methyl-7-(2-fluoro-5-methyl-4-(4,4,5,5-tetramethyl-1,3,2-dioxaborolan-2-yl)phenyl)-6,7-dihydro-1H-pyrazolo[3,4-f][1,4]oxazepin-8(5H)-one

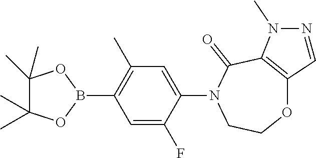 [SMILES: Cc1cc(N2CCOc3cnn(C)c3C2=O)c(F)cc1B1OC(C)(C)C(C)(C)O1]

To a stirred solution of 7-(4-bromo-2-fluoro-5-methylphenyl)-1-methyl-6,7-dihydro-1H-pyrazolo[3,4-f][1,4]oxazepin-8(5H)-one (618 mg, 1.74 mmol, 1 equiv) in dioxane was added KOAc (342 mg, 3.48 mmol, 2.00 equiv), B₂Pin₂ (665 mg, 2.62 mmol, 1.50 equiv) and Pd(dppf)Cl2(128 mg, 0.17 mmol, 0.10 equiv) and the reaction mixture was heated at 80° C. under nitrogen atmosphere. The resulting mixture was concentrated under reduced pressure and the residue was purified by silica gel column chromatography with PE/EtOAc (10:1) as eluent to afford the title compound (714 mg, 101.98%) as a white solid. LC-MS: (ES, m/z): [M+H]⁺ 402.

### Step 6: Synthesis of 7-(2-fluoro-5-methyl-4-(6-(trifluoromethyl)-1,5-naphthyridin-2-yl)phenyl)-1-methyl-6,7-dihydro-1H-pyrazolo[3,4-f][1,4]oxazepin-8(5H)-one

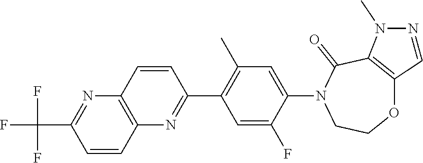 [SMILES: Cc1cc(N2CCOc3cnn(C)c3C2=O)c(F)cc1-c1ccc2nc(C(F)(F)F)ccc2n1]

To a stirred solution of 1-methyl-7-(2-fluoro-5-methyl-4-(4,4,5,5-tetramethyl-1,3,2-dioxaborolan-2-yl)phenyl)-6,7-dihydro-1H-pyrazolo[3,4-f][1,4]oxazepin-8(5H)-one (714 mg, 1.78 mmol, 1 equiv) and 2-chloro-6-(trifluoromethyl)-1,5-naphthyridine (415 mg, 1.78 mmol, 1.00 equiv) in DME (14.3 mL) were added Pd(PPh₃)₄ (205.8 mg, 0.18 mmol, 0.10 equiv), Na2CO3 (566 mg, 5.34 mmol, 3.00 equiv) and H2O (3.6 mL) at 80° C. under nitrogen atmosphere. The reaction was quenched by the addition of water at room temperature. The aqueous layer was extracted with EtOAc and the combined organic layers were concentrated under reduced pressure. The residue was purified by silica gel column chromatography, with PE/EtOAc (5/1) as eluent to afford the title compound (0.4245 g, 50.60%) as a white solid. LC-MS: (ES, m/z): [M+H]⁺ 472; ¹H-NMR: (300 MHz, CDCl₃, ppm): δ2.43 (s, 3H), δ3.99-4.01 (t, 2H), δ4.16 (s, 3H), δ4.51-4.53 (t, 2H), δ7.26-7.32 (m, 2H), δ7.40-7.44 (d, 1H), δ7.87-7.90 (d, 1H), δ8.02-8.04 (d, 1H), δ8.60-8.65 (m, 2H).

### Example 13

### Synthesis of 5-(2-fluoro-4-(6-(trifluoromethyl)quinazolin-2-yl)phenyl)-3-methyl-6,7-dihydropyrazolo[1,5-a]pyrazin-4(5H)-one

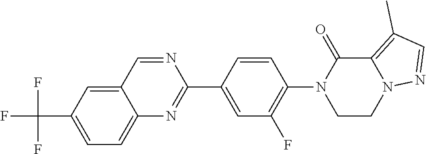 [SMILES: Cc1cnn2c1C(=O)N(c1ccc(-c3ncc4cc(C(F)(F)F)ccc4n3)cc1F)CC2]

### Step 1: Synthesis of ethyl 1-(2-(benzyloxy)ethyl)-4-methyl-1H-pyrazole-5-carboxylate

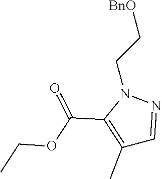 [SMILES: CCOC(=O)c1c(C)cnn1CCOCc1ccccc1]

To a stirred solution of ethyl 4-methyl-H-pyrazole-5-carboxylate (50 g, 324.32 mmol, 1 equiv) and 2-(benzyloxy)ethan-1-ol (49.4 g, 324.59 mmol, 1.00 equiv) in THF (500 mL) were added DIAD (137.7 g 681.07 mmol, 2.1 equiv) and PPh₃(221.2 g, 843.23 mmol, 2.6 equiv) in portions at 0° C. under nitrogen atmosphere. The resulting mixture was stirred overnight at room temperature. The residue was purified by silica gel column chromatography with PE/EtOAc (50/1) as eluent to afford the title compound (73 g, 78.06%) as a yellow oil. LC-MS: (ES, m/z): [M+H]⁺ 289.

### Step 2: Synthesis of 1-(2-(benzyloxy)ethyl)-4-methyl-1H-pyrazole-5-carboxylic Acid

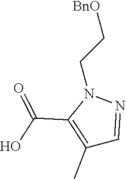 [SMILES: Cc1cnn(CCOCc2ccccc2)c1C(=O)O]

To a stirred solution of ethyl 1-(2-(benzyloxy)ethyl)-4-methyl-1H-pyrazole-5-carboxylate (36.7 g 127.28 mmol, 1 equiv) in EtOH (367 mL) was added NaOH (4M) (63.64 mL, 2 equiv). The resulting mixture was stirred for 1 h at 40° C. and then quenched with water at room temperature. The reaction mixture was acidified to pH 4 with HCl (2M) (aq.) and the resulting mixture was extracted with EtOAc. The combined organic layers were dried over anhydrous Na₂SO₄. After filtration, the filtrate was concentrated under reduced pressure to give the title compound (30 g 90.55%) as a white solid. LC-MS: (ES, m/z): [M+H]⁺ 261.

### Step 3: Synthesis of 1-(2-(benzyloxy)ethyl)-4-methyl-1H-pyrazole-5-carbonyl Chloride

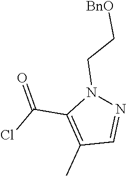 [SMILES: Cc1cnn(CCOCc2ccccc2)c1C(=O)Cl]

To a stirred solution of 1-(2-(benzyloxy)ethyl)-4-methyl-1H-pyrazole-5-carboxylic acid (15 g 57.63 mmol, 1 equiv) and DMF (0.4 g, 5.76 mmol, 0.1 equiv) in DCM (300 mL) was added (COCl)₂ (11.0 g 86.44 mmol, 1.5 equiv) dropwise at 0° C. The resulting mixture was stirred for 3 h at room temperature and then concentrated under reduced pressure to give the title compound (17 g, crude) as a white solid.

### Step 4: Synthesis of 1-(2-(benzyloxy)ethyl)-N-(2-fluoro-4-bromophenyl)-4-methyl-1H-pyrazole-5-carboxamide

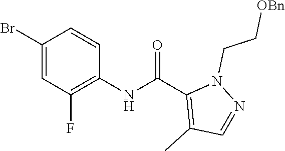 [SMILES: Cc1cnn(CCOCc2ccccc2)c1C(=O)Nc1ccc(Br)cc1F]

Into a 50-mL 3-necked round-bottom flask under N₂ atmosphere, was placed 1-(2-(benzyloxy)ethyl)-4-methyl-1H-pyrazole-5-carbonyl chloride (2 g, 7.18 mmol, 1 equiv), 4-bromo-2-fluoroaniline (1.6 g, 8.61 mmol, 1.2 equiv), Et₃N (1.1 g, 10.76 mmol, 1.5 equiv), and DCM (20 mL). The resulting solution was stirred for 2 h at 25° C. The reaction was then quenched with water and the resulting solution was extracted with dichloromethane. The organic layers were concentrated under vacuum. The crude product was purified by re-crystallization from CH₃OH to give (2.5 g, 80.60%) of the title compound as a white solid. LC-MS: (ES, m/z): [M+H]⁺ 432, 434.

### Step 5: Synthesis of N-(4-bromo-2-fluorophenyl)-1-(2-hydroxyethyl)-4-methyl-1H-pyrazole-5-carboxamide

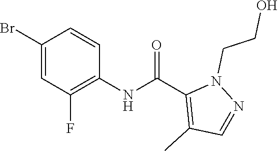 [SMILES: Cc1cnn(CCO)c1C(=O)Nc1ccc(Br)cc1F]

Into a 50-mL 3-necked round-bottom flask under N₂ atmosphere, was placed 1-(2-(benzyloxy)ethyl)-N-(2-fluoro-4-bromophenyl)-4-methyl-1H-pyrazole-5-carboxamide (2.5 g, 5.78 mmol, 1 equiv), DCM (25 mL), and BCl₃ (1M) (8.7 mL, 1.5 equiv) was added at 0° C. The resulting solution was stirred for 1 h at 0° C. The reaction was then quenched with NaHCO₃. The resulting solution was extracted with dichloromethane dried over anhydrous sodium sulfate and concentrated under vacuum to give (1.8 g, 94.85%) of the title compound as a white solid. LC-MS: (ES, m/z): [M+H]⁺ 342, 344.

### Step 6: Synthesis of 2-(5-((4-bromo-2-fluorophenyl)carbamoyl)-4-methyl-1H-pyrazol-1-yl)ethyl Methanesulfonate

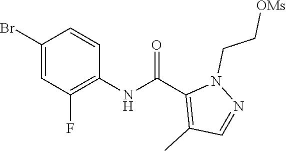 [SMILES: Cc1cnn(CCOS(C)(=O)=O)c1C(=O)Nc1ccc(Br)cc1F]

Into a 100-mL 3-necked round-bottom flask under N₂ atmosphere, was placed N-(4-bromo-2-fluorophenyl)-1-(2-hydroxyethyl)-4-methyl-1H-pyrazole-5-carboxamide (1.8 g, 5.26 mmol, 1 equiv), Et₃N (0.8 g, 7.89 mmol, 1.5 equiv), and DCM (40 mL), and MsCl (0.9 g, 7.89 mmol, 1.5 equiv) was added at 0° C. The resulting solution was stirred for 1 h at 0° C. The reaction mixture was then quenched with water and the resulting solution was extracted with dichloromethane dried over anhydrous sodium sulfate and concentrated to give (2.2 g, 99.51%) the title compound as a white solid. LC-MS: (ES, m/z): [M+H]⁺ 420, 422.

### Step 7: Synthesis of 5-(4-bromo-2-fluorophenyl)-3-methyl-6,7-dihydropyrazolo[1,5-a]pyrazin-4(5H)-one

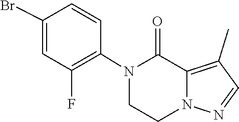 [SMILES: Cc1cnn2c1C(=O)N(c1ccc(Br)cc1F)CC2]

Into a 100-mL 3-necked round-bottom flask under N₂ atmosphere, was placed 2-(5-((4-bromo-2-fluorophenyl)carbamoyl)-4-methyl-1H-pyrazol-1-yl)ethyl methanesulfonate (2.2 g, 5.23 mmol, 1 equiv), DMF (45 mL), and NaH (0.33 g, 13.75 mmol, 2.63 equiv) was added at 0° C. The resulting solution was stirred for 1 h at 0° C. The reaction was then quenched with NH₄Cl, the resulting solution was extracted with ethyl acetate and the organic layer concentrated. The residue was applied onto a silica gel column and eluted with dichloromethane/methanol (50/1) to give (1.65 g, 97.23%) of the title compound as a yellow solid. LC-MS: (ES, m/z): [M+H]⁺ 324, 326.

### Step 8: Synthesis of 5-(2-fluoro-4-(4,4,5,5-tetramethyl-1,3,2-dioxaborolan-2-yl)phenyl)-3-methyl-6,7-dihydropyrazolo[1,5-a]pyrazin-4(5H)-one

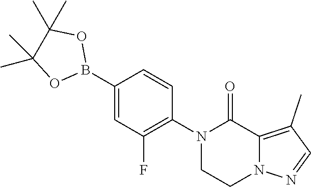 [SMILES: Cc1cnn2c1C(=O)N(c1ccc(B3OC(C)(C)C(C)(C)O3)cc1F)CC2]

Into a 50-mL 3-necked round-bottom flask under N₂ atmosphere, was placed 5-(4-bromo-2-fluorophenyl)-3-methyl-6,7-dihydropyrazolo[1,5-a]pyrazin-4(5H)-one (1.65 g, 5.09 mmol, 1 equiv), B₂Pin₂ (1.9 g, 7.64 mmol, 1.5 equiv), KOAc (1.0 g, 10.18 mmol, 2 equiv), dioxane (20 mL), and Pd(dppf)Cl₂ (0.6 g, 0.76 mmol, 0.15 equiv). The resulting solution was stirred for 12 h at 80° C. The resulting mixture was concentrated and the residue was applied onto a silica gel column and eluted with ethyl acetate/petroleum ether (1/10) to give (1.85 g, 97.91%) the title compound as a yellow solid. LC-MS: (ES, m/z): [M+H]⁺ 372.

### Step 9: Synthesis of 5-(2-fluoro-4-(6-(trifluoromethyl)quinazolin-2-yl)phenyl)-3-methyl-6,7-dihydropyrazolo[1,5-a]pyrazin-4(5H)-one

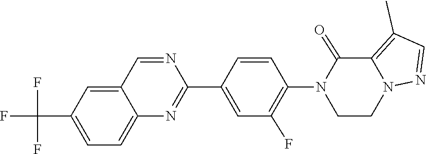 [SMILES: Cc1cnn2c1C(=O)N(c1ccc(-c3ncc4cc(C(F)(F)F)ccc4n3)cc1F)CC2]

Into a 50-mL 3-necked round-bottom flask under N₂ atmosphere, was placed 5-(2-fluoro-4-(4,4,5,5-tetramethyl-1,3,2-dioxaborolan-2-yl)phenyl)-3-methyl-6,7-dihydropyrazolo[1,5-a]pyrazin-4(5H)-one (1 g, 2.69 mmol, 1 equiv), 2-chloro-6-(trifluoromethyl)quinazoline (0.8 g, 3.23 mmol, 1.2 equiv), K₂CO₃ (1.1 g, 8.08 mmol, 3 equiv), toluene (16 mL), EtOH (8 mL), and Pd(PPh₃)₄ (0.5 g, 0.40 mmol, 0.15 equiv). The resulting solution was stirred for 12 h at 80° C. The reaction was then quenched with water and the resulting solution was extracted with ethyl acetate. The organics were concentrated and the residue was applied onto a silica gel column and eluted with ethyl acetate/petroleum ether (1/5) to give (554.4 mg, 46.63%) of the title compound as a white solid. LC-MS: (ES, m/z): [M+H]⁺ 442. ¹H-NMR: (300 MHz, DMSO, ppm): δ2.27 (s, 3H), δ4.21-4.25 (dd, 2H), δ4.51-4.55 (dd, 2H), δ7.56-7.57 (d, 1H), δ7.50 (s, 1H), δ7.72-7.77 (m, 1H), δ8.27-8.34 (m, 2H), δ8.38-8.39 (m, 1H), δ8.42-8.43 (m, 1H), δ8.49-8.51 (d, 1H), δ8.75 (s, 1H), δ9.92 (s, 1H).

### Example 14

### Synthesis of 1-methyl-5-(2-methyl-4-(6-(trifluoromethyl)quinazolin-2-yl)phenyl)-4,5-dihydropyrrolo[3,4-c]pyrazol-6(1H)-one

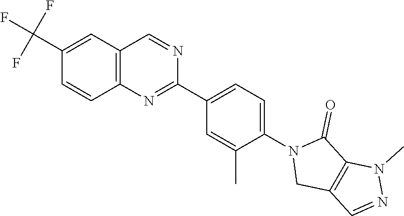 [SMILES: Cc1cc(-c2ncc3cc(C(F)(F)F)ccc3n2)ccc1N1Cc2cnn(C)c2C1=O]

### Step 1: Synthesis of methyl 1-methyl-4-vinyl-H-pyrazole-5-carboxylate

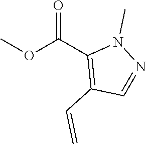

Into a 50-mL 3-necked round-bottom flask under N₂ atmosphere, was placed methyl 4-bromo-1-methyl-1H-pyrazole-5-carboxylate (1 g, 4.57 mmol, 1 equiv), tributyl(ethenyl)stannane (1.4 g, 4.41 mmol, 0.97 equiv), Pd(PPh₃)₄ (0.5 g, 0.43 mmol, 0.09 equiv) in DMF (10 mL). The resulting solution was stirred overnight at 100° C. The resulting solution was diluted with H₂O and extracted with ethyl acetate and the organic layer concentrated. The residue was applied onto a silica gel column and eluted with EA/PE (1:1) to give the title compound (580 mg 76.45%) as colorless oil. LC-MS: (ES, m/z): [M+H]⁺ 167.

### Step 2: Synthesis of 1-methyl-4-vinyl-1H-pyrazole-5-carboxylic Acid

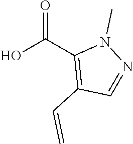

Into a 25-mL round-bottom flask, was placed methyl 1-methyl-4-vinyl-H-pyrazole-5-carboxylate (560 mg, 3.37 mmol, 1 equiv), LiOH (96.8 mg, 4.04 mmol, 1.20 equiv), THF (5 mL, 61.71 mmol, 18.31 equiv), and H₂O (5 mL, 277.54 mmol, 82.36 equiv). The resulting solution was stirred overnight at room temperature and then diluted with H₂O. The resulting mixture was concentrated, extracted with ethyl acetate, dried over anhydrous sodium sulfate and concentrated to give (380 mg, 74.11%) the title compound as a white solid. LC-MS: (ES, m/z): [M+H]⁺ 153.

### Step 3: Synthesis of 1-methyl-4-vinyl-1H-pyrazole-5-carbonyl Chloride

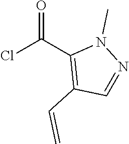

Into a 100 mL 3-necked round-bottom flask was added 1-methyl-4-vinyl-H-pyrazole-5-carboxylic acid (1.92 g, 12.62 mmol, 1 equiv), DCM (40 mL), DMF (0.1 g, 1.26 mmol, 0.1 equiv) and (COCl)₂ (2.4 g, 18.91 mmol, 1.50 equiv) was added at 0° C. The resulting mixture was stirred for 2 h at room temperature and then concentrated under reduced pressure to afford the title compound (2.3 g, 106.84%) as a yellow solid. The crude product was used in the next step directly without further purification.

### Step 4: Synthesis of N-(4-bromo-2-methylphenyl)-1-methyl-4-vinyl-H-pyrazole-5-carboxamide

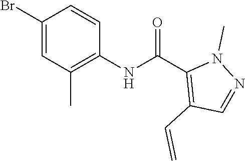 [SMILES: C=Cc1cnn(C)c1C(=O)Nc1ccc(Br)cc1C]

Into a 100 mL 3-necked round-bottom flask was added 4-bromo-2-methylaniline (2.4 g, 12.97 mmol, 1.02 equiv), DCM (40 mL), Et₃N (1.9 g, 18.78 mmol, 1.48 equiv) and 4-ethenyl-1-methyl-1H-pyrazole-5-carbonyl chloride (2.16 g, 12.66 mmol, 1 equiv) at 0° C. The resulting mixture was stirred for 2 h at room temperature and then diluted with water. The aqueous layer was extracted with CH₂Cl₂ and the organics were separated and concentrated under vacuum. The crude product was recrystallized from MeOH (20 mL) to give the title compound (3.8 g, 93.73%) as a light yellow solid. LC-MS: (ES, m/z): [M+H]⁺ 320, 322.

### Step 5: Synthesis of N-(4-bromo-2-methylphenyl)-4-(1,2-dihydroxyethyl)-1-methyl-1H-pyrazole-5-carboxamide

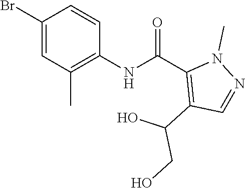 [SMILES: Cc1cc(Br)ccc1NC(=O)c1c(C(O)CO)cnn1C]

Into a 8 mL sealed tube were added N-(4-bromo-2-methylphenyl)-1-methyl-4-vinyl-1H-pyrazole-5-carboxamide (200 mg, 0.62 mmol, 1 equiv), NMO (219.5 mg, 1.87 mmol, 3.00 equiv), THF (2 mL), H₂O (0.2 mL) and OsO₄ (7.9 mg, 0.03 mmol, 0.05 equiv). The resulting mixture was stirred for 4 h at room temperature under nitrogen atmosphere. The reaction was quenched with Na2S204 (aq.) at room temperature. The aqueous layer was extracted with EtOAc and the resulting mixture was concentrated under reduced pressure to afford the title compound (208 mg, 94.01%) as a yellow solid. LC-MS: (ES, m/z): [M+H]⁺ 354, 356.

### Step 6: Synthesis of N-(4-bromo-2-methylphenyl)-4-formyl-1-methyl-1H-pyrazole-5-carboxamide

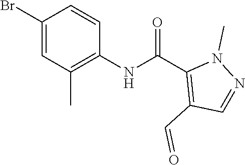 [SMILES: Cc1cc(Br)ccc1NC(=O)c1c(C=O)cnn1C]

Into a 8 mL sealed tube were added N-(4-bromo-2-methylphenyl)-4-(1,2-dihydroxyethyl)-1-methyl-1H-pyrazole-5-carboxamide (208 mg, 0.59 mmol, 1 equiv), MeOH (2 mL), H₂O (0.2 mL) and NaIO₄ (251.2 mg, 1.17 mmol, 2.00 equiv). The resulting mixture was stirred for 4 h at room temperature and then diluted with water. The aqueous layer was extracted with EtOAc and the resulting mixture was concentrated under vacuum to afford the title compound (180 mg, 95.15%) as a yellow solid. LC-MS: (ES, m/z): [M+H]⁺ 322, 324.

### Step 7: Synthesis of N-(4-bromo-2-methylphenyl)-4-(hydroxymethyl)-1-methyl-1H-pyrazole-5-carboxamide

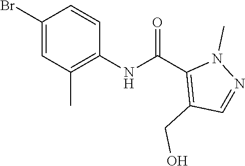 [SMILES: Cc1cc(Br)ccc1NC(=O)c1c(CO)cnn1C]

Into a 8 mL sealed tube were added N-(4-bromo-2-methylphenyl)-4-formyl-1-methyl-1H-pyrazole-5-carboxamide (180 mg, 0.56 mmol, 1 equiv), MeOH (1.8 mL) and NaBH₄ (25.4 mg, 0.67 mmol, 1.20 equiv) was added at 0° C. The resulting mixture was stirred for 4 h at room temperature under nitrogen atmosphere. The reaction was quenched with sat. NH₄Cl (aq.). The resulting mixture was concentrated under vacuum and the aqueous layer was extracted with EtOAc. The organic layer was concentrated under vacuum to the title compound (161 mg, 88.89%) as a white solid. LC-MS: (ES, m/z): [M+H]⁺ 324, 326.

### Step 8: Synthesis of (5-((4-bromo-2-methylphenyl)carbamoyl)-1-methyl-1H-pyrazol-4-yl)methyl Methanesulfonate

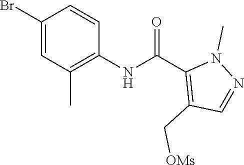 [SMILES: Cc1cc(Br)ccc1NC(=O)c1c(COS(C)(=O)=O)cnn1C]

Into a 8 mL sealed tube were added N-(4-bromo-2-methylphenyl)-4-(hydroxymethyl)-1-methyl-1H-pyrazole-5-carboxamide (161 mg, 0.50 mmol, 1 equiv), DCM (3.22 mL), and Et₃N (75.4 mg, 0.74 mmol, 1.50 equiv) and MsCl (87.6 mg, 0.76 mmol, 1.54 equiv) was added at 0° C. The resulting mixture was stirred for 2 h at room temperature and then diluted with water. The aqueous layer was extracted with CH₂Cl₂ and the organics were concentrated under reduced pressure to afford the title compound (230 mg, 115.13%) as a yellow solid. The crude product was used in the next step directly without further purification.

### Step 9: Synthesis of 5-(4-bromo-2-methylphenyl)-1-methyl-4,5-dihydropyrrolo[3,4-c]pyrazol-6(1H)-one

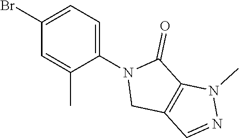 [SMILES: Cc1cc(Br)ccc1N1Cc2cnn(C)c2C1=O]

Into a 25 mL 3-necked round-bottom flask were added (5-((4-bromo-2-methylphenyl)carbamoyl)-1-methyl-1H-pyrazol-4-yl)methyl methanesulfonate (220 mg, 0.55 mmol, 1 equiv), and DMF (4.4 mL) and NaH (19.7 mg, 0.82 mmol, 1.50 equiv) was added at 0° C. in portions. The resulting mixture was stirred for 2 h at room temperature under nitrogen atmosphere. The reaction was quenched with sat. NH₄Cl (aq.). The aqueous layer was extracted with EtOAc and the organics were concentrated under reduced pressure. The residue was purified by Prep-TLC (PE/EtOAc 2:1) to afford the title compound (110 mg, 65.69%) as a yellow solid. LC-MS: (ES, m/z): [M+H]⁺ 306, 308.

### Step 10: Synthesis of 1-methyl-5-(2-methyl-4-(4,4,5,5-tetramethyl-1,3,2-dioxaborolan-2-yl)phenyl)-4,5-dihydropyrrolo[3,4-c]pyrazol-6(1H)-one

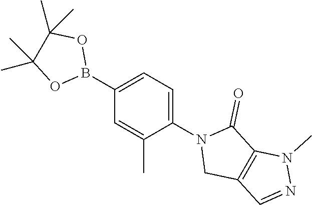

Into a 8 mL sealed tube were added 5-(4-bromo-2-methylphenyl)-1-methyl-4,5-dihydropyrrolo[3,4-c]pyrazol-6(1H)-one (110 mg, 0.36 mmol, 1 equiv), 4,4,5,5-tetramethyl-2-(4,4,5,5-tetramethyl-1,3,2-dioxaborolan-2-yl)-1,3,2-dioxaborolane (100.4 mg, 0.40 mmol, 1.10 equiv), KOAc (70.5 mg, 0.72 mmol, 2 equiv), dioxane (2.2 mL) and Pd(dppf)Cl₂ (26.3 mg, 0.04 mmol, 0.1 equiv). The reaction mixture was stirred overnight at 80° C. under nitrogen atmosphere and concentrated under reduced pressure. The residue was purified by silica gel column chromatography, eluting with PE/EtOAc (1:1) to afford the title compound (120 mg, 94.55%) as a yellow solid. LC-MS: (ES, m/z): [M+H]⁺ 354.

### Step 11: Synthesis of 1-methyl-5-(2-methyl-4-(6-(trifluoromethyl)quinazolin-2-yl)phenyl)-4,5-dihydropyrrolo[3,4-c]pyrazol-6(1H)-one

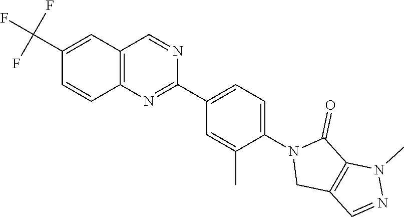 [SMILES: Cc1cc(-c2ncc3cc(C(F)(F)F)ccc3n2)ccc1N1Cc2cnn(C)c2C1=O]

Into a 8 mL sealed tube were added 1-methyl-5-(2-methyl-4-(4,4,5,5-tetramethyl-1,3,2-dioxaborolan-2-yl)phenyl)-4,5-dihydropyrrolo[3,4-c]pyrazol-6(1H)-one (110 mg, 0.31 mmol, 1 equiv), 2-chloro-6-(trifluoromethyl)quinazoline (72.4 mg, 0.31 mmol, 1.00 equiv), K₂CO₃ (129.1 mg, 0.93 mmol, 3 equiv), toluene (2.2 mL), EtOH (1.1 mL) and Pd(PPh₃)₄ (36.0 mg, 0.03 mmol, 0.1 equiv). The resulting mixture was stirred overnight at 80° C. under nitrogen atmosphere, then concentrated under reduced pressure. The residue was purified by Prep-TLC (PE/EtOAc 1:1) to afford the title compound (57.8 mg, 43.84%) as a light yellow solid. LC-MS: (ES, m/z): [M+H]⁺ 424; ¹H-NMR: (300 MHz, DMSO, ppm): δ2.3 (s, 3H), δ4.0 (s, 3H), δ4.7 (s, 2H), δ7.6 (m, 2H), δ8.26 (s, 2H), δ8.5 (d, 1H), δ8.57 (s, 1H), δ8.73 (s, 1H), δ9.9 (s, 1H).

### Example 15

### Synthesis of 1-methyl-6-(2-methyl-4-(6-(trifluoromethyl)quinazolin-2-yl)phenyl)-1,4,5,6-tetrahydro-7H-pyrazolo[3,4-c]pyridin-7-one

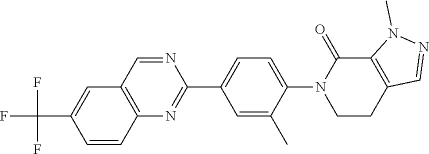 [SMILES: Cc1cc(-c2ncc3cc(C(F)(F)F)ccc3n2)ccc1N1CCc2cnn(C)c2C1=O]

Into a 50-mL 3-necked round-bottom flask under N₂ atmosphere, was placed 1-methyl-6-(2-methyl-4-(4,4,5,5-tetramethyl-1,3,2-dioxaborolan-2-yl)phenyl)-1,4,5,6-tetrahydro-7H-pyrazolo[3,4-c]pyridin-7-one (1 g, 2.72 mmol, 1 equiv), 2-chloro-6-(trifluoromethyl)quinazoline (633.3 mg, 2.72 mmol, 1 equiv), K₂CO₃ (1128.9 mg, 8.17 mmol, 3.00 equiv), toluene (10 mL), EtOH (5 mL), and Pd(PPh₃)₄ (314.6 mg, 0.27 mmol, 0.1 equiv). The resulting solution was stirred overnight at 80° C., diluted with EtOAc and washed with H₂O. The organic layer was concentrated and the residue was applied onto a silica gel column and eluted with ethyl acetate/petroleum ether (1:2) to give (516.6 mg, 43.37%) of the title compound as a white solid. LC-MS: (ES, m/z): [M+H]⁺ 438; ¹H-NMR: (400 MHz, DMSO-d6, ppm): δ 9.90 (s, 1H), 8.72 (s, 1H), 8.54-8.55 (d, 1H), 8.47-8.49 (m, 1H), 8.25-8.30 (m, 2H), 7.48-7.53 (m, 2H), 4.05-4.12 (m, 4H), 3.72-3.78 (m, 1H), 2.91-3.05 (m, 2H), 2.34 (s, 3H).

### Example 16

### Synthesis of 3-methyl-5-(2-methyl-4-(6-(trifluoromethyl)pyrido[3,2-d]pyrimidin-2-yl)phenyl)-6,7-dihydropyrazolo[1,5-a]pyrazin-4(5H)-one

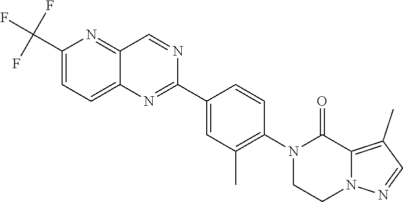 [SMILES: Cc1cc(-c2ncc3nc(C(F)(F)F)ccc3n2)ccc1N1CCn2ncc(C)c2C1=O]

To a stirred solution of 2-chloro-6-(trifluoromethyl)quinazoline (1.5 g, 6.42 mmol, 1 equiv) and 3-methyl-5-(2-methyl-4-(4,4,5,5-tetramethyl-1,3,2-dioxaborolan-2-yl)phenyl)-6,7-dihydropyrazolo[1,5-a]pyrazin-4(5H)-one (2.4 g, 6.42 mmol, 1 equiv) in t-BuOH (27 mL) and H₂O (3 mL) were added K₂CO₃ (2.7 g, 19.27 mmol, 3 equiv) and AMPhosPdCl₂ (1.4 g, 1.93 mmol, 0.3 equiv). The resulting mixture was stirred overnight at 80° C. under nitrogen atmosphere and then diluted with water. The resulting mixture was extracted with EtOAc and the combined organic layers were concentrated under reduced pressure. The residue was purified by silica gel column chromatography, by eluting with PE/EtOAc (1/2) to give crude product. The residue was purified by reverse flash chromatography with the following conditions: column, C18 gel; mobile phase, ACN and water (10 mmol/LNH₄HCO₃), 0% to 65% gradient in 40 min; detector, UV 254 nm to give the title compound (535.6 mg, 19.02%) as a yellow solid. LC-MS: (ES, m/z): [M+H]⁺ 439; ¹H-NMR: (300 MHz, DMSO, ppm): δ2.26 (s, 3H), δ2.36 (s, 3H), δ3.91-3.95 (m, 1H), δ4.28-4.36 (m, 1H), δ4.51-4.55 (m, 2H), δ7.48 (s, 1H), δ7.57-7.60 (d, 1H), δ8.46-8.57 (m, 3H), 8.81-8.84 (d, 1H), δ9.97 (s, 1H).

### Example 17

### Synthesis of 3-methyl-5-(2-methyl-4-(8-methyl-6-(trifluoromethyl)-1,5-naphthyridin-2-yl)phenyl)-6,7-dihydropyrazolo[1,5-a]pyrazin-4(5H)-one

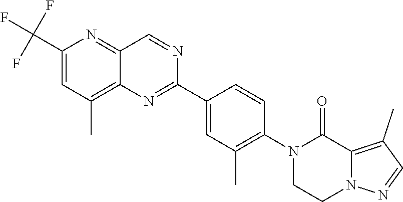 [SMILES: Cc1cc(-c2ncc3nc(C(F)(F)F)cc(C)c3n2)ccc1N1CCn2ncc(C)c2C1=O]

To a stirred solution of 6-chloro-4-methyl-2-(trifluoromethyl)-1,5-naphthyridine (500 mg, 2.03 mmol, 1 equiv) and 3-methyl-5-(2-methyl-4-(4,4,5,5-tetramethyl-1,3,2-dioxaborolan-2-yl)phenyl)-6,7-dihydropyrazolo[1,5-a]pyrazin-4(5H)-one (744.6 mg, 2.03 mmol, 1.00 equiv) in DME (10 mL) and H₂O (2.5 mL) were added Na₂CO₃ (429.8 mg, 4.05 mmol, 2 equiv) and Pd(PPh₃)₄ (234.3 mg, 0.20 mmol, 0.1 equiv) at room temperature under nitrogen atmosphere. The resulting mixture was stirred overnight at 80° C. under nitrogen atmosphere. The reaction mixture was diluted with water at room temperature and extracted with EtOAc. The organic layer was concentrated under vacuum and the residue was purified by silica gel column chromatography by eluting with PE/EtOAc (1:2) to afford crude product. The crude product was re-crystallized from MeOH (10 mL) to afford the title compound (652 mg, 71.23%) as a white solid. LC-MS: (ES, m/z): [M+H]⁺ 452; ¹H-NMR: δ2.26 (s, 3H), δ2.35 (s, 3H), δ2.97 (s, 3H), δ3.94-3.98 (m, 1H), δ4.28-4.37 (m, 1H), δ4.51-4.55 (t, 2H), δ7.47 (s, 1H), δ7.54-7.57 (d, 1H), δ8.16 (s, 1H), δ8.26-8.33 (m, 2H), δ8.57-8.66 (m, 2H).

### Example 18

### Synthesis of 5-(2,5-dimethyl-4-(6-(trifluoromethyl)-1,5-naphthyridin-2-yl)phenyl)-3-methyl-6,7-dihydropyrazolo[1,5-a]pyrazin-4(5H)-one

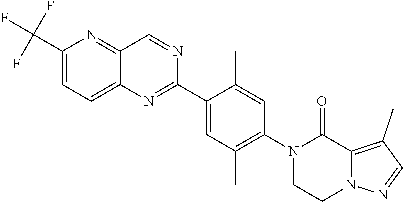 [SMILES: Cc1cc(N2CCn3ncc(C)c3C2=O)c(C)cc1-c1ncc2nc(C(F)(F)F)ccc2n1]

Proceeding as described in Example 5, Step 12 above, but substituting 6-chloro-4-methyl-2-(trifluoromethyl)-1,5-naphthyridine with 2-chloro-6-(trifluoromethyl)-1,5-naphthyridine and 7-(2,5-dimethyl-4-(4,4,5,5-tetramethyl-1,3,2-dioxaborolan-2-yl)phenyl)-1-methyl-6,7-dihydro-1H-pyrazolo[3,4-f][1,4]oxazepin-8(5H)-one with 5-(2,5-dimethyl-4-(4,4,5,5-tetramethyl-1,3,2-dioxaborolan-2-yl)phenyl)-3-methyl-6,7-dihydropyrazolo[1,5-a]pyrazin-4(5H)-one gave crude product. Purification by silica gel column chromatography using ethyl acetate/petroleum ether (1/1) as eluent gave the title compound as a white solid in 51.79% yield. LC-MS: (ES, m/z): [M+H]⁺ 452; ¹H-NMR: (300 MHz, DMSO, ppm): δ2.19 (s, 6H), δ2.40 (s, 3H), δ3.89-3.94 (m, 1H), δ4.22-4.31 (m, 1H), δ4.50-4.54 (m, 2H), δ7.37-7.56 (m, 3H), δ8.16-8.29 (m, 2H), δ8.67-8.78 (m, 2H).

### Example 19

### Synthesis of 5-(2,5-dimethyl-4-(6-(trifluoromethyl)pyrido[3,2-d]pyrimidin-2-yl)phenyl)-3-methyl-6,7-dihydropyrazolo[1,5-a]pyrazin-4(5H)-one

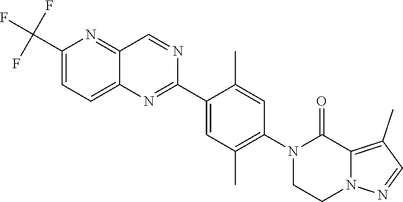 [SMILES: Cc1cc(N2CCn3ncc(C)c3C2=O)c(C)cc1-c1ncc2nc(C(F)(F)F)ccc2n1]

To a stirred solution of 5-(2,5-dimethyl-4-(4,4,5,5-tetramethyl-1,3,2-dioxaborolan-2-yl)phenyl)-3-methyl-6,7-dihydropyrazolo[1,5-a]pyrazin-4(5H)-one (3.5 g, 9.18 mmol, 1 equiv) and 2-chloro-6-(trifluoromethyl)pyrido[3,2-d]pyrimidine (2.6 g, 11.02 mmol, 1.2 equiv) in dioxane (260 mL) were added K₃PO₄ (3.9 g, 18.36 mmol, 2 equiv), H₂O (50 mL) and AMPhosPdCl₂ (1.5 g, 2.12 mmol, 0.23 equiv). The resulting mixture was stirred overnight at 60° C. under nitrogen atmosphere. The resulting mixture was diluted with water and the aqueous layer was extracted with EtOAc. The combined organic layer was concentrated under reduced pressure. The residue was purified by silica gel column chromatography, by eluting with PE/EtOAc (3/1). The crude product was re-crystallized from MeOH to afford the title compound (501.0 mg, 12.06%) as a yellow solid. LC-MS: (ES, m/z): [M+H]⁺ 453; ¹H-NMR: (300 MHz, DMSO, ppm): δ2.26-2.28 (d, 6H), δ2.62 (s, 3H), δ3.91-3.95 (m, 1H), δ4.24-4.33 (m, 1H), δ4.50-4.54 (m, 2H), δ7.39 (s, 1H), δ7.48 (s, 1H), δ8.01 (s, 1H), δ8.48-8.51 (d, 1H), δ8.81-8.84 (d, 1H), δ9.98 (s, 1H).

### Example 20

### Synthesis of 5-(2,5-dimethyl-4-(8-methyl-6-(trifluoromethyl)-1,5-naphthyridin-2-yl)phenyl)-3-methyl-6,7-dihydropyrazolo[1,5-a]pyrazin-4(5H)-one

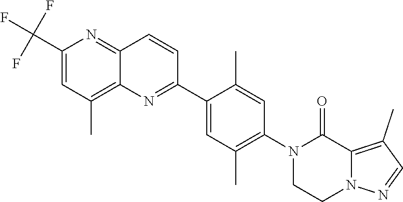 [SMILES: Cc1cc(N2CCn3ncc(C)c3C2=O)c(C)cc1-c1ccc2nc(C(F)(F)F)cc(C)c2n1]

Proceeding as described in Example 5, Step 12 above, but substituting 7-(2,5-dimethyl-4-(4,4,5,5-tetramethyl-1,3,2-dioxaborolan-2-yl)phenyl)-1-methyl-6,7-dihydro-1H-pyrazolo[3,4-f][1,4]oxazepin-8(5H)-one with 5-(2,5-dimethyl-4-(4,4,5,5-tetramethyl-1,3,2-dioxaborolan-2-yl)phenyl)-3-methyl-6,7-dihydropyrazolo[1,5-a]pyrazin-4(5H)-one gave crude product. Purification by silica gel column chromatography using ethyl acetate/petroleum ether (1:1) as eluent gave the title compound as a yellow solid in 53.48% yield. LC-MS: (ES, m/z): [M+H]⁺ 466; ¹H-NMR: (300 MHz, DMSO, ppm): δ2.26 (s, 6H), δ2.46 (s, 3H), δ2.89 (s, 3H), δ3.91-3.95 (m, 1H), δ4.23-4.31 (m, 1H), δ4.51-4.55 (m, 2H), δ7.38 (s, 1H), δ7.49 (s, 1H), δ7.60 (s, 1H), δ8.16-8.19 (m, 1H), δ8.63-8.66 (d, 1H).

### Example 21

### Synthesis of 5-(2-fluoro-5-methyl-4-(6-(trifluoromethyl)pyrido[3,2-d]pyrimidin-2-yl)phenyl)-3-methyl-6,7-dihydropyrazolo[1,5-a]pyrazin-4(5H)-one

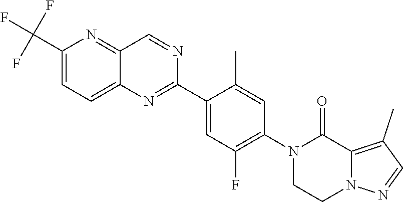 [SMILES: Cc1cc(N2CCn3ncc(C)c3C2=O)c(F)cc1-c1ncc2nc(C(F)(F)F)ccc2n1]

Proceeding as described in Example 19 above but substituting 7-(2,5-dimethyl-4-(4,4,5,5-tetramethyl-1,3,2-dioxaborolan-2-yl)phenyl)-1-methyl-6,7-dihydro-1H-pyrazolo[3,4-f][1,4]oxazepin-8(5H)-one with 5-(2-fluoro-5-methyl-4-(4,4,5,5-tetramethyl-1,3,2-dioxaborolan-2-yl)phenyl)-3-methyl-6,7-dihydropyrazolo[1,5-a]pyrazin-4(5H)-one (2 g, 5.19 mmol, 1 equiv) in dioxane (150 mL) gave the title compound as a yellow solid after purification by silica gel column chromatography, using PE/EtOAc (1:1) as an eluent. LC-MS: (ES, m/z): [M+H]⁺ 457; ¹H-NMR: (300 MHz, DMSO, ppm) δ 10.00 (s, 1H), 8.82-8.86 (d, 1H), 8.50-8.53 (d, 1H), 7.95-7.99 (d, 1H), 7.56-7.59 (d, 1H), 7.50 (d, 1H), 4.50-4.54 (t, 2H), 4.19-4.23 (t, 2H), 2.66 (s, 3H), 2.26 (s, 3H).

### Example 22

### Synthesis of 5-(2-fluoro-5-methyl-4-(8-methyl-6-(trifluoromethyl)-1,5-naphthyridin-2-yl)phenyl)-3-methyl-6,7-dihydropyrazolo[1,5-a]pyrazin-4(5H)-one

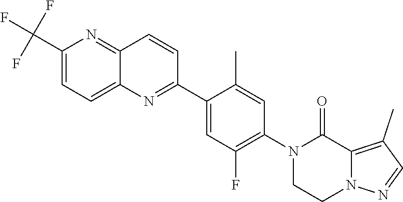 [SMILES: Cc1cc(N2CCn3ncc(C)c3C2=O)c(F)cc1-c1ccc2nc(C(F)(F)F)ccc2n1]

Proceeding as described in Example 5, Step 12 above, but substituting 7-(2,5-dimethyl-4-(4,4,5,5-tetramethyl-1,3,2-dioxaborolan-2-yl)phenyl)-1-methyl-6,7-dihydro-1H-pyrazolo[3,4-f][1,4]oxazepin-8(5H)-one with 5-(2-fluoro-5-methyl-4-(4,4,5,5-tetramethyl-1,3,2-dioxaborolan-2-yl)phenyl)-3-methyl-6,7-dihydropyrazolo[1,5-a]pyrazin-4(5H)-one and 2-chloro-6-trifluoromethyl)-1,5-naphthyridine 6-chloro-4-methyl-2-(trifluoromethyl)-1,5-naphthyridine gave crude product. Purification by silica gel column chromatography using ethyl acetate/petroleum ether (1:5) as eluent gave the title compound as a yellow solid in 59.39% yield. LC-MS: (ES, m/z): [M+H]⁺ 470; ¹H-NMR: (400 MHz, DMSO, ppm): δ2.27 (s, 3H), δ2.49 (s, 3H), δ2.91 (s, 3H), δ4.18-4.21 (t, 2H), δ4.50-4.53 (m, 2H), δ7.50 (s, 1H), δ7.55-7.57 (d, 1H), δ7.64-7.67 (d, 1H), δ8.12-8.23 (m, 2H), δ8.66-8.68 (d, 1H).

### Example 23

### Synthesis of 5-(2,5-dimethyl-4-(6-(trifluoromethyl)quinolin-2-yl)phenyl)-3-methyl-6,7-dihydropyrazolo[1,5-a]pyrazin-4(5H)-one

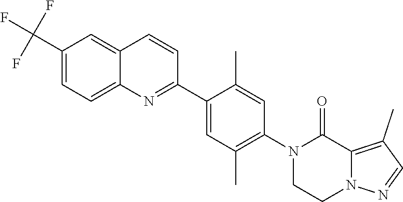 [SMILES: Cc1cc(N2CCn3ncc(C)c3C2=O)c(C)cc1-c1ccc2cc(C(F)(F)F)ccc2n1]

Proceeding as described in Example 5, Step 12 above, but substituting 7-(2,5-dimethyl-4-(4,4,5,5-tetramethyl-1,3,2-dioxaborolan-2-yl)phenyl)-1-methyl-6,7-dihydro-1H-pyrazolo[3,4-f][1,4]oxazepin-8(5H)-one with 5-(2,5-dimethyl-4-(4,4,5,5-tetramethyl-1,3,2-dioxaborolan-2-yl)phenyl)-3-methyl-6,7-dihydropyrazolo[1,5-a]pyrazin-4(5H)-one and 6-chloro-4-methyl-2-(trifluoromethyl)-1,5-naphthyridine with 2-chloro-6-(trifluoromethyl)quinoline gave crude product. Purification by silica gel column chromatography using ethyl acetate/petroleum ether (1:1) as eluent, followed by recrystallization from methanol gave the title compound as a yellow solid in 63.90% yield. LC-MS: (ES, m/z): [M+H]⁺ 451; ¹H-NMR: (300 MHz, DMSO, ppm): δ2.25-2.26 (d, 6H), δ2.38 (s, 3H), δ3.89-3.95 (m, 1H), δ4.21-4.30 (m, 1H), δ4.50-4.54 (t, 2H), δ7.35 (s, 1H), δ7.47 (s, 1H), δ7.51 (s, 1H), δ7.90-7.93 (d, 1H), δ8.03-8.06 (m, 1H), δ8.23-8.26 (d, 1H), δ8.59 (s, 1H), δ8.67-8.70 (d, 1H).

### Example 24

### Synthesis of 5-(2-fluoro-5-methyl-4-(6-(trifluoromethyl)quinolin-2-yl)phenyl)-3-methyl-6,7-dihydropyrazolo[1,5-a]pyrazin-4(5H)-one

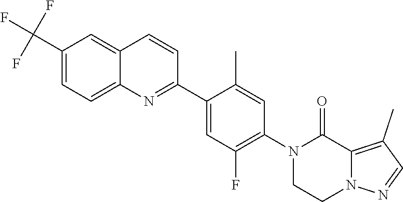 [SMILES: Cc1cc(N2CCn3ncc(C)c3C2=O)c(F)cc1-c1ccc2cc(C(F)(F)F)ccc2n1]

Proceeding as described in Example 5, Step 12 above, but substituting 7-(2,5-dimethyl-4-(4,4,5,5-tetramethyl-1,3,2-dioxaborolan-2-yl)phenyl)-1-methyl-6,7-dihydro-1H-pyrazolo[3,4-f][1,4]oxazepin-8(5H)-one with 5-(2-fluoro-5-methyl-4-(4,4,5,5-tetramethyl-1,3,2-dioxaborolan-2-yl)phenyl)-3-methyl-6,7-dihydropyrazolo[1,5-a]pyrazin-4(5H)-one and 6-chloro-4-methyl-2-(trifluoromethyl)-1,5-naphthyridine with 2-chloro-6-(trifluoromethyl)quinoline gave crude product. Purification by silica gel column chromatography using ethyl acetate/petroleum ether (1:1) as eluent, followed by recrystallization from PE/EtOAc gave the title compound as a white solid in 53.70% yield. LC-MS: (ES, m/z): [M+H]⁺ 455; ¹H-NMR: (400 MHz, DMSO-d6, ppm): δ2.27 (s, 3H), δ2.41 (s, 3H), δ4.16-4.19 (t, 2H), δ4.50-4.53 (t, 2H), δ7.49-7.57 (m, 3H), δ7.94-7.96 (d, 1H), δ8.05-8.07 (m, 1H), δ8.25-8.27 (d, 1H), δ8.60 (s, 1H), δ8.70-8.72 (d, 1H).

### Example 25

### Synthesis of 5-(2-fluoro-5-methyl-4-(6-(trifluoromethyl)quinazolin-2-yl)phenyl)-3-methyl-6,7-dihydropyrazolo[1,5-a]pyrazin-4(5H)-one

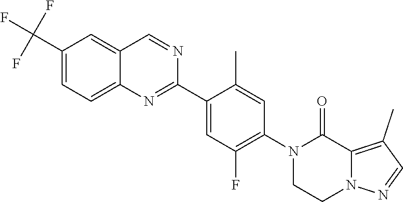 [SMILES: Cc1cc(N2CCn3ncc(C)c3C2=O)c(F)cc1-c1ncc2cc(C(F)(F)F)ccc2n1]

Proceeding as described in Example 2, Step 11 above, but substituting 3-methyl-5-(2-methyl-4-(4,4,5,5-tetramethyl-1,3,2-dioxaborolan-2-yl)phenyl)-6,7-dihydro-pyrazolo[1,5-a]pyrazin-4(5H)-one with 5-(2-fluoro-5-methyl-4-(4,4,5,5-tetramethyl-1,3,2-dioxaborolan-2-yl)phenyl)-3-methyl-6,7-dihydropyrazolo[1,5-a]pyrazin-4(5H)-one gave the title compound. Recrystallization from methanol gave pure product as a white solid in 56.68% yield. LC-MS: (ES, m/z): [M+H]⁺ 456; ¹H-NMR: (300 MHz, DMSO, ppm): δ2.27 (s, 3H), δ2.73 (s, 3H), δ4.18-4.22 (t, 2H), δ4.50-4.54 (t, 2H), δ7.50-7.63 (m, 2H), δ7.92-7.96 (d, 1H), δ8.27-8.35 (m, 2H), δ8.78 (s, 1H), δ9.94 (s, 1H).

### Example 26

### Synthesis of 5-(2-fluoro-5-methyl-4-(8-methyl-6-(trifluoromethyl)quinazolin-2-yl)phenyl)-3-methyl-6,7-dihydropyrazolo[1,5-a]pyrazin-4(5H)-one

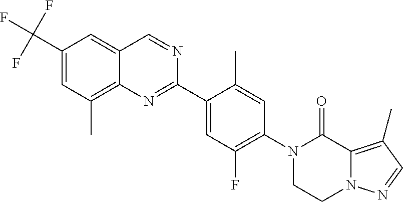 [SMILES: Cc1cc(N2CCn3ncc(C)c3C2=O)c(F)cc1-c1ncc2cc(C(F)(F)F)cc(C)c2n1]

Proceeding as described in Example 2, Step 11 above but substituting 3-methyl-5-(2-methyl-4-(4,4,5,5-tetramethyl-1,3,2-dioxaborolan-2-yl)phenyl)-6,7-dihydro-pyrazolo[1,5-a]pyrazin-4(5H)-one 3-methyl-5-(2-methyl-4-(4,4,5,5-tetramethyl-1,3,2-dioxaborolan-2-yl)phenyl)-6,7-dihydro-pyrazolo[1,5-a]pyrazin-4(5H)-one with 5-(2-fluoro-5-methyl-4-(4,4,5,5-tetramethyl-1,3,2-dioxaborolan-2-yl)phenyl)-3-methyl-6,7-dihydropyrazolo[1,5-a]pyrazin-4(5H)-one and 2-chloro-6-(trifluoromethyl)-quinazoline with 2-chloro-8-methyl-6-(trifluoromethyl)-quinazoline gave the title compound as a white solid in 71.50% yield. LC-MS: (ES, m/z): [M+H]⁺ 470; ¹H-NMR: (300 MHz, DMSO, ppm): δ2.27 (s, 3H), δ2.71 (s, 3H), δ2.82 (s, 3H), δ4.18-4.22 (m, 2H), δ4.50-4.54 (m, 2H), δ7.49 (s, 1H), δ7.54-7.57 (d, 1H), δ8.03-8.07 (d, 1H), 68.20 (s 1H), δ8.56 (s, 1H), δ9.88 (s, 1H).

### Example 27

### Synthesis of 1-methyl-6-(2-methyl-4-(6-(trifluoromethyl)-1,5-naphthyridin-2-yl)phenyl)-1,4,5,6-tetrahydro-7H-pyrazolo[3,4-c]pyridin-7-one

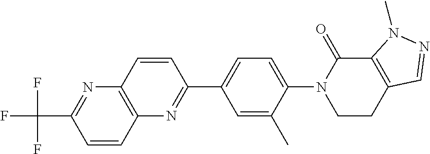 [SMILES: Cc1cc(-c2ccc3nc(C(F)(F)F)ccc3n2)ccc1N1CCc2cnn(C)c2C1=O]

Proceeding as described in Example 5, Step 12 above, but substituting 7-(2,5-dimethyl-4-(4,4,5,5-tetramethyl-1,3,2-dioxaborolan-2-yl)phenyl)-1-methyl-6,7-dihydro-1H-pyrazolo[3,4-f][1,4]oxazepin-8(5H)-one with 1-methyl-6-(2-methyl-4-(4,4,5,5-tetramethyl-1,3,2-dioxaborolan-2-yl)phenyl)-1,4,5,6-tetrahydro-7H-pyrazolo[3,4-c]pyridin-7-one and 6-chloro-4-methyl-2-(trifluoromethyl)-1,5-naphthyridine with 2-chloro-6-(trifluoromethyl)-1,5-naphthyridine gave crude product. Purification by silica gel column chromatography using ethyl acetate/petroleum ether (1:5) as eluent gave the title compound as a white solid in 49% yield. LC-MS: (ES, m/z): [M+H]⁺ 438; ¹H-NMR: (400 MHz, DMSO, ppm): δ2.34 (s, 3H), δ2.91-3.05 (m, 2H), δ3.72-3.77 (m, 1H), δ4.05-4.12 (m, 4H), δ7.49-7.54 (m, 2H), δ8.24-8.26 (d, 2H), δ8.33 (s, 1H), δ8.60-8.62 (d, 1H), δ8.68-8.80 (d, 1H), δ8.79-8.80 (d, 1H).

### Example 28

### Synthesis of 1-methyl-6-(2-methyl-4-(8-methyl-6-(trifluoromethyl)-1,5-naphthyridin-2-yl)phenyl)-1,4,5,6-tetrahydro-7H-pyrazolo[3,4-c]pyridin-7-one

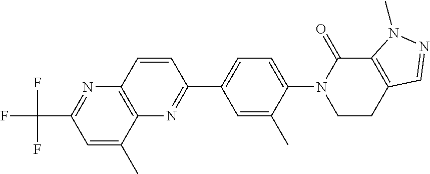 [SMILES: Cc1cc(-c2ccc3nc(C(F)(F)F)cc(C)c3n2)ccc1N1CCc2cnn(C)c2C1=O]

Proceeding as described in Example 5, Step 12 above, but substituting 7-(2,5-dimethyl-4-(4,4,5,5-tetramethyl-1,3,2-dioxaborolan-2-yl)phenyl)-1-methyl-6,7-dihydro-1H-pyrazolo[3,4-f][1,4]oxazepin-8(5H)-one with 1-methyl-6-(2-methyl-4-(4,4,5,5-tetramethyl-1,3,2-dioxaborolan-2-yl)phenyl)-1,4,5,6-tetrahydro-7H-pyrazolo[3,4-c]pyridin-7-one gave crude product. Purification by silica gel column chromatography using ethyl acetate/petroleum ether (1:2) as eluent gave the title compound as a white solid in 72.20% yield. LC-MS: (ES, m/z): [M+H]⁺ 452; ¹H-NMR: (400 MHz, DMSO-d6, ppm): δ 8.62-8.65 (d, 1H), 8.56-8.59 (d, 1H), 8.32 (s, 1H), 8.25-8.27 (m, 1H), 8.15 (s, 1H), 7.51-7.53 (d, 1H), 7.48 (s, 1H), 4.05-4.12 (m, 4H), 3.72-3.78 (m, 1H), 2.90-3.03 (m, 5H), 2.34 (s, 3H).

### Example 29

### Synthesis of 1-methyl-7-(2-methyl-4-(6-(trifluoromethyl)quinazolin-2-yl)phenyl)-6,7-dihydro-1H-pyrazolo[3,4-f][1,4]oxazepin-8(5H)-one

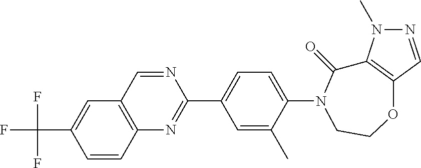 [SMILES: Cc1cc(-c2ncc3cc(C(F)(F)F)ccc3n2)ccc1N1CCOc2cnn(C)c2C1=O]

Proceeding as described in Example 2, Step 11 above but substituting 3-methyl-5-(2-methyl-4-(4,4,5,5-tetramethyl-1,3,2-dioxaborolan-2-yl)phenyl)-6,7-dihydro-pyrazolo[1,5-a]pyrazin-4(5H)-one with 1-methyl-7-(2-methyl-4-(4,4,5,5-tetramethyl-1,3,2-dioxaborolan-2-yl)phenyl)-6,7-dihydro-1H-pyrazolo[3,4-f][1,4]oxazepin-8(5H)-one provide crude product. Purification of the crude product by silica gel column chromatography, by eluting with PE/EtOAc (3/1), followed by re-crystallized from PE/EtOAc (10/1, 50 mL) provided the title compound as an off-white solid in 48.59% yield. LC-MS: (ES, m/z): [M+H]⁺ 454; ¹H-NMR: (300 MHz, DMSO, ppm): δ2.34 (s, 3H), δ3.97-4.05 (m, 5H), δ4.46-4.58 (m, 2H), δ7.37 (s, 1H), δ7.46-7.49 (d, 1H), δ8.25-8.32 (m, 2H), δ8.46-8.50 (m, 1H), δ8.54-8.55 (d, 1H), δ8.73 (s, 1H), δ9.90 (s, 1H).

### Example 30

### Synthesis of 1-methyl-7-(2-methyl-4-(6-(trifluoromethyl)-1,5-naphthyridin-2-yl)phenyl)-6,7-dihydro-1H-pyrazolo[3,4-f][1,4]oxazepin-8(5H)-one

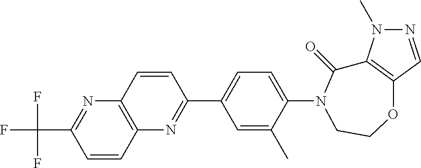 [SMILES: Cc1cc(-c2ccc3nc(C(F)(F)F)ccc3n2)ccc1N1CCOc2cnn(C)c2C1=O]

Proceeding as described in Example 5, Step 12 above, but substituting 7-(2,5-dimethyl-4-(4,4,5,5-tetramethyl-1,3,2-dioxaborolan-2-yl)phenyl)-1-methyl-6,7-dihydro-1H-pyrazolo[3,4-f][1,4]oxazepin-8(5H)-one with 1-methyl-7-(2-methyl-4-(4,4,5,5-tetramethyl-1,3,2-dioxaborolan-2-yl)phenyl)-6,7-dihydro-1H-pyrazolo[3,4-f][1,4]oxazepin-8(5H)-one and 6-chloro-4-methyl-2-(trifluoromethyl)-1,5-naphthyridine with 2-chloro-6-(trifluoromethyl)-1,5-naphthyridine gave crude product. Purification by silica gel column chromatography using ethyl acetate/petroleum ether (1:3) as eluent, followed by recrystallization from PE/EtOAc (10:1) gave the title compound as an off-white solid in 69.10% yield. LC-MS: (ES, m/z): [M+H]⁺ 454; ¹H-NMR: (300 MHz, DMSO, ppm): δ2.34 (s, 3H), δ3.99-4.04 (m, 5H), δ4.46-4.59 (m, 2H), δ7.37 (s, 1H), δ7.46-7.49 (d, 1H), δ8.22-8.26 (m, 2H), δ8.32-8.33 (d, 1H), δ8.59-8.62 (d, 1H), δ8.68-8.71 (d, 1H), δ8.78-8.81 (d, 1H).

### Example 31

### Synthesis of 1-methyl-7-(2-methyl-4-(8-methyl-6-(trifluoromethyl)-1,5-naphthyridin-2-yl)phenyl)-6,7-dihydro-1H-pyrazolo[3,4-f][1,4]oxazepin-8(5H)-one

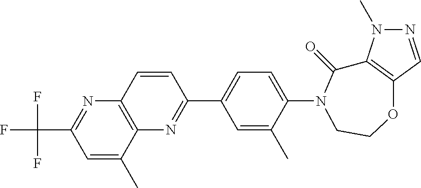 [SMILES: Cc1cc(-c2ccc3nc(C(F)(F)F)cc(C)c3n2)ccc1N1CCOc2cnn(C)c2C1=O]

Proceeding as described in Example 5, Step 12 above, but substituting 7-(2,5-dimethyl-4-(4,4,5,5-tetramethyl-1,3,2-dioxaborolan-2-yl)phenyl)-1-methyl-6,7-dihydro-1H-pyrazolo[3,4-f][1,4]oxazepin-8(5H)-one with 1-methyl-7-(2-methyl-4-(4,4,5,5-tetramethyl-1,3,2-dioxaborolan-2-yl)phenyl)-6,7-dihydro-1H-pyrazolo[3,4-f][1,4]oxazepin-8(5H)-one gave crude product. Purification by silica gel column chromatography using ethyl acetate/petroleum ether (1:3) as eluent, followed by recrystallization from PE/EtOAc (10:1) gave the title compound as an off-white solid in 82% yield. LC-MS: (ES, m/z): [M+H]⁺ 468; ¹H-NMR: (300 MHz, DMSO, ppm): δ2.34 (s, 3H), δ2.96 (s, 3H), δ3.99-4.05 (m, 5H), δ4.46-4.59 (m, 2H), δ7.37 (s, 1H), δ7.46-7.49 (d, 1H), δ8.15-8.16 (d, 2H), δ8.24-8.32 (m, 2H), δ8.55-8.65 (m, 2H).

### Example 32

### Synthesis of 7-(2,5-dimethyl-4-(6-(trifluoromethyl)-1,5-naphthyridin-2-yl)phenyl)-1-methyl-6,7-dihydro-1H-pyrazolo[3,4-f][1,4]oxazepin-8(5H)-one

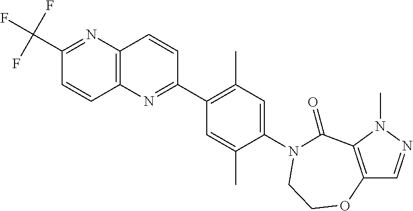 [SMILES: Cc1cc(N2CCOc3cnn(C)c3C2=O)c(C)cc1-c1ccc2nc(C(F)(F)F)ccc2n1]

Proceeding as described in Example 5, Step 12 above, but substituting 6-chloro-4-methyl-2-(trifluoromethyl)-1,5-naphthyridine with 6-chloro-2-(trifluoromethyl)-1,5-naphthyridine gave crude product. Purification by silica gel column chromatography using ethyl acetate/petroleum ether (1:10) as eluent gave the title compound as a white solid in 77.20% yield. LC-MS: (ES, m/z): [M+H]⁺ 468; ¹H-NMR: (300 MHz, DMSO, ppm): δ2.24 (s, 3H), δ2.40 (s, 3H), δ3.97-4.01 (m, 5H), δ4.46-4.58 (m, 2H), δ7.29 (s, 1H), δ7.36 (s, 1H), δ7.54 (s, 1H), δ8.14-8.17 (d, 1H), δ8.25-8.28 (d, 1H), δ8.67-8.70 (d, 1H), δ8.75-8.78 (d, 1H).

### Example 33

### Synthesis of 7-(2-methyl-4-(6-(trifluoromethyl)quinazolin-2-yl)phenyl)-6,7-dihydro-1H-pyrazolo[3,4-f][1,4]oxazepin-8(5H)-one

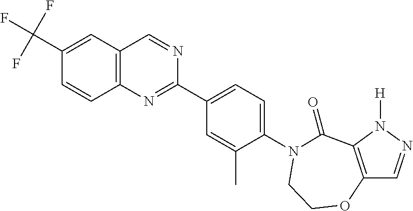 [SMILES: Cc1cc(-c2ncc3cc(C(F)(F)F)ccc3n2)ccc1N1CCOc2cn[nH]c2C1=O]

### Step 1: Synthesis of methyl 4-bromo-1-(tetrahydro-2H-pyran-2-yl)-1H-pyrazole-5-carboxylate

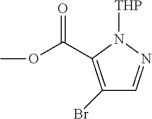

Into a 250-mL 3-necked round-bottom flask under nitrogen, was placed methyl 4-bromo-1H-pyrazole-5-carboxylate (7 g, 34.14 mmol, 1 equiv), 3,4-dihydro-2H-pyran (5.7 g, 68.29 mmol, 2 equiv), EA (70 mL), and PTSA (0.9 g, 5.12 mmol, 0.15 equiv). The resulting solution was stirred for 12 h at 80° C. and then quenched with water. The resulting solution was extracted with ethyl acetate. The residue was applied onto a silica gel column and eluted with ethyl acetate/petroleum ether (1:20) to give (7.1 g, 71.92%) of the title compound as a yellow liquid. LC-MS: (ES, m/z): [M+H]⁺ 289.

### Step 2: Synthesis of 4-(2-(benzyloxy)ethoxy)-1-(tetrahydro-2H-pyran-2-yl)-1H-pyrazole-5-carboxylic Acid

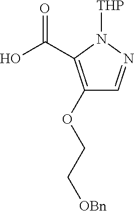

Into a 100-mL 3-necked round-bottom flask purged and maintained with an inert atmosphere of nitrogen, was placed methyl 4-bromo-1-(tetrahydro-2H-pyran-2-yl)-1H-pyrazole-5-carboxylate (7 g, 24.21 mmol, 1 equiv), 2-(benzyloxy)ethan-1-ol (25 mL), Cs₂CO₃ (23.7 g, 72.63 mmol, 3 equiv), CuCl₂ (0.3 g, 2.42 mmol, 0.1 equiv). The resulting solution was stirred for 12 h at 130° C. The resulting solution was extracted with ethyl acetate and the pH value of the solution was adjusted to 5 with HOAc. The resulting solution was extracted with ethyl acetate and the organic layer was concentrated. The residue was applied onto a silica gel column and eluted with ethyl acetate/petroleum ether (1:1) to give (3.1 g, 36.97%) of the title compound as a yellow liquid. LC-MS: (ES, m/z): [M+H]⁺ 347.

### Step 3: Synthesis of 4-(2-(benzyloxy)ethoxy)-N-(4-bromo-2-methylphenyl)-1-(tetrahydro-2H-pyran-2-yl)-1H-pyrazole-5-carboxamide

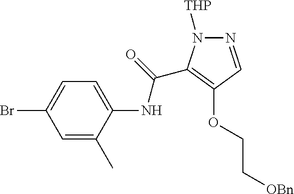

Into a 100-mL 3-necked round-bottom flask under nitrogen, was placed 4-(2-(benzyloxy)ethoxy)-1-(tetrahydro-2H-pyran-2-yl)-1H-pyrazole-5-carboxylic acid (3.07 g, 8.86 mmol, 1 equiv), 4-bromo-2-methylaniline (2.0 g, 10.64 mmol, 1.2 equiv), DIEA (2.3 g, 17.73 mmol, 2 equiv), DMF (30 mL), and HATU (5.1 g, 13.29 mmol, 1.5 equiv). The resulting solution was stirred for 12 h at 25° C. and then quenched with water. The resulting solution was extracted with ethyl acetate. The residue was applied onto a silica gel column and eluted with ethyl acetate/petroleum ether (1:5). The crude product was purified by re-crystallization from MeOH to give (2.88 g, 63.17%) of the title compound as a white solid. LC-MS: (ES, m/z): [M+H]⁺ 514.

### Step 4: Synthesis of 4-(2-(benzyloxy)ethoxy)-N-(2-methyl-4-(4,4,5,5-tetramethyl-1,3,2-dioxaborolan-2-yl)phenyl)-1-(tetrahydro-2H-pyran-2-yl)-1H-pyrazole-5-carboxamide

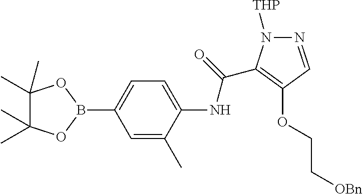

Into a 100-mL 3-necked round-bottom flask purged under nitrogen, was placed 4-(2-(benzyloxy)ethoxy)-N-(4-bromo-2-methylphenyl)-1-(tetrahydro-2H-pyran-2-yl)-1H-pyrazole-5-carboxamide (2.88 g, 5.60 mmol, 1 equiv), B₂Pin₂ (2.1 g, 8.27 mmol, 1.48 equiv), KOAc (1.1 g, 11.20 mmol, 2 equiv), dioxane (30 mL), and Pd(dppf)Cl₂ (0.6 g, 0.84 mmol, 0.15 equiv). The resulting solution was stirred for 12 h at 80° C. and the resulting mixture was concentrated. The residue was applied onto a silica gel column and eluted with ethyl acetate/petroleum ether (1:5) to give (2.5 g, 81.57%) of the title compound as a yellow solid. LC-MS: (ES, m/z): [M+H]⁺ 562.

### Step 5: Synthesis of 4-(2-hydroxyethoxy)-N-(2-methyl-4-(4,4,5,5-tetramethyl-1,3,2-dioxaborolan-2-yl)phenyl)-1-(tetrahydro-2H-pyran-2-yl)-1H-pyrazole-5-carboxamide

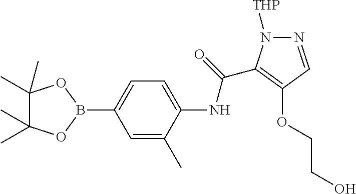

Into a 250-mL 3-necked round-bottom flask, was placed 4-(2-(benzyloxy)ethoxy)-N-(2-methyl-4-(4,4,5,5-tetramethyl-1,3,2-dioxaborolan-2-yl)phenyl)-1-(tetrahydro-2H-pyran-2-yl)-1H-pyrazole-5-carboxamide (2.46 g, 4.49 mmol, 1 equiv), MeOH (100 mL), and Pd/C (0.5 g,), H2 (g) passed through the reaction mixture. The resulting solution was stirred for 2 h at 70° C. under the atmosphere of H2. The solids were filtered out and the filtrate was concentrated to give (1.7 g, 80.26%) of the title compound as a white solid. LC-0575-5: (ES, m/z): [M+H]⁺ 472.

### Step 6: Synthesis of 2-((5-((2-methyl-4-(4,4,5,5-tetramethyl-1,3,2-dioxaborolan-2-yl)phenyl)carbamoyl)-1-(tetrahydro-2H-pyran-2-yl)-1H-pyrazol-4-yl)oxy)ethyl methanesulfonate

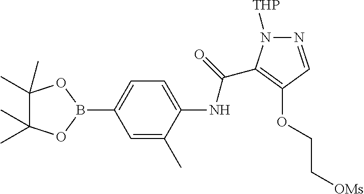

Into a 100-mL 3-necked round-bottom flask purged and maintained with an inert atmosphere of nitrogen, was placed 4-(2-hydroxyethoxy)-N-(2-methyl-4-(4,4,5,5-tetramethyl-1,3,2-dioxaborolan-2-yl)phenyl)-1-(tetrahydro-2H-pyran-2-yl)-1H-pyrazole-5-carboxamide (1.68 g, 3.56 mmol, 1 equiv), Et₃N (0.7 g, 7.13 mmol, 2 equiv), and DCM (40 mL), and MsCl (0.8 g, 7.13 mmol, 2 equiv) was added dropwise. The resulting solution was stirred for 1 h at 0° C. and then quenched with water. The resulting solution was extracted with dichloromethane and the organic layer was concentrated. The crude product was purified by re-crystallization from MeOH to give (1.85 g, 94.47%) of the title compound as a yellow solid. LC-MS: (ES, m/z): [M+H]⁺ 550.

### Step 7: Synthesis of 7-(2-methyl-4-(4,4,5,5-tetramethyl-1,3,2-dioxaborolan-2-yl)phenyl)-1-(tetrahydro-2H-pyran-2-yl)-6,7-dihydro-1H-pyrazolo[3,4-f][1,4]oxazepin-8(5H)-one

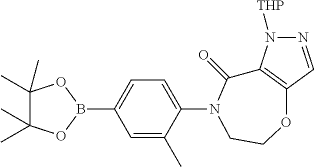

Into a 100-mL 3-necked round-bottom flask purged and maintained with an inert atmosphere of nitrogen, was placed 2-((5-((2-methyl-4-(4,4,5,5-tetramethyl-1,3,2-dioxaborolan-2-yl)phenyl)carbamoyl)-1-(tetrahydro-2H-pyran-2-yl)-1H-pyrazol-4-yl)oxy)ethyl methanesulfonate (1.84 g, 3.35 mmol, 1 equiv) and DMF (40 mL), and NaH (60%) (0.27 g, 11.25 mmol, 3.36 equiv) was added at 0° C. The resulting solution was stirred for 1 h at 0° C. and then quenched with NH₄Cl (aq.). The resulting solution was extracted with ethyl acetate and the organic layer was concentrated. The residue was applied onto a silica gel column and eluted with dichloromethane/methanol (50:1) to give (1.45 g, 95.51%) of the title compound as a yellow solid. LC-MS: (ES, m/z): [M+H]⁺ 454.

### Step 8: Synthesis of 7-(2-methyl-4-(6-(trifluoromethyl)quinazolin-2-yl)phenyl)-1-(tetrahydro-2H-pyran-2-yl)-6,7-dihydro-1H-pyrazolo[3,4-f][1,4]oxazepin-8(5H)-one

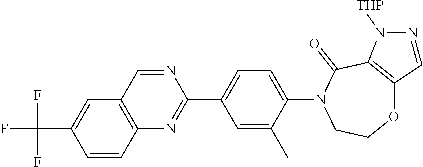

Into a 100-mL 3-necked round-bottom flask purged and maintained with an inert atmosphere of nitrogen, was placed 7-(2-methyl-4-(4,4,5,5-tetramethyl-1,3,2-dioxaborolan-2-yl)phenyl)-1-(tetrahydro-2H-pyran-2-yl)-6,7-dihydro-1H-pyrazolo[3,4-f][1,4]oxazepin-8(5H)-one (1.42 g, 3.13 mmol, 1 equiv), 2-chloro-6-(trifluoromethyl)quinazoline (0.9 g, 3.76 mmol, 1.2 equiv), K₂CO₃ (1.3 g, 9.40 mmol, 3 equiv), toluene (20 mL), EtOH (10 mL), and Pd(PPh₃)₄ (0.5 g, 0.47 mmol, 0.15 equiv). The resulting solution was stirred for 12 h at 80° C. and then concentrated under vacuum. The residue was applied onto a silica gel column and eluted with ethyl acetate/petroleum ether (1:1). The crude product was purified by re-crystallization from MeOH to give (1.4 g, 85.38%) of the title compound as a white solid. LC-MS: (ES, m/z): [M+H]⁺ 524.

### Step 9: Synthesis of 7-(2-methyl-4-(6-(trifluoromethyl)quinazolin-2-yl)phenyl)-6,7-dihydro-1H-pyrazolo[3,4-f][1,4]oxazepin-8(5H)-one

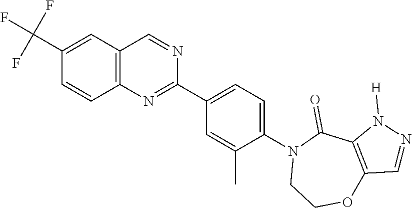 [SMILES: Cc1cc(-c2ncc3cc(C(F)(F)F)ccc3n2)ccc1N1CCOc2cn[nH]c2C1=O]

Into a 25-mL 3-necked round-bottom flask purged under nitrogen, was placed 7-(2-methyl-4-(6-(trifluoromethyl)quinazolin-2-yl)phenyl)-1-(tetrahydro-2H-pyran-2-yl)-6,7-dihydro-1H-pyrazolo[3,4-f][1,4]oxazepin-8(5H)-one (1.4 g, 2.67 mmol, 1 equiv) in HCl/dioxane (10 mL). The resulting solution was stirred for 1 h at 25° C. and then quenched by the addition of 20 mL of NaHCO₃ (aq.). The resulting solution was extracted with dichloromethane. The residue was applied onto a silica gel column and eluted with ethyl acetate/petroleum ether (1:1) to give (0.8 g, 68.08%) of the title compound as a white solid. LC-MS: (ES, m/z): [M+H]⁺ 440. H-NMR: (300 MHz, DMSO, ppm): δ2.27 (s, 3H), δ3.93-4.09 (m, 2H), δ4.41-4.61 (m, 2H), 7.37-7.64 (m, 2H), δ8.28 (s, 2H), δ8.46-8.54 (m, 2H), δ8.73 (s, 1H), δ9.90 (s, 1H), δ13.13-13.20 (brss, 1H).

### Example 34

### Synthesis 1-(2-hydroxyethyl)-7-(2-methyl-4-(6-(trifluoromethyl)quinazolin-2-yl)phenyl)-6,7-dihydro-1H-pyrazolo[3,4-f][1,4]oxazepin-8(5H)-one

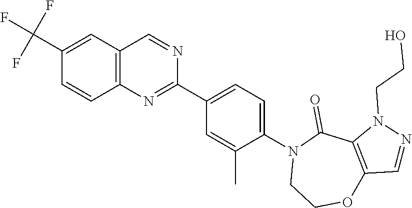 [SMILES: Cc1cc(-c2ncc3cc(C(F)(F)F)ccc3n2)ccc1N1CCOc2cnn(CCO)c2C1=O]

### Step 1: Synthesis of 1-(2-(benzyloxy)ethyl)-7-(2-methyl-4-(6-(trifluoromethyl)quinazolin-2-yl)phenyl)-6,7-dihydro-1H-pyrazolo[3,4-f][1,4]oxazepin-8(5H)-one

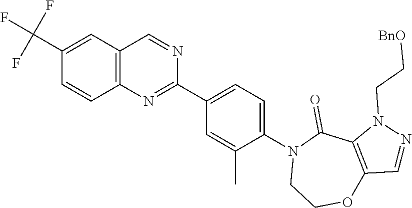 [SMILES: Cc1cc(-c2ncc3cc(C(F)(F)F)ccc3n2)ccc1N1CCOc2cnn(CCOCc3ccccc3)c2C1=O]

Into a 8-mL sealed tube purged and maintained with an inert atmosphere of nitrogen, was placed 7-(2-methyl-4-(6-(trifluoromethyl)quinazolin-2-yl)phenyl)-6,7-dihydro-1H-pyrazolo[3,4-f][1,4]oxazepin-8(5H)-one (200 mg, 0.46 mmol, 1 equiv), [(2-bromoethoxy)methyl]benzene (107.7 mg, 0.50 mmol, 1.1 equiv), and K₂CO₃ (125.8 mg, 0.91 mmol, 2 equiv) in DMF (2 mL). The resulting solution was stirred for 12 h at 80° C. and then quenched with water. The resulting solution was extracted with ethyl acetate and the organic layer was concentrated. The residue was applied onto Prep-TLC and eluted with ethyl acetate/petroleum ether (1:1) to give (100 mg, 38.30%) of the title compound as a yellow solid. LC-MS-PH: (ES, m/z): [M+H]⁺ 574.

### Step 2: Synthesis 1-(2-hydroxyethyl)-7-(2-methyl-4-(6-(trifluoromethyl)quinazolin-2-yl)phenyl)-6,7-dihydro-1H-pyrazolo[3,4-f][1,4]oxazepin-8(5H)-one

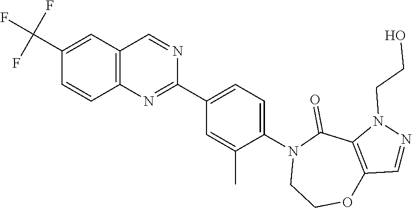 [SMILES: Cc1cc(-c2ncc3cc(C(F)(F)F)ccc3n2)ccc1N1CCOc2cnn(CCO)c2C1=O]

Into a 8-mL sealed tube purged and maintained with an inert atmosphere of nitrogen, was placed 1-(2-(benzyloxy)ethyl)-7-(2-methyl-4-(6-(trifluoromethyl)quinazolin-2-yl)phenyl)-6,7-dihydro-1H-pyrazolo[3,4-f][1,4]oxazepin-8(5H)-one (100 mg, 0.17 mmol, 1 equiv), DCM (2 mL), and BCl₃ (0.26 mL, 0.26 mmol, 1.5 equiv). The resulting solution was stirred for 1 hr at 0° C. The reaction was then quenched with 20 mL of NaHCO₃ and the resulting solution was extracted with ethyl acetate. The residue was applied onto Prep-TLC and eluted with dichloromethane/methanol (20:1) to give (48.2 mg, 57.19%) of the title compound as a white solid. LC-MS: (ES, m/z): [M+H]⁺ 484. H-NMR-PH-IDE-0578-0: (300 MHz, DMSO, ppm): δ2.33 (s, 3H), δ3.62-3.68 (m, 2H), δ3.99-4.06 (m, 2H), δ4.37-4.44 (m, 1H), δ4.46-4.63 (m, 3H), δ4.75-4.78 (m, 1H), δ7.39 (s, 1H), δ7.46-7.48 (d, 1H), δ8.25 (s, 1H), δ8.28-8.32 (d, 1H), δ8.50 (s, 1H), δ8.73 (s, 1H), δ9.91 (s, 1H).

### Example 35

### Synthesis of 2-(7-(2-methyl-4-(6-(trifluoromethyl)quinazolin-2-yl)phenyl)-1-oxo-5,6,7,8-tetrahydro-2H-pyrazolo[3,4-f][1,4]oxazepin-2-yl)acetonitrile

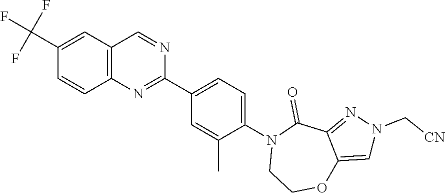 [SMILES: Cc1cc(-c2ncc3cc(C(F)(F)F)ccc3n2)ccc1N1CCOc2cn(CC#N)nc2C1=O]

To a stirred mixture of 7-(2-methyl-4-(6-(trifluoromethyl)quinazolin-2-yl)phenyl)-6,7-dihydro-1H-pyrazolo[3,4-f][1,4]oxazepin-8(5H)-one (240 mg, 0.55 mmol, 1 equiv) and 2-bromoacetonitrile (131.0 mg, 1.09 mmol, 2 equiv) in DMF (4.8 mL) were added K₂CO₃ (226.5 mg, 1.64 mmol, 3 equiv). The resulting mixture was stirred for overnight at 80° C. in an oil bath. The reaction was quenched with water and extracted with EtOAc. The combined organic layers were concentrated under reduced pressure. The residue was purified by Prep-TLC (CH₂Cl₂/MeOH 30/1) to afford the title compound (50 mg, 19.13%) as a grey solid. LC-MS: (ES, m/z): [M+H]⁺ 479. H-NMR: (400 MHz, DMSO, ppm): δ2.32 (s, 3H), δ3.95-4.06 (m, 2H), δ4.44-4.58 (m, 2H), δ5.54 (s, 2H), δ7.45-7.47 (d, 1H), δ7.82 (s, 1H), δ8.28 (s, 2H), δ8.47-8.49 (d, 1H), δ8.54 (s, 1H), δ8.73 (s, 1H), δ9.91 (s, 1H).

### Example 36

### Synthesis of 2-(7-(2-methyl-4-(6-(trifluoromethyl)quinazolin-2-yl)phenyl)-1-oxo-5,6,7,8-tetrahydro-1H-pyrazolo[3,4-f][1,4]oxazepin-1-yl)acetonitrile

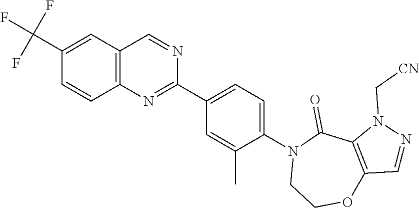 [SMILES: Cc1cc(-c2ncc3cc(C(F)(F)F)ccc3n2)ccc1N1CCOc2cnn(CC#N)c2C1=O]

To a stirred mixture of 7-(2-methyl-4-(6-(trifluoromethyl)quinazolin-2-yl)phenyl)-6,7-dihydro-1H-pyrazolo[3,4-f][1,4]oxazepin-8(5H)-one (240 mg, 0.55 mmol, 1 equiv) and 2-bromoacetonitrile (131.0 mg, 1.09 mmol, 2 equiv) in DMF (4.8 mL) were added K₂CO₃ (226.5 mg, 1.64 mmol, 3 equiv). The resulting mixture was stirred overnight at 80° C. in an oil bath. The reaction was quenched with water and the resulting mixture was extracted with EtOAc. The combined organic layers were concentrated under reduced pressure. The residue was purified by Prep-TLC (CH₂Cl₂/MeOH 30/1) to afford the title compound (80 mg, 30.61%) as a yellow solid. LC-MS: (ES, m/z): [M+H]⁺ 479. H-NMR: (300 MHz, DMSO, ppm): δ2.35 (s, 3H), δ4.04-4.10 (m, 2H), δ4.53-4.65 (m, 2H), δ5.55-5.68 (m, 2H), δ7.49-7.51 (d, 1H), δ7.63 (s, 1H), δ8.28 (s, 2H), δ8.47-8.51 (m, 1H), δ8.55 (s, 1H), δ8.73 (s, 1H), δ9.91 (s, 1H).

### Example 37

### Synthesis of 2-(2-hydroxyethyl)-7-(2-methyl-4-(6-(trifluoromethyl)quinazolin-2-yl)phenyl)-6,7-dihydro-2H-pyrazolo[3,4-f][1,4]oxazepin-8(5H)-one

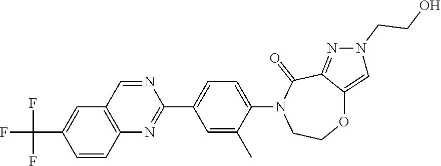 [SMILES: Cc1cc(-c2ncc3cc(C(F)(F)F)ccc3n2)ccc1N1CCOc2cn(CCO)nc2C1=O]

### Step 1: Synthesis of 2-(2-(benzyloxy)ethyl)-7-(2-methyl-4-(6-(trifluoromethyl)quinazolin-2-yl)phenyl)-6,7-dihydro-2H-pyrazolo[3,4-f][1,4]oxazepin-8(5H)-one

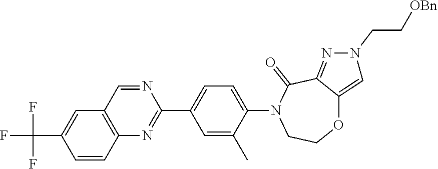 [SMILES: Cc1cc(-c2ncc3cc(C(F)(F)F)ccc3n2)ccc1N1CCOc2cn(CCOCc3ccccc3)nc2C1=O]

Into a 8-mL sealed tube purged and maintained with an inert atmosphere of nitrogen, was placed 7-(2-methyl-4-(6-(trifluoromethyl)quinazolin-2-yl)phenyl)-6,7-dihydro-1H-pyrazolo[3,4-f][1,4]oxazepin-8(5H)-one (200 mg, 0.46 mmol, 1 equiv), [(2-bromoethoxy)methyl]benzene (107.7 mg, 0.50 mmol, 1.1 equiv), and K₂CO₃ (125.8 mg, 0.91 mmol, 2 equiv) in DMF (2 mL). The resulting solution was stirred for 12 h at 80° C. The reaction was then quenched with water. The resulting solution was extracted with ethyl acetate and the organic layer concentrated. The residue was applied onto Prep-TLC and eluted with ethyl acetate/petroleum ether (1:1) to give (100 mg, 38.30%) of the title compound as a yellow solid. LC-MS: (ES, m/z): [M+H]⁺ 574.

### Step 2: Synthesis of 2-(2-hydroxyethyl)-7-(2-methyl-4-(6-(trifluoromethyl)quinazolin-2-yl)phenyl)-6,7-dihydro-2H-pyrazolo[3,4-f][1,4]oxazepin-8(5H)-one

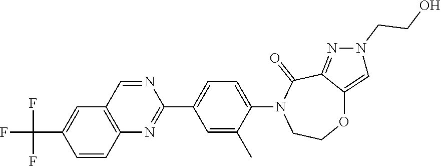 [SMILES: Cc1cc(-c2ncc3cc(C(F)(F)F)ccc3n2)ccc1N1CCOc2cn(CCO)nc2C1=O]

Into a 8-mL sealed tube purged and maintained with an inert atmosphere of nitrogen, was placed 2-(2-(benzyloxy)ethyl)-7-(2-methyl-4-(6-(trifluoromethyl)quinazolin-2-yl)phenyl)-6,7-dihydro-2H-pyrazolo[3,4-f][1,4]oxazepin-8(5H)-one (100 mg, 0.17 mmol, 1 equiv) in DCM (2 mL) and BCl₃ (30.3 mg, 0.26 mmol, 1.5 equiv) was added. The resulting solution was stirred for 1 h at 0° C. The reaction mixture was then quenched with 20 mL of NaHCO₃ and the resulting solution was extracted with ethyl acetate. The residue was applied onto Prep-TLC and eluted with dichloromethane/methanol (20:1) to give (19.7 mg, 23.37%) of the title compound as a yellow solid. LC-MS: (ES, m/z): [M+H]⁺ 484. H-NMR: (300 MHz, DMSO, ppm): δ2.35 (s, 3H), δ3.66-3.78 (m, 2H), δ3.76-3.97 (m, 2H), δ4.15-4.25 (m, 2H), δ4.43-4.68 (m, 2H), δ4.88-5.10 (m, 1H), δ7.42-7.45 (m, 1H), δ7.65 (s, 1H), δ8.27 (s, 2H), δ8.48-8.52 (m, 2H), δ8.82 (s, 1H), δ9.98 (s, 1H).

Additional compounds of Formula (I) are disclosed on pages 137 to 226 of International Application No. PCT/US19/16705, filed on Feb. 5, 2019, and the disclosure of pages 137 to 226 is incorporated herein by reference in its entirety.

### BIOLOGICAL EXAMPLES

### Example 1

The ability of compounds of formula (I) to antagonize AhR was tested as follow. HEPG2 and HEPA1C1C7 cells were maintained in MEM and αMEM without nucleosides supplemented with 10% heat inactivated FBS respectively. Stably integrated DRE-luciferase cell lines were generated by transducing the both cell lines with Cignal XRE luciferase reporter (Qiagen) lentiviral particles according to the manufacturer protocol. For both cell lines, stably integrated reporter cell lines were selected for the presence of 2 μg/mL puromycin. Following selection of stably integrated cell line pools, clonal cell lines were isolated by limiting dilution in 96-well plates. Transcriptional assays were performed by seeding 100 μL of cells at a density of 250,000 or 100,000 cells/mL, for HEPG2 and HEPA1C1C7 DRE-Luc cells respectively, into 96-well cell culture plates in OptiMEM supplemented with 0.5% heat inactivated FBS and allowed to attach overnight.

To determine AhR antagonist activity of compound of formula (I), the compounds were added in a semi-log dose response using a D300e Digital Dispenser (Tecan) followed normalization with vehicle (DMSO). Immediately following compound addition 10 μL of 2,3,7,8-tetrachlorodibenzo-p-dioxin (TCDD) was added to the cells to a final concentration of 3 nM or 0.3 nM for the HEPG2 and HEPA1C1C7 DRE-Luc cells respectively. Following 24 h incubation, the medium was removed, and the cells were lysed in 25 μL of Reporter Lysis Buffer (Promega). Firefly luciferase activity was measured immediately following the addition of 50 μL Luciferase Assay Reagent (Promega). The percent maximal activity for each point was determined using the following equation: (RLUₛₐₘₚₗₑ−RLU_{vehicle-TCDD})/(RLU_{vehicle+TCDD} RLU_{vehicle-TCDD})*100. The relative IC₅₀, defined as the compound concentration required to reduce the TCDD induced response between the top and bottom plateau of each individual dose response curve by half, for each compound was determined using Prism7 (GraphPad Software).

TABLE 1

| EXAMPLE NUMBER | IC50 hAhR (antagonist mode) |
|---|---|
| 001 | ++++ |
| 002 | ++++ |
| 003 | +++ |
| 004 | ++++ |
| 005 | ++++ |
| 006 | ++++ |
| 007 | ++++ |
| 008 | ++++ |
| 009 | ++++ |
| 010 | + |
| 011 | ++++ |
| 012 | ++ |
| 013 | ++++ |
| 014 | ++++ |
| 015 | ++++ |
| 016 | ++++ |
| 017 | ++++ |
| 018 | +++ |
| 019 | ++++ |
| 020 | ++++ |
| 021 | ++++ |
| 022 | ++++ |
| 023 | + |
| 024 | ++++ |
| 025 | ++++ |
| 026 | ++++ |
| 027 | ++++ |
| 028 | ++++ |
| 029 | ++++ |
| 030 | ++++ |
| 031 | ++++ |
| 032 | + |
| 033 | ++++ |
| 034 | +++ |
| 035 | ++++ |
| 036 | ++++ |
| 037 | ++++ |

(+) IC₅₀ = 10 μM-1 μM;
(++) IC₅₀ = 1 μM-500 nM;
(+++) IC₅₀ = 500 nM-200 nM;
(++++) IC₅₀ < 200 nM

### Example 2

The ability of compounds of formula (I) to facilitate hematopoietic stem cell expansion was determined by testing 1-methyl-7-(2-methyl-4-(6-(trifluoromethyl)pyrido[3,2-d]pyrimidin-2-yl)phenyl)-6,7-dihydro-1H-pyrazolo[3,4-f][1,4]oxazepin-8(5H)-one (compound 1) and 3-methyl-5-(2-methyl-4-(6-(trifluoromethyl)quinazolin-2-yl)phenyl)-6,7-dihydropyrazolo[1,5-a]pyrazin-4(5H)-one (compound 2) as follows.

Isolated human CD34⁺ hematopoietic stem cells (HSC) were obtained from AllCells (Alameda, Calif., USA). 5,000 HSC per well were cultivated in 100 μL HSC expansion media StemSpan SFEM (StemCell Technologies; Vancouver, BC, Canada) containing 1× antibiotics (Life Technologies, Carlsbad, Calif., USA), 100 ng/mL thrombopoietin, 100 ng/mL IL-6, 100 ng/mL Flt3 ligand and 100 ng/mL stem cell factor (all R&D Systems, Minneapolis, Minn., USA) in a flat bottom 96 well tissue culture plate. 100 μL of 3-fold serial diluted compound 1 or 2 (final concentration range 0.169-10,000 nM) was added at 2× of the final concentration to HSC culture and HSC culture plates were incubated in a humidity-controlled incubator at 370° C., 5% CO₂. After 12 days, the absolute viable cell counts from each well were determined. HSC were then stained in staining buffer composed of Hank's balanced salt solution with 2% fetal bovine serum and 2 mM EDTA (all Life Technologies) at 4° C. for 1 hour with anti-CD90 APC, anti-CD34 PerCP, anti-CD38 FITC, CD45RA PE-Cy7 and anti-CD133 PE antibodies (all BD Biosciences, San Jose, Calif., USA). HSC were washed with staining buffer and HSC were analyzed by multicolor phenotype flow cytometry. The percentages of CD34⁺ cells were determined as percentage of total population. Absolute counts of each population were calculated from the total number of viable cells multiplied by the percentage of each population. The EC₅₀ value for the frequency of CD34+ cells, defined as the compound concentration with half maximal response on the dose response curve, was calculated using Prism 7 (GraphPad Software, La Jolla, Calif., USA).

### Results:

After 12-day culture, compounds 1 and 2 increased the CD34⁺ cell counts in a concentration-dependent manner, i.e. 2.4-fold to 4.0-fold expansion at compound concentrations of 3,333 nM and 10,000 nM, respectively. In addition, the frequency of CD34⁺ HSC increased in a concentration-dependent manner. i.e. 3.1-fold to 3.6-fold at compound concentrations of 3,333 nM and 1,111 nM, respectively.

What is claimed is:

1. A method for producing an expanded population of stem cells and/or lineage committed progenitor cells in vitro or ex vivo comprising culturing a population of the stem cells and/or lineage committed progenitor cells in a medium comprising a compound of formula (I): 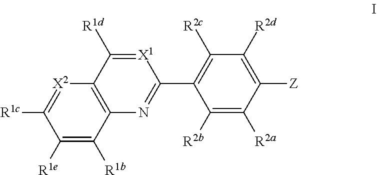
or a pharmaceutically acceptable salt, solvate or hydrate thereof, wherein:
each of ring vertices X¹ and X² is independently selected from the group consisting of C(R^{1a}) and N;
Z is selected from the group consisting of: 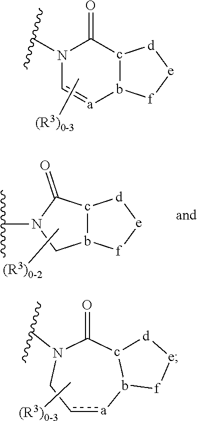
wherein the dashed bonds are single or double bonds, each of ring vertices a, b, c, d, e and f are independently selected from the group consisting of O, S, N, CH, C(R⁴) and N(R⁴), and the bonds joining the ring vertices are independently single or double bonds;
each R^{1a}, R^{1b}, R^{1c}, R^{1d} and R^{1e} is independently selected from the group consisting of hydrogen, deuterium, halogen, —CN, —NO₂, —R^{c}, —CO₂R^{a}, —CONR^{a}R^{b}, —C(O)R^{a}, —OC(O)NR^{a}R^{b}, —NR^{b}C(O)R^{a}, —NR^{b}C(O)₂R^{c}, —NR^{a}C(O)NR^{a}R^{b}, —NR^{a}R^{b}, —OR^{a}, and —S(O)₂NR^{a}R^{b}; wherein each R^{a} and R^{b} is independently selected from hydrogen, C₁₋₈ alkyl, C₃₋₆ cycloalkyl and C₁₋₈ haloalkyl, or when attached to the same nitrogen atom can be combined with the nitrogen atom to form a four-, five- or six-membered ring having from 0 to 2 additional heteroatoms as ring members selected from N, O, S, SO and SO₂; each R^{c} is independently selected from the group consisting of C₁₋₈ alkyl, C₁₋₈ haloalkyl, C₁₋₈ deuteroalkyl, C₂₋₆ alkenyl, C₂₋₆ alkynyl, and C₃₋₆ cycloalkyl, and wherein the aliphatic and cyclic portions of R^{a}, R^{b} and R^{c} can be further substituted with from one to three halogen, hydroxy, C₁₋₄ alkyl, C₁₋₄ alkoxy, amino, C₁₋₄ alkylamino, di C₁₋₄ alkylamino and carboxylic acid groups;
each R^{2a}, R^{2b}, R^{2c} and R^{2d} is independently selected from the group consisting of hydrogen, halogen, C₁₋₃ alkyl, C₁₋₃ deuteroalkyl, C₁₋₃ haloalkyl, C₁₋₃ alkoxy, C₁₋₃ deuteroalkoxy and C₁₋₃ haloalkoxy;
R³ is selected from the group consisting of hydrogen, deuterium, C₁₋₃ alkyl, C₁₋₃ deuteroalkyl, C₁₋₃ alkylene-OR^{d}, C₁₋₃ alkylene-CO₂R^{d}, C₁₋₃ alkylene-NR^{d}R^{e}, C₁₋₃ alkylene-CONR^{d}R^{e}, C₁₋₃ alkylene-OC(O)NR^{d}R^{e}, and C₁₋₃ alkylene-NR^{e}C(O)₂R^{f}; or two R³ groups are combined to form oxo (═O);
each R⁴ is independently selected from the group consisting of hydrogen, halogen, —CN, —R^{f}, —CO₂R^{d}, —CONR^{d}R^{e}, —C(O)R^{d}, —OC(O)NR^{d}R^{e}, —NR^{e}C(O)R^{d}, —NR^{e}C(O)₂R^{f}, —NR^{d}C(O)NR^{d}R^{e}, —NR^{d}R^{e}, —OR^{d}, —S(O)₂NR^{d}R^{e}, —X^{a}—CN, —X^{a}—CO₂R^{d}, —X^{a}—CONR^{d}R^{e}, —X^{a}—C(O)R^{d}, —X^{a}—OC(O)NR^{d}R^{e}, —X^{a}—NR^{e}C(O)R^{d}, —X^{a}—NR^{e}C(O)₂R^{f}, —X^{a}—NR^{d}C(O)NR^{d}R^{e}, —X^{a}—NR^{d}R^{e}, —X^{a}—OR^{d}, and —X^{a}—S(O)₂NR^{d}R^{e}; wherein each X^{a} is independently C₁₋₆alkylene; and
each R^{d} and R^{e} is independently selected from hydrogen, C₁₋₈ alkyl, and C₁₋₈ haloalkyl, or when attached to the same nitrogen atom can be combined with the nitrogen atom to form a four-, five- or six-membered ring having from 0 to 2 additional heteroatoms as ring members selected from N, O, S, SO and SO₂;
each R^{f} is independently selected from the group consisting of C₁₋₈ alkyl, C₁₋₈ haloalkyl, C₁₋₈ deuteroalkyl, C₃₋₆ cycloalkyl, C₃₋₆ heterocycloalkyl, phenyl and 5- or 6-membered heteroaryl;
wherein the aliphatic and cyclic portions of R^{d}, R^{e} and R^{f} are can be further substituted with from one to three halogen, hydroxy, C₁₋₄ alkyl, C₁₋₄ alkoxy, amino, C₁₋₄ alkylamino, di C₁₋₄ alkylamino and carboxylic acid groups; and
wherein the compound of formula (I) antagonizes the activity of aryl hydrocarbon receptor; and
the stem cells and/or progenitor cells are cultured under conditions allowing expansion of the stem cells and/or lineage committed progenitor cells.

2. The method of claim 1, further comprising differentiating the expanded stem cells to lineage committed progenitor cells thereof under conditions that cause differentiation of the expanded stem cells to lineage committed progenitor cells thereof.

3. The method of claims 1 or 2, wherein the stem cells and/or lineage committed progenitor cells are human cells.

4. The method of any one of claims 1 to 3, wherein the stem cells and/or lineage committed progenitor cells are hematopoietic stem cells and/or lineage committed hematopoietic progenitor cells.

5. The method of any one of claims 1 to 3, wherein the stem cells and/or lineage committed progenitor cells are genetically modified hematopoietic stem cells and/or lineage committed hematopoietic progenitor cells thereof.

6. The method of claim 5, wherein the genetically modified hematopoietic stem cells and/or lineage committed hematopoietic progenitor cells thereof comprise an exogenous nucleic acid.

7. The method of any one of claims 4 to 6, further comprising culturing the population of:
(i) hematopoietic stem cells and/or lineage committed hematopoietic progenitor cells; or
(ii) genetically modified hematopoietic stem cells and/or lineage committed genetically modified hematopoietic progenitor cells;
in the presence of an agent that inhibits TGFβ signaling;

8. The method of any one of claims 4 to 7, further comprising culturing the population of:
(i) hematopoietic stem cells and/or lineage committed hematopoietic progenitor cells; or
(ii) genetically modified hematopoietic stem cells and/or lineage committed genetically modified hematopoietic progenitor cells;
in the presence of a histone demethylase inhibitor.

9. The method of any one of claims 4 to 8, further comprising culturing the population of:
(i) hematopoietic stem cells and/or lineage committed hematopoietic progenitor cells; or
(ii) genetically modified hematopoietic stem cells and/or lineage committed genetically modified hematopoietic progenitor cells;
in the presence of a histone deacetylation inhibitor.

10. The method of any one of claims 4 to 9, further comprising culturing the population of:
(i) hematopoietic stem cells and/or lineage committed hematopoietic progenitor cells; or
(ii) genetically modified hematopoietic stem cells and/or lineage committed genetically modified hematopoietic progenitor cells;
in the presence of an agent that inhibits p38 signaling.

11. The method of any one of claims 4 to 10, further comprising culturing the population of hematopoietic stem cells and/or lineage committed hematopoietic progenitor cells or genetically modified hematopoietic stem cells and/or lineage committed genetically modified hematopoietic progenitor cells in the presence of a Notch agonist.

12. The method of claim 11, wherein the Notch Agonist is Delta-^{ext-IgG}.

13. The method of any one of claims 4 to 12, wherein the hematopoietic stem cells are from bone marrow, umbilical cord blood, or mobilized peripheral blood.

14. The method of claim 13, wherein the hematopoietic stem cells and genetically modified hematopoietic stem cells are enriched in Endothelial Protein C Receptor (EPCR+) and/or CD34+, CD38+, CD90+, CD45RA+, CD133 and/or CD49f+.

15. The method of claim 14, wherein the hematopoietic stem cells and genetically modified hematopoietic stem cells consist essentially of CD34+ cells.

16. The method of any one of claims 4 to 15, further comprising culturing the population of:
(i) hematopoietic stem cells and/or lineage committed hematopoietic progenitor cells; or
(ii) genetically modified hematopoietic stem cells and/or lineage committed genetically modified hematopoietic progenitor cells;
in the presence of a sufficient amount of one or more of IL6, Flt-3-L, TPO, and SCF.

17. The method of any one of claims 1 to 16, wherein the amount of compound of formula (I) in the cell culture is from about 100 pM to about 10 μM.

18. The method of any one of claims 1 to 17, wherein the stem cells and/or lineage committed progenitor cells are cultured in the presence of a compound of formula (I), from about 2 to about 35 days.

19. The method of any one of claims 4 to 18, wherein the starting cell population is cultured in the presence of a compound of formula (I) during a time sufficient for about 2 to 50,000 fold expansion of hematopoietic cells and/or lineage committed hematopoietic progenitor cells or genetically modified hematopoietic stem cells and/or lineage committed genetically modified hematopoietic progenitor cells as compared to a population of hematopoietic stem cells and/or lineage committed hematopoietic progenitor cells or genetically modified hematopoietic stem cells and/or lineage committed genetically modified hematopoietic progenitor cells thereof cultured under the same conditions in the absence of a compound of formula (I).

20. The method of any one of claims 7 to 19, wherein the hematopoietic stem cells and/or lineage committed hematopoietic progenitor cells or genetically modified hematopoietic stem cells and/or lineage committed genetically modified hematopoietic progenitor cells thereof are contacted with said one or more agents simultaneously or at different times.

21. The method of any one of claims 4 to 12 and 16 to 20, wherein said hematopoietic stem cells and/or lineage committed hematopoietic progenitor cells are originally within a mononuclear cell fraction prior to treatment with said one or more agents.

22. The method of any one of claims 4 to 12 and 16 to 20, wherein said hematopoietic stem cells are originally within a CD34+, CD34+/CD38−, CD34+/CD38−/CD90+, CD34+/CD38−/CD90+/CD45RA−, or CD34+/CD38−/CD90+/CD45RA−/CD49F+ enriched cell fraction prior to contacting said one or more agents.

23. The method of any one of claims 1 to 22, wherein the compound of formula (I) is a compound of formula (IIa), (IIb), (IIc), (IId), (IIe) or (IIf):

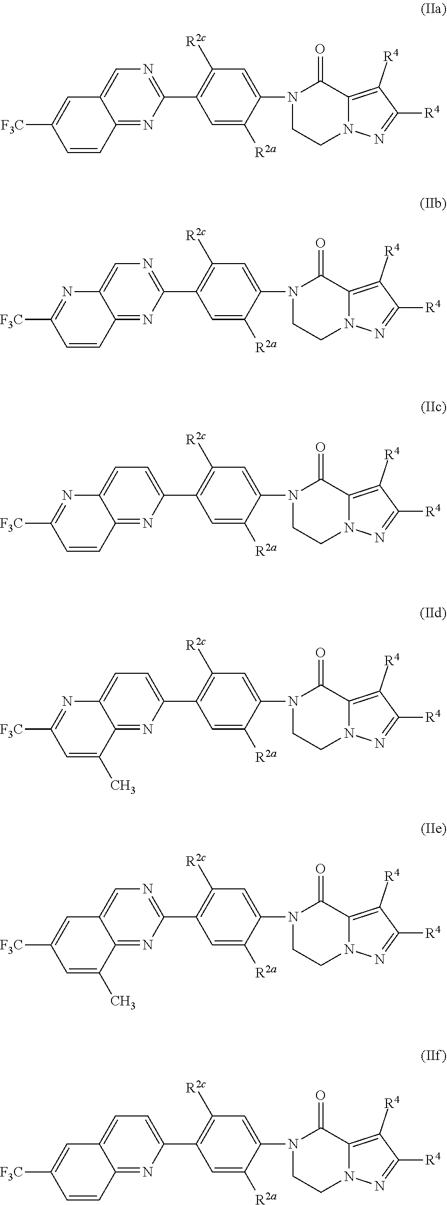

24. The method of any one of claims 1 to 22, wherein the compound of formula (I) is a compound of formula (IIIa), (IIIb), (IIIc), or (IIId):

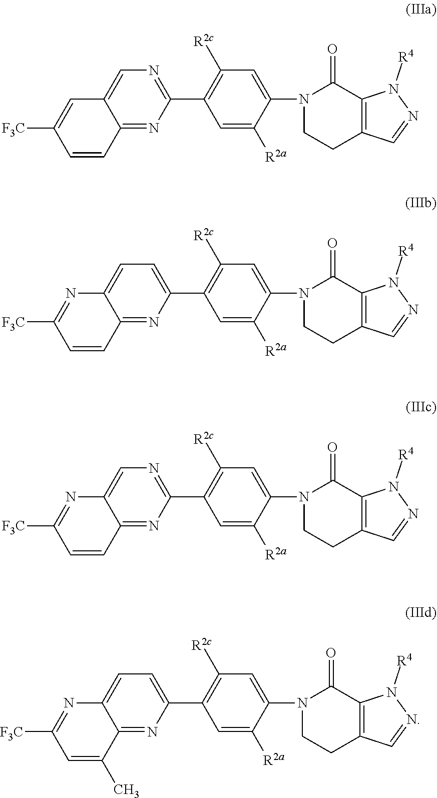

25. The method of any one of claims 1 to 22, wherein the compound of formula (I) is a compound of formula (IVa), (IVb), (IVc) or (IVd):

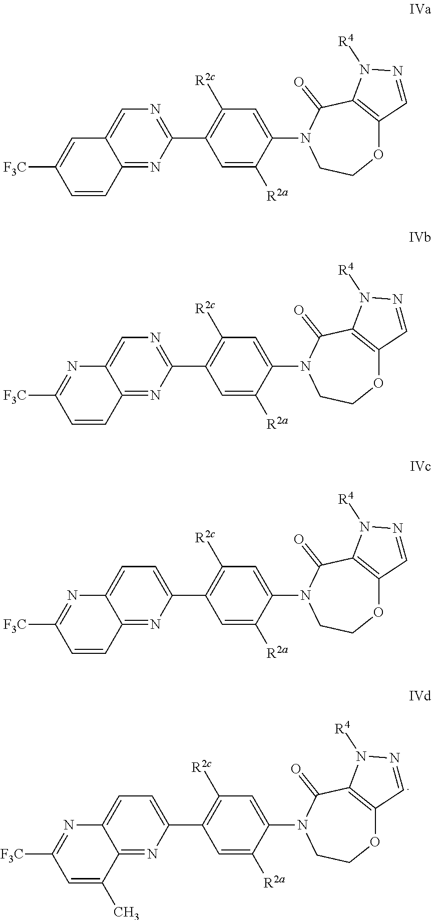

26. The method of any one of claims 1 to 22, wherein the compound of formula (I) is selected from Table 1.

27. An ex vivo or in vitro composition comprising a cell population of expanded hematopoietic stem cells and/or lineage committed progenitor cells thereof and a compound of formula (I), (IIa), (IIb), (IIc), (IId), (IIe), (IIf) (IIIa), (IIIb), (IIIc), (IIId), (IVa), (IVb), (IVc), (IVd) or a compound disclosed in Table 1.

28. A composition comprising a cell population of expanded hematopoietic stem cells and/or lineage committed hematopoietic progenitor cells obtained or obtainable by culturing ex vivo a starting population of cells comprising hematopoietic stem cells and/or lineage committed hematopoietic progenitor cells and a compound of formula (I) according to the method of any one of claims 3 to 26.

29. The composition of claim 27 or 28, substantially free of a compound of formula (I), (IIa), (IIb), (IIc), (IId), (IIe), (IIf) (IIIa), (IIIb), (IIIc), (IIId), (IVa), (IVb), (IVc), (IVd) or a compound disclosed in Table 1 and/or any other component of the cell culture medium.

30. The composition of any one of claims 27 to 29, further comprising a pharmaceutically acceptable medium.

31. A method of treating a disease treatable by hematopoietic stem cell and/or lineage committed hematopoietic progenitor cell therapy comprising administering to a patient in need thereof a composition of any one of claims 27 to 30.

32. The method of claim 31, wherein the disease is selected from the group consisting of Acute Lymphoblastic Leukemia (ALL), Acute Myelogenous Leukemia (AML), Chronic Myelogenous Leukemia (CML), Chronic Lymphocytic Leukemia (CLL), Hodgkin Lymphoma (HL), Non-Hodgkin Lymphoma (NHL), Myelodysplastic Syndrome (MDS), Multiple myeloma, Aplastic anemia, Bone marrow failure, Myeloproliferative disorders such as Myelofibrosis, Essential thrombocytopenia or Polycythemia vera, Fanconi anemia, Dyskeratosis congenita, Common variable immune deficiency (CVID, such as CVID 1, CVID 2, CVID 3, CVID 4, CVID 5, and CVID 6), Human immunodeficiency virus (HIV), Hemophagocytic lymphohistiocystosis, Amyloidosis, Solid tumors such as Neuroblastoma, Germ cell tumors, Breast cancer, Wilms' tumor, Medulloblastoma, and Neuroectodermal tumors, Autoimmune diseases such as Scleroderma, Multiple sclerosis, Ulcerative colitis, Systemic lupus erythematosus and Type I diabetes, or protein deficiencies such as Adrenoleukodystrophy (ALD), Metachromatic leukodystrophy (MLD), Hemophilia A & B, Hurler syndrome, Hunter syndrome, Fabry disease, Gaucher disease, Epidermolysis bullosa, Globoid Cell Leukodystrophy, Sanfillipo syndrome, and Morquio syndrome.

33. The method of claim 31, wherein the disease is selected from Sickle cell anemia, Alpha thalassemia, Beta thalassemia, Delta thalassemia, Hemoglobin E/thalassemia, Hemoglobin S/thalassemia, Hemoglobin C/thalassemia, Hemoglobin D/thalassemia, Chronic granulomatous disease (X-linked Chronic granulomatous disease, autosomal recessive (AR) chronic granulomatous disease, chronic granulomatous disease ARI NCF1, Chronic granulomatous disease AR CYBA, Chronic granulomatous disease AR II NCF2, Chronic granulomatous disease AR III NCF4, X-linked Severe Combined Immune Deficiency (SCID), ADA SCID, IL7-RA SCID, CD3 SCID, Rag1/Rag2 SCID, Artemis SCID, CD45 SCID, Jak3 SCID, Congenital agranulocytosis, Congenital agranulocytosis-congenital neutropenia-SCN1, Congenital agranulocytosis-congenital neutropenia-SCN2, Familial hemophagocytic lymphohistiocystosis (FHL), Familial hemophagocytic lymphohistiocytosis type 2 (FHL2, perforin mutation), Agammaglobulinemia (X-linked Agammaglobulinemia), Wiskott-Aldrich syndrome, Chediak-Higashi syndrome, Hemolytic anemia due to red cell pyruvate kinase deficiency, Paroxysmal nocturnal hemoglobinuria, X-linked Adrenoleukodystrophy (X-ALD), X-linked lymphoproliferative disease, Unicentric Castleman's Disease, Multicentric Castleman's Disease, Congenital amegakaryocytic, thrombocytopenia (CAMT) type I, Reticular dysgenesis, Fanconi anemia, Acquired idiopathic sideroblastic anemia, Systemic mastocytosis, Von willebrand disease (VWD), Congenital dyserythropoietic anemia type 2, Cartilage-hair hypoplasia syndrome, Hereditary spherocytosis, Blackfan-Diamond syndrome, Shwachman-Diamond syndrome, Thrombocytopenia-absent radius syndrome, Osteopetrosis, Infantile osteopetrosis, Mucopolysaccharidoses, Lesch-Nyhan syndrome, Glycogen storage disease, Congenital mastocytosis, Omenn syndrome, X-linked Immunodysregulation, polyendocrinopathy, and enteropathy (IPEX), IPEX characterized by mutations in FOXP3, X-linked syndrome of polyendocrinopathy, immune dysfunction, and diarrhea (XPID), X-Linked Autoimmunity-Allergic Dysregulation Syndrome (XLAAD), IPEX-like syndrome, Hyper IgM type 1, Hyper IgM type 2, Hyper IgM type 3, Hyper IgM type 4, Hyper IgM type 5, X linked hyperimmunoglobulin M, Bare lymphocyte Syndrome type I, and Bare lymphocyte Syndrome type II (Bare lymphocyte Syndrome type IL, MHC class I deficiency; Bare lymphocyte Syndrome type II, complementation group A; Bare lymphocyte Syndrome type II, complementation group C; Bare lymphocyte Syndrome type II complementation group D; Bare lymphocyte Syndrome type IL, complementation group E).

34. A catheter comprising a cell population of expanded hematopoietic stem cells and/or lineage committed hematopoietic progenitor cells obtained or obtainable by culturing ex vivo a starting population of cells comprising hematopoietic stem cells and/or lineage committed hematopoietic progenitor cells and a compound of formula (I) according to the method of any one of claims 3 to 26.

35. A syringe comprising a cell population of expanded hematopoietic stem cells and/or lineage committed hematopoietic progenitor cells obtained or obtainable by culturing ex vivo a starting population of cells comprising hematopoietic stem cells and/or lineage committed hematopoietic progenitor cells and a compound of formula (I) according to the method of any one of claims 3 to 26.
